(19) United States
(12) Patent Application Publication
(10) Pub. No.: US 2022/0348917 A1
(43) Pub. Date: Nov. 3, 2022

(54) LIPOSOMAL SPHERICAL NUCLEIC ACID (SNA) CONSTRUCTS FOR SPLICE MODULATION

(71) Applicant: Exicure Operating Company, Chicago, IL (US)

(72) Inventor: Samantha M. Sarett, Cambridge, MA (US)

(73) Assignee: Exicure Operating Company, Chicago, IL (US)

(21) Appl. No.: 17/639,938

(22) PCT Filed: Sep. 3, 2020
(86) PCT No.: PCT/US2020/049254

§ 371 Date: Mar. 3, 2022

Related U.S. Application Data
(60) Provisional application No. 62/895,937, filed on Sep. 4, 2019.

(51) Int. Cl. C12N 15/113; A61K 31/712; A61K 47/54; A61K 47/69; A61K 9/127; A61P 25/00; A61P 35/00

(52) U.S. Cl. CPC C12N 15/113; A61K 31/712; A61K 47/543; A61K 47/6911; A61K 9/127; A61P 25/00; A61P 35/00; C12N 2310/11; C12N 2310/3233; C12N 2320/33

(57) ABSTRACT

Compositions related to spherical nucleic acids (SNAs) with antisense oligonucleotides and methods of treatment of diseases and disorders are disclosed therein. In particular, the antisense oligonucleotides are targeted to a region in a pre-mRNA of interest to regulate pre-mRNA splicing.

### RELATED APPLICATIONS

This application claims priority under 35 U.S.C. § 119(e) to U.S. Provisional Application Ser. No. 62/895,937, filed on Sep. 4, 2019, which is herein incorporated by reference in its entirety.

### BACKGROUND

The process of pre-mRNA splicing is a precise and complex process that involves removal of introns, which were first reported as interrupting the coding information of metazoan genes (Berget et al. PNAS (1977) 74:3171-5; Chow et al. Cell (1977) 12:1-8). Most human genes are able to express more than one mRNA variant by alternative splicing, a process that produces functionally diverse protein isoforms expressed according to different regulatory programs. Since the vast majority of human genes include introns and that most pre-mRNAs undergo alternative splicing, the disruption of normal splicing patterns can be the cause or modify human disease.

### SUMMARY

According to some aspects, spherical nucleic acids (SNAs) for regulating pre-mRNA splicing are contemplated herein. In some embodiments, the SNA comprises a core and an antisense oligonucleotide comprised of 8 to 50 linked nucleosides in length targeted to a region in a pre-mRNA of interest to regulate pre-mRNA splicing, and wherein the antisense oligonucleotide is attached to the core and forms an oligonucleotide shell.

In some embodiments, the pre-mRNA of interest is obtained from the genomic sequence of interleukin 17 receptor A (IL17RA), RE1 Silencing Transcription Factor (REST), IL1 receptor accessory protein (IL1RAP), or signal transducer and activator of transcription 3 (STAT3).

In some embodiments, the core is a solid core or a hollow core. In some embodiments, the core is a liposomal core.

In some embodiments, the core has a diameter of or about 5 nm to about 150 nm. In some embodiments, the core has a diameter of or about 5 nm, of or about 6 nm, of or about 7 nm, of or about 8 nm, of or about 9 nm, of or about 10 nm, of or about 11 nm, of or about 12 nm, of or about 13 nm, of or about 14 nm, of or about 15 nm, of or about 16 nm, of or about 17 nm, of or about 18 nm, of or about 19 nm, of or about 20 nm, of or about 21 nm, of or about 22 nm, of or about 23 nm, of or about 24 nm, of or about 25 nm, of or about 26 nm, of or about 27 nm, of or about 28 nm, of or about 29 nm, of or about 30 nm, of or about 31 nm, of or about 32 nm, of or about 33 nm, of or about 34 nm, of or about 35 nm, of or about 36 nm, of or about 37 nm, of or about 38 nm, of or about 39 nm, of or about 40 nm, of or about 41 nm, of or about 42 nm, of or about 43 nm, of or about 44 nm, of or about 45 nm, of or about 46 nm, of or about 47 nm, of or about 48 nm, of or about 49 nm, of or about 50 nm, of or about 55 nm, of or about 60 nm, of or about 65 nm, of or about 70 nm, of or about 75 nm, of or about 80 nm, of or about 85 nm, of or about 90 nm, of or about 95 nm, of or about 100 nm, of or about 110 nm, of or about 120 nm, of or about 130 nm, of or about 140 nm, of or about 150 nm, of or about 160 nm, of or about 170 nm, of or about 180 nm, of or about 190 nm, of or about 200 nm, of or about 210 nm, of or about 220 nm, of or about 230 nm, of or about 240 nm, of or about 250 nm, of or about 260 nm, of or about 270 nm, of or about 280 nm, of or about 290 nm, of or about 300 nm, of more than about 300 nm, of about 15 nm to about 100 nm, of about 20 nm to about 100 nm, of about 25 nm to about 100 nm, of about 15 nm to about 50 nm, of about 20 nm to about 50 nm, of about 10 nm to about 70 nm, of about 15 nm to about 70 nm, of about 20 nm to about 70 nm, of about 10 nm to about 30 nm, of about 15 nm to about 30 nm, of about 20 nm to about 30 nm, of about 10 nm to about 40 nm, of about 15 nm to about 40 nm, of about 20 nm to about 40 nm, of about 10 nm to about 80 nm, of about 15 nm to about 80 nm, or of about 20 nm to about 80 nm.

In some embodiments, the SNA has a diameter of or about 5 nm, of or about 6 nm, of or about 7 nm, of or about 8 nm, of or about 9 nm, of or about 10 nm, of or about 11 nm, of or about 12 nm, of or about 13 nm, of or about 14 nm, of or about 15 nm, of or about 16 nm, of or about 17 nm, of or about 18 nm, of or about 19 nm, of or about 20 nm, of or about 21 nm, of or about 22 nm, of or about 23 nm, of or about 24 nm, of or about 25 nm, of or about 26 nm, of or about 27 nm, of or about 28 nm, of or about 29 nm, of or about 30 nm, of or about 31 nm, of or about 32 nm, of or about 33 nm, of or about 34 nm, of or about 35 nm, of or about 36 nm, of or about 37 nm, of or about 38 nm, of or about 39 nm, of or about 40 nm, of or about 41 nm, of or about 42 nm, of or about 43 nm, of or about 44 nm, of or about 45 nm, of or about 46 nm, of or about 47 nm, of or about 48 nm, of or about 49 nm, of or about 50 nm, of or about 55 nm, of or about 60 nm, of or about 65 nm, of or about 70 nm, of or about 75 nm, of or about 80 nm, of or about 85 nm, of or about 90 nm, of or about 95 nm, of or about 100 nm, of or about 110 nm, of or about 120 nm, of or about 130 nm, of or about 140 nm, of or about 150 nm, of or about 160 nm, of or about 170 nm, of or about 180 nm, of or about 190 nm, of or about 200 nm, of or about 210 nm, of or about 220 nm, of or about 230 nm, of or about 240 nm, of or about 250 nm, of or about 260 nm, of or about 270 nm, of or about 280 nm, of or about 290 nm, of or about 300 nm, of more than about 300 nm, of about 15 nm to about 100 nm, of about 20 nm to about 100 nm, of about 25 nm to about 100 nm, of about 15 nm to about 50 nm, of about 20 nm to about 50 nm, of about 10 nm to about 70 nm, of about 15 nm to about 70 nm, of about 20 nm to about 70 nm, of about 10 nm to about 30 nm, of about 15 nm to about 30 nm, of about 20 nm to about 30 nm, of about 10 nm to about 40 nm, of about 15 nm to about 40 nm, of about 20 nm to about 40 nm, of about 10 nm to about 80 nm, of about 15 nm to about 80 nm, or of about 20 nm to about 80 nm.

In some embodiments, the region is a regulatory site or a site at which a splicing factor interacts.

In some embodiments, the liposomal core comprises a lipid bilayer and the antisense oligonucleotide is attached to the lipid bilayer.

In some embodiments, the antisense oligonucleotide is eight to 100 linked nucleosides in length, eight linked nucleosides in length, nine linked nucleosides in length, 10 linked nucleosides in length, 11 linked nucleosides in length, 12 linked nucleosides in length, 13 linked nucleosides in length, 14 linked nucleosides in length, 15 linked nucleosides in length, 16 linked nucleosides in length, 17 linked nucleosides in length, 18 linked nucleosides in length, 19 linked nucleosides in length, 20 linked nucleosides in length, 21 linked nucleosides in length, 22 linked nucleosides in length, 23 linked nucleosides in length, 24 linked nucleosides in length, 25 linked nucleosides in length, 26 linked nucleosides in length, 27 linked nucleosides in length, 28 linked nucleosides in length, 29 linked nucleosides in length, 30 linked nucleosides in length, 31 linked nucleosides in length, 32 linked nucleosides in length, 33 linked nucleosides in length, 34 linked nucleosides in length, 35 linked nucleosides in length, 36 linked nucleosides in length, 37 linked nucleosides in length, 38 linked nucleosides in length, 39 linked nucleosides in length, 40 linked nucleosides in length, 41 linked nucleosides in length, 42 linked nucleosides in length, 43 linked nucleosides in length, 44 linked nucleosides in length, 45 linked nucleosides in length, 46 linked nucleosides in length, 47 linked nucleosides in length, 49 linked nucleosides in length, 50 linked nucleosides in length, 52 linked nucleosides in length, 54 linked nucleosides in length, 56 linked nucleosides in length, 58 linked nucleosides in length, 60 linked nucleosides in length, 62 linked nucleosides in length, 64 linked nucleosides in length, 66 linked nucleosides in length, 68 linked nucleosides in length, 70 linked nucleosides in length, 72 linked nucleosides in length, 74 linked nucleosides in length, 76 linked nucleosides in length, 78 linked nucleosides in length, 80 linked nucleosides in length, 82 linked nucleosides in length, 84 linked nucleosides in length, 86 linked nucleosides in length, 88 linked nucleosides in length, 90 linked nucleosides in length, 92 linked nucleosides in length, 94 linked nucleosides in length, 96 linked nucleosides in length, 100 linked nucleosides in length, or any range or combination thereof.

In some embodiments, less than all of the internucleoside linkages in the antisense oligonucleotide are phosphodiester linkages. In some embodiments, all of the internucleoside linkages in the antisense oligonucleotide are phosphodiester linkages. In some embodiments, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 99% of the internucleoside linkages in the antisense oligonucleotide are phosphodiester linkages.

In some embodiments, the antisense oligonucleotide has phosphorothioate internucleoside linkages. In some embodiments, less than all of the internucleoside linkages in the antisense oligonucleotide are phosphorothioate linkages.

In some embodiments, all of the internucleoside linkages in the antisense oligonucleotide are phosphorothioate linkages. In some embodiments, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 99% of the internucleoside linkages in the antisense oligonucleotide are phosphorothioate linkages.

In some embodiments, the antisense oligonucleotide has 2′O (2-methoxyethyl) modifications. In some embodiments, less than all of the nucleotides in the antisense oligonucleotide include a 2′O (2-methoxyethyl) modification. In some embodiments, all of the nucleotides in the antisense oligonucleotide include a 2′O methyl modification. In some embodiments, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 99% of the nucleotides in the antisense oligonucleotide include a 2′O methyl modification.

In some embodiments, the antisense oligonucleotide has LNA modifications. In some embodiments, less than all of the nucleotides include a LNA modification. In some embodiments, the antisense oligonucleotide has morpholino modifications. In some embodiments, less than all of the nucleotides include a morpholino modification.

In some embodiments, the antisense oligonucleotide has 2′O methyl modifications. In some embodiments, less than all of the nucleotides include a 2′O methyl modification.

In some embodiments, the antisense oligonucleotide is comprised of 18 to 21 linked nucleosides in length.

In some embodiments, the antisense oligonucleotides of the oligonucleotide shell are directly attached to the lipid bilayer of the liposomal core. In some embodiments, the antisense oligonucleotides of the oligonucleotide shell are indirectly attached to the lipid bilayer of the liposomal core through a linker moiety.

In some embodiments, the linker moiety comprises a molecular species at the 3′ or 5′ terminus of the antisense oligonucleotide, wherein the molecular species is positioned in the liposomal core and the antisense oligonucleotide extends radially from the liposomal core.

In some embodiments, the molecular species is at the 5′ terminus of the antisense oligonucleotide. In some embodiments, the molecular species is attached to the linker moiety.

In some embodiments, the molecular species is a hydrophobic group.

In some embodiments, the hydrophobic group is selected from the group consisting of cholesterol, a cholesteryl or modified cholesteryl residue, tocopherol, adamantine, dihydrotesterone, long chain alkyl, long chain alkenyl, long chain alkynyl, olely-lithocholic, cholenic, oleoyl-cholenic, decane, dodecane, docosahexaenoyl, palmityl, C6-palmityl, heptadecyl, myrisityl, arachidyl, stearyl, behenyl, linoleyl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen. In some embodiments, the hydrophobic group is cholesterol.

In some embodiments, the linker moiety comprises a non-nucleotidic linker moiety attached to the molecular species. In some embodiments, the non-nucleotidic linker moiety is selected from the group consisting of an abasic residue (dSpacer), oligoethyleneglycol, triethyleneglycol, hexaethylenegylcol, alkane-diol, or butanediol. In some embodiments, the non-nucleotidic linker moiety is a double linker. In some embodiments, the double linker is two oligoethyleneglycols. In some embodiments, the two oligoethyleneglycols are triethyleneglycol.

In some embodiments, the two oligoethyleneglycols are hexaethylenegylcol.

In some embodiments, the double linker is two alkane-diols. In some embodiments, the two alkane-diols are butanediol.

In some embodiments, the double linker is linked in the center by a phosphodiester, phosphorothioate, methylphosphonate, or amide linkage.

In some embodiments, the non-nucleotidic linker moiety is a triple linker. In some embodiments, the triple linker is three oligoethyleneglycols. In some embodiments, the three oligoethyleneglycols are triethyleneglycol. In some embodiments, the three oligoethyleneglycols are hexaethylenegylcol. In some embodiments, the triple linker is three alkane-diols. In some embodiments, the three alkane-diols are butanediol.

In some embodiments, the triple linker is linked in between each single linker by a phosphodiester, phosphorothioate, methylphosphonate, or amide linkage.

In some embodiments, the antisense oligonucleotides comprise the entire SNA such that no other structural components are part of the SNA and wherein the antisense oligonucleotide includes a molecular species and non-nucleotidic linker moiety that form the core, with the oligonucleotides extending radially from the core.

In some embodiments, the SNA is free of lipids, polymers or solid cores.

According to some aspects, the SNA comprises a core and a first antisense oligonucleotide comprised of 8 to 50 linked nucleosides in length targeted to a first region in a pre-mRNA of interest and a second antisense oligonucleotide comprised of 8 to 50 linked nucleosides in length targeted to second region in a pre-mRNA of interest to regulate pre-mRNA splicing, and wherein the antisense oligonucleotides are attached to the core and form an oligonucleotide shell.

In some embodiments, the first region in the pre-mRNA of interest is a regulatory site. In some embodiments, the second region in the pre-mRNA of interest is a long non-coding RNA (lncRNA).

In some embodiments, the oligonucleotide shell has a surface density of 5-1,000 oligonucleotides per SNA. In some embodiments, the oligonucleotide shell has a surface density of 100-1,000 oligonucleotides per SNA. In some embodiments, the oligonucleotide shell has a surface density of 500-1,000 oligonucleotides per SNA.

In some embodiments, the oligonucleotide shell has a surface density of at least 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 oligonucleotides per SNA. In some embodiments, the oligonucleotide shell has a surface density of about 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 oligonucleotides per SNA. It will be recognized that the oligonucleotide shell surface density can be expressed as molar ratio of oligonucleotides to lipid which forms the liposome core. In certain embodiments, the lipid to oligonucleotide ratio is 200:1, 150:1, 100:1, 75:1, 70:1, 65:1, 60:1, 55:1, 50:1, 45:1, 40:1, 35:1, 30:1, 25:1, 20:1, 15:1, 10:1, and 5:1.

In some embodiments, the lipid bilayer comprises one or more lipids selected from the group consisting of: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and derivatives thereof.

In some embodiments, the lipid bilayer is comprised of 1,2-dioleoyl-sn-glycero-3-phosphocholine (DOPC).

According to some aspects, the SNA comprises a core and a first antisense oligonucleotide comprised of 8 to 50 linked nucleosides in length targeted to a regulatory site and a second antisense oligonucleotide comprised of 8 to 50 linked nucleosides in length targeted to a region of a lncRNA, and wherein the antisense oligonucleotides are attached to the core and form an oligonucleotide shell.

According to some aspects, the SNA comprises a core and antisense oligonucleotides arranged in an oligonucleotide shell, wherein the oligonucleotides comprise a nucleotide backbone comprising a modification in one or more of the carbons in the five-carbon sugar, and wherein five nucleotides or fewer than five nucleotides do not comprise a modification in the five-carbon sugar.

In some embodiments, four nucleotides or fewer than four nucleotides do not comprise a modification in the five-carbon sugar. In some embodiments, three nucleotides or fewer than three nucleotides do not comprise a modification in the five-carbon sugar. In some embodiments, two nucleotides or fewer than two nucleotides do not comprise a modification in the five-carbon sugar. In some embodiments, one nucleotide does not comprise a modification in the five-carbon sugar. In some embodiments, the modification is not at the 2′-carbon position of the five-carbon sugar.

In some embodiments, all of the nucleotides in the nucleotide backbone of the antisense oligonucleotides comprise a modification in one or more of the carbons in the five-carbon sugar.

In some embodiments, the modification is at the 2′-carbon position of the five-carbon sugar. In some embodiments, the modification is a 2′-O-methylated nucleotide. In some embodiments, the antisense oligonucleotide comprises the nucleic acid sequence CCCACAGGGGCATGUAGU (SEQ ID NO: 58). In some embodiments, the antisense oligonucleotide comprises or consists of the nucleic acid sequence mCmCmCmAmCmAmGmG*mG*mG*mC*mA*mT*mGmUmAmGmU (SEQ ID NO: 59), wherein * is a phosphorothioate linkage and m is a 2′-O-methylated nucleotide. In some embodiments, the antisense oligonucleotide comprises the nucleic acid sequence mCmCmCmAmCmAmGmG*mG*mG*mC*mA*mT*mGmUmAmGmU/Spacer18/Spacer18/3 CholTEG (SEQ ID NO: 211), wherein * is a phosphorothioate linkage, m is a 2′-O-methylated nucleotide, Spacer18 is a hexa(ethylene glycol) spacer, and 3CholTEG is tri(ethylene glycol) bound to a cholesterol.

According to some aspects, the SNA comprises an antisense oligonucleotide comprised of 8 to 50 linked nucleosides in length targeted to a regulatory site of a pre-mRNA of interest and a linker moiety comprising a molecular species at the 3′-end or the 5′-end of the antisense oligonucleotide, wherein the molecular species is a hydrophobic group comprising a stearyl.

In some embodiments, the stearyl is a distearyl.

According to some aspects, the SNA is an SNA for regulating pre-mRNA splicing, comprising a core and an antisense oligonucleotide comprised of 8 to 50 linked nucleosides in length targeted to a regulator of splicing of a pre-mRNA of interest to regulate pre-mRNA splicing, and wherein the antisense oligonucleotide is attached to the core and forms an oligonucleotide shell.

In some embodiments, the regulator regulates the inclusion of exons and/or introns in a mRNA of interest.

In some embodiments, the regulator is an RNA binding protein, a splicing factor or a ribonucleoprotein.

According to some aspects, a composition is contemplated herein. In some embodiments, the composition comprises a SNA disclosed herein in a pharmaceutically acceptable carrier.

In some embodiments, the composition comprises a first spherical nucleic acid (SNA) comprising a core and a first antisense oligonucleotide comprised of 10 to 40 linked nucleosides in length targeted to a first region in a pre-mRNA of interest, and a second SNA comprising a core and a second antisense oligonucleotide comprised of 10 to 40 linked nucleosides in length targeted to a second region in the pre-mRNA of interest.

According to some aspects, methods for treating a subject having a disease or disorder related to an abnormality in splice modulation are contemplated herein.

In some embodiments, the method comprises administering to a subject having the disease or disorder related to an abnormality in splice modulation a spherical nucleic acid (SNA) described herein in an effective amount to increase expression levels of a protein of interest or corrected mRNA over a baseline level in the subject in order to treat the disease or disorder related to an abnormality in splice modulation.

In some embodiments, the disease or disorder related to an abnormality in splice modulation is Stargardt Disease (Juvenile Macular Degeneration), Usher Syndrome, X-Linked Retinoschisis, macular corneal dystrophy, Congenital stromal corneal dystrophy, Congenital hereditary endothelial corneal dystrophy, Fleck corneal dystrophy, lattice corneal dystrophy type I, lattice corneal dystrophy type II, granular corneal dystrophy type I, granular corneal dystrophy type II (Avellino), Epithelial recurrent erosion dystrophy, Stocker-Holt corneal dystrophy, Duchennes Muscular Dystrophy, Leber Congenital Amaurosis, B-Thalassemia, Meesmann Endothelial Corneal Dystrophy, Menkes Disease, Nijmegen Breakage Syndrome, Hutchison-Gilford Progeria Syndrome, Gelatinous droplike corneal dystrophy, Reis-Buckler corneal dystrophy, Schnyder crystalline corneal dystrophy, Subepithelial mucinous corneal dystrophy, Lisch corneal dystrophy, Posterior amorphous corneal dystrophy, X-linked endothelial corneal dystrophy, Thiel-Behnke corneal dystrophy, Posterior polymorphous corneal dystrophy, Pompe, Familial Hypertrophic Cardiomyopathy, X-Linked Aggamaglobulinemia, Oculopharyngeal Muscular Dystrophy, Dilated Cardiomyopathy, Frontotemporal Dementia, epithelial basement membrane corneal dystrophy, Alzheimer's Disease, Familial Hypercholesterolemia, a pro-inflammatory disease, Huntington's disease, Fukuyama congenital muscular dystrophy, Myotonic Dystrophy Type I or II, Cancer, Neovascularization, Ataxia telangiectasia, Congenital disorder of glycosylation, FTD/Parkinsonism linked to chr17, Niemann Pick disease type C, Neurofibromatosis type 1, Neurofibromatosis type 2, Megalencephalic leukoencephalopathy with subcortical cysts type 1, Pelizaeus-Merzbacher disease, Spinocerebellar Ataxia Type 7, Spinocerebellar Ataxia Type 17, Huntington's disease, Spinocerebellar Ataxia Type 1, Spinocerebellar Ataxia Type 12, Spinal and bulbar muscular atrophy, Spinocerebellar Ataxia Type 2, Spinocerebellar Ataxia Type 6, Dentatorubral-pallidoluysian atrophy, Spinocerebellar Ataxia Type 3, Spinocerebellar Ataxia Type 8, Huntington disease-like-2, Myotonic Dystrophy Type I, Fuchs Endothelial Corneal Dystrophy. Fragile X syndrome, fragile X-associated tremor/ataxia syndrome, Fragile XE syndrome, Friedreich ataxia, Myotonic Dystrophy Type II, Spinocerebellar Ataxia Type 10, Spinocerebellar Ataxia Type 31, Spinocerebellar Ataxia Type 36, C9orf72-ALS/FTD, or Prader-Willi syndrome.

In some embodiments, the disease or disorder related to an abnormality in splice modulation is Duchennes Muscular Dystrophy, Leber Congenital Amaurosis, B-Thalassemia, a pro-inflammatory disease, Huntington's disease, Spinocerebellar Ataxia Type 7, Spinocerebellar Ataxia Type 17, Huntington's disease, Spinocerebellar Ataxia Type 1, Huntington disease-like-2, or Prader-Willi syndrome.

In some embodiments, the baseline level is the level of the protein of interest in the subject prior to treatment with the SNA. In some embodiments, the baseline level is the level of the protein of interest in a subject having the disease or disorder related to an abnormality in splice modulation and treated with a linear antisense oligonucleotide targeted to a region in a pre-mRNA of interest to regulate pre-mRNA splicing.

In some embodiments, the SNA is delivered by a route of administration selected from the group consisting of intrathecal, oral, nasal, sublingual, intravenous, subcutaneous, mucosal, respiratory, direct injection, and dermal routes of administration.

In some embodiments, the SNA is a SNA disclosed herein.

According to some aspects, methods for treating a subject having the disease or disorder related to an abnormality in splice modulation comprises administering to a subject having a disease or disorder related to an abnormality in splice modulation at least two doses of a spherical nucleic acid (SNA), in an effective amount to increase expression levels of a protein of interest or corrected mRNA over a baseline level in the subject in order to treat the disease or disorder related to an abnormality in splice modulation, wherein the second dose is administered about 3 months to 2 years after the first dose, and wherein the SNA comprises a core and an antisense oligonucleotide comprised of 8 to 50 linked nucleosides in length targeted to a region in a pre-mRNA of interest, such that a level of a protein of interest or a level of a corrected mRNA relative to a defective mRNA associated with the disease or disorder related to an abnormality in splice modulation in the subject is enhanced. In some embodiments, the oligonucleotides are attached to the core and thus form an oligonucleotide shell. In some embodiments, the corrected mRNA produces a functional protein of interest to treat the subject having the disease or disorder related to an abnormality in splice modulation.

In some embodiments, the region is a regulatory region or regulatory site.

In some embodiments, the disease or disorder related to an abnormality in splice modulation is Stargardt Disease (Juvenile Macular Degeneration), Usher Syndrome, X-Linked Retinoschisis, macular corneal dystrophy, Congenital stromal corneal dystrophy, Congenital hereditary endothelial corneal dystrophy, Fleck corneal dystrophy, lattice corneal dystrophy type I, lattice corneal dystrophy type II, granular corneal dystrophy type I, granular corneal dystrophy type II (Avellino), Epithelial recurrent erosion dystrophy, Stocker-Holt corneal dystrophy, Duchennes Muscular Dystrophy, Leber Congenital Amaurosis, B-Thalassemia, Meesmann Endothelial Corneal Dystrophy, Menkes Disease, Nijmegen Breakage Syndrome, Hutchison-Gilford Progeria Syndrome, Gelatinous droplike corneal dystrophy, Reis-Buckler corneal dystrophy, Schnyder crystalline corneal dystrophy, Subepithelial mucinous corneal dystrophy, Lisch corneal dystrophy, Posterior amorphous corneal dystrophy, X-linked endothelial corneal dystrophy, Thiel-Behnke corneal dystrophy, Posterior polymorphous corneal dystrophy, Pompe, Familial Hypertrophic Cardiomyopathy, X-Linked Aggamaglobulinemia, Oculopharyngeal Muscular Dystrophy, Dilated Cardiomyopathy, Frontotemporal Dementia, epithelial basement membrane corneal dystrophy, Alzheimer's Disease, Familial Hypercholesterolemia, a pro-inflammatory disease, Huntington's disease, Fukuyama congenital muscular dystrophy, Myotonic Dystrophy Type I or II, Cancer, Neovascularization, Ataxia telangiectasia, Congenital disorder of glycosylation, FTD/Parkinsonism linked to chr17, Niemann Pick disease type C, Neurofibromatosis type 1, Neurofibromatosis type 2, Megalencephalic leukoencephalopathy with subcortical cysts type 1, Pelizaeus-Merzbacher disease, Spinocerebellar Ataxia Type 7, Spinocerebellar Ataxia Type 17, Huntington's disease, Spinocerebellar Ataxia Type 1, Spinocerebellar Ataxia Type 12, Spinal and bulbar muscular atrophy, Spinocerebellar Ataxia Type 2, Spinocerebellar Ataxia Type 6, Dentatorubral-pallidoluysian atrophy, Spinocerebellar Ataxia Type 3, Spinocerebellar Ataxia Type 8, Huntington disease-like-2, Myotonic Dystrophy Type I, Fuchs Endothelial Corneal Dystrophy, Fragile X syndrome, fragile X-associated tremor/ataxia syndrome, Fragile XE syndrome, Friedreich ataxia, Myotonic Dystrophy Type II, Spinocerebellar Ataxia Type 10, Spinocerebellar Ataxia Type 31, Spinocerebellar Ataxia Type 36. C9orf72-ALS/FTD, or Prader-Willi syndrome.

In some embodiments, the disease or disorder related to an abnormality in splice modulation is Duchennes Muscular Dystrophy, Leber Congenital Amaurosis, B-Thalassemia, a pro-inflammatory disease, Huntington's disease, Spinocerebellar Ataxia Type 7, Spinocerebellar Ataxia Type 17, Huntington's disease. Spinocerebellar Ataxia Type 1, Huntington disease-like-2, or Prader-Willi syndrome.

According to some aspects, methods of enhancing the level of the corrected mRNA relative to a defective mRNA associated with or related to an abnormality in splice modulation in a cell are contemplated herein.

In some embodiments, the cell is contacted with a spherical nucleic acid (SNA) comprising a core and an antisense oligonucleotide comprised of 10 to 40 linked nucleosides in length targeted to a region in a pre-mRNA of interest, such that the level of a corrected mRNA relative to a defective mRNA in the cell is enhanced.

In some embodiments, the SNA is a SNA disclosed herein.

According to some aspects, a spherical nucleic acid (SNA) for regulating pre-mRNA splicing, comprising a core and an antisense oligonucleotide comprised of 8 to 50 linked nucleosides in length targeted to a regulator of splicing of a pre-mRNA of interest to regulate pre-mRNA splicing, and wherein the antisense oligonucleotide is attached to the core and forms an oligonucleotide shell is contemplated herein.

In some embodiments, the regulator regulates the inclusion of exons and/or introns in a mRNA of interest. In some embodiments, the regulator is an RNA binding protein, a splicing factor or a ribonucleoprotein.

According to some aspects, methods for treating a subject having a disease or disorder related to an abnormality in splice modulation are contemplated herein. In some embodiments, the method comprises administering to a subject having the disease or disorder related to an abnormality in splice modulation a SNA in an effective amount to decrease expression levels of a protein of interest under a baseline level in the subject in order to treat the disease or disorder related to an abnormality in splice modulation.

In some embodiments, the SNA is a SNA disclosed herein.

In some embodiments, the antisense oligonucleotide has locked nucleic acid (LNA) modifications. In some embodiments, less than all of the nucleotides in the antisense oligonucleotide include a LNA modification. In some embodiments, all of the nucleotides in the antisense oligonucleotide include a LNA modification. In some embodiments, the antisense oligonucleotide has morpholino modifications. In some embodiments, less than all of the nucleotides in the antisense oligonucleotide include a morpholino modification. In some embodiments, all of the nucleotides in the antisense oligonucleotide include a morpholino modification.

According to some aspects, methods of producing a splice variant susceptible to nonsense-mediated decay are contemplated herein. In some embodiments, the method comprises contacting a cell with a SNA comprising oligonucleotides arranged in an oligonucleotide shell and a core in an affective amount to produce a splice variant susceptible to nonsense-mediated decay.

According to some aspects, methods of treating a disease or disorder in a subject are contemplated herein. In some embodiments, the method comprises administering to a subject an effective amount of a SNA comprising oligonucleotides arranged in an oligonucleotide shell and a core to produce a splice variant susceptible to nonsense-mediated decay in order to treat the disease or disorder in the subject.

In some embodiments, the SNA is administered to the subject by an administration route selected from the group consisting of intrathecal, oral, nasal, sublingual, intravenous, subcutaneous, mucosal, respiratory, direct injection, and dermal route of administration.

In some embodiments, the SNA is a SNA disclosed herein.

In some embodiments, the disease or disorder is cancer. In some embodiments, the cancer is selected from the group consisting of melanoma, renal cancer, clear cell carcinoma, prostate cancer, hormone refractory prostate adenocarcinoma, breast cancer, colon cancer, lung cancer, non-small cell lung cancer, small cell lung cancer, bone cancer, pancreatic cancer, pancreatic adenocarcinoma, skin cancer, cancer of the head or neck, cutaneous or intraocular malignant melanoma, uterine cancer, ovarian cancer, rectal cancer, stomach cancer, testicular cancer, thyroid cancer, anaplastic thyroid cancer, uterine cancer, carcinoma of the fallopian tubes, carcinoma of the endometrium, carcinoma of the cervix, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, biliary tract cancer, brain cancer, breast cancer, cervical cancer, choriocarcinoma, esophageal cancer, gastric cancer, an intraepithelial neoplasm, lymphoma, liver cancer, neuroblastoma, oral cancer, sarcoma, hairy cell leukemia, chronic myelogenous leukemia, cutaneous T-cell leukemia, multiple myeloma, renal cell carcinoma, lymphoma, bladder cancer, glioblastoma multiforme, Merkel cell carcinoma, cutaneous squamous cell carcinoma, melanoma or squamous cell carcinoma of the head and neck. In some embodiments, the cancer is environmentally-induced cancers, including those induced by asbestos, or any combinations thereof. In some embodiments, the cancer is not melanoma.

In some embodiments, the cancer is selected from the group consisting of pleomorphic sarcoma, gastrointestinal stromal tumor (GIST), liposarcoma, leiomyosarcoma, synovial sarcoma, malignant peripheral nerve sheath tumor, rhabdomyosarcoma, angiosarcoma, fibrosarcoma, dermatofibrosarcoma protuberans, epithelioid sarcoma, myxoma, mesenchymoma, vascular sarcoma, neurilemmoma, bone sarcoma, osteosarcoma, Ewing's sarcoma, chondrosarcoma, Kaposi sarcoma, solitary fibrous tumor, chordoma, desmoid-type fibromatosis, fibroblastic sarcoma, giant cell tumor of the bone, gynaecological sarcoma, soft tissue sarcoma, angioleiomyoma, leiomyoma, smooth muscle sarcoma, fibrohistiocytic sarcoma, sebaceous cell carcinoma and eccrine carcinoma. In some embodiments, the cancer is characterized as microsatellite instability high, or MSI-H, or mismatch repair deficient, or dMMR. MSI-H or dMMR cancers are characterized by defects in DNA replication, particularly in the microsatellite regions. The presence of MSI-H and dMMR tumors has been reported in diverse cancer types, including colon, colorectal, endometrial, biliary, gastric, gastroesophageal junction, pancreatic, small intestinal, breast, triple negative breast, prostate, bladder, esophageal, sarcoma, thyroid, retroperitoneal adenocarcinoma, small cell lung, ovarian, pancreatic, prostate, central nervous system, and non-small cell lung cancers.

In some embodiments, the disease or disorder is an inflammatory disease or disorder.

In some embodiments, the inflammatory disease or disorder is selected from the group consisting of an autoimmune disease, an infectious disease, transplant rejection or graft-versus-host disease, a pulmonary disorder, an intestinal disorder, a cardiac disorder, sepsis, a spondyloarthropathy, a metabolic disorder, a hepatic disorder, a skin disorder and a nail disorder.

In some embodiments, the inflammatory disease or disorder is selected from the group consisting of atopic dermatitis, epidermolysis bullosa, uveitis, gout, polymyalgia rheumatica, osteoarthritis, systemic-onset juvenile idiopathic arthritis, schnitzler syndrome, familial mediterranean fever, cryopyrin-associated periodic syndrome (CAPS), hyper-igd syndrome (HIDS), TNF receptor-associated periodic syndrome (TRAPs), type 2 diabetes, proliferative diabetic retinopathy, wet age-related macular degeneration, chronic obstructive pulmonary disease, type 1 diabetes, pyoderma gangrenosum, dry eye syndrome, and acne vulgaris, rheumatoid arthritis, psoriasis, psoriatic arthritis, psoriasis in combination with psoriatic arthritis, ulcerative colitis, Crohn's disease, vasculitis, Behcet's disease, ankylosing spondylitis, asthma, chronic obstructive pulmonary disorder (COPD), idiopathic pulmonary fibrosis (IPF), restenosis, anemia, pain and hepatitis C virus infection.

In some embodiments, the autoimmune disease is selected from the group consisting of rheumatoid arthritis, rheumatoid spondylitis, osteoarthritis, gouty arthritis, allergy, multiple sclerosis, autoimmune uveitis, and nephritic syndrome.

According to some aspects, methods of increasing the levels of a soluble variant of a transmembrane receptor in a cell are contemplated herein. In some embodiments, the method comprises contacting a cell with an effective amount of a SNA that modulates splicing of the pre-mRNA of a transmembrane receptor to produce a soluble variant of the transmembrane receptor such that the level of soluble variant of the transmembrane receptor is increased relative to a cell that has not been contacted with the SNA or relative to a cell contacted with the corresponding linear oligonucleotide not in a SNA, wherein the levels of the mRNA encoding the transmembrane receptor are not decreased through RNAse-H mediated degradation.

In some embodiments, the transmembrane receptor is an ion channel linked receptor, and enzyme-linked receptor, or a G protein-coupled receptor. In some embodiments, the transmembrane receptor is an adrenergic receptor, an olfactory receptor, a receptor tyrosine kinase, an epidermal growth factor receptor, an insulin receptor, a fibroblast growth factor receptor, a neurotrophin receptor, an ephrin receptor, an integrin, a low affinity nerve growth factor receptor, a N-methyl-D-aspartate (NMDA) receptor, or an immune receptor. In some embodiments, the transmembrane receptor is a toll-like receptor, a T-cell receptor, a cluster of differentiation 28 (CD28), or a csk-interacting membrane (SCIMP) protein.

In some embodiments, the immune receptor is a pattern recognition receptor, a killer activated receptor, a killer inhibitor receptor, a complement receptor, an Fc receptor, a B cell receptor, a T cell receptor, or a cytokine receptor.

In some embodiments, the SNA is a SNA disclosed herein.

In some embodiments, the SNA is in a solution at a concentration of between about 100 nM to 1 μM. In some embodiments, the SNA is in a solution at a concentration of or about 0.5 μM, of or about 1 μM, or of or about 5 μM.

In some embodiments, the cell is brain cell, liver cell, lung cell, gut cell, stomach cell, intestine cell, fat cell, muscle cell, uterine cell, skin cell, spleen cell, endocrine organ cell, or bone cell.

In some embodiments, the SNA is in a pharmaceutically acceptable carrier that is a gel formulation.

In some embodiments, the cell is contacted with the SNA in vitro. In some embodiments, the cell is contacted with the SNA in vivo. In some embodiments, the cell is contacted with the SNA ex vivo.

According to some aspects, methods of treating a disease or disorder in a subject are contemplated herein. In some embodiments, the method comprises administering to a subject with a disease or disorder associated with abnormal transmembrane receptor activity or abnormal transmembrane receptor expression in a cell of the subject an effective amount of a spherical nucleic acid (SNA) to produce or increase the levels of a soluble variant of the transmembrane receptor in the subject relative to a subject with a disease or disorder associated with abnormal transmembrane receptor activity or abnormal transmembrane receptor expression who has not been administered a SNA or relative to a subject with a disease or disorder associated with abnormal transmembrane receptor activity or abnormal transmembrane receptor expression who has been administered a corresponding linear oligonucleotide that is not in a SNA, in order to treat the disease or disorder in the subject.

In some embodiments, the total levels of the transmembrane receptor in the cell of the subject remains stable or the total levels of the mRNA encoding the transmembrane receptor are not decreased through RNAse-H mediated degradation.

In some embodiments, the SNA is a SNA disclosed herein.

In some embodiments, the disease or disorder is atopic dermatitis or psoriasis.

In some embodiments, the transmembrane receptor is interleukin 17 receptor α (IL17RA) or IL1 receptor accessory protein (IL1RAP).

In some embodiments, the SNA is administered to the subject by an administration route selected from the group consisting of intrathecal, oral, nasal, sublingual, intravenous, subcutaneous, mucosal, respiratory, direct injection, and dermal route of administration.

According to some aspects, methods of increasing the levels of a mRNA of interest in a cell are contemplated herein. In some embodiments, the method comprises contacting the cell with a SNA disclosed herein, wherein the levels of the mRNA of interest in the cell is increased relative to a cell that has not been contacted with the SNA or relative to a cell contacted with the corresponding linear oligonucleotide not in a SNA.

According to some aspects, methods of inducing exon skipping in a pre-mRNA of interest in a cell are contemplated herein. In some embodiments, the method comprises contacting a cell with a SNA disclosed herein to induce exon skipping in a pre-mRNA of interest in the cell.

According to some aspects, methods of inducing exon inclusion in a pre-mRNA of interest in a cell are contemplated herein. In some embodiments, the method comprises contacting a cell with a SNA disclosed herein to induce exon inclusion in a pre-mRNA of interest in the cell.

According to some aspects, methods for delivering a stable level of antisense oligonucleotides to a central nervous system (CNS) of a subject having a CNS disease or disorder are contemplated herein. In some embodiments, the method comprises administering to a subject having a neurodegenerative disease or disorder a spherical nucleic acid (SNA) in an effective amount to deliver antisense oligonucleotides to the CNS of the subject, wherein the administration of SNA delivers about 2% to about 150% more antisense oligonucleotides to one or more tissues or regions of the CNS of the subject than administration of linear antisense oligonucleotides that are not in a SNA, wherein the SNA comprises a core and antisense oligonucleotides comprised of 10 to 60 linked nucleosides in length, wherein the antisense oligonucleotides are attached to the core and thus form an oligonucleotide shell, wherein the CNS disease or disorder is not autism. Alzheimer's disease. Parkinson's disease, spinal muscular atrophy, or characterized by muscle wasting and/or loss of muscle function.

In some embodiments, the CNS disease or disorder is encephalitis, poliomyelitis, essential tremor, multiple sclerosis, cancer of the nervous system, addiction, attention deficit/hyperactivity disorder (ADHD), bipolar disorder, catalepsy, depression, epilepsy/seizures, infection, locked-in syndrome, meningitis, migraine, myelopathy or Tourette's syndrome.

In some embodiments, the SNA is administered intrathecally (IT). In some embodiments, the SNA is administered in the lower lumbar region. In some embodiments, the SNA is IT-administered through a lumbar puncture.

In some embodiments, the subject is a mammal. In some embodiments, the subject is a rat or mouse. In some embodiments, the subject is a human.

In some embodiments, a stable level is achieved when at least 50% of the antisense oligonucleotides are present in a tissue of the CNS within three days of administration of the SNA to the subject, relative to the amount of antisense oligonucleotides present in the tissue of the CNS within one hour of administration of the SNA to the subject. In some embodiments, a stable level is achieved when at least 50% of the antisense oligonucleotides are present in a tissue of the CNS within 48 hours of administration of the SNA to the subject, relative to the amount of antisense oligonucleotides present in the tissue of the CNS within one hour of administration of the SNA to the subject. In some embodiments, a stable level is achieved when at least 50% of the antisense oligonucleotides are present in a tissue of the CNS within 24 hours of administration of the SNA to the subject, relative to the amount of antisense oligonucleotides present in the tissue of the CNS within one hour of administration of the SNA to the subject.

In some embodiments, less than 50% of the antisense oligonucleotides are detectable within six hours of administration to the subject in one or both kidneys of the subject. In some embodiments, less than 40% of the antisense oligonucleotides are detectable within six hours of administration to the subject in one or both kidneys of the subject. In some embodiments, less than 30% of the antisense oligonucleotides are detectable within six hours of administration to the subject in one or both kidneys of the subject. In some embodiments, less than 20% of the antisense oligonucleotides are detectable within six hours of administration to the subject in one or both kidneys of the subject. In some embodiments, less than 10% of the antisense oligonucleotides are detectable within six hours of administration to the subject in one or both kidneys of the subject. In some embodiments, less than 5% of the antisense oligonucleotides are detectable within six hours of administration to the subject in one or both kidneys of the subject.

In some embodiments, the SNA is a SNA disclosed herein.

In some embodiments, the SNA is in a formulation and wherein the formulation comprises artificial cerebral spinal fluid (aCSF).

In some embodiments, the one or more tissues or regions of the CNS is one or more regions of the brain.

In some embodiments, the one or more regions of the brain is selected from the group consisting of the amygdala, basal ganglia, cerebellum, corpus callosum, cortex, hippocampus, hypothalamus, midbrain, olfactory region, one or more ventricles, septal area, white matter and thalamus.

In some embodiments, the one or more tissues or regions of the CNS are the cervical cerebral spinal fluid (CSF) or thoracic CSF.

In some embodiments, the antisense oligonucleotides in the SNA have different routes of distribution and clearance from the corresponding linear antisense oligonucleotides that are not in a SNA.

According to some aspects, methods of increasing expression of a mRNA of interest in a cell are contemplated herein. In some embodiments, the method comprises contacting the cell with a first spherical nucleic acid (SNA) comprising a core and a first antisense oligonucleotide comprised of 10 to 40 linked nucleosides in length targeted to a first region in a pre-mRNA of interest, and contacting the cell with a second SNA comprising a core and a second antisense oligonucleotide comprised of 10 to 40 linked nucleosides in length targeted to a second region in the pre-mRNA of interest, wherein the first antisense oligonucleotide in the first SNA and the second antisense oligonucleotide in the second SNA modulate splicing of the pre-mRNA of interest to increase the levels of the mRNA of interest in the cell relative to a cell that has not been contacted with the SNA or relative to a cell contacted with the corresponding linear oligonucleotide not in a SNA.

In some embodiments, the first antisense oligonucleotide in the first SNA and the second antisense oligonucleotide in the second SNA work synergistically.

In some embodiments, the first SNA or the second SNA is a SNA disclosed herein.

In some embodiments, the first SNA and the second SNA is a SNA disclosed herein.

In some embodiments, the SNA comprises a core and the first antisense oligonucleotide and the second antisense oligonucleotide attached to the same core.

According to some aspects, methods of increasing the levels of a mRNA of interest in a cell are contemplated herein. In some embodiments, the method comprises contacting a cell with a spherical nucleic acid (SNA) comprising a core and a first antisense oligonucleotide comprised of 10 to 40 linked nucleosides in length targeted to a first region in a pre-mRNA of interest and a second antisense oligonucleotide comprised of 10 to 40 linked nucleosides in length targeted to a second region in the pre-mRNA of interest, wherein the first antisense oligonucleotide and the second antisense oligonucleotide modulate splicing of the pre-mRNA of interest to increase the levels of the mRNA of interest in the cell, relative to a cell that has not been contacted with the SNA or relative to a cell contacted with the corresponding linear oligonucleotide not in a SNA.

In some embodiments, the first antisense oligonucleotide and the second antisense oligonucleotide work synergistically.

In some embodiments, the SNA is a SNA disclosed herein.

In some embodiments, the SNA comprises a core and the first antisense oligonucleotide and the second antisense oligonucleotide attached to the same core.

According to some aspects, methods are disclosed for delivering a stable level of antisense oligonucleotides to a central nervous system (CNS) of a subject having a CNS disease or disorder comprises administering to a subject having a neurodegenerative disease or disorder a spherical nucleic acid (SNA) in an effective amount to deliver a first antisense oligonucleotide and a second antisense oligonucleotide to the CNS of the subject, wherein the administration of SNA delivers about 2% to about 150% more antisense oligonucleotides to one or more tissues or regions of the CNS of the subject than administration of linear antisense oligonucleotides that are not in a SNA, wherein the SNA comprises a core and antisense oligonucleotides comprised of 10 to 60 linked nucleosides in length, wherein the antisense oligonucleotides are attached to the core and thus form an oligonucleotide shell.

According to some aspects, methods are disclosed for delivering a stable level of antisense oligonucleotides to a central nervous system (CNS) of a subject having a CNS disease or disorder, the method comprising administering to a subject having a neurodegenerative disease or disorder a first spherical nucleic acid (SNA) in an effective amount to deliver a first antisense oligonucleotide and a second SNA to deliver a second antisense oligonucleotide to the CNS of the subject, wherein the administration of SNA delivers about 2% to about 150% more antisense oligonucleotides to one or more tissues or regions of the CNS of the subject than administration of linear antisense oligonucleotides that are not in a SNA, wherein the SNA comprises a core and antisense oligonucleotides comprised of 10 to 60 linked nucleosides in length, wherein the antisense oligonucleotides are attached to the core and thus form an oligonucleotide shell.

In some embodiments, the CNS disease or disorder is SMA.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B show the fold increase of SMN2 mRNA over SMN Δ7 mRNA following 48 hour treatment of SMA patient-derived fibroblasts with the compounds. FIG. 1A (Full-length SMN mRNA) and FIG. 1B (Δ7 SMN mRNA) FIGS. 2A-2B show SMN2 protein, mRNA detection and quantification (72 hours). FIG. 2A Western blot showing total SMN protein and loading control GRP94. FIG. 2B is a densitometric quantification of SMN western blot (solid bars) and qRT-PCR of full-length SMN mRNA (hashed bars) from identically treated wells.

FIGS. 3A-3D show that SNAs designed to induce exon skipping of IL17RA are more potent than linear oligonucleotides in vitro. FIG. 3A shows expression of transmembrane IL17RA after 48 hours in vitro treatment of HaCaTs, FIG. 3B shows expression of transmembrane IL17RA after 48 hours in vitro treatment of HFKs FIG. 3C shows endpoint PCR readout of HaCaT samples showing clear shift to soluble isoform following IL17RA SNA treatment, FIG. 3D shows quantification of band intensity for corresponding endpoint PCR.

FIGS. 4A-4B show that SNAs designed to induce exon skipping of IL17RA are more potent than linear oligonucleotides ex vivo. FIG. 4A shows expression of transmembrane IL17RA after 1.7 mM ex vivo, gel-based, topical treatment of human skin biopsies. FIG. 4B shows expression of transmembrane IL17RA after 1.7 mM ex vivo, gel-based, topical treatment of human skin equivalents stimulated to an atopic dermatitis-like phenotype. The gel-based topical treatment consists of the ingredients listed in Table 3.

FIGS. 5A-5B show that SNAs designed to induce exon skipping of REST are more potent than linear oligonucleotides. FIG. 5A shows the ratio of gene expression of full-length to exon 3-deficient transcript (quantified with qPCR) after 48 hours in vitro treatment of MEFs at 5 μM, FIG. 5B shows the endpoint PCR readout of corresponding samples showing production of exon 3-skipped, not naturally occurring transcript.

FIGS. 6A-6C show that SNAs designed to induce exon skipping of IL1RAP are more potent as SNAs than linear splice-switching oligonucleotides (SSOs) and more potent in co-delivery in SNA form. Ratio of transmembrane to total IL1RAP after 48 hours in vitro treatment of HFKs shown. FIG. 6A and FIG. 6B show that SNA form is superior to linear for oligonucleotide 1, 2 (IC₅₀ of 11.1, 4.4 μM for SNA 1, 2; cannot be determined for linear). FIG. 6C shows co-delivery of I and 2 in SNA form is superior to the linear combination (IC₅₀ 0.36 μM for bispecific SNA vs. 200 μM for linear) and to monospecific SNA1 or SNA2. 4PL logistic fit was used for IC₅₀ calculations.

FIG. 7 shows that SNAs designed to induce exon skipping of IL1RAP are more potent as SNAs than linear splice-switching oligonucleotides (SSOs) and more potent in co-delivery in SNA form. Endpoint PCR data showing appearance of soluble isoform following IL1 RAP SNA treatment.

FIG. 8 shows that SNAs comprised of a fully 2′-methoxyethyl (MOE) modified oligonucleotide reduce total STAT3 levels, indicating production of an out-of-frame transcript that undergoes nonsense-mediated decay. Gene expression of STAT3 (quantified with qPCR) after 72 hours treatment of Δ549s is shown.

FIGS. 9A-9C show that SNAs designed to induce exon inclusion of SMN2 are more potent when delivered in SNA form than as linear oligonucleotides. FIG. 9A shows that SNAs are 16-fold more potent at reducing del7 SMN2 transcript (IC50 1.8 and 19.2 μM, respectively) and FIG. 9B shows that SNAs are more potent at increasing full-length SMN2 transcript (EC₅₀ of 0.8 μM for SNA; linear could not be determined. FIG. 9C shows that neither linear nor SNA effected total SMN2 transcript. Patient fibroblasts were treated and a 4PL, 3PL logistic fit was used for IC₅₀ calculations, EC₅₀ calculations.

FIGS. 10A-10B show Kaplan-Meier survival plots of SMA mice treated with a single intracerebro-ventricular (ICV) injection of SNA-ASO or linear ASO at 10, 20 or 30 μg doses at age P0 (post-natal day 0). Linear represents linear ASO and SNA represents SNA-ASO. FIG. 10A shows Δ7SMA mice treated with the 30 μg dose Nusinersen-SNA had increased survival to a maximum of 82 days while scramble SNA has no effect on survival. FIG. 10B shows that linear Nusinersen improved survival of Δ7 SMA mice to a maximum of 28 days.

FIGS. 11A-11B show increase in body weight of SMA mice treated with a single ICV injection of SNA-ASO at 10, 20 or 30 μg or linear ASO at 10 or 20 μg doses. Mice in 30 μg SNA-ASO group have not reached end point. Linear represents linear ASO and SNA represents SNA-ASO. FIG. 11A shows that weights are similar in Δ7SMA mice treated with linear or Nusinersen-SNA treated mice. FIG. 11B shows that weights are similar in Δ7SMA mice treated with morpholino to ISS-N1 or Nusinersen-SNA.

FIG. 12 shows a bar graph depicting increased exon 7 incorporation in SMN2 mRNA transcript in SMA mice treated with SNA-ASO (30 μg single dose on P0) compared with untreated mice on P10. SNA ISS-N1 represents SNA-ASO.

FIG. 13 shows distribution of linear ASO and SNA ASO in the whole brain over 7 day period following single intrathecal administration in lower lumbar region of SD rat. ID=injected dose.

FIG. 14 shows ¹²⁵I-ASO distribution in Sprague Dawley rats.

FIG. 15 shows ¹²⁵I-ASO concentration in kidneys (% ID/g).

FIG. 16 shows ¹²⁵I-ASO group mean for all brain regions in % ID/g.

FIG. 17 shows ¹²⁵I-ASO concentration in the olefactory region (% ID/g).

FIG. 18 shows ¹²⁵I-ASO concentration in the whole brain (% ID/g).

FIG. 19 shows ¹²⁵I-ASO concentration in the ventricles (% ID/g).

FIG. 20 shows ¹²⁵I-ASO concentration in whole blood and plasma at 168h.

FIG. 21 shows ¹²⁵I-ASO concentration in the spleen (% ID/g).

FIG. 22 shows ¹²⁵I-ASO concentration in the liver (% ID/g).

FIG. 23 shows ¹²⁵I-ASO concentration in the thyroid (% ID/g).

FIG. 24 shows ¹²⁵I-ASO in superficial cervical lymph nodes (% ID/g).

FIG. 25 shows ¹²⁵I-ASO in deep cervical lymph nodes (% ID/g).

FIG. 26 shows ¹²⁵I-ASO concentration in the CSF and thoracic region (% ID/g).

FIG. 27 shows ¹²⁵I-ASO in lumbar CSF (% ID/g).

FIG. 28 shows ¹²⁵I-ASO in cervical CSF (% ID/g).

FIG. 29 shows ¹²⁵I-ASO concentration in the septal area (% ID/g).

FIGS. 30A-30B are a table showing the average percent injected dose per gram of tissue over 7 days for various organs and regions of brain and spinal cord in rats. The top third of the table shows the values for linear ASO, middle third for SNA ASO and bottom third shows the ratio of SNA ASO to linear ASO.

FIGS. 31A-31B show quantification of full-length and Δ7 variants of SMN2 mRNA transcripts in SMA patient fibroblasts after treatment with liposomal or gold SNAs. Fold changes in SMN2 mRNA levels were calculated relative to the untreated fibroblasts. Lipidated oligonucleotides were also tested alone without being functionalized on a SNA core. FIG. 31A shows Liposomal vs Gold SNA; Full-length SMN2 mRNA and FIG. 31B shows Liposomal vs Gold SNA; Δ7 SMN2 mRNA.

FIG. 32 shows SPECT/CT images 168 hours post-intrathecal administration of ¹²⁵I-ASOs in rats. Subject 4001 will be excluded from quantitative analysis as the injection appears to be in the epidural space.

FIG. 33 shows SPECT/CT images of IT injection of ¹²⁵I-ASOs in rat at 6 and 168 hours.

FIGS. 34A-34B show decay-corrected SPECT/CT images of intrathecally administered ASO for ¹²⁵I-ASO linear in subject 4001 across timepoints. This subject will be excluded from quantitative analysis as the injection appears to be in the epidural space.

FIGS. 35A-35B show decay-corrected SPECT/CT images of intrathecally administered ASO for ¹²⁵I-ASO linear in subject 4002 across timepoints.

FIGS. 36A-36B show decay-corrected SPECT/CT images of intrathecally administered ASO for ¹²⁵I-ASO linear in subject 4007 across timepoints.

FIGS. 37A-37B show decay-corrected SPECT/CT images of intrathecally administered ASO for ¹²⁵I-ASO SNA in subject 4006 across timepoints. Distribution and persistence of SNA-ASO in SD rats after single bolus, intrathecal administration. The SNA-ASO contains iodine-125 radioactive label and is visualized using whole body SPECT/CT imaging. After administration in the lower lumbar region, the SNA-ASO distributes through the spinal cord (H0.0) and parts of the brain (H0.25 through H0.75). Compared with linear ASO, the signal intensity is lower for SNA-ASO at H0.75 but rises to comparable levels by 6 hours. Unlike with linear ASO, clearance through the kidneys is not detectable at any time point during the 7-day monitoring period. Also unlike linear ASO, by day 7, high amount of SNA-ASO is still present in the brain.

FIGS. 38A-38B show decay-corrected SPECT/CT images of intrathecally administered ASO for ¹²⁵I-ASO SNA in subject 4004 across timepoints.

FIGS. 39A-39B show decay-corrected SPECT/CT images of intrathecally administered ASO for ¹²⁵I-ASO SNA in subject 4005 across timepoints.

FIGS. 40A-40B show distribution and persistence of linear ASO in SD rats after single bolus, intrathecal administration. The linear ASO contains iodine-125 radioactive label and is visualized using whole body SPECT/CT imaging. Shortly after administration in the lower lumbar region, the oligonucleotide distributes through the spinal cord (H0.0) and parts of the brain (H0.25 through H0.75). Beginning at 6-hours after administration, the clearance through kidney is clearly visible (H6). Kidneys are much easier to visual at 24 hours due to high level of clearance (H24). By day 7, majority of the oligonucleotide has cleared out of the CNS. Only limited punctate pattern remains throughout CNS indicating low amount of oligonucleotide.

FIG. 41 shows an ROI analysis key.

FIG. 42 shows IT injection of ¹²⁵I-ASOs in rat in SPECT/CT images at 6 and 168h.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving.” and variations thereof herein, is meant to encompass the items listed thereafter and equivalents thereof as well as additional items.

Genetic diseases and disorders are often associated with genetic abnormalities and splicing errors. The modulation of splicing using antisense oligonucleotides provides an attractive therapeutic option for tailored interventions in diseases or disorders associated with abnormalities in splicing of pre-mRNA, such as genetic diseases or disorders.

It has been discovered, quite unexpectedly, that compounds referred to as antisense oligonucleotides which function in modulating splicing described herein are more potent when arranged in a spherical nucleic acid (SNA) format. It was discovered that these splice modulating antisense oligonucleotides are more active in a SNA relative to the same linear antisense oligonucleotides. This unexpected finding, demonstrated herein, shows that splice modulating antisense oligonucleotides comprised of a variety of lipid-containing cores or other cores, oligonucleotide sequences, oligonucleotide lengths, and oligonucleotide densities are capable of enhancing the expression of corrected mRNA variants containing a desired sequence, in some embodiments, for expression of functional protein.

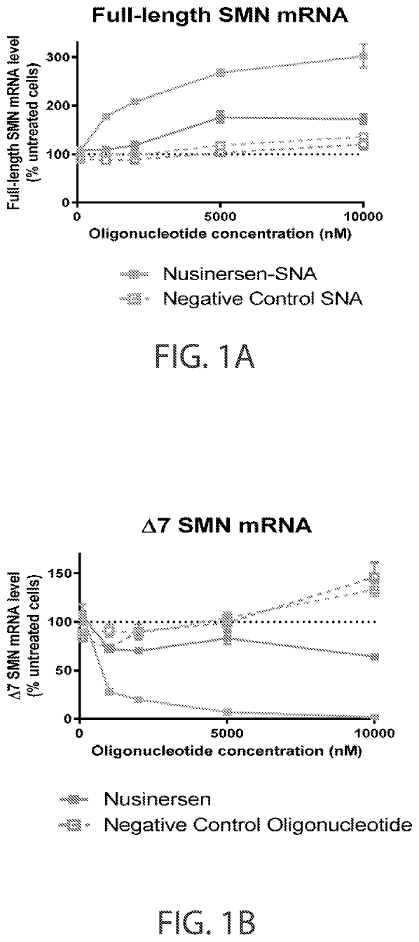

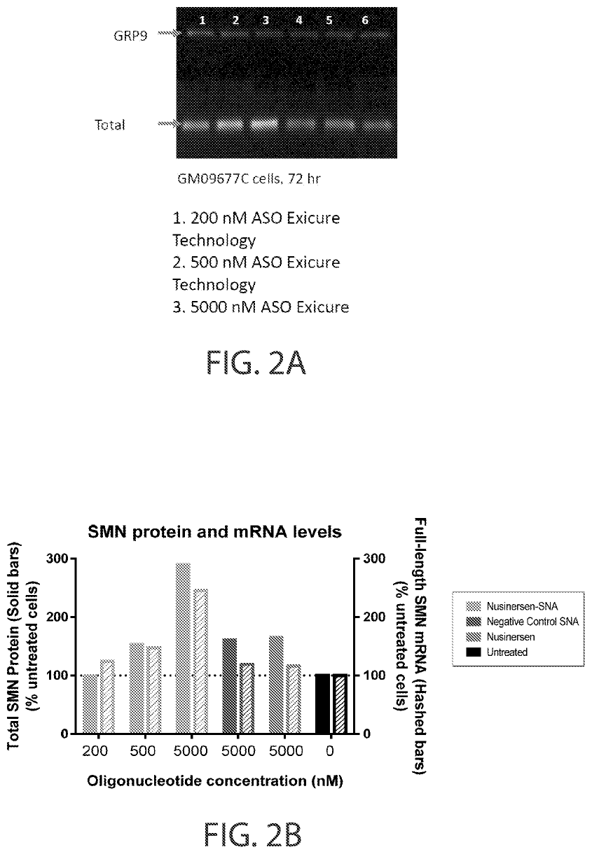

In a non-limiting and exemplary embodiment, the data presented herein show that having the antisense oligonucleotide in a SNA enhanced the inclusion of an exon normally excluded from the survival motor neuron (SMN) 2 gene in the genetic disorder spinal muscular atrophy (SMA). SMA is an autosomal recessive neurodegenerative disorder characterized by progressive muscle wasting and loss of muscle function due to severe motor neuron dysfunction. SMA is caused by low levels of survival of motor neuron (SMN) due to deletion or loss of function of SMN I gene. It was found, unexpectedly, that linear antisense oligonucleotides which lack the oligonucleotide shell do not show similar activity (FIGS. 1A-1B). The results suggest that antisense oligonucleotide SNAs are uniquely able to achieve the desired inclusion of exon 7 in the SMN2 gene and ultimately lead to increased expression of SMN protein for the treatment of SMA. In some embodiments, a SNA disclosed herein modulates splicing for the production of a mRNA transcript variant targeted for nonsense mediated decay (NMD). In some embodiments, such mRNA transcript variant is produced for the treatment of a disease or disorder disclosed herein. In a non-limiting embodiment, the disease or disorder is cancer.

Also, unexpectedly, the antisense oligonucleotides disclosed herein have a different distribution and persistence compared to the corresponding linear or free antisense oligonucleotides in vivo. Exemplary antisense oligonucleotides in the SNA disclosed herein are distributed away from the site of administration relatively slowly and are maintained in the target region/organ for a time longer than the corresponding linear or free antisense oligonucleotide. Furthermore, less antisense oligonucleotide in the SNA is observed in the kidneys which, without wishing to be bound by theory, likely indicates a relatively slow clearance rate from the CNS. The slower clearance and accumulation in the kidneys of antisense oligonucleotide in a SNA relative to linear or free antisense oligonucleotide could also result in lower renal toxicity. Overall, disclosed herein is that antisense oligonucleotides in a SNA persist in the CNS longer and at higher levels compared to the corresponding free or linear antisense oligonucleotides.

Furthermore, in non-limiting aspects of the disclosure, the data presented herein show that having the splice modulating antisense oligonucleotides in a SNA enhanced the inclusion of an exon normally excluded from the SMN2 gene in spinal muscular atrophy (SMA); that the splice modulating antisense oligonucleotides in a SNA enhance switching of a transmembrane receptor to a soluble variant of the transmembrane receptor (e.g., transmembrane receptor involved in inflammation) via exon skipping, which provides a mechanism for treating diseases and disorders associated with increased transmembrane receptor expression or activation; that two or more different oligonucleotides can mediate a desired effect, and in some instances can produce a synergistic or additive effect; that the antisense oligonucleotides disclosed herein decrease expression of certain splice variants via nonsense-mediated decay, which has potential for treating diseases and disorders associated with overexpression of proteins, such as anti-cancer therapies; that the antisense oligonucleotide in a SNA also persists in the CNS longer and at higher levels compared to the corresponding free or linear antisense oligonucleotide; and that the antisense oligonucleotides in a SNA have decreased renal accumulation which results in reduced renal toxicity compared to the corresponding free or linear antisense oligonucleotide. Linear splice modulating antisense oligonucleotides which lack the oligonucleotide shell do not show similar activity. The results suggest that antisense oligonucleotide SNAs are uniquely able to achieve the desired splice modulation to modulate the levels of expression of a mRNA of interest for the expression of a protein of interest. In a non-limiting example, a SNA disclosed herein achieves increased inclusion of exon 7 in the SMN2 gene and ultimately lead to increased expression of SMN protein for the treatment of SMA.

Further, in vivo data (described in the Examples) has demonstrated that the splice modulating oligonucleotide SNA (also referred to as Nusinersen-SNA or Spinraza-SNA) exhibits significantly improved therapeutic properties as compared with the linear oligonucleotide (nusinersen) in a mouse model of SMA. Because nusinersen is clinically administered to the CSF, the constructs were delivered to the CSF via intracerebral ventricular (lCV) injection in post-natal day 0 (P0) mice. Mice treated with 20 μg of nusinersen had a median survival of 17 days, compared to 14 days in untreated mice. In contrast, 10 μg of nusinersen-SNA increased median survival to 26 days whereas 20 μg increased survival to 69 days. Increasing the nusinersen dose to 30 μg resulted in toxicity and a median survival of 2 days. Thus, nusinersen-SNA treatment resulted in substantially increased median survival over nusinersen at the same dose. Unlike Nusinersen and quite unexpectedly, administration of nusinersen-SNA by ICV injection to the CSF at 30 μg dose did not lead to acute toxicity. In vitro and in vivo, nusinersen-SNA treatment elicited more full length SMN mRNA compared to nusinersen. Given that SNAs improve the efficacy and safety of nusinersen in the central nervous system (CNS), the SNAs of the invention may improve the therapeutic window of existing splice modulating oligonucleotides and thus, may be used as novel therapies for CNS diseases and disorders, such as those disclosed herein. SNAs may also provide an opportunity for the delivery of therapeutics, such as free or linear oligonucleotides, which are cleared relatively quickly from the body and/or cause renal toxicity. In some embodiments, a SNA disclosed herein costs reduced renal toxicity in a subject compared to the corresponding linear or free oligonucleotide.

The data show that the SNAs disclosed herein demonstrated increased survival and decreased toxicity in a translationally-relevant SNA mouse model. In brief, the data demonstrated prolonged survival by four-fold (maximal survival of 115 days compared to 28 days for nusinersen-treated mice), doubled the levels of healthy full-length SMN2 mRNA and protein in SMA patient fibroblasts when compared to nusinersen, doubled the quantity of healthy full-length SMN mRNA levels in spinal cord tissue compared to untreated mice and mitigated toxicity of nusinersen at the highest dose tested in mice.

Overall, the present application demonstrates the potential of SNAs to be transformative therapies for diseases including inflammatory conditions, cancer and rare neurological and ophthalmological disorders.

SNA are three-dimensional arrangements of nucleic acids, with densely packed and radially arranged oligonucleotides on a central nanoparticle core. In its simplest form the SNA is composed of oligonucleotides and a core. The core may be a hollow core which is produced by a 3-dminensional arrangement of molecules which form the outer boundary of the core. For instance, the molecules may be in the form of a lipid layer or bilayer which has a hollow center. Alternatively, the molecules may be in the form of lipids, such as amphipathic lipids, i.e., sterols which are linked to an end the oligonucleotide. Sterols such as cholesterol linked to an end of an oligonucleotide may associate with one another and form the outer edge of a hollow core with the oligonucleotides radiating outward from the core. The core may also be a solid or semi-solid core.

In some embodiments, the core comprises or consists of a metal core. In some embodiments, the core is an inorganic metal core. Non-limiting examples of a metal core include gold, silver, platinum, aluminum, palladium, copper, cobalt, indium, nickel and mixtures thereof. In some embodiments, the core comprises or consists of gold. In some embodiments, a SNA disclosed herein is degradable. In some embodiments, the core is a solid core. In some embodiments, the core is a hollow core. In some embodiments, a SNA or core disclosed herein comprises a semiconductor or magnetic material. In some embodiments, the core is a liposomal core.

In some embodiments, an oligonucleotide disclosed herein is attached to the core through a covalent interaction (e.g., thiol-gold interaction). In some embodiments, an oligonucleotide disclosed herein is attached to the core through a non-covalent interaction (e.g., van der Waals interaction, ionic interaction or electrostatic interaction). In some embodiments, an oligonucleotide disclosed herein is uniformly dispersed or suspended around a core, such as a liposomal core or a gold core. In some embodiments, the oligonucleotide is not uniformly dispersed or suspended around a core, such as a liposomal core or gold core. In some embodiments, the non-covalent interaction is reversible.

The oligonucleotides are associated with the core. An oligonucleotide that is associated with the core may be covalently linked to the core or non-covalently linked to the core, i.e., potentially through hydrophobic interactions. For instance, when a sterol forms the outer edge of the core an oligonucleotide may be covalently linked to the sterol directly or indirectly. When a lipid layer forms the core, the oligonucleotide may be covalently linked to the lipid or may be non-covalently linked to the lipids e.g., by interactions with the oligonucleotide or a molecule such as a cholesterol attached to the oligonucleotide directly or indirectly through a linker or linker moiety.

SNAs are taken up by cells to a greater extent than the same oligonucleotides that are not in the SNA. Nontoxic, biocompatible, and biodegradable lipid-containing SNAs that are useful for treating diseases and disorders, such as diseases and disorders related or associated with an abnormality in splice modulation, are disclosed herein. Antisense technology is an effective means for modulating the expression of one or more specific gene products, including alternative splice products, and is uniquely useful in a number of therapeutic, diagnostic, and research applications. The principle behind antisense technology is that an antisense compound, which hybridizes to a target nucleic acid, modulates gene expression activities such as transcription, splicing or translation through one of a number of antisense mechanisms. The sequence specificity of antisense compounds makes them extremely attractive as tools for target validation and gene functionalization, as well as therapeutics to selectively modulate the expression of genes involved in disease and disorders.

As used herein, “antisense activity” refers to any detectable and/or measurable change attributable to the hybridization of an antisense compound to its target nucleic acid. In some embodiments, antisense activity is a decrease in the amount or expression of a target nucleic acid or protein encoded by such target nucleic acid compared to target nucleic acid levels or target protein levels in the absence of the antisense compound. In some embodiments, antisense activity is an increase in the amount or expression of a target nucleic acid or protein encoded by such target nucleic acid compared to target nucleic acid levels or target protein levels in the absence of the antisense compound.

As used herein, “antisense compound” refers to a compound comprising a splice modulating antisense oligonucleotide in a spherical nucleic acid (SNA). The terms “antisense compound” or “oligonucleotide” or “splice modulating compound” or are used interchangeably to refer to a splice modulating oligonucleotide. As used herein, “antisense oligonucleotide” refers to an oligonucleotide having a nucleobase sequence that is at least partially complementary to a target nucleic acid. In some embodiments, the antisense oligonucleotide contains one or more additional features, or one or more additional modifications.

Splice modulating oligonucleotides direct pre-mRNA splicing by binding sequence elements and blocking access to the transcript by the spliceosome and other splicing factors. They can be applied to (1) restore correct splicing of an aberrantly spliced transcript, (2) produce a novel splice variant that is not normally expressed, or (3) manipulate alternative splicing from one splice variant to another. Through the latter mechanism, splice-switching oligonucleotides may therefore downregulate a deleterious transcript while simultaneously upregulating expression of a preferred transcript. Notably, their activity is enhanced with increased target gene expression because this enables increased production of the preferred splice variant. This is in contrast to traditional anti-sense approaches and small-interfering RNA, which exhibit decreased potency with increased target gene expression.

In some embodiments, an antisense oligonucleotide refers to a splice modulating antisense oligonucleotide that comprises or consists of the nucleic acid sequence of SEQ ID NO: 1 below.

(SEQ ID NO: 1)
5′ - TCA CTT TCA TAA TGC TGG - (Spacer 18)₂ -
3CholTEG.

In some embodiments, a SNA described herein comprises an oligonucleotide that is 50% to 100% identical to the nucleic acid sequence of SEQ ID NO: 1. In some embodiments, a SNA described herein comprises an oligonucleotide that is 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 1.

In some embodiments, the antisense oligonucleotide refers to the nucleic acid sequence of ISIS 396443. As used herein, “ISIS 396443” refers to an oligonucleotide having the following structure:

(SEQ ID NO: 16)
Tes ^{m}Ces Aes ^{m}Ces Tes Tes Tes ^{m}Ces Aes Tes Aes Aes
Tes Ges ^{m}Ces Tes Ges Ge,

wherein “^{m}C” indicates 5-methyl cytidine; “e” indicates a 2′-MOE modification; “C” indicates cytidine, “T” indicates thymidine. “A” indicates adenosine, “G” indicates guanosine, and “s” indicates phosphorothioate linkage. Isis 396443 is also referred to in the art as Nusinersen, which is the International Nonproprietary Name (INN), Ionis-SMNRx, and as BIIB058. As used herein, “MOE” refers to methoxyethyl. “2′-MOE” means a —OCH₂CH₂OCH₃ group at the 2′ position of a furanosyl ring.

In some embodiments, a SNA described herein comprises an oligonucleotide that is 50% to 100% identical to the nucleic acid sequence of SEQ ID NO: 16. In some embodiments, a SNA described herein comprises an oligonucleotide that is 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 16.

In some embodiments, the antisense oligonucleotide refers to an antisense oligonucleotide that comprises or consists of the nucleic acid sequence of SEQ ID NO: 17 below.

(SEQ ID NO: 17)
5′-CUA UAU AUA GAU AGU UAU UCA ACA AA-3′.

The following oligos were modified at every base with Morpholino chemistry groups:

(SEQ ID NO: 17)
EIMO-ASO (26-mer)
5’ CUA UAU AUA GAU AGU UAU UCA ACA AA 3’.

In some embodiments, a SNA described herein comprises an oligonucleotide that is 50% to 100% identical to the nucleic acid sequence of SEQ ID NO: 17. In some embodiments, a SNA described herein comprises an oligonucleotide that is 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 17.

In some embodiments, each base of the antisense oligonucleotide of SEQ ID NO: 17 is modified with morpholino chemistry groups. A “morpholino oligomer” or “PMO” refers to an oligonucleotide having a backbone which supports a nucleobase capable of hydrogen bonding to typical polynucleotides, wherein the polymer lacks a pentose Sugar backbone moiety, but instead contains a morpholino ring. An exemplary “morpholino oligomer comprises morpholino subunit structures linked together by phosphoramidate or phosphorodiamidate linkages, joining the morpholino nitrogen of one subunit to the 4′ exocyclic carbon of an adjacent subunit, each subunit comprising a purine or pyrimidine nucleobase effective to bind, by base-specific hydrogen bonding, to a base in a polynucleotide. Morpholino oligomers (including antisense oligomers) are detailed, for example, in U.S. Pat. Nos. 5,698,685; 5,217,866; 5,142,047; 5,034,506; 5,166,315; 5,185,444; 5,521,063; 5,506,337 and pending U.S. patent application Ser. Nos. 12/271,036; 12/271,040; and PCT publication number WO/2009/064471 all of which are incorporated herein by reference in their entirety.

In some embodiments, each base of the antisense oligonucleotide of SEQ ID NO: 17 is modified with locked nucleic acid (LNA), in which the 2′-hydroxyl group of the RNA is linked to the 4′ carbon atom of the sugar ring, thereby forming a bicyclic sugar moiety. The linkage is in certain aspects is a methylene (—CH2-)n group bridging the 2 oxygen atom and the 4′ carbon atom wherein n is 1, 2 or 3. LNAs and preparation thereof are described in WO 98/39352 and WO 99/14226. In some embodiments, the antisense oligonucleotide has locked nucleic acid (LNA) modifications. In some embodiments, less than all of the nucleotides in the antisense oligonucleotide include a LNA modification. In some embodiments, all of the nucleotides in the antisense oligonucleotide include a LNA modification. In some embodiments, the antisense oligonucleotide has morpholino modifications. In some embodiments, less than all of the nucleotides in the antisense oligonucleotide include a morpholino modification. In some embodiments, all of the nucleotides in the antisense oligonucleotide include a morpholino modification.

In other embodiments, each base of the antisense oligonucleotide of SEQ ID NO:17 is a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In some embodiments, the present invention provides antisense compounds, which comprise or consist of an oligomeric compound comprising a splice modulating antisense oligonucleotide, having a nucleobase sequences complementary to that of a target nucleic acid.

In some embodiments, an antisense oligonucleotide described herein is used for the treatment of a disease or disorder, such as a genetic disorder, associated with abnormalities in splice modulation.

Splice-switching oligonucleotides direct pre-mRNA splicing by binding sequence elements and blocking access to the transcript by the spliceosome and other splicing factors. They can be applied to (1) restore correct splicing of an aberrantly spliced transcript, (2) produce a novel splice variant that is not normally expressed, or (3) manipulate alternative splicing from one splice variant to another. Through the latter mechanism, splice-switching oligonucleotides may therefore downregulate a deleterious transcript while simultaneously upregulating expression of a preferred transcript. Notably, their activity is enhanced with increased target gene expression because this enables increased production of the preferred splice variant. This is in contrast to traditional anti-sense approaches and small-interfering RNA, which exhibit decreased potency with increased target gene expression.

Non-limiting examples of gene targets and diseases or disorders amenable to splice modulation are listed in Table 1.

TABLE 1
Disease/Target Combinations Amenable to Splice Modulation

| Target | Indication, Disease or | NCBI NG_ | NCBI NM_ |
|---|---|---|---|
| | Disorder | (genome) | (mRNA) |
| IL-1RAP | Various pro- | NG_029105.1 | NM_001167928.1 |
| | inflammatory diseases | | |
| STAT3 | Cancer | NG_007370.1 | NM_003150.3 |

In some embodiments, the pre-mRNA of interest is obtained or derived from the genomic sequence of a target of interest or a gene of interest. For instance, the pre-mRNA listed in Table 1 is obtained or derived by transcribing the genomic sequence provided by an NCBI ID.

In some embodiments, the pre-mRNA of interest is obtained from the genomic sequence of interleukin 17 receptor A (IL17RA), RE1 Silencing Transcription Factor (REST), IL1 receptor accessory protein (IL1RAP), or signal transducer and activator of transcription 3 (STAT3). The nucleic acid and amino acid sequences of genes, mRNA and protein variants of IL17RA, REST, IL1RAP, and STAT3 are provided in Table 13.

According to some aspects, methods of producing a splice variant susceptible to nonsense-mediated decay are contemplated herein. In some embodiments, the method comprises contacting a cell with a SNA comprising oligonucleotides arranged in an oligonucleotide shell and a core in an affective amount to produce a splice variant susceptible to nonsense-mediated decay (NMD). According to some aspects, methods of treating a disease or disorder in a subject are contemplated herein. In some embodiments, the method comprises administering to a subject an effective amount of a SNA comprising oligonucleotides arranged in an oligonucleotide shell and a core to produce a splice variant susceptible to NMD in order to treat the disease or disorder disclosed herein in the subject. In some embodiments, the method comprises modulating splicing of a pre-mRNA of interest to produce a splice variant susceptible to NMD in order to reduce the levels of a protein of interest in a subject with a disease or disorder disclosed herein, such as cancer, in order to treat the disease or disorder, relative to a baseline (e.g., relative to the levels of the protein of interest in a subject who does not have the disease, relative to the levels of the protein of interest in a subject who has the disease and has not been administered the SNA, or relative to the levels of the protein of interest in a subject who has the disease and has been administered the corresponding linear oligonucleotide not in an SNA).

NMD is a mRNA quality control mechanism in eukaryotes to downregulate premature termination codons (PTC)-containing mRNAs generated by cells through errors made during gene expression and that also naturally exist or are generated as part of autoregulatory mechanisms to maintain cellular homeostasis. Although many cellular NMD targets are derived from aberrant pre-mRNA splicing and, possibly, transcription initiation, NMD also targets ˜10% of normal physiological mRNAs so as to promote an appropriate cellular response to changing environmental milieus, including those that induce apoptosis, maturation or differentiation. NMD selectively degrades mRNAs harboring PTCs to prevent the production of truncated proteins that could result in disease, such as dominantly inherited diseases due to PTC-containing mRNAs that escape NMD or to regulate the abundance of a large number of cellular RNAs. The central role of NMD in the control of gene expression requires the existence of buffering mechanisms that tightly regulate the magnitude of this pathway. A SNA disclosed herein can be used to modulate splicing such that a mRNA of interest including a PTC is produced and is degraded via NMD. (See e.g., Hug et al., Nucleic Acids Res (2016): 44(4): 1483-95, which is incorporated by reference herein in its entirety). The use of NMD can be used to treat a disease or disorder disclosed herein, such as cancer.

In some embodiments, a SNA disclosed herein induces the production of a mRNA harboring a PTC situated approximately 50-55 nucleotides or more than ˜50-55 nucleotides upstream of an exon-exon junction. (See e.g., Nagy E. et al. (1998) Trends Biochem. Sci 23:198-9). In this is the production of an mRNA harboring a PTC situated approximately at or more than 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 110, 120, 130, 140, 150, 200, 250, 300, 350, 400, 450, 500, 550, 600, 650, 700, 750, 800, 850, 900, 950, 1000 nucleotides upstream of an exon-exon junction, or any ranges or combinations thereof. In some embodiments, a SNA disclosed herein for inducing an NMD to treat the disease or disorder disclosed herein includes an ‘EJC’ mark, which is deposited ˜20-24 nucleotides upstream of ˜80% of exon-exon junctions. EJCs consist of four core components; eukaryotic translation initiation factor 4Δ3 (eIF4Δ3), cancer susceptibility candidate 3 (CASC3), RNA-binding motif protein 8A (RBM8A or Y14), and either mago-nashi homolog (MAGOH) or MAGOHB. (See e.g., Le Hir et al. EMBO J. (2000) 19(24):6860-9; Le Hir et al. EMBO J. (2001) 20(17):4987-97; Saulière et al. (2012) Nat Struct Mol Biol 19(11):1124-31; Singh et al. Cell (2012) 151(4):750-64, the contents of which are incorporated herein by reference in their entireties).

Various Pro-Inflammatory Diseases (IL-1RAP)

Several inflammatory diseases, such as rheumatoid arthritis (RA) and dermatitis, are associated with an excessive concentration of interleukin-1 (IL-1) in the plasma. IL-1 is an important mediator controlling local and systemic effects on a wide variety of target cells, there by regulating immunity and inflammation. It is believed to mediate inflammation by recruitment of neutrophils, activation of macrophages and stimulation of T and B cells. IL-1 binds to IL-1 receptor type 1 (IL-1RI), which results in the recruitment of the IL-1 receptor accessory protein (IL-1RAcP).

sIL-1RAcP can be produced by modulating IL-1RAcP splicing using antisense oligonucleotide-mediated exon skipping. The SNA constructs comprising a IL-1RAcP splice modulating antisense oligonucleotide is disclosed herein. For instance, SNAs can be used to mediate skipping of exon 9 to induce the production of a novel form of sIL-1RAcP.

Interleukin 17A Receptor (IL17RA)

Interleukin 17A (IL17A) is a proinflammatory cytokine secreted by activated T-lymphocytes. It is a potent inducer of the maturation of CD34-positive hematopoietic precursors into neutrophils. The transmembrane protein encoded by the gene interleukin 17A receptor (IL17RA; also referred to as CDw217, IL17R, Interleukin 17 Receptor, CD217 Antigen, HIL-17R, CANDF5, CD217, IMD51) is a ubiquitous type I membrane glycoprotein that binds with low affinity to interleukin 17A.

Interleukin 17A and its receptor play a pathogenic role in many inflammatory and autoimmune diseases such as rheumatoid arthritis. Like other cytokine receptors, this receptor likely has a multimeric structure. Alternative splicing results in multiple transcript variants encoding different isoforms.

Transcript variant 2 (SEQ ID NO: 93) lacks an alternate in-frame exon in the coding region, compared to variant 1. It encodes a soluble isoform, isoform 2 which is shorter and lacks a transmembrane region, when compared to isoform 1. The nucleotide sequence of SEQ ID NO: 93 includes the genomic, coding sequence and amino acid sequences of the two isoforms of IL17RA produced by alternative splicing.

Abnormalities in IL17RA are associated with diseases and disorders, such as immunodeficiency 51 (IMD51). The disease is caused by mutations in the gene. IMD51 is a primary immunodeficiency disorder with altered immune responses and impaired clearance of fungal infections, selective against Candida. It is characterized by persistent and/or recurrent infections of the skin, nails and mucous membranes caused by organisms of the genus Candida, mainly Candida albicans. (See e.g., Puel et al. (Science (2011) 332(6025):65-8. doi: 10.1126/science.1200439. Epub 2011 Feb. 24).

In some embodiments, the treatment of a disease or disorder is associated with increased IL17RA expression or activation. In some embodiments, a SNA disclosed herein is used to treat the disease or disorder associated with increased IL17RA expression or activation. In some embodiments, a SNA disclosed herein promotes exclusion of an exon in IL17RA that expresses a transmembrane region or domain in IL17RA to produce a soluble IL17RA, which is not anchored to the cell membrane. In some embodiments, the souble IL17RA variant corresponds to IL17RA isoform 2 precursor (NCBI Ref. Seq.: NP_001276834.1; UniProtKB: Q96F46-2, whose amino acid sequence is disclosed in SEQ ID NO: 93) In some embodiments, the disease or disorder is a cancer or inflammatory disorder disclosed herein, which is associated with increased IL17RA expression or activity, to be treated with a SNA disclosed herein. In some embodiments, a SNA disclosed herein decreases the ratio of a transmembrane receptor disclosed herein to a soluble variant of the receptor. (See. e.g., Gaffen et al. Nat Rev Inununol (2009) 9(8):556).

In some embodiments, the transmembrane receptor is IL1RAP or any of the IL1RAP variants disclosed herein.

According to some aspects, methods of treating a disease or disorder in a subject are contemplated herein. In some embodiments, the method comprises administering to a subject with a disease or disorder associated with abnormal transmembrane receptor activity or abnormal transmembrane receptor expression in a cell of the subject an effective amount of a spherical nucleic acid (SNA) to produce or increase the levels of a soluble variant of the transmembrane receptor in the subject relative to a subject with a disease or disorder associated with abnormal transmembrane receptor activity or abnormal transmembrane receptor expression who has not been administered a SNA or relative to a subject with a disease or disorder associated with abnormal transmembrane receptor activity or abnormal transmembrane receptor expression who has been administered a corresponding linear oligonucleotide that is not in a SNA, or relative to a baseline level in order to treat the disease or disorder in the subject.

In some embodiments, the total levels of the transmembrane receptor in the cell of the subject remains stable (not significantly changed according to statistical analysis known to one of ordinary skill in the art) or the total levels of the mRNA encoding the transmembrane receptor are not decreased through RNAse-H mediated degradation.

In some embodiments, other transmembrane receptors are also contemplated herein. Ion channel linked receptors have ion channels for anions and cations, and constitute a large family of multipass transmembrane proteins. They participate in rapid signaling events usually found in electrically active cells such as neurons. They are also called ligand-gated ion channels. Opening and closing of ion channels is controlled by neurotransmitters.

Enzyme-linked receptors are either enzymes themselves, or directly activate associated enzymes. These are typically single-pass transmembrane receptors, with the enzymatic component of the receptor kept intracellular. The majority of enzyme-linked receptors are, or associate with, protein kinases.

G protein-coupled receptors are integral membrane proteins that possess seven transmembrane helices. These receptors activate a G protein upon agonist binding, and the G-protein mediates receptor effects on intracellular signaling pathways. A SNA disclosed herein can be used to produce a soluble variant of a transmembrane receptor belonging to the families of ion channel linked receptors, enzyme-linked receptors and/or G protein-coupled receptors.

According to some aspects, methods of increasing the levels of a mRNA of interest in a cell are contemplated herein. In some embodiments, the method comprises contacting the cell with a SNA disclosed herein, wherein the levels of the mRNA of interest in the cell is increased relative to a cell that has not been contacted with the SNA or relative to a cell contacted with the corresponding linear oligonucleotide not in a SNA or relative to a baseline level.

According to some aspects, methods of inducing exon skipping in a pre-mRNA of interest in a cell are contemplated herein. In some embodiments, the method comprises contacting a cell with a SNA disclosed herein to induce exon skipping in a pre-mRNA of interest in the cell. In some embodiments, antisense oligonucleotide on a SNA induces exon skipping by binding to pre-mRNA at a site usually occupied by a regulatory splicing factor or at a site usually associated with splicing machinery. (See e.g., Bremer et al. Molecular Therapy—Nucleic Acids (2016) 5, e379); Ramsbottom et al. PNAS (2018) 115:12489-94; Wang et al. Nat Rev Genet (2007) 8(10):749-61, the contents of which are incorporated herein by reference in their entireties).

According to some aspects, methods of inducing exon inclusion in a pre-mRNA of interest in a cell are contemplated herein. In some embodiments, the method comprises contacting a cell with a SNA disclosed herein to induce exon inclusion in a pre-mRNA of interest in the cell. In some embodiments, antisense oligonucleotide on a SNA induces exon skipping by binding to pre-mRNA at a site usually occupied by a regulatory splicing factor or at a site usually associated with splicing machinery.

In some embodiments, antisense compounds are single-stranded. Such single-stranded antisense compounds typically comprise or consist of an oligomeric compound that comprises or consists of a modified oligonucleotide and optionally a conjugate group. In some embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of a modified oligonucleotide and optionally a conjugate group. The oligonucleotide of the second oligomeric compound of such double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

In some embodiments, oligomeric compounds of antisense compounds are capable of hybridizing to a target nucleic acid, resulting in at least one antisense activity. In some embodiments, antisense compounds selectively affect one or more target nucleic acid. Such selective antisense compounds comprises a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In some embodiments, hybridization of an antisense compound to a target nucleic acid results in alteration of processing, e.g., splicing, of the target precursor transcript. In some embodiments, hybridization of an antisense compound to a target precursor transcript results in inhibition of a binding interaction between the target nucleic acid and a protein or other nucleic acid. In certain such embodiments, hybridization of an antisense compound to a target precursor transcript results in alteration of translation of the target nucleic acid.

Antisense activities may be observed directly or indirectly. In some embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

In some embodiments, antisense compounds and/or oligomeric compounds comprise antisense oligonucleotides that are complementary to the target nucleic acid over the entire length of the oligonucleotide. In some embodiments, such oligonucleotides are 99% complementary to the target nucleic acid. In some embodiments, such oligonucleotides are 95% complementary to the target nucleic acid. In some embodiments, such oligonucleotides are 90% complementary to the target nucleic acid. In some embodiments, such oligonucleotides are 85% complementary to the target nucleic acid. In some embodiments, such oligonucleotides are 80% complementary to the target nucleic acid. In some embodiments, antisense oligonucleotides are at least 80% complementary to the target nucleic acid over the entire length of the oligonucleotide and comprise a region that is 100% or fully complementary to a target nucleic acid. In certain such embodiments, the region of full complementarity is from 6 to 20 nucleobases in length. In certain such embodiments, the region of full complementarity is from 10 to 18 nucleobases in length. In certain such embodiments, the region of full complementarity is from 18 to 20 nucleobases in length.

In some embodiments, oligomeric compounds and/or antisense compounds comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the antisense compound is improved. In some embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region.

In some embodiments, oligomeric compounds comprise or consist of a modified oligonucleotide that is complementary to a target precursor transcript. In certain such embodiments, the target precursor transcript is a target pre-mRNA. In some embodiments, contacting a cell with a compound complementary to a target precursor transcript modulates processing of the target precursor transcript. In certain such embodiments, the resulting target processed transcript has a different nucleobase sequence (e.g., a corrected mRNA sequence) from the target processed transcript that is produced in the absence of the compound (e.g., defective mRNA sequence). The terms “corrected mRNA” refers to a mRNA sequence that has a different nucleobase sequence from the mRNA produced in the absence of an antisense oligonucleotide in a SNA described herein, which is referred to herein as “defective mRNA”. In some embodiments, the corrected mRNA sequence produces a functional protein (e.g., due to inclusion or exclusion of an exon and/or an intron). In some embodiments, the “defective mRNA” produces a mutated or dysfunctional protein (e.g., due to erroneous inclusion or exclusion of an exon and/or an intron). In some embodiments, the mutated or dysfunctional protein is associated with a disease or disorder related to an abnormality in splice modulation.

In some embodiments, the target precursor transcript is a target pre-mRNA and contacting a cell with a compound complementary to the target pre-mRNA modulates splicing of the target pre-mRNA. In certain such embodiments, the resulting target mRNA has a different nucleobase sequence than the target mRNA that is produced in the absence of the compound. In certain such embodiments, an exon is excluded from the target mRNA. In some embodiments, an exon is included in the target mRNA. In some embodiments, the exclusion or inclusion of an exon induces or prevents nonsense mediated decay of the target mRNA, removes or adds a premature termination codon from the target mRNA, and/or changes the reading frame of the target mRNA.

As used herein, “double-stranded antisense compound” refers to an antisense compound comprising two oligomeric compounds that are complementary to each other and form a duplex, and wherein one of the two said oligomeric compounds comprises an antisense oligonucleotide.

As used herein, “hybridization” refers to the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression of activity in an untreated or control sample and does not necessarily indicate a total elimination of expression or activity. In some embodiments, total elimination of expression or activity is obtained.

As used herein, “lower”, “reduced”, “reduction” or “decrease” or “inhibit” means a decrease by at least 10% as compared to a reference sample or baseline, for example a decrease of reduction by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample or baseline), or any decrease between 10-100% as compared to a reference level. When “decrease” or “inhibition” is used in the context of the level of expression or activity of a gene or a protein, it refers to a reduction in protein or nucleic acid level or activity in a cell, a cell extract, or a cell supernatant. For example, such a decrease may be due to reduced RNA stability, transcription, or translation, increased protein degradation, or RNA interference.

As used herein, “up-regulate”, “increase” or “higher” means an increase of at least 10% as compared to a reference level, for example an increase of at least about 20%, or at least about 30%, or at least about 40%, or at least about 50%, or at least about 60%, or at least about 70%, or at least about 80%, or at least about 90% or a 100% increase or more, or any increase between 10-100% as compared to a reference sample or baseline, or an increase greater than 100%, for example, an increase at least about a 2-fold, or at least about a 3-fold, or at least about a 4-fold, or at least about a 5-fold or at least about a 10-fold increase, or any increase between 2-fold and 10-fold or greater as compared to a reference sample or baseline. When “increase” is used in the context of the expression or activity of a gene or protein, it refers to a positive change in protein or nucleic acid level or activity in a cell, a cell extract, or a cell supernatant. For example, such an increase may be due to increased RNA stability, transcription, or translation, or decreased protein degradation. Preferably, this increase is at least 5%, at least about 10%, at least about 25%, at least about 50%, at least about 75%, at least about 80%, at least about 100%, at least about 200%, or even about 500% or more over the level of expression or activity under control conditions.

An oligonucleotide disclosed herein, such as an antisense oligonucleotide, includes a strand of linked nucleosides connected via internucleoside linkages, wherein each nucleoside and internucleoside linkage may be modified or unmodified. In some embodiments, the length of an oligonucleotide described herein, such as an antisense oligonucleotide, is of 2-500 linked nucleosides. In some embodiments, the length of an oligonucleotide described herein, is of 2-200, 2-195, 2-190, 2-185, 2-180, 2-175, 2-170, 2-165, 2-160, 2-155, 2-150, 2-145, 2-140, 2-135, 2-130, 2-125, 2-120, 2-115, 2-110, 2-105, 2-100, 2-95, 2-90, 2-85, 2-80, 2-75, 2-70, 2-65, 2-60, 2-55, 2-50, 2-45, 2-40, 2-39, 2-38, 2-37, 2-36, 2-35, 2-34, 2-33, 2-32, 2-31, 2-30, 2-29, 2-28, 2-27, 2-26, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 2-7, 2-6, 2-5, 2-4, 4-200, 4-195, 4-190, 4-185, 4-180, 4-175, 4-170, 4-165, 4-160, 4-155, 4-150, 4-145, 4-140, 4-135, 4-130, 4-125, 4-120, 4-115, 4-110, 4-105, 4-100, 4-95, 4-90, 4-85, 4-80, 4-75, 4-70, 4-65, 4-60, 4-55, 4-50, 4-45, 4-40, 4-39, 4-38, 4-37, 4-36, 4-35, 4-34, 4-33, 4-32, 4-31, 4-30, 4-29, 4-28, 4-27, 4-26, 4-25, 4-24, 4-23, 4-22, 4-21, 4-20, 4-19, 4-18, 4-17, 4-16, 4-15, 4-14, 4-13, 4-12, 4-11, 4-10, 4-9, 4-8, 4-7, 4-6, 4-5, 6-200, 6-195, 2-190, 6-185, 6-180, 6-175, 6-170, 6-165, 6-160, 6-155, 6-150, 6-145, 6-140, 6-135, 6-130, 6-125, 6-120, 6-115, 6-110, 6-105, 6-100, 6-95, 6-90, 6-85, 6-80, 6-75, 6-70, 6-65, 6-60, 6-55, 6-50, 6-45, 6-40, 6-39, 6-38, 6-37, 6-36, 6-35, 6-34, 6-33, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 2-200, 8-195, 8-190, 8-185, 8-180, 8-175, 8-170, 8-165, 8-160, 8-155, 8-150, 8-145, 8-140, 8-135, 8-130, 8-125, 8-120, 8-115, 8-110, 8-105, 8-100, 8-95, 8-90, 8-85, 8-80, 8-75, 8-70, 8-65, 8-60, 8-55, 8-50, 8-45, 8-40, 8-39, 8-38, 8-37, 8-36, 8-35, 8-34, 8-33, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 2-200, 10-195, 10-190, 10-185, 10-180, 10-175, 10-170, 10-165, 10-160, 10-155, 10-150, 10-145, 10-140, 10-135, 10-130, 10-125, 10-120, 10-115, 10-110, 10-105, 10-100, 10-95, 10-90, 10-85, 10-80, 10-75, 10-70, 10-65, 10-60, 10-55, 10-50, 10-45, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, or 10-12 linked nucleosides or linked nucleosides in length.

In some embodiments, an oligonucleotide (e.g., antisense oligonucleotide) is two to 100 linked nucleosides in length, two linked nucleosides in length, three linked nucleosides in length, four linked nucleosides in length, five linked nucleosides in length, six linked nucleosides in length, seven linked nucleosides in length, eight linked nucleosides in length, nine linked nucleosides in length, 10 linked nucleosides in length, 11 linked nucleosides in length, 12 linked nucleosides in length, 13 linked nucleosides in length, 14 linked nucleosides in length, 15 linked nucleosides in length, 16 linked nucleosides in length, 17 linked nucleosides in length, 18 linked nucleosides in length, 19 linked nucleosides in length, 20 linked nucleosides in length, 21 linked nucleosides in length, 22 linked nucleosides in length, 23 linked nucleosides in length, 24 linked nucleosides in length, 25 linked nucleosides in length, 26 linked nucleosides in length, 27 linked nucleosides in length, 28 linked nucleosides in length, 29 linked nucleosides in length, 30 linked nucleosides in length, 31 linked nucleosides in length, 32 linked nucleosides in length, 33 linked nucleosides in length, 34 linked nucleosides in length, 35 linked nucleosides in length, 36 linked nucleosides in length, 37 linked nucleosides in length, 38 linked nucleosides in length, 39 linked nucleosides in length, 40 linked nucleosides in length, 41 linked nucleosides in length, 42 linked nucleosides in length, 43 linked nucleosides in length, 44 linked nucleosides in length, 45 linked nucleosides in length, 46 linked nucleosides in length, 47 linked nucleosides in length, 49 linked nucleosides in length, 50 linked nucleosides in length, 52 linked nucleosides in length, 54 linked nucleosides in length, 56 linked nucleosides in length, 58 linked nucleosides in length, 60 linked nucleosides in length, 62 linked nucleosides in length, 64 linked nucleosides in length, 66 linked nucleosides in length, 68 linked nucleosides in length, 70 linked nucleosides in length, 72 linked nucleosides in length, 74 linked nucleosides in length, 76 linked nucleosides in length, 78 linked nucleosides in length, 80 linked nucleosides in length, 82 linked nucleosides in length, 84 linked nucleosides in length, 86 linked nucleosides in length, 88 linked nucleosides in length, 90 linked nucleosides in length, 92 linked nucleosides in length, 94 linked nucleosides in length, 96 linked nucleosides in length, 100 linked nucleosides in length, or any range or combination thereof.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. In some embodiments, modified oligonucleotides having one or more modified sugar moieties at the 2′ position have enhanced pharmacologic activity for modulation of splicing of a pre-mRNA of interest, including increasing the percentage of transcripts containing a corrected mRNA sequence.

As used herein, “mismatch” or “non-complementary” means a nucleobase of a first oligonucleotide that is not complementary with the corresponding nucleobase of a second oligonucleotide or target nucleic acid when the first and second oligomeric compound are aligned. As used herein, “naturally occurring” means found in nature.

As used herein, “ameliorate” in reference to a treatment improvement in at least one symptom relative to the same symptom in the absence of the treatment. In some embodiments, the treatment is of a neurodegenerative disorder described herein, such as treatment of disease or disorder disclosed in Table 1. In some embodiments, amelioration is the reduction in the severity or frequency of a symptom or the delayed onset or slowing of progression in the severity or frequency of a symptom associated with a disease or disorder described herein.

As used herein, a “cell-targeting moiety” refers to a conjugate group or portion of a conjugate group that results in improved uptake to a particular cell type and/or distribution to a particular tissue relative to an oligomeric compound lacking the cell-targeting moiety.

As used herein, “complementary” to an oligonucleotide described means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to an oligonucleotide described herein means that such oligonucleotides are complementary to another oligonucleotide or nucleic acid at each nucleoside of the oligonucleotide.

As used herein, the terms “internucleoside linkage” refers to a group or bond that forms a covalent linkage between adjacent nucleosides in an oligonucleotide. As used herein, a “modified internucleoside linkage” refers to any internucleoside linkage other than a naturally occurring, phosphate internucleoside linkage or phosphodiester linkage. Non-phosphate linkages are referred to herein as modified internucleoside linkages.

In some embodiments, the internucleoside linkage is a phosphorothioate linkage. As used herein, “phosphorothioate linkage” refers to a modified phosphate linkage in which one of the non-bridging oxygen atoms is replaced with a sulfur atom. A phosphorothioate internucleoside linkage is a modified internucleoside linkage. In some embodiments, all or 100% of the internucleoside linkages of an antisense oligonucleotide described herein are phosphodiesters. In some embodiments, less than all or less than 100% of the internucleoside linkages of an antisense oligonucleotide described herein are phosphodiester linkages. In some embodiments, 5-20%, 5-50%, 5-75%, 5-100%, 10-20%, 10-50%, 10-75% or 10-100% of the internucleoside linkages are phosphodiester linkages. In some embodiments, less than all of the internucleoside linkages in the antisense oligonucleotide are phosphodiester linkages. In some embodiments, all of the internucleoside linkages in the antisense oligonucleotide are phosphodiester linkages. In some embodiments, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 99% of the internucleoside linkages in the antisense oligonucleotide are phosphodiester linkages.

In some embodiments, one of the internucleoside linkages, two of the internucleoside linkages, three of the internucleoside linkages, four of the internucleoside linkages, five of the internucleoside linkages, six of the internucleoside linkages, seven of the internucleoside linkages, eight of the internucleoside linkages, nine of the internucleoside linkages, 10 of the internucleoside linkages, 11 of the internucleoside linkages, 12 of the internucleoside linkages, 13 of the internucleoside linkages, 14 of the internucleoside linkages, 15 of the internucleoside linkages, 16 of the internucleoside linkages, 17 of the internucleoside linkages, 18 of the internucleoside linkages, 19 of the internucleoside linkages, 20 of the internucleoside linkages, 21 of the internucleoside linkages, 22 of the internucleoside linkages, 23 of the internucleoside linkages, 24 of the internucleoside linkages, 25 of the internucleoside linkages, 26 of the internucleoside linkages, 27 of the internucleoside linkages, 28 of the internucleoside linkages, 29 of the internucleoside linkages, 30 of the internucleoside linkages, 31 of the internucleoside linkages, 32 of the internucleoside linkages, 33 of the internucleoside linkages, 34 of the internucleoside linkages, 35 of the internucleoside linkages, 36 of the internucleoside linkages, 37 of the internucleoside linkages, 38 of the internucleoside linkages, 39 of the internucleoside linkages, 40 of the internucleoside linkages, 41 of the internucleoside linkages, 42 of the internucleoside linkages, 43 of the internucleoside linkages, 44 of the internucleoside linkages, 45 of the internucleoside linkages, 46 of the internucleoside linkages, 47 of the internucleoside linkages, 49 of the internucleoside linkages, 50 of the internucleoside linkages, 52 of the internucleoside linkages, 54 of the internucleoside linkages, 56 of the internucleoside linkages, 58 of the internucleoside linkages, 60 of the internucleoside linkages, 62 of the internucleoside linkages, 64 of the internucleoside linkages, 66 of the internucleoside linkages, 68 of the internucleoside linkages, 70 of the internucleoside linkages, 72 of the internucleoside linkages, 74 of the internucleoside linkages, 76 of the internucleoside linkages, 78 of the internucleoside linkages, 80 of the internucleoside linkages, 82 of the internucleoside linkages, 84 of the internucleoside linkages, 86 of the internucleoside linkages, 88 of the internucleoside linkages, 90 of the internucleoside linkages, 92 of the internucleoside linkages, 94 of the internucleoside linkages, 96 of the internucleoside linkages, 100 nucleotides or more than 100 of the internucleoside linkages, or any range or combination thereof of an antisense oligonucleotide described herein are phosphodiester linkages.

In some embodiments, 5-20%, 5-50%, 5-75%, 5-100%, 10-20%, 10-50%, 10-75% or 10-100% of the internucleoside linkages of an antisense oligonucleotide described herein are phosphorothioate linkages. In some embodiments, all of the internucleoside linkages in the antisense oligonucleotide are phosphorothioate linkages. In some embodiments, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 99% of the internucleoside linkages in the antisense oligonucleotide are phosphorothioate linkages. In some embodiments, one of the internucleoside linkages, two of the internucleoside linkages, three of the internucleoside linkages, four of the internucleoside linkages, five of the internucleoside linkages, six of the internucleoside linkages, seven of the internucleoside linkages, eight of the internucleoside linkages, nine of the internucleoside linkages, 10 of the internucleoside linkages, 11 of the internucleoside linkages, 12 of the internucleoside linkages, 13 of the internucleoside linkages, 14 of the internucleoside linkages, 15 of the internucleoside linkages, 16 of the internucleoside linkages, 17 of the internucleoside linkages, 18 of the internucleoside linkages, 19 of the internucleoside linkages, 20 of the internucleoside linkages, 21 of the internucleoside linkages, 22 of the internucleoside linkages, 23 of the internucleoside linkages, 24 of the internucleoside linkages, 25 of the internucleoside linkages, 26 of the internucleoside linkages, 27 of the internucleoside linkages, 28 of the internucleoside linkages, 29 of the internucleoside linkages, 30 of the internucleoside linkages, 31 of the internucleoside linkages, 32 of the internucleoside linkages, 33 of the internucleoside linkages, 34 of the internucleoside linkages, 35 of the internucleoside linkages, 36 of the internucleoside linkages, 37 of the internucleoside linkages, 38 of the internucleoside linkages, 39 of the internucleoside linkages, 40 of the internucleoside linkages, 41 of the internucleoside linkages, 42 of the internucleoside linkages, 43 of the internucleoside linkages, 44 of the internucleoside linkages, 45 of the internucleoside linkages, 46 of the internucleoside linkages, 47 of the internucleoside linkages, 49 of the internucleoside linkages, 50 of the internucleoside linkages, 52 of the internucleoside linkages, 54 of the internucleoside linkages, 56 of the internucleoside linkages, 58 of the internucleoside linkages, 60 of the internucleoside linkages, 62 of the internucleoside linkages, 64 of the internucleoside linkages, 66 of the internucleoside linkages, 68 of the internucleoside linkages, 70 of the internucleoside linkages, 72 of the internucleoside linkages, 74 of the internucleoside linkages, 76 of the internucleoside linkages, 78 of the internucleoside linkages, 80 of the internucleoside linkages, 82 of the internucleoside linkages, 84 of the internucleoside linkages, 86 of the internucleoside linkages, 88 of the internucleoside linkages, 90 of the internucleoside linkages, 92 of the internucleoside linkages, 94 of the internucleoside linkages, 96 of the internucleoside linkages, 100 nucleotides or more than 100 of the internucleoside linkages, or any range or combination thereof of an antisense oligonucleotide described herein are phosphorothioate linkages.

As used herein, “phosphodiester internucleoside linkage” means a phosphate group that is covalently bonded to two adjacent nucleosides of a modified oligonucleotide.

In some embodiments, an antisense oligonucleotide described herein is attached or inserted in to the surface of the lipid-containing core through conjugation to one or more linker or linker moieties. Non-limiting examples of linker or linker moieties contemplated herein include: tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrianosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

A spherical nucleic acid (SNA) can be functionalized in order to attach a polynucleotide. Alternatively or additionally, the polynucleotide can be functionalized. One mechanism for functionalization is the alkanethiol method, whereby oligonucleotides are functionalized with alkanethiols at their 3′ or 5′ termini prior to attachment to gold nanoparticles or nanoparticles comprising other metals, semiconductors or magnetic materials. Such methods are described, for example Whitesides, Proceedings of the Robert A. Welch Foundation 39th Conference On Chemical Research Nanophase Chemistry, Houston. Tex., pages 109-121 (1995), and Mucic et al. Chem. Commun. 555-557 (1996). Oligonucleotides can also be attached to nanoparticles using other functional groups such as phosophorothioate groups, as described in and incorporated by reference from U.S. Pat. No. 5,472,881, or substituted alkylsiloxanes, as described in and incorporated by reference from Burwell, Chemical Technology, 4, 370-377 (1974) and Matteucci and Caruthers, J. Am. Chem. Soc., 103, 3185-3191 (1981). In some instances, polynucleotides are attached to nanoparticles by terminating the polynucleotide with a 5′ or 3′ thionucleoside. In other instances, an aging process is used to attach polynucleotides to nanoparticles as described in and incorporated by reference from U.S. Pat. Nos. 6,361,944, 6,506,569, 6,767,702 and 6,750,016 and PCT Publication Nos. WO 1998/004740, WO 2001/000876. WO 2001/051665 and WO 2001/073123.

In some instances, the oligonucleotide is attached or inserted in the SNA. A spacer can be included between the attachment site and the oligonucleotide. In some embodiments, a spacer comprises or consists of an oligonucleotide, a peptide, a polymer or an oligoethylene glycol. In a preferred embodiment, the spacer is oligoethylene glycol and more preferably, hexaethyleneglycol. In some embodiments, a spacer does not comprise or does not consist of an oligonucleotide (e.g., non-nucleotidic linker), a peptide, a polymer or an oligoethylene.

As used herein, “precursor transcript” means a coding or non-coding RNA that undergoes processing to form a processed or mature form of the transcript. Precursor transcripts include but are not limited to pre-mRNAs, long non-coding RNAs, pri-miRNAs, and intronic RNAs.

As used herein, “processing” in reference to a precursor transcript means the conversion of a precursor transcript to form the corresponding processed transcript. Processing of a precursor transcript includes but is not limited to nuclease cleavage events at processing sites of the precursor transcript.

The terms “oligonucleotide”, “polynucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis). In some embodiments, an oligonucleotide is an antisense oligonucleotide.

A polynucleotide of the SNA and optionally attached to a SNA core can be single stranded or double stranded. A double stranded polynucleotide is also referred to herein as a duplex. Double-stranded oligonucleotides of the invention can comprise two separate complementary nucleic acid strands.

As used herein, “duplex” includes a double-stranded nucleic acid molecule(s) in which complementary sequences are hydrogen bonded to each other. The complementary sequences can include a sense strand and an antisense strand. The antisense nucleotide sequence can be identical or sufficiently identical to the target gene to mediate effective target gene inhibition (e.g., at least about 98% identical, 96% identical, 94%, 90% identical, 85% identical, or 80% identical) to the target gene sequence.

A double-stranded polynucleotide can be double-stranded over its entire length, meaning it has no overhanging single-stranded sequences and is thus blunt-ended. In other embodiments, the two strands of the double-stranded polynucleotide can have different lengths producing one or more single-stranded overhangs. A double-stranded polynucleotide of the invention can contain mismatches and/or loops or bulges. In some embodiments, it is double-stranded over at least about 70%, 80%, 90%, 95%, 96%, 97%, 98% or 99% of the length of the oligonucleotide. In some embodiments, the double-stranded polynucleotide of the invention contains at least or up to 1, 2, 3, 4, 5, 6, 7, 8, 9, 10, 11, 12, 13, 14, or 15 mismatches.

Polynucleotides associated with the invention can be modified such as at the sugar moiety, the phosphodiester linkage, and/or the base. As used herein, “sugar moieties” includes natural, unmodified sugars, including pentose, hexose, conformationally flexible sugars, conformationally locked sugars, arabinose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2-O-methyl nucleotides, which are referred to as “methylated.” In some instances, polynucleotides associated with the invention may only contain modified or unmodified sugar moieties, while in other instances, polynucleotides contain some sugar moieties that are modified and some that are not. In some embodiments, all of the nucleotides in the oligonucleotide (e.g., antisense oligonucleotide) include a 2′O methyl modification. In some embodiments, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 99% of the nucleotides include a 2′O methyl modification.

In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′-OH group replaced by an H, alkoxy (or OR), R or alkyl, halogen, SH, SR, amino (such as NH2, NHR, NR2,), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

In some aspects, 2′-O-methyl modifications can be beneficial for reducing undesirable cellular stress responses, such as the interferon response to double-stranded nucleic acids. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH2CH═CH2), 2′-propargyl, 2′-propyl, ethynyl, ethenyl, propenyl, and cyano and the like. The sugar moiety can also be a hexose or arabinose.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In some embodiments, a straight chain or branched chain alkyl has 6 or fewer carbon atoms in its backbone (e.g., C1-C6 for straight chain, C3-C6 for branched chain), and more preferably 4 or fewer. Likewise, preferred cycloalkyls have from 3-8 carbon atoms in their ring structure, and more preferably have 5 or 6 carbons in the ring structure. The term C1-C6 includes alkyl groups containing 1 to 6 carbon atoms. Unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In some embodiments, a straight chain or branched chain alkenyl group has 6 or fewer carbon atoms in its backbone (e.g., C2-C6 for straight chain, C3-C6 for branched chain). Likewise, cycloalkenyl groups may have from 3-8 carbon atoms in their ring structure, and more preferably have 5 or 6 carbons in the ring structure. The term C2-C6 includes alkenyl groups containing 2 to 6 carbon atoms.

Unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “hydrophobic modifications” refers to modification of bases such that overall hydrophobicity is increased and the base is still capable of forming close to regular Watson-Crick interactions. Non-limiting examples of base modifications include 5-position uridine and cytidine modifications like phenyl, 4-pyridyl, 2-pyridyl, indolyl, and isobutyl, phenyl (C6H5OH); tryptophanyl (C8H6N)CH2CH(NH2)CO), Isobutyl, butyl, aminobenzyl; phenyl; naphthyl,

The term “heteroatom” includes atoms of any element other than carbon or hydrogen. In some embodiments, preferred heteroatoms are nitrogen, oxygen, sulfur and phosphorus. The term “hydroxy” or “hydroxyl” includes groups with an —OH or —O— (with an appropriate counterion). The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)0-3NR′R″, (CR′R″)0-3CN, NO2, halogen, (CR′R″)0-3C(halogen)3, (CR′R″)0-3CH(halogen)2, (CR′R″)0-3CH2(halogen), (CR′R″)0-3CONR′R″, (CR′R″)0-3S(O)0-2NR′R″, (CR′R″)0-3CHO, (CR′R″)0-30(CR′R″)0-3H, (CR′R″)0-3S(O)0-2R′, (CR′R″)0-30(CR′R″)0-3H, (CR′R″)0-3COR′, (CR′R″)0-3CO2R′, or (CR′R″)0-30R′ groups; wherein each R′ and R″ are each independently hydrogen, a C1-C5 alkyl, C2-C5 alkenyl, C2-C5 alkynyl, or aryl group, or R′ and R″ taken together are a benzylidene group or a —(CH2)2O(CH2)2-group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N6-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In some aspects, the nucleomonomers of a polynucleotide of the invention are RNA nucleotides, including modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2nd Ed., Wiley-Interscience, New York, 1999).

The term “nucleotide” includes nucleosides which further comprise a phosphate group or a phosphate analog.

As used herein, the term “linkage” includes a naturally occurring, unmodified phosphodiester moiety (—O—(PO2-)-O—) that covalently couples adjacent nucleoside monomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In some embodiments, non-hydrolysable linkages are preferred, such as phosphorothioate linkages.

In some aspects, polynucleotides of the invention comprise 3′ and 5′ termini (except for circular oligonucleotides). The 3′ and 5′ termini of a polynucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH2-CH2-CH3), glycol (—O—CH2-CH2-O—) phosphate (PO32-), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linker or linker moieties, amino linker or linker moieties, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′-+3′ internucleotide linkage. For example, the alkyloxy radical can be methoxy, ethoxy, or isopropoxy, and preferably, ethoxy. Optionally, the 3′→3′linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

In some embodiments, modified oligonucleotides comprise one or more nucleoside comprising an unmodified nucleobase. In some embodiments, modified oligonucleotides comprise one or more nucleoside comprising a modified nucleobase.

In some embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In some embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

In some embodiments, oligonucleotides comprise modified and/or unmodified nucleobases arranged along the oligonucleotide or region thereof in a defined pattern or motif. In some embodiments, each nucleobase is modified. In some embodiments, none of the nucleobases are modified. In some embodiments, each purine or each pyrimidine is modified. In some embodiments, each adenine is modified. In some embodiments, each guanine is modified. In some embodiments, each thymine is modified. In some embodiments, each uracil is modified. In some embodiments, each cytosine is modified. In some embodiments, some or all of the cytosine nucleobases in a modified oligonucleotide are 5-methylcytosines.

In some embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In some embodiments, the block is at the 5′-end of the oligonucleotide. In certain embodiments the block is within 3 nucleosides of the 5′-end of the oligonucleotide.

In some embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In some embodiments, the modified nucleobase is selected from: a 2-thiopyrimidine and a 5-propynepyrimidine.

In some aspects, polynucleotides can comprise both DNA and RNA.

In some aspects, at least a portion of the contiguous polynucleotides are linked by a substitute linkage, e.g., a phosphorothioate linkage. The presence of substitute linkages can improve pharmacokinetics due to their higher affinity for serum proteins.

In some embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P=0”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH2-N(CH3)-0-CH2-), thiodiester, thionocarbamate (-0-C(=0)(NH)—S—); siloxane (—O—S1H2-O—); and N,N′-dimethylhydrazine (—CH2-N(CH3)-N(CH3)-). Modified internucleoside linkages, compared to naturally occurring phosphate linkages, can be used to alter, typically increase, nuclease resistance of the oligonucleotide. In some embodiments, internucleoside linkages having a chiral atom can be prepared as a racemic mixture, or as separate enantiomers. Representative chiral internucleoside linkages include but are not limited to alkylphosphonates and phosphorothioates.

Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH2-N(CH3)-0-5′), amide-3 (3′-CH2-C(═O)—N(H)-5′), amide-4 (3′-CH2-N(H)—C(=0)-5′), formacetal (3′-0-CH2-0-5′), methoxypropyl, and thioformacetal (3′-S-CH2-0-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research: Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In some embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

In some embodiments, modified oligonucleotides comprise one or more modified nucleoside comprising a modified sugar. In some embodiments, modified oligonucleotides comprise one or more modified nucleosides comprising a modified nucleobase. In some embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In some embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

In some embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In some embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) are modified sugar moieties and differ from the sugar moieties of the neighboring gap nucleosides, which are unmodified sugar moieties, thus defining the boundary between the wings and the gap (i.e., the wing/gap junction). In some embodiments, the sugar moieties within the gap are the same as one another. In some embodiments, the gap includes one or more nucleoside having a sugar moiety that differs from the sugar moiety of one or more other nucleosides of the gap. In some embodiments, the sugar motifs of the two wings are the same as one another (symmetric gapmer). In some embodiments, the sugar motif of the 5′-wing differs from the sugar motif of the 3′-wing (asymmetric gapmer).

In some embodiments, the wings of a gapmer comprise 1-5 nucleosides. In some embodiments, the wings of a gapmer comprise 2-5 nucleosides. In some embodiments, the wings of a gapmer comprise 3-5 nucleosides. In some embodiments, the nucleosides of a gapmer are all modified nucleosides.

In some embodiments, the gap of a gapmer comprises 7-12 nucleosides. In some embodiments, the gap of a gapmer comprises 7-10 nucleosides. In some embodiments, the gap of a gapmer comprises 8-10 nucleosides. In some embodiments, the gap of a gapmer comprises 10 nucleosides. In certain embodiment, each nucleoside of the gap of a gapmer is an unmodified 2′-deoxy nucleoside.

In some embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In some embodiments, modified oligonucleotides comprise or consist of a region having a fully modified sugar motif. In such embodiments, each nucleoside of the fully modified region of the modified oligonucleotide comprises a modified sugar moiety. In certain such embodiments, each nucleoside in the entire modified oligonucleotide comprises a modified sugar moiety. In some embodiments, modified oligonucleotides comprise or consist of a region having a fully modified sugar motif, wherein each nucleoside within the fully modified region comprises the same modified sugar moiety, referred to herein as a uniformly modified sugar motif. In some embodiments, a fully modified oligonucleotide is a uniformly modified oligonucleotide. In some embodiments, each nucleoside of a uniformly modified oligonucleotide comprises the same 2′-modification. In some embodiments, each nucleoside of a uniformly modified oligonucleotide comprises a 2′-0-(N-alkyl acetamide) group. In some embodiments, each nucleoside of a uniformly modified oligonucleotide comprises a 2′-0-(N-methyl acetamide) group.

In some embodiments, the invention provides oligomeric compounds, which consist of an oligonucleotide (modified or unmodified) and optionally one or more conjugate groups and/or terminal groups. Conjugate groups consist of one or more conjugate moiety and a conjugate linker or linker moiety which links the conjugate moiety to the oligonucleotide. Conjugate groups may be attached to either or both ends of an oligonucleotide and/or at any internal position. In some embodiments, conjugate groups are attached to the 2′-position of a nucleoside of a modified oligonucleotide. In some embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In some embodiments, conjugate groups are attached near the 3′-end of oligonucleotides. In some embodiments, conjugate groups (or terminal groups) are attached at the 5′-end of oligonucleotides. In some embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

In some embodiments, oligonucleotides are covalently attached to one or more conjugate groups. In some embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In some embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-0-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In some embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In some embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In some embodiments, conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In some embodiments, a conjugate moiety comprises an active drug substance, for example, aspirin, warfarin, phenylbutazone, ibuprofen, suprofen, fen-bufen, ketoprofen, (<S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In some embodiments, antisense oligonucleotide SNAs are nanoscale constructs composed of: (1) a lipid-containing core, which is formed by arranging non-toxic carrier lipids into a small hollow structure, (2) a shell of oligonucleotides, which is formed by arranging oligonucleotides such that they point radially outwards from the core, and (3) optionally a hydrophobic (e.g. lipid) anchor group, or molecular species as also referred to herein, attached to either the 5′- or 3′-end of the oligonucleotide, depending on whether the oligonucleotides are arranged with the 5′- or 3′-end facing outward from the core. The anchor or molecular species drives the insertion into the liposome and to anchor the oligonucleotides to the lipid-containing core or lipid bilayer.

A liposomal core as used herein refers to a centrally located core compartment formed by a component of the lipids or phospholipids that form a lipid bilayer. “Liposomes” are artificial, self-closed vesicular structure of various sizes and structures, where one or several membranes encapsulate an aqueous core. Most typically liposome membranes are formed from lipid bilayers membranes, where the hydrophilic head groups are oriented towards the aqueous environment and the lipid chains are embedded in the lipophilic core. Liposomes can be formed as well from other amphiphilic monomeric and polymeric molecules, such as polymers, like block copolymers, or polypeptides. Unilamellar vesicles are liposomes defined by a single membrane enclosing an aqueous space. In contrast, oligo- or multilamellar vesicles are built up of several membranes. Typically, the membranes are roughly 4 nm thick and are composed of amphiphilic lipids, such as phospholipids, of natural or synthetic origin. Optionally, the membrane properties can be modified by the incorporation of other lipids such as sterols or cholic acid derivatives.

The lipid bilayer is composed of two layers of lipid molecules. Each lipid molecule in a layer is oriented substantially parallel to adjacent lipid bilayers, and two layers that form a bilayer have the polar ends of their molecules exposed to the aqueous phase and the non-polar ends adjacent to each other. The central aqueous region of the liposomal core may be empty or filled fully or partially with water, an aqueous emulsion, oligonucleotides, or other therapeutic or diagnostic agent.

The lipid-containing core or the lipid bilayer of a liposomal core can be constructed from a wide variety of lipids known to those in the art including but not limited to: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, 1,2-dioleoyl-sn-glycero-3-phosphocholine (DOPC) and polyunsaturated sterols of different lengths, saturation states, and their derivatives.

In some embodiments, the oligonucleotides may be positioned on the exterior of the core, within the walls of the core and/or in the center of the core. An oligonucleotide that is positioned on the core is typically referred to as coupled to the core. Coupled may be direct or indirect. In some embodiments at least 5, 10, 15, 25, 50, 75, 100, 200, 300, 400, 500, 600, 700, 800, 900, 1000, 5000 or 10,000 oligonucleotides or any range combination thereof are on the exterior of the core. In some embodiments, 1-1000, 10-500, 50-250, or 50-300 oligonucleotides are present on the surface.

The oligonucleotides of the oligonucleotide shell may be oriented in a variety of directions. In some embodiments the oligonucleotides are oriented radially outwards. The orientation of these oligonucleotides can be either 5′ distal/3′ terminal in relation to the core, or 3′ distal/5′terminal in relation to the core, or laterally oriented around the core. In one embodiment one or a multiplicity of different oligonucleotides are present on the same surface of a single SNA. In all cases, at least 1 oligonucleotide is present on the surface but up to 10,000 can be present.

In some embodiments, an oligonucleotide or antisense oligonucleotide disclosed herein comprises a linker moiety. The oligonucleotides may be linked or attached to the core or to a lipid bilayer or to one another and/or to other molecules such an active agents either directly or indirectly through a linker or linker moiety. The oligonucleotides may be conjugated to a linker or linker moiety via the 5′ end or the 3′ end. Some or all of the oligonucleotides of the SNA may be linked to one another or the core either directly or indirectly through a covalent or non-covalent linkage. The linkage of one oligonucleotide to another oligonucleotide may be in addition to or alternatively to the linkage of that oligonucleotide to a liposomal core.

In some embodiments, the linker moiety comprises a molecular species at the 3′ or 5′ terminus of the antisense oligonucleotide, wherein the molecular species is positioned in the liposomal core and the antisense oligonucleotide extends radially from the liposome core.

In some embodiments, the molecular species is at the 5′ terminus of the antisense oligonucleotide. In some embodiments, the molecular species is attached to the linker moiety.

In some embodiments, the molecular species is positioned in a core and the oligonucleotide extends radially from the core. In some embodiments, the molecular species is at the 5′ end of the oligonucleotide (e.g., antisense oligonucleotide). In some embodiments, the molecular species is a hydrophobic group. In some embodiments, the hydrophobic group is selected from the group consisting of cholesterol, a cholesteryl or modified cholesteryl residue, stearyl, distearyl, tocopherol, adamantine, dihydrotesterone, long chain alkyl, long chain alkenyl, long chain alkynyl, olely-lithocholic, cholenic, oleoyl-cholenic, decane, dodecane, docosahexaenoyl, palmityl, C6-palmityl, heptadecyl, myrisityl, arachidyl, stearyl, behenyl, linoleyl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen.

The oligonucleotide shell may be anchored to the surface of the core through one or multiple of linker or linker moiety molecules, including but not limited to: any chemical structure containing one or multiple thiols, such as the various chain length alkane thiols, cyclic dithiol, lipoic acid, or other thiol linkers or linker moieties known to those skilled in the art. In some embodiments, the exterior of the lipid-containing core has an oligonucleotide shell. The oligonucleotide shell can be constructed from a wide variety of nucleic acids including, but not limited to: single-stranded deoxyribonucleotides, ribonucleotides, and other single-stranded oligonucleotides incorporating one or a multiplicity of modifications known to those in the art, double-stranded deoxyribonucleotides, ribonucleotides, and other double-stranded oligonucleotides incorporating one or a multiplicity of modifications known to those in the art, oligonucleotide triplexes incorporating deoxyribonucleotides, ribonucleotides, or oligonucleotides that incorporate one or a multiplicity of modifications known to those in the art. The SNAs described herein are constructed from oligonucleotides that are not as potent on their own. The surface density of the oligonucleotides may depend on the size and type of the core and on the length, sequence and concentration of the oligonucleotides. A surface density adequate to make the nanoparticles or SNA stable and the conditions necessary to obtain it for a desired combination of nanoparticles and oligonucleotides can be determined empirically. In some embodiments, the surface density is about 5, 10, 15, 20, 25, 30, 35, 40, 45, 50, 55, 60, 65, 70, 75, 80, 85, 100, 200, 300, 400, 500, 600, 700, 800, 900, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 1 to 17, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 196, 197, 198, 199, 200, 201, 202, 203, 204, 205, 210, 215, 220, 225, 230, 235, 240, 245, 250, 300, 350, 400, 450, 500, 550, 600, 650, 700, 750, 800, 850, 900, 950, 1,000, 1,200, 1,400, 1,600, 1,800, 2,000, 3,000, 4,000, 5,000, 6,000, 7,000, 8,000, 9,000 or 10,000 oligonucleotides (e.g., antisense oligonucleotides) or any range combination thereof per SNA. In some embodiments, the surface density is at least 5, 10, 15, 20, 25, 30, 35, 40, 45, 50, 55, 60, 65, 70, 75, 80, 85, 100, 200, 300, 400, 500, 600, 700, 800, 900, 1,000, 1,200, 1,400, 1,600, 1,800, 2,000, 3,000, 4,000, 5,000, 6,000, 7,000, 8,000, 9,000 or 10.000 oligonucleotides or any range combination thereof per SNA. In some embodiments, the surface density is 1-10,000, 1-9,000, 1-8,000, 1-7,000, 1-6,000, 1-5,000, 1-4,000, 1-3,000, 1-2,000, 1-1,000, 5-10,000, 5-9,000, 5-8,000, 5-7,000, 5-6,000, 5-5,000, 5-4,000, 5-3,000, 5-2,000, 5-1,000, 100-10,000, 100-9,000, 100-8,000, 100-7,000, 100-6,000, 100-5,000, 100-4,000, 100-3,000, 100-2,000, 100-1,000, 500-10,000, 500-9,000, 500-8,000, 500-7,000, 500-6,000, 500-5,000, 500-4,000, 500-3,000, 500-2,000, 500-1,000, 10-10,000, 10-500, 50-10,000, 50-300, or 50-250 oligonucleotides per SNA. In some embodiments, the oligonucleotide shell has a surface density of at least 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 oligonucleotides per SNA. In some embodiments, the oligonucleotide shell has a surface density of about 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 oligonucleotides per SNA. It will be recognized that the oligonucleotide shell surface density can be expressed as molar ratio of oligonucleotides to lipid which forms the liposome core. In certain embodiments, the lipid to oligonucleotide ratio is 1000:1, 900:1, 800:1, 700:1, 600:1, 500:1, 400:1, 300:1, 250:1, 200:1, 150:1, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, and 5:1.

In some embodiments, the surface density is at least 5, 10, 15, 20, 25, 30, 35, 40, 45, 50, 55, 60, 65, 70, 75, 80, 85, 100, 200, 300, 400, 500, 600, 700, 800, 900, 1,000, 1,200, 1,400, 1,600, 1,800, 2,000, 3,000, 4,000, 5,000, 6,000, 7,000, 8,000, 9,000 or 10,000 oligonucleotides or any range combination thereof per 20 nm liposome. In some embodiments, the surface density is 1-10,000, 1-9,000, 1-8,000, 1-7,000, 1-6,000, 1-5,000, 1-4,000, 1-3,000, 1-2,000, 1-1,000, 5-10,000, 5-9,000, 5-8,000, 5-7,000, 5-6,000, 5-5,000, 5-4,000, 5-3,000, 5-2,000, 5-1,000, 100-10,000, 100-9,000, 100-8,000, 100-7,000, 100-6,000, 100-5,000, 100-4,000, 100-3,000, 100-2,000, 100-1,000, 500-10,000, 500-9,000, 500-8,000, 500-7,000, 500-6,000, 500-5,000, 500-4,000, 500-3,000, 500-2,000, 500-1,000, 10-10,000, 10-500, 50-10,000, 50-300, or 50-250 oligonucleotides per 20 nm liposome.

In some embodiments, the core has a diameter of or about 5 nm to about 150 nm. In some embodiments, the core has a diameter of or about 5 nm, of or about 6 nm, of or about 7 nm, of or about 8 nm, of or about 9 nm, of or about 10 nm, of or about 11 nm, of or about 12 nm, of or about 13 nm, of or about 14 nm, of or about 15 nm, of or about 16 nm, of or about 17 nm, of or about 18 nm, of or about 19 nm, of or about 20 nm, of or about 21 nm, of or about 22 nm, of or about 23 nm, of or about 24 nm, of or about 25 nm, of or about 26 nm, of or about 27 nm, of or about 28 nm, of or about 29 nm, of or about 30 nm, of or about 31 nm, of or about 32 nm, of or about 33 nm, of or about 34 nm, of or about 35 nm, of or about 36 nm, of or about 37 nm, of or about 38 nm, of or about 39 nm, of or about 40 nm, of or about 41 nm, of or about 42 nm, of or about 43 nm, of or about 44 nm, of or about 45 nm, of or about 46 nm, of or about 47 nm, of or about 48 nm, of or about 49 nm, of or about 50 nm, of or about 55 nm, of or about 60 nm, of or about 65 nm, of or about 70 nm, of or about 75 nm, of or about 80 nm, of or about 85 nm, of or about 90 nm, of or about 95 nm, of or about 100 nm, of or about 110 nm, of or about 120 nm, of or about 130 nm, of or about 140 nm, of or about 150 nm, of or about 160 nm, of or about 170 nm, of or about 180 nm, of or about 190 nm, of or about 200 nm, of or about 210 nm, of or about 220 nm, of or about 230 nm, of or about 240 nm, of or about 250 nm, of or about 260 nm, of or about 270 nm, of or about 280 nm, of or about 290 nm, of or about 300 nm, of more than about 300 nm, of about 15 nm to about 100 nm, of about 20 nm to about 100 nm, of about 25 nm to about 100 nm, of about 15 nm to about 50 nm, of about 20 nm to about 50 nm, of about 10 nm to about 70 nm, of about 15 nm to about 70 nm, of about 20 nm to about 70 nm, of about 10 nm to about 30 nm, of about 15 nm to about 30 nm, of about 20 nm to about 30 nm, of about 10 nm to about 40 nm, of about 15 nm to about 40 nm, of about 20 nm to about 40 nm, of about 10 nm to about 80 nm, of about 15 nm to about 80 nm, or of about 20 nm to about 80 nm.

In some embodiments, a SNA described herein has an average diameter on the order of nanometers (i.e., between about 1 nm and about 1 micrometer). For example, in some instances, the diameter of the nanoparticle is from about I nm to about 250 nm in mean diameter, about I nm to about 240 nm in mean diameter, about 1 nm to about 230 nm in mean diameter, about 1 nm to about 220 nm in mean diameter, about I nm to about 210 nm in mean diameter, about I nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about I nm to about 140 nm in mean diameter, about 1 nm to about 130 nm in mean diameter, about I nm to about 120 nm in mean diameter, about 1 nm to about 110 nm in mean diameter, about I nm to about 100 nm in mean diameter, about 1 nm to about 90 nm in mean diameter, about I nm to about 80 nm in mean diameter, about I nm to about 70 nm in mean diameter, about I nm to about 60 nm in mean diameter, about 1 nm to about 50 nm in mean diameter, about 1 nm to about 40 nm in mean diameter, about I nm to about 30 nm in mean diameter, about I nm to about 20 nm in mean diameter, about 1 nm to about 10 nm in mean diameter, about 5 nm to about 150 nm in mean diameter, about 5 to about 50 nm in mean diameter, about 10 to about 30 nm in mean diameter, about 10 to 150 nm in mean diameter, about 10 to about 100 nm in mean diameter, about 10 to about 50 nm in mean diameter, about 30 to about 100 nm in mean diameter, or about 40 to about 80 nm in mean diameter.

In some embodiments, the SNA has a diameter of or about 5 nm, of or about 6 nm, of or about 7 nm, of or about 8 nm, of or about 9 nm, of or about 10 nm, of or about 11 nm, of or about 12 nm, of or about 13 nm, of or about 14 nm, of or about 15 nm, of or about 16 nm, of or about 17 nm, of or about 18 nm, of or about 19 nm, of or about 20 nm, of or about 21 nm, of or about 22 nm, of or about 23 nm, of or about 24 nm, of or about 25 nm, of or about 26 nm, of or about 27 nm, of or about 28 nm, of or about 29 nm, of or about 30 nm, of or about 31 nm, of or about 32 nm, of or about 33 nm, of or about 34 nm, of or about 35 nm, of or about 36 nm, of or about 37 nm, of or about 38 nm, of or about 39 nm, of or about 40 nm, of or about 41 nm, of or about 42 nm, of or about 43 nm, of or about 44 nm, of or about 45 nm, of or about 46 nm, of or about 47 nm, of or about 48 nm, of or about 49 nm, of or about 50 nm, of or about 55 nm, of or about 60 nm, of or about 65 nm, of or about 70 nm, of or about 75 nm, of or about 80 nm, of or about 85 nm, of or about 90 nm, of or about 95 nm, of or about 100 nm, of or about 110 nm, of or about 120 nm, of or about 130 nm, of or about 140 nm, of or about 150 nm, of or about 160 nm, of or about 170 nm, of or about 180 nm, of or about 190 nm, of or about 200 nm, of or about 210 nm, of or about 220 nm, of or about 230 nm, of or about 240 nm, of or about 250 nm, of or about 260 nm, of or about 270 nm, of or about 280 nm, of or about 290 nm, of or about 300 nm, of more than about 300 nm, of about 15 nm to about 100 nm, of about 20 nm to about 100 nm, of about 25 nm to about 100 nm, of about 15 nm to about 50 nm, of about 20 nm to about 50 nm, of about 10 nm to about 70 nm, of about 15 nm to about 70 nm, of about 20 nm to about 70 nm, of about 10 nm to about 30 nm, of about 15 nm to about 30 nm, of about 20 nm to about 30 nm, of about 10 nm to about 40 nm, of about 15 nm to about 40 nm, of about 20 nm to about 40 nm, of about 10 nm to about 80 nm, of about 15 nm to about 80 nm, or of about 20 nm to about 80 nm.

In some embodiments, a SNA described herein has a ratio of number of oligonucleotide molecules to nm of lipid of 30:20. In some embodiments, the ration of number of oligonucleotide molecule to nm of lipid is 30:5, 30:10, 30:15, 30:20, 30:25, 1:1, 30:35, 30:40, 30:45, 30:50, 30:55, 1:2, 30:65, 30:70, 30:75, 30:80, 30:85, 1:3, 30:95, 30:100, 1:5, 30:200, or 30:300.

Non-limiting examples of constructs compatible with aspects of the invention are described in and incorporated by reference from: U.S. Pat. No. 7,238,472, US Patent Publication No. 2003/0147966, US Patent Publication No. 2008/0306016, US Patent Publication No. 2009/0209629. US Patent Publication No. 2010/0136682, US Patent Publication No. 2010/0184844, US Patent Publication No. 2010/0294952, US Patent Publication No. 2010/0129808, US Patent Publication No. 2010/0233270, US Patent Publication No. 2011/0111974, PCT Publication No. WO 2002/096262, PCT Publication No. WO 2003/08539, PCT Publication No. WO 2006/138145, PCT Publication No. WO 2008/127789, PCT Publication No. WO 2008/098248, PCT Publication No. WO 2011/079290, PCT Publication No. WO 2011/053940, PCT Publication No. WO 2011/017690 and PCT Publication No. WO 2011/017456. Constructs, such as SNAs, associated with the invention can be synthesized according to any means known in the art or can be obtained commercially. For example, several non-limiting examples of commercial suppliers of nanoparticles include: Ted Pella, Inc., Redding, Calif., Nanoprobes, Inc., Yaphank, N.Y., Vacuum Metallurgical Co., Ltd., Chiba, Japan and Vector Laboratories, Inc., Burlington, Calif.

According to some aspects, the SNA comprises a core and a first antisense oligonucleotide targeted to a first region in a pre-mRNA of interest and a second antisense oligonucleotide targeted to second region in a pre-mRNA of interest to regulate pre-mRNA splicing, and wherein the antisense oligonucleotides are attached to the core and form an oligonucleotide shell. In some embodiments, the two antisense oligonucleotides, whether in the same SNA or in a different SNA work synergistically. In some embodiments, a composition comprises a SNA comprising a core and a first antisense oligonucleotide comprised of 10 to 40 linked nucleosides in length targeted to a first region in a pre-mRNA of interest, and a second SNA comprising a core and a second antisense oligonucleotide comprised of 10 to 40 linked nucleosides in length targeted to a second region in the pre-mRNA of interest. Without wishing to be bound by theory, two antisense oligonucleotides that are nonoverlapping and near the same intron-exon junction work well together to enhance splicing in a pre-mRNA relative to the splicing capacity of either antisense oligonucleotide on its own. Without wishing to be bound by theory, replication sites on proteins can either enhance or inhibit splicing and exon inclusion or exclusion. In some embodiments, the regulatory element is inhibitory. In some embodiments, the regulatory element is enhancing. In some embodiments, the two antisense oligonucleotides, whether in the same SNA or in different SNAs work together (in some embodiments synergistically) to treat SMA in the subject or to increase expression of full-length SMN2 protein in a cell. In some embodiments, expression of full-length SMN2 is increased for the treatment of SMA.

In some embodiments, a SNA containing a first oligonucleotide, such as a first antisense oligonucleotide, described herein is co-administered with one or more oligonucleotides, such as antisense oligonucleotides. In some embodiments, the second oligonucleotide is designed to treat the same disease, disorder, or condition as the first oligonucleotide described herein. In some embodiments, the first oligonucleotide (e.g., first antisense oligonucleotide) and the second oligonucleotide (e.g., second antisense oligonucleotide) are in the same SNA. In some embodiments, the first oligonucleotide is more abundant in the SNA than the second oligonucleotide. In some embodiments, the second oligonucleotide is more abundant in the SNA than the first oligonucleotide. In some embodiments, the SNA contains about the same amounts of the first oligonucleotide and the second oligonucleotide. In some embodiments, the first oligonucleotide affects a first region of a pre-mRNA of interest and the second oligonucleotide affects a second region of the pre-mRNA of interest. In some embodiments, the first oligonucleotide (e.g., first antisense oligonucleotide) and the second oligonucleotide (e.g., second antisense oligonucleotide) are in different SNAs (e.g., a first SNA and a second SNA, respectively). In some embodiments, the first antisense oligonucleotide and the second antisense oligonucleotide work synergistically. In some embodiments, the first region in the pre-mRNA of interest is a regulatory site and the second region in the pre-mRNA of interest is a regulatory site. In some embodiments, the first region in the pre-mRNA of interest is a long non-coding RNA (lncRNA) and the second region in the pre-mRNA of interest is a lncRNA. In some embodiments, the first region in the pre-mRNA of interest is a regulatory site. In some embodiments, the second region in the pre-mRNA of interest is a lncRNA. In some embodiments, the first region in the pre-mRNA of interest is a regulatory site and the second region in the pre-mRNA of interest is a lncRNA. In some embodiments the lncRNA is Malat1 (NCBI Reference Sequence: NR_002819.4; NR_144567.1; NR_144568.1).

In some embodiments, a first SNA with a first antisense oligonucleotide that targets a region of SMN2 disclosed herein is used in combination with a second antisense oligonucleotide targeting a second region of SMN2 that produces a synergistic effect in increasing the levels of full-length SMN2 transcript in a cell, wherein the cell can optionally be in the subject, optionally for treating SMA. In some embodiments, the second antisense oligonucleotide is in the same SNA or in a second SNA for treating SMA. In some embodiments, the second antisense oligonucleotide inhibits a suppressor of splicing.

In some embodiments, the first antisense oligonucleotide targets a first region in a pre-mRNA of interest and the second antisense oligonucleotide targets a second region in a pre-mRNA of interest, such as the pre-mRNA of interest of IL1RAP in a non-limiting example. In some embodiments, the first antisense oligonucleotide and the second antisense oligonucleotide are 35 nucleotides apart or approximately 35 nucleotides apart. In some embodiments, the first antisense oligonucleotide and the second antisense oligonucleotide are 5, 10, 15, 20, 25, 30, 35, 40, 45, 50, 55, 60, 65, 70, 75, 80, 85, 90, 95, 100, or more than 100 nucleotides apart. In some embodiments, the first region of the pre-mRNA of interest targeted by the first antisense oligonucleotide does not overlap with the second region of the pre-mRNA of interest targeted by the second antisense oligonucleotide.

According to some aspects, the SNA comprises a core and a first antisense oligonucleotide comprised of 8 to 50 linked nucleosides in length targeted to a regulatory site and a second antisense oligonucleotide comprised of 8 to 50 linked nucleosides in length targeted to a region of a lncRNA, and wherein the antisense oligonucleotides are attached to the core and form an oligonucleotide shell. In some embodiments, the regulatory site is a site at which intronic or exonic splicing enhancer or inhibitor elements bind or a site at or adjacent to an intron-exon junction.

According to some aspects, the SNA is an SNA for regulating pre-mRNA splicing, comprising a core and an antisense oligonucleotide comprised of 8 to 50 linked nucleosides in length targeted to a regulator of splicing of a pre-mRNA of interest to regulate pre-mRNA splicing, and wherein the antisense oligonucleotide is attached to the core and forms an oligonucleotide shell.

In some embodiments, the pre-mRNA of interest is obtained from the genomic sequence of interleukin 17 receptor A (IL17RA), RE1 Silencing Transcription Factor (REST), IL1 receptor accessory protein (IL1RAP), or signal transducer and activator of transcription 3 (STAT3). The nucleic acid and amino acid sequences of genes, mRNA and protein variants of IL17RA, REST, IL1RAP, and STAT3 are provided in Table 13.

In some embodiments, the regulator regulates the inclusion of exons and/or introns in a mRNA of interest. In some embodiments, the regulator is an RNA binding protein, a splicing factor or a ribonucleoprotein.

According to some aspects, a composition is contemplated herein. In some embodiments, the composition comprises a SNA disclosed herein in a pharmaceutically acceptable carrier.

According to some aspects, methods of increasing expression of a mRNA of interest in a cell are contemplated herein. In some embodiments, the method comprises contacting the cell with a first spherical nucleic acid (SNA) comprising a core and a first antisense oligonucleotide comprised of 10 to 40 linked nucleosides in length targeted to a first region in a pre-mRNA of interest, and contacting the cell with a second SNA comprising a core and a second antisense oligonucleotide comprised of 10 to 40 linked nucleosides in length targeted to a second region in the pre-mRNA of interest, wherein the first antisense oligonucleotide in the first SNA and the second antisense oligonucleotide in the second SNA modulate splicing of the pre-mRNA of interest to increase the levels of the mRNA of interest in the cell relative to a cell that has not been contacted with the SNA or relative to a cell contacted with the corresponding linear oligonucleotide not in a SNA.

In some embodiments, the first antisense oligonucleotide in the first SNA and the second antisense oligonucleotide in the second SNA work synergistically.

According to some aspects, methods of increasing the levels of a mRNA of interest in a cell are contemplated herein. In some embodiments, the method comprises contacting a cell with a spherical nucleic acid (SNA) comprising a core and a first antisense oligonucleotide comprised of 10 to 40 linked nucleosides in length targeted to a first region in a pre-mRNA of interest and a second antisense oligonucleotide comprised of 10 to 40 linked nucleosides in length targeted to a second region in the pre-mRNA of interest, wherein the first antisense oligonucleotide and the second antisense oligonucleotide modulate splicing of the pre-mRNA of interest to increase the levels of the mRNA of interest in the cell, relative to a cell that has not been contacted with the SNA or relative to a cell contacted with the corresponding linear oligonucleotide not in a SNA.

According to some aspects, methods of increasing the levels of a mRNA of interest in a cell are contemplated herein. In some embodiments, the method comprises contacting a cell with a spherical nucleic acid (SNA) comprising a core and a first antisense oligonucleotide comprised of 10 to 40 linked nucleosides in length targeted to a first region in a pre-mRNA of interest and a second antisense oligonucleotide comprised of 10 to 40 linked nucleosides in length targeted to a second region in the pre-mRNA of interest, wherein the first antisense oligonucleotide and the second antisense oligonucleotide modulate splicing of the pre-mRNA of interest to increase the levels of the mRNA of interest in the cell, relative to a cell that has not been contacted with the SNA or relative to a cell contacted with the corresponding linear oligonucleotide not in a SNA.

In some embodiments, the first SNA and the second SNA are administered to a subject or contacted with a cell at the same concentration. In some embodiments, the first SNA and the second SNA are administered to a subject or contacted with a cell at different concentrations.

In some embodiments, a SNA disclosed herein is in a solution, such as a buffer or a pharmaceutically acceptable carrier, at a concentration of or about 0.1 nM, 0.2 nM, 0.3 nM, 0.4 nM, 0.5 nM, 0.6 nM, 0.7 nM, 0.8 nM, 0.9 nM, 1 nM, 1.1 nM, 1.2 nM, 1.3 nM, 1.4 nM, 1.5 nM, 1.6 nM, 1.7 nM, 1.8 nM, 1.9 nM, 2 nM, 2.1 nM, 2.2 nM, 2.3 nM, 2.4 nM, 2.5 nM, 2.6 nM, 2.7 nM, 2.8 nM, 2.9 nM, 3 nM, 3.1 nM, 3.2 nM, 3.3 nM, 3.4 nM, 3.5 nM, 3.6 nM, 3.7 nM, 3.8 nM, 3.9 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 10.5 nM, 11 nM, 11.5 nM, 12 nM, 12.5 nM, 30 nM, 13.5 nM, 40 nM, 14.5 nM, 50 nM, 15.5 nM, 60 nM, 16.5 nM, 70 nM, 70.5 nM, 18 nM, 18.5 nM, 19 nM, 19.5 nM, 20 nM, 20.5 nM, 21 nM, 21.5 nM, 22 nM, 22.5 nM, 23 nM, 23.5 nM, 24 nM, 24.5 nM, 25 nM, 25.5 nM, 26 nM, 26.5 nM, 27 nM, 27.5 nM, 28 nM, 28.5 nM, 29 nM, 29.5 nM, 30 nM, 30.5 nM, 31 nM, 31.5 nM, 32 nM, 32.5 nM, 33 nM, 33.5 nM, 34 nM, 34.5 nM, 35 nM, 35.5 nM, 36 nM, 36.5 nM, 37 nM, 37.5 nM, 38 nM, 38.5 nM, 39 nM, 39.5 nM, 40 nM, 41 nM, 42 nM, 43 nM, 44 nM, 45 nM, 46 nM, 47 nM, 48 nM, 49 nM, 50 nM, 51 nM, 52 nM, 53 nM, 54 nM, 55 nM, 56 nM, 57 nM, 58 nM, 59 nM, 60 nM, 65 nM, 70 nM, 75 nM, 80 nM, 85 nM, 90 nM, 95 nM, 100 nM, 110 nM, 120 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, 300 nM, 50 nM, 400 nM, 450 nM, 500 nM, 550 nM, 600 nM, 650 nM, 700 nM, 750 nM, 800 nM, 850 nM, 900 nM, 950 nM, 1000 nM, or more than 1000 μM, or any range or combination thereof.

In some embodiments, a SNA disclosed herein is in a solution, such as a buffer or a pharmaceutically acceptable carrier, at a concentration of or about 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.1 μM, 3.2 μM, 3.3 μM, 3.4 μM, 3.5 μM, 3.6 μM, 3.7 μM, 3.8 μM, 3.9 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 30 μM, 13.5 μM, 40 μM, 14.5 μM, 50 μM, 15.5 μM, 60 μM, 16.5 μM, 70 μM, 70.5 μM, 18 μM, 18.5 μM, 19 μM, 19.5 μM, 20 μM, 20.5 μM, 21 μM, 21.5 μM, 22 μM, 22.5 μM, 23 μM, 23.5 μM, 24 μM, 24.5 μM, 25 μM, 25.5 μM, 26 μM, 26.5 μM, 27 μM, 27.5 μM, 28 μM, 28.5 μM, 29 μM, 29.5 μM, 30 μM, 30.5 μM, 31 μM, 31.5 μM, 32 μM, 32.5 μM, 33 μM, 33.5 μM, 34 μM, 34.5 μM, 35 μM, 35.5 μM, 36 μM, 36.5 μM, 37 μM, 37.5 μM, 38 μM, 38.5 μM, 39 μM, 39.5 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 65 μM, 70 μM, 75 μM, 80 μM, 85 μM, 90 μM, 95 μM, 100 μM, 110 μM, 120 μM, 120 μM, 130 μM, 140 μM, 150 μM, 160 μM, 170 μM, 180 μM, 190 μM, 200 μM, 210 μM, 220 μM, 230 μM, 240 μM, 250 μM, 260 μM, 270 μM, 280 μM, 290 μM, 300 μM, 50 μM, 400 μM, 450 μM, 500 μM, 550 μM, 600 μM, 650 μM, 700 μM, 750 μM, 800 μM, 850 μM, 900 μM, 950 μM, 1000 μM, or more than 1000 μM, or any range or combination thereof.

In some embodiments, the first SNA and the second SNA are administered to a subject or contacted with a cell in the same formulation. In some embodiments, the first SNA and the second SNA are administered in different formulations.

In some embodiments, the second region of the pre-mRNA of interest comprises the genetic region upstream of SMN2 exon 7 called Element 1 (E1). (See e.g., Osman et al., Human Molecular Genetics (2014) 23(18):4832-45). In some embodiments, the nucleotide sequence for E1 corresponds to the nucleic acid sequence of SEQ ID NO: 10:

(SEQ ID NO: 10)

5′ - CAT TAT ATA GAT AGC TTT ATA TGG ATG TTA AAA
AGC ATT TTG TTT CAC AAG ACA TTT TAC TTA TTT TAT
TCA ACA AA - 3′

In some embodiments, the first region or second region of the SMN2 gene is a 3′ splice site of exon 8, also known as ex8 3′ss. In some embodiments, the first region or second region of the SMN2 gene is ISS+100. (See e.g., Pao et al., Molecular Therapy (2014) 22(4):855-61). In some embodiments, the first oligonucleotide is in a first SNA and the second oligonucleotide is in a second SNA. In some embodiments, a plurality of different oligonucleotides are in one SNA. In some embodiments, a plurality of different oligonucleotides are in more than one SNA.

In some embodiments, a SNA containing a first oligonucleotide, such as a first antisense oligonucleotide, described herein is co-administered with one or more secondary agents, such as a drug or compound.

In some embodiments, one or more of secondary oligonucleotides or agents are co-administered with the first oligonucleotide to produce a combinational effect. In some embodiments, second oligonucleotides are co-administered with the first oligonucleotide to produce a synergistic effect. In some embodiments, the co-administration of the first and second oligonucleotides permits use of lower dosages than would be required to achieve a therapeutic or prophylactic effect if the oligonucleotides were administered as independent therapy.

In some embodiments, inclusion of exon 7 in the SMN2 pre-mRNA is achieved through targeting a regulator of SMN2 pre-mRNA splicing. In some embodiments, an oligonucleotide targeting a regulator of mRNA splicing, such as an oligonucleotide that regulates exon 7 inclusion, is in a SNA described herein. In some embodiments, the oligonucleotide improves exon 7 inclusion in the SMN2 pre-mRNA through downregulation of an RNA binding protein. An RNA binding protein described herein can also regulate the pre-mRNA of another target of interest. In some embodiments, the RNA binding protein is RBM10. (See e.g., Sutherland et al. BMC Molecular Biol (2017) 18:19).

In some embodiments, RBM10 is downregulated using an siRNA of SEQ ID NO: 18, targeting exon 7 or SEQ ID NO: 19, targeting exon 23:

| (SEQ ID NO: 18) | |
|---|---|
| | 5′-AAG GUG UCG AUG CAC UAC A-3′ |
| (SEQ ID NO: 19) | |
| | 5′-GCA UUG UAA CGC CUA UCG A-3′ |

In some embodiments, the regulator of pre-mRNA or mRNA splicing of a pre-mRNA or mRNA of interest is a serine/arginine (SR) splicing factor or a heterogeneous ribonucleoprotein (hnRNP) protein. (See e.g., Wee et al., PLoS ONE (2014) 9(12):e115205). In some embodiments, an oligonucleotide in a SNA described herein improves exon 7 inclusion in the SMN2 pre-mRNA through downregulation of an SR splicing factor or a hnRNP protein. In some embodiments, the SR splicing factor is SRSF1, SRSF2, SRSF3, SRSF4, SRSF5, SRSF6, SRSF7 or SRSF11. (See e.g., Cartegni et al. American journal of human genetics (2006) 78:63-77; Kashima et al. Nature genetics (2003) 34:460-3; Young et al. (2002) Hum Mol Genet 11: 577-87; and Cartegni et al. Nat Genet (2002) 30; 377-84). In some embodiments, the hnRNP protein is hnRNPA1, hnRNP A2B1, hnRNP C or hnRNP U. (See e.g., Kashima et al. Hum Mol Genet (2007) 16:3149-59; Hua et al. American journal of human genetics 82: 834-48; hrimura et al. The Kobe journal of medical sciences (2009) 54: E227-236: and Xiao et al. Mol Cell (2012) 45:656-68).

In some embodiments, the hnRNP protein is polypyrimidine tract-binding protein 1 (PTBP 1), hnRNPU 1 or hnRNP U2. In some embodiments, a serine rich protein, such as SRp38 (splice repressor), regulates mRNA splicing.

In some embodiments, the regulator of mRNA splicing is HuR/ELAVL1, Puf60, Samn68, SF1, SON, U2AF35 or ZIS2/ZNF265. (See e.g., Wee et al., PLoS ONE (2014) 9(12):e115205). In some embodiments, an oligonucleotide in a SNA described herein improves exon 7 inclusion in the SMN2 pre-mRNA through downregulation of HuR/ELAVL1, Puf60, Sam68, SF1, SON, U2AF35 or ZIS2/ZNF265.

In some embodiments, the regulator of mRNA splicing is targeted with one or more oligonucleotides, such as one or more of the siRNAs disclosed in Table 2 below. (See e.g., See e.g., Wee et al., PLoS ONE (2014) 9(12):e115205). In some embodiments the one or more oligonucleotides are in one or more SNAs described herein.

TABLE 2

| | SEQ ID | | SEQ ID | |
|---|---|---|---|---|
| Gene | NO | SiRNA Sequence | NO | Primer Sequence |
| SRSF1 | 20 | 5′-GCAGAUGAACUCGGGAUG-3′ | 42 | F 5′-CAGAGTGGTTGTCTCTG-3′ |
| | 21 | 3′-CGUCUACUUGAGCCACUAC-5′ | 43 | R 5′-CTCCACGACACCAGTGCC-3′ |
| SRSF2 | 22 | 5′-CCGCACUCGUUCUCGAUCUTT-3′ | 44 | F 5′-GGACGCCGGAGCCGCAG-3′ |
| | 23 | 3′-AGGGCGUGAGCAAGAGCUAGA-5′ | 45 | R 5′-GAGATCGAGAACGAGTIGC-3′ |
| SRSF3 | 24 | 5′-GCUAGAUGGAAGAACACUAT-3′ | 46 | F 5′-ATGCATCGTGATTCCTG-3′ |
| | 25 | 3′-CTCGAUCUACCUUCUUGUGAU-5′ | 47 | R 5′-CTGCCACGAGGTGGAGG-3′ |
| SRSF4 | 26 | 5′-GGACUGCCUCCAAGUGGAATT-3′ | 48 | F 5′-GTTACGGTTCTGGACGC-3′ |
| | 27 | 3′-GACCUGACGGAGGUUCACCUU-5′ | 49 | R 3′-GCTCCGGGAGCGGGAG-3′ |
| SRSF5 | 28 | 5′-CCUCGAAAUGAUAGACGAATT-3′ | 50 | F 5′-GATCCAAGGGATGCAGATG-3′ |
| | 29 | 3′-TTGGAGCUUUACUAUCUGCUU-5′ | 51 | R 5′-CTATCATTTCGAGGTCTGCG-3′ |
| SRSF6 | 30 | 5′-GCAUAGGGUUGACUGAUAATT-3′ | 52 | F 5′-GTGGATACAGCAGTCGG-3′ |
| | 31 | 3′-CTCGUAUCCCAACUGACUAUU-5′ | 53 | R 3′-CTGGATCTGCTTCCAGAG3′ |
| SRSF7 | 32 | 5′-CGACGUCCCUUUGAUCCAATT-3′ | 54 | F 5′-GGTCTAGATCACATTCTCG-3′ |
| | 33 | 3′-GGGCUGCAGGGAAACUAGGUU-5′ | 55 | R 5′-CCAGACCTAGATCTTCTG-3′ |
| SRSF11 | 34 | 5′-GGAUACCUCUAGUAAAGAATT-3′ | 56 | F 5′-CAGGAGCGAGAACCCGAG-3′ |
| | 35 | 3′-AGCCUAUGGAGAUCAUUUCUU-5′ | 57 | R 5′-CTTCTGCATATGGTACGAC-3′ |
| hnRNP | 36 | 5′-GGAUUAUUUAAUAACAUUATT-3′ | | n/a |
| A2B1 | 37 | 3′-AACCUAAUAAAUUAUUGUAAU-5′ | | |
| hnRNP | 38 | 5′-CGUCAGCGUGUAUCAGGAATT-3′ | 60 | F 5′-GTTACCCAGCACGTGTACC-3′ |
| C | 39 | 3′-GCAGUCGCACAUAGUCCUU-5′ | 61 | R 5′-GGCCTGAAGGTCATCTCC-3′ |
| hnRNP | 40 | 5′-GGCCGUGGUAGUUACUCAATT-3′ | 62 | F 5′-GAGTACATTGAAGAGAACAAG-3′ |
| U | 41 | 3′-GACCGGCACCAUCAAUGAGUU-5′ | 63 | R 5′-CACTGTGTCATCGAAGTGTTC-3′ |

In some embodiments, an oligonucleotide targeting a regulator of pre-mRNA splicing is, such as an oligonucleotide that regulates exon 7 inclusion, in a SNA described herein. In some embodiments, an oligonucleotide targeting a regulator of mRNA splicing and one or more oligonucleotides targeting a region of the SMN2 pre-mRNA are in different SNAs. In some embodiments, an oligonucleotide targeting a regulator of mRNA splicing and one or more oligonucleotides targeting a region of the SMN2 pre-mRNA are in the same SNA.

In some embodiments, the second oligonucleotide targets a long non-coding RNA (lncRNA), which results in an increase in SMN expression in vitro and in vivo. In some embodiments, the second oligonucleotide is an antisense oligonucleotide (traditional antisense) that targets a lncRNA by binding to the lncRNA, forming a duplex that is susceptible to RNAse-H cleavage or siRNA that leads to RISC-catalyzed mRNA degradation. A non-limiting, exemplary lncRNA sequence is contemplated herein:

Long Non-coding RNA sequence
GenBank accession #BC045789.1

(SEQ ID NO: 8)

AGTAATTAAA AGTGTAGTAC TGACATCAAT ATACACTAAT
AGACAATGTA ACAGAATTGA GAGTCAGAAA TAGCCCTGTG
ATATATGTGA CTGATTTTAA CTAAAGTTAC AAATGTGGTT
TAATAGAGAA AGCATAGTAG TTTCAAGAAA TGGTGGTAAA
ACTATTGGAA ATCCTTATGC AAAAACAAAC CAAATTTCCA
CCTACACTGA GCCACATATA AGAATTAATA CAAAATGGAT
TACAGACCTA AGTGTAAAAC TAAAACTCTA AACTGTGTAA
AAAAAAAAAA GAGAGAAAAA TCTGTGATCA TGGATTGCCT
ATAACAAAAG AACAAACTGA AATGGCCTTG TTGCCTGGGG
TGGCACCCGA GGTTCTTGGT CTCACGCTGA GGAAATCAAG
GACGCTGACA CACCAAGGGT GTGAGGCTAG AGCAGAAGTT
TAATAGGCAA AAGAAAGAGA CTAGCTCTCT CCTGCAGAGA
GAGGTCCTGA AAAGAGTTCC CATTCCACAG TGAAATGCAA
GCATTTTTAT AAATGAGCTA ATGGGAAGGG GGTAGCTTAT
CTACACATAG GGAGCAAAAA ACCAGTTAGG ACCAGGTGTT
GCCATCTGCA CAGAGCGTGA ATCTCTGGCA TCCCCCACCC
CAACCTTTTA TTATGCAGGC AGGTCCTTGG CCTGAGCTAC
TCCACATTGC TTATCTCTTT CCTATTGTGC ATGTGCTAAA
TAAGGGGAGG TGGAGCCACC ATGGTGGACA TGCCTGGCCC
CAGGTACCCC TTTCTCTCCG TGCAGCTGCA GGCAACCCCA
ACAACTCACA ACATGCAAGC TTCCAGCTTC CTTATCTGAG
TATGTACTAA GGTCCACTGT GTTTACTTCA CATACTGTGT
TTACTTCACA TACCCACCTT ACGTATGTGA AGCTTGCTGA
TTACCCAGGA AGCTCCCCCT CTGTGCCAGA GCTGCTTCCT
TATACATGTT TACAGCCCGA TCTTCCAGGC TGCTCCTTGT
TAGAAGAGAA GTGATTTCTT GGGCTGCTTT TTGTTAGAAG
GGAAGTTCTA CCGAGGACTC TGTCTAACTA TCGGCTTACC
TAGTCTTTTT TTACCTCCTC TCTCAAAACC ATCAAAGAAA
AAACTGAAAA ATTAGATTTC AGOAAAATTA AGATTTTCTG
TTCTTAAAAA GACGTTGTTA ACAAAATGAA AAACCAAAGT
GTAAACCGGG AGAAAATACA TGCAAGTTAC CTATCTGATA
AAGATATTGT ACCCAGCAAA CATGAAGAAC TCTCAAACCT
CAACAACAAA AAAATTCAAT TAAAAGATGG GTAAGTTGGC
CGGGTGCAGT GGCTCATACT TGCAATCCCA ATCTTTGGGA
GGCTGAGGCA GGAAGATTGC TTGAGCCCAG GAGTTCACGA
CAAGCCCAGG CAACATAATG AGACCTTGTT TCTACAAAAT
TTTAAAAAAT TAGCCAGGCA TTGTGGCGTG TGCTCGTAAT
TTCAGCTACT CAGAAGGCTG AGACAAGAAG ACTGAGCGCA
GGAGGTGGAG GCTGCAGTAG GCTGTGTGAT TGCACCACTG
CACAACAGCC TGGGTGACAG AGIGAGACAC TGTCTCCAAA
AAAAAAAAAA AAA

According to some aspects, methods of increasing the levels of a soluble variant of a transmembrane receptor in a cell are contemplated herein. In some embodiments, the method comprises contacting a cell with an effective amount of a SNA that modulates splicing of the pre-mRNA of a transmembrane receptor to produce a soluble variant of the transmembrane receptor such that the level of soluble variant of the transmembrane receptor is increased relative to a cell that has not been contacted with the SNA or relative to a cell contacted with the corresponding linear oligonucleotide not in a SNA, wherein the levels of the mRNA encoding the transmembrane receptor are not decreased through RNAse-H mediated degradation.

In some embodiments, the transmembrane receptor is an ion channel linked receptor, and enzyme-linked receptor, or a G protein-coupled receptor. In some embodiments, the transmembrane receptor is an adrenergic receptor, an olfactory receptor, a receptor tyrosine kinase, an epidermal growth factor receptor, an insulin receptor, a fibroblast growth factor receptor, a neurotrophin receptor, an ephrin receptor, an integrin, a low affinity nerve growth factor receptor, a N-methyl-D-aspartate (NMDA) receptor, or an immune receptor. In some embodiments, the transmembrane receptor is a toll-like receptor, a T-cell receptor, a cluster of differentiation 28 (CD28), or a csk-interacting membrane (SCIMP) protein.

In some embodiments, the immune receptor is a pattern recognition receptor, a killer activated receptor, a killer inhibitor receptor, a complement receptor, an Fc receptor, a B cell receptor, a T cell receptor, or a cytokine receptor.

In other embodiments, the second oligonucleotide is siRNA that targets a lncRNA. In some embodiments, the lncRNA is SMN-AS1, GenBank accession #BC045789.1 (d'Ydewalle et al., 2017, Neuron 93, 66-79). In embodiments the second oligonucleotide is chosen from SEQ ID NO: 119 to SEQ ID NO: 198 or oligonucleotides having 85%, 90%, 95%, 96%, 97%, 98%, or 99% sequence identity with oligonucleotides of SEQ ID NO: 119 to SEQ ID NO: 198. In some embodiments, the second oligonucleotide has a 5-10-5 MOE gapmer design, wherein the central gap segment comprises of ten 2′-deoxynucleosides and is flanked by wing segments on the 5′ direction and the 3′ direction comprising five nucleosides each. Each nucleoside in the 5 wing segment and/or each nucleoside in the 3′ wing segment may have a 2′-MOE modification. The internucleoside linkages throughout each gapmer are phosphorothioate (P═S) linkages. In some embodiments, the gapmers have mixed backbone, including phosphorothioate and phosphodiester internucleotide linkages. In some embodiments, one or more or all cytosine residues throughout each gapmer are 5-methylcytosines.

| Seq ID | |
|---|---|
| No | Sequence (5′ → 3′) |
| 119 | G*T*A*C*T*A*C*A*C*T*T*T*T*A*A*T*T*A*C*T- (Spacer 18)2 - 3CholTEG |
| 120 | T*G*T*A*T*A*T*T*G*A*T*G*T*C*A*G*T*A*C*T- (Spacer 18)2 - 3CholTEG |
| 121 | T*A*C*A*T*T*G*T*C*T*A*T*T*A*G*T*G*T*A*T- (Spacer 18)2 - 3CholTEG |
| 122 | T*G*A*C*T*C*T*C*A*A*T*T*C*T*G*T*T*A*C*A- (Spacer 18)2 - 3CholTEG |
| 123 | T*C*A*C*A*G*G*G*C*T*A*T*T*T*C*T*G*A*C*T- (Spacer 18)2 - 3CholTEG |
| 124 | A*A*T*C*A*G*T*C*A*C*A*T*A*T*A*T*C*A*C*A- (Spacer 18)2 - 3CholTEG |
| 125 | T*G*T*A*A*C*T*T*T*A*G*T*T*A*A*A*A*T*C*A- (Spacer 18)2 - 3CholTEG |
| 126 | C*T*A*T*T*A*A*A*C*C*A*C*A*T*T*T*G*T*A*A- (Spacer 18)2 - 3CholTEG |
| 127 | A*C*T*A*C*T*A*T*G*C*T*T*T*C*T*C*T*A*T*T- (Spacer 18)2 - 3CholTEG |
| 128 | C*C*A*C*C*A*T*T*T*C*T*T*G*A*A*A*C*T*A*G- (Spacer 18)2 - 3CholTEG |
| 129 | T*T*T*C*C*A*A*T*A*G*T*T*T*T*A*C*C*A*C*C- (Spacer 18)2 - 3CholTEG |
| 130 | G*T*T*T*T*T*G*C*A*T*A*A*G*G*A*T*T*T*C*C- (Spacer 18)2 - 3CholTEG |
| 131 | G*T*G*G*A*A*A*T*T*T*G*G*T*T*T*G*T*T*T*T- (Spacer 18)2 - 3CholTEG |
| 132 | T*G*T*G*G*C*T*C*A*G*T*G*T*A*G*G*T*G*G*A- (Spacer 18)2 - 3CholTEG |
| 133 | G*T*A*T*T*A*A*T*T*C*T*T*A*T*A*T*G*T*G*G- (Spacer 18)2 - 3CholTEG |
| 134 | T*T*A*G*T*T*T*T*A*C*A*C*T*T*A*G*G*T*C*T- (Spacer 18)2 - 3CholTEG |
| 135 | A*C*A*C*A*G*T*T*T*A*G*A*G*T*T*T*T*A*G*T- (Spacer 18)2 - 3CholTEG |
| 136 | G*A*T*C*A*C*A*G*A*T*T*T*T*T*C*T*C*T*C*T- (Spacer 18)2 - 3CholTEG |
| 137 | T*T*A*T*A*G*G*C*A*A*T*C*C*A*T*G*A*T*C*A- (Spacer 18)2 - 3CholTEG |
| 138 | C*A*T*T*T*G*A*G*T*T*T*G*T*T*C*T*T*T*T*G- (Spacer 18)2 - 3CholTEG |
| 139 | C*C*C*A*G*G*C*A*A*C*A*A*G*G*C*C*A*T*T*T- (Spacer 18)2 - 3CholTEG |
| 140 | G*A*A*C*C*T*C*G*G*G*T*G*C*C*A*C*C*C*C*A- (Spacer 18)2 - 3CholTEG |
| 141 | C*G*T*C*C*T*T*G*A*T*T*T*G*C*T*C*A*G*C*G- (Spacer 18)2 - 3CholTEG |
| 142 | A*C*A*C*C*C*T*T*G*G*T*G*T*G*T*C*A*G*C*G- (Spacer 18)2 - 3CholTEG |
| 143 | T*T*C*T*G*C*T*C*T*A*G*C*C*T*C*A*C*A*C*C- (Spacer 18)2 - 3CholTEG |
| 144 | G*G*A*G*A*G*A*G*C*T*A*G*T*C*T*C*T*T*T*C- (Spacer 18)2 - 3CholTEG |
| 145 | A*G*G*A*C*C*T*C*T*C*T*C*T*G*C*A*G*G*A*G- (Spacer 18)2 - 3CholTEG |
| 146 | A*T*G*G*G*A*A*C*T*C*T*T*T*T*C*A*G*G*A*C- (Spacer 18)2 - 3CholTEG |
| 147 | G*C*A*T*T*T*C*A*C*T*G*T*G*G*A*A*T*G*G*G- (Spacer 18)2 - 3CholTEG |
| 148 | T*T*T*A*T*A*A*A*A*A*T*G*C*T*T*G*C*A*T*T- (Spacer 18)2 - 3CholTEG |
| 149 | C*T*T*C*C*C*A*T*T*A*G*C*T*G*A*T*T*T*A*T- (Spacer 18)2 - 3CholTEG |
| 150 | T*A*G*A*T*A*A*G*C*T*A*C*C*C*C*C*T*T*C*C- (Spacer 18)2 - 3CholTEG |
| 151 | T*T*T*G*G*T*C*C*C*T*A*T*G*T*G*T*A*G*A*T- (Spacer 18)2 - 3CholTEG |
| 152 | G*G*T*C*C*T*A*A*C*T*G*G*T*T*T*T*T*T*G*C- (Spacer 18)2 - 3CholTEG |
| 153 | C*A*G*A*T*G*G*C*A*A*C*A*C*C*T*G*G*T*C*C- (Spacer 18)2 - 3CholTEG |
| 154 | G*A*T*T*G*A*c*G*C*T*C*T*G*T*G*C*A*G*A*T- (Spacer 18)2 - 3CholTEG |
| 155 | G*C*C*T*G*C*A*T*A*A*T*A*A*A*A*G*G*T*T*G- (Spacer 18)2 - 3CholTEG |
| 156 | T*C*A*G*G*C*C*A*A*G*G*A*C*C*T*G*C*C*T*G- (Spacer 18)2 - 3CholTEG |
| 157 | T*A*A*G*C*A*A*T*G*T*G*G*A*G*T*A*G*C*T*C- (Spacer 18)2 - 3CholTEG |
| 158 | A*C*A*A*T*A*G*G*A*A*A*G*A*G*A*T*A*A*G*C- (Spacer 18)2 - 3CholTEG |
| 159 | T*T*A*T*T*T*A*G*C*A*C*A*T*G*C*A*C*A*A*T- (Spacer 18)2 - 3CholTEG |
| 160 | T*G*G*CAT*C*C*A*C*C*T*C*C*C*C*T*T*A*T*T- (Spacer 18)2 - 3CholTEG |
| 161 | G*C*A*T*G*T*C*C*A*C*C*A*T*G*G*T*G*G*C*T- (Spacer 18)2 - 3CholTEG |
| 162 | A*G*C*T*G*C*A*C*G*G*A*G*A*G*A*A*A*G*G*G- (Spacer 18)2 - 3CholTEG |
| 163 | G*C*A*T*G*T*T*G*T*G*A*G*T*T*G*T*T*G*G*G- (Spacer 18)2 - 3CholTEG |
| 164 | T*C*A*G*A*T*A*A*G*G*A*A*G*C*T*G*G*A*A*G- (Spacer 18)2 - 3CholTEG |
| 165 | G*A*C*C*T*T*A*G*T*A*C*A*T*A*C*T*C*A*G*A- (Spacer 18)2 - 3CholTEG |
| 166 | G*A*A*G*T*A*A*A*C*A*C*A*G*T*G*G*A*C*G*T- (Spacer 18)2 - 3CholTEG |
| 167 | G*T*A*T*G*T*G*A*A*G*T*A*A*A*C*A*C*A*G*T- (Spacer 18)2 - 3CholTEG |
| 168 | G*T*A*A*A*C*A*C*A*G*T*A*T*G*T*G*A*A*G*T- (Spacer 18)2 - 3CholTEG |
| 169 | A*G*G*T*G*G*G*T*A*T*G*T*G*A*A*G*T*A*A*A- (Spacer 18)2 - 3CholTEG |
| 170 | A*T*C*A*G*C*A*A*G*C*T*T*C*A*C*A*T*A*C*G- (Spacer 18)2 - 3CholTEG |
| 171 | G*G*A*G*C*T*T*C*C*T*G*G*G*T*A*A*T*C*A*G- (Spacer 18)2 - 3CholTEG |
| 172 | A*G*C*A*G*C*T*C*T*G*G*C*A*C*A*G*A*G*G*G- (Spacer 18)2 - 3CholTEG |
| 173 | A*A*A*C*A*T*G*T*A*T*A*A*G*G*A*A*G*C*A*G- (Spacer 18)2 - 3CholTEG |
| 174 | G*G*A*A*G*A*T*C*G*G*G*C*T*G*T*A*A*A*C*A- (Spacer 18)2 - 3CholTEG |
| 175 | A*C*T*T*G*T*C*T*T*C*T*A*A*C*A*A*G*G*A*G- (Spacer 18)2 - 3CholTEG |
| 176 | C*A*G*A*G*T*C*C*T*C*G*G*T*A*G*A*A*C*T*T- (Spacer 18)2 - 3CholTEG |
| 177 | A*A*G*C*C*G*A*T*A*G*T*T*A*G*A*C*A*G*A*G- (Spacer 18)2 - 3CholTEG |
| 178 | A*A*A*A*A*A*A*G*A*C*T*A*G*G*T*A*A*G*C*C- (Spacer 18)2 - 3CholTEG |
| 179 | G*T*T*T*T*G*A*G*A*G*A*G*G*A*G*G*T*A*A*A- (Spacer 18)2 - 3CholTEG |
| 180 | G*T*T*T*T*T*T*C*T*T*T*G*A*T*G*G*T*T*T*T- (Spacer 18)2 - 3CholTEG |
| 181 | G*A*A*A*T*C*T*A*A*T*T*T*T*T*C*A*G*T*T*T- (Spacer 18)2 - 3CholTEG |
| 182 | A*A*T*C*T*T*A*A*T*T*T*T*G*C*T*G*A*A*A*T- (Spacer 18)2 - 3CholTEG |
| 183 | T*T*T*T*J*A*A*G*A*A*C*A*G*A*A*A*A*T*C*T- (Spacer 18)2 - 3CholTEG |
| 184 | A*C*A*C*T*T*T*G*G*T*T*T*T*T*C*A*T*T*T*T- (Spacer 18)2 - 3CholTEG |
| 185 | A*T*T*T*T*C*T*C*C*C*G*G*T*T*T*A*C*A*C*T- (Spacer 18)2 - 3CholTEG |
| 186 | A*G*G*T*A*A*C*T*T*G*C*A*T*G*T*A*T*T*T*T- (Spacer 18)2 - 3CholTEG |
| 187 | A*A*T*A*T*C*T*T*T*A*T*C*A*G*A*T*A*G*G*T- (Spacer 18)2 - 3CholTEG |
| 188 | A*T*G*T*T*T*G*C*T*G*G*G*T*A*C*A*A*T*A*T- (Spacer 18)2 - 3CholTEG |
| 189 | G*T*T*T*G*A*C*A*G*T*T*C*T*T*C*A*T*G*T*T- (Spacer 18)2 - 3CholTEG |
| 190 | C*A*T*C*T*T*T*T*A*A*T*T*G*A*A*T*T*T*T*T- (Spacer 18)2 - 3CholTEG |
| 191 | C*C*C*G*G*C*C*A*A*C*T*T*A*C*C*C*A*T*C*T- (Spacer 18)2 - 3CholTEG |
| 192 | G*A*T*T*G*G*G*A*T*T*G*C*A*A*G*T*A*T*G*A- (Spacer 18)2 - 3CholTEG |
| 193 | G*A*G*C*A*C*A*C*G*C*C*A*C*A*A*T*G*C*C*T- (Spacer 18)2 - 3CholTEG |
| 194 | A*G*T*C*T*T*C*T*T*G*T*C*T*C*A*G*C*C*T*T- (Spacer 18)2 - 3CholTEG |
| 195 | C*C*A*C*C*T*C*C*T*G*C*G*C*T*C*A*G*T*C*T- (Spacer 18)2 - 3CholTEG |
| 196 | T*C*A*C*A*C*A*G*C*C*T*A*C*T*G*C*A*G*C*C- (Spacer 18)2 - 3CholTEG |
| 197 | C*A#G#A#G#T*C*C*T*C*G*G*T*A*G*A#A#C*T*T- (Spacer 18)2 - 3CholTEG |
| 198 | G*A#A#A#T#C*T*A*A*T*T*T*T*T*C*A#G#T*T*T- (Spacer 18)2 - 3CholTEG |

* = phosphorothioate internucleotide linkage;
# = phosphodiester internucleotide linkage;
Spacer 18 = internal hexaethyleneglycol spacer;
3CholTEG = 3′-Cholesteryl-TEG;
TEG = tetraethylene glycol

In some embodiments, the first oligonucleotide and the second oligonucleotides are in the same SNA. In some embodiments, the first oligonucleotide and the second oligonucleotide are in different SNAs, where such SNAs can be administered as a mixture in the same composition, wherein the composition optionally comprises a pharmaceutically acceptable carrier, or one SNA after the other. In some embodiments, the SNA contains more than two distinct oligonucleotides. In some embodiments, the SNA contains oligonucleotides that target more than two distinct targets.

In some embodiments, a SNA described herein comprises or consists of an oligonucleotide (e.g., antisense oligonucleotide) that is 50% to 100% identical to the nucleic acid sequence of CCCACAGGGGCATGUAGU (SEQ ID NO: 58). In some embodiments, a SNA described herein comprises an oligonucleotide that is 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 58.

In some embodiments, a SNA described herein comprises or consists of an oligonucleotide (e.g., antisense oligonucleotide) that is 50% to 100% identical to the nucleic acid sequence of mCmCmCmAmCmAmGmG*mG*mG*mC*mA*mT*mGmUmAmGmU (SEQ iD NO: 59), wherein * is a phosphorothioate linkage and m is a 2′-O-methylated nucleotide. In some embodiments, a SNA described herein comprises an oligonucleotide that is 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 59.

In some embodiments, a SNA described herein comprises or consists of an oligonucleotide (e.g., antisense oligonucleotide) that is 50% to 100% identical to the nucleic acid sequence of mCmCmCmAmCmAmGmG*mG*mG*mC*mA*mT*mGmUmAmGmU/Spacer18/Spacer18/3 CholTEG (SEQ ID NO: 211), wherein * is a phosphorothioate linkage, m is a 2′-O-methylated nucleotide, Spacer18 is a hexa(ethylene glycol) spacer, and 3CholTEG is tri(ethylene glycol) bound to a cholesterol. In some embodiments, a SNA described herein comprises an oligonucleotide that is 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 211.

| Name | SEQ ID NO | Sequence | Target | Lipidated? |
|---|---|---|---|---|
| hIL17RA1 | 199 | mCmCmCmAmCmAmGG*G*G*C*A* | HJ7RA | Yes |
| 077 (7/6/5) | | T*mGmUmAmGmU/isp18//isp1 | | |
| (5′ to 3′) | | 8//3CholTEG/ | | |
| hIL17RA_ | 200 | mCmCmCmAmCmAmGG*G*G*C*A* | IL17RA | No |
| 1077 | | T*mGmUmAmGmU | | |
| (7/6/5) | | | | |
| Linear (3′- | | | | |
| DMT) | | | | |
| hIL1RAPv6 | 201 | moeA*moeT*MoeG*moeG*moeT | IL1RAP | Yes |
| _1377- | | *moeA*moeA*moeA*/5-Me- | | |
| moePS | | moeC/*moeA*moeA*/5-Me- | | |
| | | moeC/*moeA*moeA*moeT*moe | | |
| | | G*moeA*moeG/isp18//isp18 | | |
| | | //3CholTEG/ | | |
| hILIRAPv | 202 | moeA*moeT*moeG*moeG*moeT | IL1RAP | No |
| -1377- | | *moeA*moeA*moeA*/5-Me- | | |
| moePS | | moeC/*moeA*moeA*/5-Me- | | |
| | | moeC/*moeA*moeA*moeT*moe | | |
| | | G*moeA*moeG | | |
| IL 1 RAP | 203 | moeU*moeU*moeU*moeC*moeA | IL1RAP | Yes |
| PS-373 | | moeUmoeC*moeU*moeG*moe | | |
| MOE Choi | | U*moeU*moeC*moeC*moeA*mo | | |
| | | eA*moeA*moeA*moeU*moeG*m | | |
| | | oeA*moeG/isp18//isp18//3 | | |
| | | CholTEG/ | | |
| IL1RAP | 204 | moeU*moeU*moeU*moeC*moeA | IL1RAP | No |
| PS-373moe | | moeUmoeC*moeU*moeG*moe | | |
| linear | | U*moeU*moeC*moeC*moeA*mo | | |
| | | eA*moeA*moeA*moeU*moeG*m | | |
| | | oeA*moeG | | |
| E3I3 all PS | 205 | moeG*/5-Me- | REST | Yes |
| (3′-DMT) | | moeC/*moeA*moeG*moeT*/5- | | |
| | | Me-moeC/*moeA*/5Me | | |
| | | moeC/*/5-Me- | | |
| | | moeC/*moeA*moeT*/5-Me- | | |
| | | moeC/*moeT*moeT*moeA*/5- | | |
| | | Me-moe/*/5-Me- | | |
| | | moeC/*moeA*moeA*/5-Me- | | |
| | | moeC/*/5-Me- | | |
| | | moeC/moeT*moeG*moeA*moe | | |
| | | A/iSp18//iSp18// | | |
| | | 3CholTEG/ | | |
| E3I3 MOE | 206 | moeG*/5-Me- | REST | No |
| all PS | | moeC/*moeA*moeG*moeT*/5- | | |
| linear | | Me-moeC/*moeA*/5-Me- | | |
| | | moeC/*/5-Me- | | |
| | | moeC/*moeA*moeT*/5-Me- | | |
| | | moeC/*moeT*moeT*moeA*/5- | | |
| | | Me-moeC/*/5-Me- | | |
| | | moeC/*moeA*moeA*/5-Me- | | |
| | | moeC/*/5-Me- | | |
| | | moeC/*moeT*moeG*moeA*moe | | |
| | | A | | |
| STAT3 ST7 | 207 | moeT*moeT*/5-Me- | STAT3 | Yes |
| Chol | | moeC/*moeA*/5Me | | |
| (3′-DMT) | | moeC/*moeT*moeT*moeG*/5- | | |
| | | Me-moeC/*/5-Me- | | |
| | | moeC/*moeT*/5-Me- | | |
| | | moeC/*/5-Me- | | |
| | | moeC/*moeT*moeT*moeG*moe | | |
| | | A*/5Me-moeC/*moeT*/5 | | |
| | | Me-moeC/*moeT*moeT* | | |
| | | moeG/isp18//isp18// | | |
| | | 3CholTEG/ | | |
| STAT3 ST7 | 208 | moeT*moeT*/5Me | STAT3 | No |
| linear | | moeC/*moeA*/5-Me- | | |
| | | moeC/*moeT*moeT*moeG*/5- | | |
| | | Me-moeC/*/5-Me- | | |
| | | moeC/*moeT*/5-Me- | | |
| | | moeC/*/5-Me- | | |
| | | moeC/*moeT*moeT*moeG*moe | | |
| | | A*/5-Me-moeC/*moeT*/5- | | |
| | | Me-moeC/*moeT*moeT*moeG | | |
| AntiControl | 209 | mGmUmUmUmCmAC*C*A*C*C*C* | Control | Yes |
| (5′-3′) | | mAmAmUmUmCmC/iSp18//iSp1 | | |
| | | 8//3CholTEG/ | | |
| SMN | 210 | moeT*moeG*moeT*moeA*moeT | Control | Yes |
| Control-1 | | */5-Me-moeC/*moeT*/5-Me- | | |
| Linear | | moeC/*moeA*moeT*moeT*moe | | |
| | | G*moeT*moeA*moeG | | |

NCBI Transcripts

| Gene Name | Accession Number |
|---|---|
| IL17RA | NC_000022.11 Reference GRCh38.p13 Primary Assembly |
| IL1RAP | NC_000003.12 Reference GRCh38.p13 Primary Assembly |
| REST | NC_000004.12 Reference GRCh38.p13 Primary Assembly |
| STAT3 | NC_000017.11 Reference GRCh38.p13 Primary Assembly |

In some embodiments, a SNA described herein comprises or consists of an oligonucleotide (e.g., antisense oligonucleotide) that targets REST and that is 50% to 100% identical to the nucleic acid sequence of GCAGTCACCATCTITACCAACCTGAA (SEQ ID NO: 213), SEQ ID NO: 205, or SEQ ID NO: 206. In some embodiments, a SNA described herein comprises an oligonucleotide that is 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 212, SEQ ID NO: 213, SEQ ID NO: 205 or SEQ ID NO: 206. (See e.g., Chen et al., J Cell Mol Med (2017) 21(11):2974-2984, which is incorporated herein by reference in its entirety).

In some embodiments, a SNA described herein comprises or consists of an oligonucleotide (e.g., antisense oligonucleotide) that targets STAT3 and is 50% to 100% identical to the nucleic acid sequence of TTICACTITGCCTCCTITGACTCTITG (SEQ ID NO: 214), SEQ ID NO: 207 or SEQ ID NO: 208. In some embodiments, a SNA described herein comprises an oligonucleotide that is 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 214, SEQ ID NO: 207 or SEQ ID NO: 208. (See e.g., Zammarchi et al., PNAS (2011) 108(43): 17779-84, which is incorporated herein by reference in its entirety).

In some embodiments, a SNA described herein comprises or consists of an oligonucleotide (e.g., antisense oligonucleotide) that targets IL1RAP and that is 50% to 100% identical to the nucleic acid sequence of CTCATTGTTGTTTACCAT (SEQ ID NO: 9), the nucleic acid sequence of ATGGTAAACAACAATGAG (SEQ ID NO: 11), the nucleic acid sequence of UUUCAUCUGUUCCAAAAUGAG (SEQ ID NO: 212), SEQ ID NO: 201, SEQ ID NO: 202, SEQ ID NO: 203, or SEQ ID NO: 204. In some embodiments, a SNA described herein comprises an oligonucleotide that is 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 9, SEQ ID NO: 11, SEQ ID NO: 212, SEQ ID NO: 201, SEQ ID NO: 202, SEQ ID NO: 203, or SEQ ID NO: 204. In some embodiments, a SNA described herein comprises or consists of an oligonucleotide (e.g., antisense oligonucleotide) that is 50% to 100% identical to the nucleic acid sequence of TGTATCTCATTGTAG (SEQ ID NO: 215). In some embodiments, a SNA described herein comprises an oligonucleotide that is 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 215. In some embodiments, the nucleic acid sequence of SEQ ID NO: 215 is used as a control.

In some embodiments, a modification to one or more of the nucleotides of an oligonucleotide or antisense oligonucleotide described herein decreases or prevents RNAse-H-catalyzed mRNA degradation. In some embodiments, the modification is a 2′-methoxyethyl (2′-MOE) modification. In some embodiments, the modification is a 2′-O-methyl modification. In some embodiments, other modifications, such as modifications known to one of ordinary skill in the art, decrease or prevent RNAse-H catalyzed mRNA degradation. Without wishing to be bound by theory, oligonucleotides or antisense oligonucleotides, such as the oligonucleotides or antisense oligonucleotides described herein, that are less prone or completely protected from RNAse-H-catalyzed mRNA degradation are useful in therapy that modifies mRNA splicing. In some embodiments, the modification is used in combination with traditional antisense/siRNA therapy. As described herein, traditional antisense/siRNA therapy relates to RNAse-H dependent cleavage of mRNA; traditional antisense/siRNA therapy is the RISC-catalyzed mRNA degradation. In exon modulation or splice modulation, the aim is not to degrade the target mRNA. In some embodiments, only the splicing patterns are altered.

In some embodiments, the present disclosure provides administration of a first SNA into the cerebrospinal fluid (CSF), in combination with systemic delivery of a second SNA. Systemic administration and CSF administration can occur simultaneously, separately or sequentially. In some embodiments, a subject receives a first dose of a SNA in the CSF and subsequently receives a second dose of a SNA through a different route of administration. In some embodiments, a subject receives a first dose of a SNA in the CSF and subsequently receives a second dose of an antisense compound systemically. In some embodiments, the SNA administered into the CSF comprises the oligonucleotide of SEQ ID NO:1 or SEQ ID NO: 16.

In some embodiments, a target precursor transcript is associated with a disease or condition. In certain such embodiments, an oligomeric compound comprising or consisting of a modified oligonucleotide that is complementary to the target precursor transcript is used to treat the disease or condition. In certain such embodiments, the compound modulates processing of the target precursor transcript to produce a beneficial target processed transcript. In certain such embodiments, the disease or condition is associated with aberrant processing of a precursor transcript. In certain such embodiments, the disease, disorder or condition is related to or associated with an abnormality in splice modulation of a pre-mRNA.

According to some aspects, methods for treating a subject having a disease or disorder related to an abnormality in splice modulation are contemplated herein. In some embodiments, the method comprises administering to a subject having the disease or disorder related to an abnormality in splice modulation a SNA in an effective amount to decrease expression levels of a protein of interest under a baseline level in the subject in order to treat the disease or disorder related to an abnormality in splice modulation.

In some embodiments, the SNA is a SNA disclosed herein.

In some embodiments, a SNA described herein is used for the treatment of a disease, disorder or condition is related to or associated with an abnormality in splice modulation of a pre-mRNA. A non-limiting example includes spinal muscular atrophy (SMA).

In some embodiments, SMA is caused by a reduction of the SMN protein. In another embodiment, SMA is caused by a mutation in the SMN1 gene. In one embodiment, the type of SMA can be SMA1, SMA2, SMA3, SMA4, SMARD, SBMA, or DSMA.

In some embodiments, a SNA described herein is used for the treatment of a genetic disorder. Non-limiting examples include achondroplasia, alpha-1 antitrypsin deficiency, antiphospholipid syndrome, autism, autosomal dominant polycystic kidney disease, breast cancer, charcot-marie-tooth, colon cancer, cri du chat, crohn's disease, cystic fibrosis, dercum disease, down syndrome, duane syndrome, duchenne muscular dystrophy, factor v leiden, thrombophilia, familial hypercholesterolemia, familial mediterranean fever, fragile x syndrome, gaucher disease, hemochromatosis, hemophilia, holoprosencephaly, huntington's disease, klinefelter syndrome, marfan syndrome, myotonic dystrophy, neurofibromatosis, noonan syndrome, osteogenesis imperfecta, parkinson's disease, phenylketonuria, poland anomaly, porphyria, progeria, prostate cancer, retinitis pigmentosa, severe combined immunodeficiency (scid), sickle cell disease, skin cancer, SMA, tay-sachs, thalassemia, trimethylaminuria, turner syndrome, velocardiofacial syndrome, wagr syndrome, and wilson disease.

In some embodiments, the disease or disorder is not achondroplasia, alpha-1 antitrypsin deficiency, antiphospholipid syndrome, autism, autosomal dominant polycystic kidney disease, breast cancer, charcot-marie-tooth, colon cancer, cri du chat, crohn's disease, cystic fibrosis, dercum disease, down syndrome, duane syndrome, duchenne muscular dystrophy, factor v leiden, thrombophilia, familial hypercholesterolemia, familial mediterranean fever, fragile x syndrome, gaucher disease, hemochromatosis, hemophilia, holoprosencephaly, huntington's disease, klinefelter syndrome, marfan syndrome, myotonic dystrophy, neurofibromatosis, noonan syndrome, osteogenesis imperfecta, parkinson's disease, phenylketonuria, poland anomaly, porphyria, progeria, prostate cancer, retinitis pigmentosa, severe combined immunodeficiency (scid), sickle cell disease, skin cancer, SMA, tay-sachs, thalassemia, trimethylaminuria, turner syndrome, velocardiofacial syndrome, wagr syndrome, or wilson disease.

Aspects of the invention relate to delivery of SNAs to a subject for therapeutic and/or diagnostic use. The SNAs may be administered alone or in any appropriate pharmaceutical carrier, such as a liquid, for example saline, or a powder, for administration in vivo. The SNAs can also be co-delivered with larger carrier particles or within administration devices. The SNAs may be formulated. The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients. It should be appreciated that any method of delivery of SNAs known in the art may be compatible with aspects of the invention.

As used herein, a “patient,” “individual,” “subject” or “host” refers to either a human, a nonhuman animal or a mammal. In some embodiments, the mammal is a vertebrate animal including but not limited to a dog, cat, horse, cow, pig, sheep, goat, turkey, chicken, primate, e.g., monkey, and fish (aquaculture species), e.g. salmon. Thus, the invention can also be used to treat diseases or disorders in non-human subjects.

In some embodiments, a SNA described herein is administered in one dose to treat a subject having the disease or disorder related to with an abnormality in splice modulation in an effective amount to increase expression levels of a protein of interest over a baseline level in the subject in order to treat the disease or disorder related to an abnormality in splice modulation. As used herein, a baseline level is the level of a protein of interest in the subject prior to treatment with a SNA described herein. In some embodiments, a subject having a disease or disorder related to an abnormality in splice modulation is administered at least two doses of a SNA, in an effective amount to increase expression levels of a protein of interest over a baseline level in the subject in order to treat a disease or disorder related to an abnormality in splice modulation. In some embodiments, the second dose is administered about 3 months, 6 months, 9 months, one year, 15 months, 18 months, 21 months or two years after the first dose. In some embodiments, the level of a corrected mRNA relative to a defective mRNA associated with a disease or disorder related to an abnormality in splice modulation in the subject is enhanced.

In some embodiments, a SNA described herein is administered at a dose between 0.1 mg and 10 mg, between 0.2 mg and 10 mg, between 0.3 mg and 10 mg, between 0.4 mg and 10 mg, between 0.5 mg and 10 mg, between 0.6 mg and 10 mg, between 0.7 mg and 10 mg, between 0.8 mg and 10 mg, between 0.9 mg and 10 mg, between 1 mg and 10 mg, between 1 mg and 1000 mg, between 1 mg and 900 mg, between 1 mg and 800 mg, between 1 mg and 700 mg, between 1 mg and 600 mg, between 1 mg and 500 mg, between 1 mg and 450 mg, between 1 mg and 400 mg, between 1 mg and 350 mg, between 1 mg and 300 mg, between 1 mg and 250 mg, between 1 mg and 200 mg, between 1 mg and 150 mg, between 1 mg and 100 mg, between 1 mg and 90 mg, between 1 mg and 80 mg, between 1 mg and 70 mg, between 1 mg and 60 mg, between 1 mg and 60 mg, between 1 mg and 50 mg, between 1 mg and 49 mg, between 1 mg and 48 mg, between 1 mg and 47 mg, between 1 mg and 46 mg, between 1 mg and 45 mg, between 1 mg and 44 mg, between 1 mg and 43 mg, between 1 mg and 42 mg, between 1 mg and 41 mg, between 1 mg and 40 mg, between 1 mg and 39 mg, between 1 mg and 38 mg, between 1 mg and 37 mg, between 1 mg and 36 mg, between 1 mg and 35 mg, between 1 mg and 34 mg, between 1 mg and 33 mg, between 1 mg and 32 mg, between 1 mg and 31 mg, between 1 mg and 30 mg, between 1 mg and 29 mg, between 1 mg in 28 mg, between 1 mg and 27 mg, between 1 mg and 26 mg, between 1 mg and 25 mg, between 1 mg and 24 mg, between 1 mg and 23 mg, between 1 mg and 22 mg, between 1 mg and 21 mg, between 1 mg and 20 mg, between 1 mg and 19 mg, between 1 mg and 18 mg, between 1 mg and 17 mg, between 1 mg and 16 mg, between 1 mg and 15 mg, between 1 mg and 14 mg, between 1 mg and 13 mg, between 1 mg and 12 mg, between 1 mg and 11 mg, between 1 mg and 10 mg, between 1 mg and 9 mg, between 1 mg and 8 mg, between 1 mg and 7 mg, between 1 mg and 6 mg, between 1 mg and 5 mg, between 1 mg and 4 mg, between 1 mg and 2 mg, between 1 mg and 1.5 mg, between 1 mg and 3 mg, between 3 mg and 5 mg, between 5 mg and 7 mg, between 7 mg and 9 mg, between 9 mg and 14 mg, between 15 mg and 17 mg, between 18 mg and 31 mg, between 31 mg and 33 mg, between 0.5 mg and 2 mg, between 2 mg and 4 mg, between 11 mg and 13 mg, between 23 mg and 25 mg, between 2 mg and 31 mg, between 2 mg and 30 mg, between 2 mg and 29 mg, between 2 mg and 28 mg, between 2 mg and 27 mg, between 2 mg and 26 mg, between 2 mg and 25 mg, between 2 mg and 24 mg, between 2 mg and 23 mg, between 2 mg and 22 mg, between 2 mg and 21 mg, between 2 mg and 20 mg, between 2 mg and 19 mg, between 2 mg and 18 mg, between 2 mg and 17 mg, between 2 mg and 16 mg, between 2 mg and 15 mg, between 2 mg and 14 mg, between 2 mg and 13 mg, between 2 mg and 12 mg, between 2 mg and 11 mg, between 2 mg and 10 mg, between 2 mg and 9 mg, between 2 mg and 8 mg, between 2 mg and 7 mg, between 2 mg and 6 mg, between 2 mg and 5 mg, between 2 mg and 3 mg.

In some embodiments, a SNA disclosed herein administered at a dose of or about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 40 mg, 14.5 mg, 50 mg, 15.5 mg, 60 mg, 16.5 mg, 70 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 50 mg, 400 mg, 450 mg, 500 mg, 550 mg, 600 mg, six and 50 mg, 700 mg, 750 mg, 800 mg, 850 mg, 900 mg, 950 mg, or 1000 mg, or any range or combination thereof.

In some embodiments, a SNA disclosed herein is administered at a dose of at least or at least about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 40 mg, 14.5 mg, 50 mg, 15.5 mg, 60 mg, 16.5 mg, 70 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 50 mg, 400 mg, 450 mg, 500 mg, 550 mg, 600 mg, 650 mg, 700 mg, 750 mg, 800 mg, 850 mg, 900 mg, 950 mg, or 1000 mg, or any range or combination thereof.

In some embodiments, a SNA disclosed herein is administered at a dose greater than or greater than about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 40 mg, 14.5 mg, 50 mg, 15.5 mg, 60 mg, 16.5 mg, 70 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 50 mg, 400 mg, 450 mg, 500 mg, 550 mg, 600 mg, 650 mg, 700 mg, 750 mg, 800 mg, 850 mg, 900 mg, 950 mg, or 1000 mg, or any range or combination thereof.

As used herein, “pharmaceutically acceptable carrier or diluent” refers to any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In some embodiments, a pharmaceutically acceptable carrier or diluent is sterile water; sterile saline; or sterile buffer solution. In some embodiments, the pharmaceutically acceptable carrier or diluent is a gel formulation. In some embodiments, the gel formulation comprises one or more of the ingredients listed in Table 3.

TABLE 3

| Ingredients | w/w % |
|---|---|
| Transcutol P | 25.00 |
| Glycerin | 5.00 |
| Methylparaben | 0.15 |
| Propylparaben | 0.05 |
| HEC (250 HHX Pharm) | 1.00 |
| Purified Water (Part I) | 5.00 |
| Monobasic Sodium Phosphate, Anhydrous | 0.037 |
| Dibasic Sodium Phosphate, Anhydrous | 0.31 |
| Sodium Metabisulfite | 0.20 |
| Disodium EDTA | 0.10 |
| Sodium Citrate | 0.25 |
| DOPC Liposomes | 5.89 |
| Purified Water (Part II) | 57.00 |
| Oligonucleotide | 0.28 |
| Sodium Hydroxide Solution, 10% (w/w) | Adjust pH to 7.5-8.0 |
| Diluted Hydrochloric Acid, 10% (w/v) | Adjust pH to 7.5-8.0 |
| Purified Water (Part III) | To 100.00 |

In some embodiments, the gel formulation consists of the ingredients listed in Table 3.

As used herein, “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein, “pharmaceutical composition” means a mixture of substances suitable for administering to a subject. For example, a pharmaceutical composition may comprise an antisense compound and a sterile aqueous solution. In some embodiments, a pharmaceutical composition shows activity in free uptake assay in certain cell lines.

For use in therapy, an effective amount of the SNAs can be administered to a subject by any mode that delivers the SNAs to the desired cell. Administering pharmaceutical compositions may be accomplished by any means known to the skilled artisan. Routes of administration include but are not limited to oral, intrathecal, respiratory, parenteral, intramuscular, intravenous, subcutaneous, mucosal, intranasal, sublingual, intratracheal, inhalation, ocular, vaginal, dermal, or rectal administration, and by direct injection.

In some embodiments, the intrathecal administration is through a lumbar puncture. (See e.g., Astrid et al. European Journal of Paediatric Neurology (2018) 22(1):122-7 and Hach6 et al. Journal of Child Neurology 31.7 (2016):899-906, the contents of which are incorporated by reference in their entirety).

In some embodiments, any of the SNAs described herein are delivered intrathecally (IT). In some embodiments, any of the SNAs described herein are in a formulation that is compatible with intrathecal administration. Non-limiting examples of formulations that are compatible with intrathecal administration include artificial cerebral spinal fluid (aCSF); 100 mM sodium phosphate, 150 mM NaCl, 0.001% P 80; 10 mM citrate, 150 mM NaCl; 5% dextran in saline (hyperbaric solution); 0.75% or 7.5% glucose; paraben (methyl—and propylparabens); glycerin (50%); isotonic mannitol in normal saline; EDTA; DepoFoam; PEG suspension 2.5% PEG (3400); and 0.9% NaCl with 2.5% PEG and 0.025% polysorbate 80. Other formulations that are known to one of ordinary skill in the art are also contemplated herein.

In some embodiments, the disease or disorder is cancer. In some embodiments, the cancer is selected from the group consisting of melanoma, renal cancer, clear cell carcinoma, prostate cancer, hormone refractory prostate adenocarcinoma, breast cancer, colon cancer, lung cancer, non-small cell lung cancer, small cell lung cancer, bone cancer, pancreatic cancer, pancreatic adenocarcinoma, skin cancer, cancer of the head or neck, cutaneous or intraocular malignant melanoma, uterine cancer, ovarian cancer, rectal cancer, stomach cancer, testicular cancer, thyroid cancer, anaplastic thyroid cancer, uterine cancer, carcinoma of the fallopian tubes, carcinoma of the endometrium, carcinoma of the cervix, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, biliary tract cancer, brain cancer, breast cancer, cervical cancer, choriocarcinoma, esophageal cancer, gastric cancer, an intraepithelial neoplasm, lymphoma, liver cancer, neuroblastoma, oral cancer, sarcoma, hairy cell leukemia, chronic myelogenous leukemia, cutaneous T-cell leukemia, multiple myeloma, renal cell carcinoma, lymphoma, bladder cancer, glioblastoma multiforme, Merkel cell carcinoma, cutaneous squamous cell carcinoma, melanoma or squamous cell carcinoma of the head and neck. In some embodiments, the cancer is environmentally-induced cancers, including those induced by asbestos, or any combinations thereof. In some embodiments, the cancer is not melanoma.

In some embodiments, the cancer is selected from the group consisting of pleomorphic sarcoma, gastrointestinal stromal tumor (GIST), liposarcoma, leiomyosarcoma, synovial sarcoma, malignant peripheral nerve sheath tumor, rhabdomyosarcoma, angiosarcoma, fibrosarcoma, dermatofibrosarcoma protuberans, epithelioid sarcoma, myxoma, mesenchymoma, vascular sarcoma, neurilemmoma, bone sarcoma, osteosarcoma, Ewing's sarcoma, chondrosarcoma, Kaposi sarcoma, solitary fibrous tumor, chordoma, desmoid-type fibromatosis, fibroblastic sarcoma, giant cell tumor of the bone, gynaecological sarcoma, soft tissue sarcoma, angioleiomyoma, leiomyoma, smooth muscle sarcoma, fibrohistiocytic sarcoma, sebaceous cell carcinoma and eccrine carcinoma. In some embodiments, the cancer is characterized as microsatellite instability high, or MSI-H, or mismatch repair deficient, or dMMR. MSI-H or dMMR cancers are characterized by defects in DNA replication, particularly in the microsatellite regions. The presence of MSI-H and dMMR tumors has been reported in diverse cancer types, including colon, colorectal, endometrial, biliary, gastric, gastroesophageal junction, pancreatic, small intestinal, breast, triple negative breast, prostate, bladder, esophageal, sarcoma, thyroid, retroperitoneal adenocarcinoma, small cell lung, ovarian, pancreatic, prostate, central nervous system, and non-small cell lung cancers.

In some embodiments, the disease or disorder is an inflammatory disease or disorder.

In some embodiments, the inflammatory disease or disorder is selected from the group consisting of an autoimmune disease, an infectious disease, transplant rejection or graft-versus-host disease, a pulmonary disorder, an intestinal disorder, a cardiac disorder, sepsis, a spondyloarthropathy, a metabolic disorder, a hepatic disorder, a skin disorder and a nail disorder.

In some embodiments, the inflammatory disease or disorder is selected from the group consisting of atopic dermatitis, epidermolysis bullosa, uveitis, gout, polymyalgia rheumatica, osteoarthritis, systemic-onset juvenile idiopathic arthritis, schnitzler syndrome, familial mediterranean fever, cryopyrin-associated periodic syndrome (CAPS), hyper-igd syndrome (HIDS), TNF receptor-associated periodic syndrome (TRAPs), type 2 diabetes, proliferative diabetic retinopathy, wet age-related macular degeneration, chronic obstructive pulmonary disease, type 1 diabetes, pyoderma gangrenosum, dry eye syndrome, and acne vulgaris, rheumatoid arthritis, psoriasis, psoriatic arthritis, psoriasis in combination with psoriatic arthritis, ulcerative colitis, Crohn's disease, vasculitis. Behcet's disease, ankylosing spondylitis, asthma, chronic obstructive pulmonary disorder (COPD), idiopathic pulmonary fibrosis (IPF), restenosis, anemia, pain and hepatitis C virus infection.

In some embodiments, the autoimmune disease is selected from the group consisting of rheumatoid arthritis, rheumatoid spondylitis, osteoarthritis, gouty arthritis, allergy, multiple sclerosis, autoimmune uveitis, and nephritic syndrome.

The term “effective amount” is used interchangeably with the term “therapeutically effective amount” and refers to the amount of at least one SNA described herein, at dosages and for periods of time necessary to achieve the desired therapeutic result, for example, to reduce or stop at least one symptom of a disease or disorder related to an abnormality in splice modulation, for instance a disease or disorder disclosed in Table 1. For example, an effective amount using the methods as disclosed herein would be considered as the amount sufficient to reduce a symptom of the disease or disorder related to an abnormality in splice modulation by at least 10%. An effective amount as used herein would also include an amount sufficient to prevent or delay the development of a symptom of the disease, alter the course of a symptom disease (for example but not limited to, slow the progression of a symptom of the disease), or reverse a symptom of the disease or disorder. Accordingly, the term “effective amount” or “therapeutically effective amount” as used herein refers to the amount of a pharmaceutical composition described herein to alleviate at least one symptom of a disease or disorder associated with or related to an abnormality in splice modulation. Stated another way, “therapeutically effective amount” of an antisense oligonucleotide SNA as disclosed herein is the amount of SNA which exerts a beneficial effect on, for example, the symptoms of a disease or disorder related to an abnormality in splice modulation. The dosage administered, as single or multiple doses, to an individual will vary depending upon a variety of factors, including pharmacokinetic properties of the SNA, the route of administration, conditions and characteristics (sex, age, body weight, health, size) of subjects, extent of symptoms, concurrent treatments, frequency of treatment and the effect desired. A therapeutically effective amount is also one in which any toxic or detrimental effects of the therapeutic agent are outweighed by the therapeutically beneficial effects. The effective amount in each individual case can be determined empirically by a skilled artisan according to established methods in the art and without undue experimentation. In general, the phrases “therapeutically-effective” and “effective for the treatment, prevention, or inhibition”, are intended to qualify the antisense oligonucleotide SNA as disclosed herein which will achieve the goal of reduction in the severity of at least one symptom of a disease or disorder related to an abnormality in splice modulation.

According to some aspects, methods for delivering a stable level of antisense oligonucleotides to a central nervous system (CNS) of a subject having a CNS disease or disorder are contemplated herein. In some embodiments, the method comprises administering to a subject having a neurodegenerative disease or disorder a spherical nucleic acid (SNA) in an effective amount to deliver antisense oligonucleotides to the CNS of the subject, wherein the administration of SNA delivers about 2% to about 150% more antisense oligonucleotides to one or more tissues or regions of the CNS of the subject than administration of linear antisense oligonucleotides that are not in a SNA, wherein the SNA comprises a core and antisense oligonucleotides comprised of 10 to 60 linked nucleosides in length, wherein the antisense oligonucleotides are attached to the core and thus form an oligonucleotide shell, wherein the CNS disease or disorder is not autism, Alzheimer's disease, Parkinson's disease, spinal muscular atrophy, or characterized by muscle wasting and loss of muscle function.

According to some aspects, methods are disclosed for delivering a stable level of antisense oligonucleotides to a central nervous system (CNS) of a subject having a CNS disease or disorder comprises administering to a subject having a neurodegenerative disease or disorder a spherical nucleic acid (SNA) in an effective amount to deliver a first antisense oligonucleotide and a second antisense oligonucleotide to the CNS of the subject, wherein the administration of SNA delivers about 2% to about 150% more antisense oligonucleotides to one or more tissues or regions of the CNS of the subject than administration of linear antisense oligonucleotides that are not in a SNA, wherein the SNA comprises a core and antisense oligonucleotides comprised of 10 to 60 linked nucleosides in length, wherein the antisense oligonucleotides are attached to the core and thus form an oligonucleotide shell.

According to some aspects, methods are disclosed for delivering a stable level of antisense oligonucleotides to a central nervous system (CNS) of a subject having a CNS disease or disorder, the method comprising administering to a subject having a neurodegenerative disease or disorder a first spherical nucleic acid (SNA) in an effective amount to deliver a first antisense oligonucleotide and a second SNA to deliver a second antisense oligonucleotide to the CNS of the subject, wherein the administration of SNA delivers about 2% to about 150% more antisense oligonucleotides to one or more tissues or regions of the CNS of the subject than administration of linear antisense oligonucleotides that are not in a SNA, wherein the SNA comprises a core and antisense oligonucleotides comprised of 10 to 60 linked nucleosides in length, wherein the antisense oligonucleotides are attached to the core and thus form an oligonucleotide shell.

In some embodiments, the CNS disease or disorder is SMA.

In some embodiments, the CNS disease or disorder is encephalitis, poliomyelitis, essential tremor, multiple sclerosis, cancer of the nervous system, addiction, attention deficit/hyperactivity disorder (ADHD), bipolar disorder, catalepsy, depression, epilepsy/seizures, infection, locked-in syndrome, meningitis, migraine, myelopathy or Tourette's syndrome.

In some embodiments, the SNA is administered intrathecally (IT). In some embodiments, the SNA is administered in the lower lumbar region. In some embodiments, the SNA is IT-administered through a lumbar puncture.

In some embodiments, the subject is a mammal. In some embodiments, the subject is a rat or mouse. In some embodiments, the subject is a human.

In some embodiments, a stable level is achieved when at least 50% of the antisense oligonucleotides are present in a tissue of the CNS within three days of administration of the SNA to the subject, relative to the amount of antisense oligonucleotides present in the tissue of the CNS within one hour of administration of the SNA to the subject. In some embodiments, a stable level is achieved when at least 50% of the antisense oligonucleotides are present in a tissue of the CNS within 48 hours of administration of the SNA to the subject, relative to the amount of antisense oligonucleotides present in the tissue of the CNS within one hour of administration of the SNA to the subject. In some embodiments, a stable level is achieved when at least 50% of the antisense oligonucleotides are present in a tissue of the CNS within 24 hours of administration of the SNA to the subject, relative to the amount of antisense oligonucleotides present in the tissue of the CNS within one hour of administration of the SNA to the subject.

In some embodiments, less than 50% of the antisense oligonucleotides are detectable within six hours of administration to the subject in one or both kidneys of the subject. In some embodiments, less than 40% of the antisense oligonucleotides are detectable within six hours of administration to the subject in one or both kidneys of the subject. In some embodiments, less than 30% of the antisense oligonucleotides are detectable within six hours of administration to the subject in one or both kidneys of the subject. In some embodiments, less than 20% of the antisense oligonucleotides are detectable within six hours of administration to the subject in one or both kidneys of the subject. In some embodiments, less than 10% of the antisense oligonucleotides are detectable within six hours of administration to the subject in one or both kidneys of the subject. In some embodiments, less than 5% of the antisense oligonucleotides are detectable within six hours of administration to the subject in one or both kidneys of the subject.

In some embodiments, the SNA is a SNA disclosed herein.

In some embodiments, the SNA is in a formulation and wherein the formulation comprises artificial cerebral spinal fluid (aCSF).

In some embodiments, the one or more tissues or regions of the CNS is one or more regions of the brain.

In some embodiments, the one or more regions of the brain is selected from the group consisting of the amygdala, basal ganglia, cerebellum, corpus callosum, cortex, hippocampus, hypothalamus, midbrain, olfactory region, one or more ventricles, septal area, white matter and thalamus.

In some embodiments, the one or more tissues or regions of the CNS are the cervical cerebral spinal fluid (CSF) or thoracic CSF.

In some embodiments, the antisense oligonucleotides in the SNA have different routes of distribution and clearance from the corresponding linear antisense oligonucleotides that are not in a SNA.

In some embodiments, any of the SNAs described herein are administered to a subject having a disease or disorder disclosed herein in an effective amount to increase expression levels of a protein or mRNA of interest over a baseline level of a protein or mRNA of interest to treat the disease or disorder in the subject, wherein the effective amount of SNA is less than 12 mg/dose. In some embodiments, the effective amount of SNA is less than 11.5 mg/dose, 11 mg/dose, 10.5 mg/dose, 10 mg/dose, 9.5 mg/dose, 9 mg/dose, 8.5 mg/dose, 8 mg/dose, 7.5 mg/dose, 7 mg/dose, 6.5 mg/dose, 6 mg/dose, 5.5 mg/dose, 5 mg/dose, 4.5 mg/dose, 3.5 mg/dose, 3 mg/dose, 2.5 mg/dose, 2 mg/dose, 1.5 mg/dose, 1 mg/dose, 0.5 mg/dose, or 0.1 mg/dose. In some embodiments, the SNA is administered to the CNS to treat a CNS disease or disorder.

In some embodiments, any of the SNAs described herein are administered to a subject having a disease or disorder disclosed herein in an effective amount to increase expression levels of a protein or mRNA of interest over a baseline level of a protein or mRNA of interest to treat the disease or disorder in the subject, wherein the effective amount of SNA is less than 12 mg/kg of body weight. In some embodiments, the effective amount of SNA is less than 11.5 mg/kg of body weight, 11 mg/kg of body weight, 10.5 mg/kg of body weight, 10 mg/kg of body weight, 9.5 mg/kg of body weight, 9 mg/kg of body weight, 8.5 mg/kg of body weight, 8 mg/kg of body weight, 7.5 mg/kg of body weight, 7 mg/kg of body weight, 6.5 mg/kg of body weight, 6 mg/kg of body weight, 5.5 mg/kg of body weight, 5 mg/kg of body weight, 4.5 mg/kg of body weight, 3.5 mg/kg of body weight, 3 mg/kg of body weight, 2.5 mg/kg of body weight, 2 mg/kg of body weight, 1.5 mg/kg of body weight, 1 mg/kg of body weight, 0.5 mg/kg of body weight, or 0.1 mg/kg of body weight. In some embodiments, the SNA is administered to the CNS to treat a CNS disease or disorder.

In some embodiments, any of the SNAs described herein are administered to a subject having a disease or disorder disclosed herein in an effective amount to increase expression levels of a protein or mRNA of interest over a baseline level of a protein or mRNA of interest to treat the disease or disorder in the subject, wherein the effective amount of SNA is more than 12 mg/dose. In some embodiments, the effective amount of SNA is more than 12.5 mg/dose, 13 mg/dose, 13.5 mg/dose, 14 mg/dose, 14.5 mg/dose, 15 mg/dose, 15.5 mg/dose, 16 mg/dose, 16.5 mg/dose, 17 mg/dose, 17.5 mg/dose, 18 mg/dose, 18.5 mg/dose, 19 mg/dose, 19.5 mg/dose, 20 mg/dose, 22 mg/dose, 24 mg/dose, 26 mg/dose, 28 mg/dose, 30 mg/dose, 40 mg/dose, 50 mg/dose, 60 mg/dose, 70 mg/dose, 80 mg/dose, 90 mg/dose, 100 mg/dose, 500 mg/dose, or 1000 mg/dose. In some embodiments, the SNA is administered to the CNS to treat a CNS disease or disorder.

In some embodiments, any of the SNAs described herein are administered to a subject having a disease or disorder disclosed herein in an effective amount to increase expression levels of a protein or mRNA of interest over a baseline level of a protein or mRNA of interest to treat the disease or disorder in the subject, wherein the effective amount of SNA is more than 12 mg/kg of body weight. In some embodiments, the effective amount of SNA is more than 12.5 mg/kg of body weight, 13 mg/kg of body weight, 13.5 mg/kg of body weight, 14 mg/kg of body weight, 14.5 mg/kg of body weight, 15 mg/kg of body weight, 15.5 mg/kg of body weight, 16 mg/kg of body weight, 16.5 mg/kg of body weight, 17 mg/kg of body weight, 17.5 mg/kg of body weight, 18 mg/kg of body weight, 18.5 mg/kg of body weight, 19 mg/kg of body weight, 19.5 mg/kg of body weight, 20 mg/kg of body weight, 22 mg/kg of body weight, 24 mg/kg of body weight, 26 mg/kg of body weight, 28 mg/kg of body weight, 30 mg/kg of body weight, 40 mg/kg of body weight, 50 mg/kg of body weight, 60 mg/kg of body weight, 70 mg/kg of body weight, 80 mg/kg of body weight, 90 mg/kg of body weight, 100 mg/kg of body weight, 500 mg/kg of body weight, or 1000 mg/kg of body weight. In some embodiments, the SNA is administered to the CNS to treat a CNS disease or disorder.

In some embodiments, any of the SNAs described herein are administered to a subject having a disease or disorder disclosed herein in an effective amount to increase expression levels of a protein or mRNA of interest over a baseline level of a protein or mRNA of interest to treat the disease or disorder in the subject, wherein the effective amount of SNA is or about 0.1 mg/dose, 0.2 mg/dose, 0.3 mg/dose, 0.4 mg/dose, 0.5 mg/dose, 0.6 mg/dose, 0.7 mg/dose, 0.8 mg/dose, 0.9 mg/dose, 1 mg/dose, 1.5 mg/dose, 2 mg/dose, 2.5 mg/dose, 3 mg/dose, 3.5 mg/dose, 4 mg/dose, 4.5 mg/dose, 5 mg/dose, 5.5 mg/dose, 6 mg/dose, 6.5 mg/dose, 7 mg/dose, 7.5 mg/dose, 8 mg/dose, 8.5 mg/dose, 9 mg/dose, 9.5 mg/dose, 10 mg/dose, 10.5 mg/dose, 11 mg/dose, 11.5 mg/dose, 12 mg/dose, 12.5 mg/dose, 13 mg/dose, 13.5 mg/dose, 14 mg/dose, 14.5 mg/dose, 15 mg/dose, 15.5 mg/dose, 16 mg/dose, 16.5 mg/dose, 17 mg/dose, 17.5 mg/dose, 18 mg/dose, 18.5 mg/dose, 19 mg/dose, 19.5 mg/dose, 20 mg/dose, 20.5 mg/dose, 21 mg/dose, 21.5 mg/dose, 22 mg/dose, 23 mg/dose, 24 mg/dose, 25 mg/dose, 26 mg/dose, 27 mg/dose, 28 mg/dose, 29 mg/dose, 30 mg/dose, 31 mg/dose, 32 mg/dose, 33 mg/dose, 34 mg/dose, 35 mg/dose, 36 mg/dose, 37 mg/dose, 38 mg/dose, 39 mg/dose, 40 mg/dose, 45 mg/dose, 50 mg/dose, 55 mg/dose, 60 mg/dose, 65 mg/dose, 70 mg/dose, 75 mg/dose, 80 mg/dose, 85 mg/dose, 90 mg/dose, 95 mg/dose, 100 mg/dose, 500 mg/dose, 1000 mg/dose or any range there of or combination thereof. In some embodiments, the SNA is administered to the CNS to treat a CNS disease or disorder.

In some embodiments, any of the SNAs described herein are administered to a subject having a disease or disorder disclosed herein in an effective amount to increase expression levels of a protein or mRNA of interest over a baseline level of a protein or mRNA of interest in the CNS of the subject to treat the disease or disorder in the subject, wherein the effective amount of SNA is or about 0.1 mg/kg of body weight, 0.2 mg/kg of body weight, 0.3 mg/kg of body weight, 0.4 mg/kg of body weight, 0.5 mg/kg of body weight, 0.6 mg/kg of body weight, 0.7 mg/kg of body weight, 0.8 mg/kg of body weight, 0.9 mg/kg of body weight, 1 mg/kg of body weight, 1.5 mg/kg of body weight, 2 mg/kg of body weight, 2.5 mg/kg of body weight, 3 mg/kg of body weight, 3.5 mg/kg of body weight, 4 mg/kg of body weight, 4.5 mg/kg of body weight, 5 mg/kg of body weight, 5.5 mg/kg of body weight, 6 mg/kg of body weight, 6.5 mg/kg of body weight, 7 mg/kg of body weight, 7.5 mg/kg of body weight, 8 mg/kg of body weight, 8.5 mg/kg of body weight, 9 mg/kg of body weight, 9.5 mg/kg of body weight, 10 mg/kg of body weight, 10.5 mg/kg of body weight, 11 mg/kg of body weight, 11.5 mg/kg of body weight, 12 mg/kg of body weight, 12.5 mg/kg of body weight, 13 mg/kg of body weight, 13.5 mg/kg of body weight, 14 mg/kg of body weight, 14.5 mg/kg of body weight, 15 mg/kg of body weight, 15.5 mg/kg of body weight, 16 mg/kg of body weight, 16.5 mg/kg of body weight, 17 mg/kg of body weight, 17.5 mg/kg of body weight, 18 mg/kg of body weight, 18.5 mg/kg of body weight, 19 mg/kg of body weight, 19.5 mg/kg of body weight, 20 mg/kg of body weight, 20.5 mg/kg of body weight, 21 mg/kg of body weight, 21.5 mg/kg of body weight, 22 mg/kg of body weight, 23 mg/kg of body weight, 24 mg/kg of body weight, 25 mg/kg of body weight, 26 mg/kg of body weight, 27 mg/kg of body weight, 28 mg/kg of body weight, 29 mg/kg of body weight, 30 mg/kg of body weight, 31 mg/kg of body weight, 32 mg/kg of body weight, 33 mg/kg of body weight, 34 mg/kg of body weight, 35 mg/kg of body weight, 36 mg/kg of body weight, 37 mg/kg of body weight, 38 mg/kg of body weight, 39 mg/kg of body weight, 40 mg/kg of body weight, 45 mg/kg of body weight, 50 mg/kg of body weight, 55 mg/kg of body weight, 60 mg/kg of body weight, 65 mg/kg of body weight, 70 mg/kg of body weight, 75 mg/kg of body weight, 80 mg/kg of body weight, 85 mg/kg of body weight, 90 mg/kg of body weight, 95 mg/kg of body weight, 100 mg/kg of body weight, 500 mg/kg of body weight, 1000 mg/kg of body weight or any range or combination thereof.

In some embodiments, at least two doses of any of the SNAs disclosed herein are administered to a subject having a disease or disorder disclosed herein in an effective amount to increase expression levels of a protein or mRNA of interest over a baseline level. In some embodiments, the second dose is administered about one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 27 days, 28 days, 29 days, 30 days, 31 days after the first dose. In some embodiments, the second dose is administered 15 days to about three months after the first dose. In some embodiments, the second dose is administered about 2 weeks, 3 weeks, 4 weeks, 5 weeks, 6 weeks, 7 weeks, 8 weeks, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 12 months, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, or 10 years after administration of the first dose.

In some embodiments, two or more doses of a SNA disclosed herein are administered at intervals of or about one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 27 days, 28 days, 29 days, 30 days, 31 days, 2 weeks, 3 weeks, 4 weeks, 5 weeks, 6 weeks, 7 weeks, 8 weeks, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 12 months, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, or 10 years or more than 10 years, or any ranges or combinations thereof.

In some embodiments, an effective amount refers to the amount that is able to deliver about 2% to about 150% more antisense oligonucleotides to one or more tissues or regions of the body of the subject than administration of a linear antisense oligonucleotide that is not in an SNA. In some embodiments, a SNA delivers about 2% to about 500%, about 2% to about 450%, about 2% to about 400%, about 2% to about 350%, about 2% to about 300%, about 2% to about 250%, about 2% to about 200%, about 2% to about 175%, about 2% to about 160%, about 2% to about 150%, about 2% to about 140%, about 2% to about 130%, about 2% to about 120%, about 2% to about 110%, about 2% to about 100%, about 2% to about 95%, about 2% to about 90% about 2% to about 85% to about 2% to about 80%, about 2% to about 75%, about 2% to about 70%, about 2% to about 65%, about 2% to about 60%, about 2% to about 55%, about 2% to about 50%, about 2% to about 45% to about 2% to about 40%, about 2% to about 35%, about 2% to about 30%, about 2% to about 25%, about 2% to about 20%, about 2% to about 15%, about 2% to about 10%, about 2% to about 5%, about 10% to about 500%, about 10% to about 450%, about 10% to about 400%, about 10% to about 350%, about 10% to about 300%, about 10% to about 250%, about 10% to about 200%, about 10% to about 175%, about 10% to about 160%, about 10% to about 150%, about 10% to about 140%, about 10% to about 130%, about 10% to about 120%, about 10% to about 110%, about 10% to about 100%, about 10% to about 95%, about 10% to about 90% about 10% to about 85% to about 10% to about 80%, about 10% to about 75%, about 10% to about 70%, about 10% to about 65%, about 10% to about 60%, about 10% to about 55%, about 10% to about 50%, about 10% to about 45% to about 10% to about 40%, about 10% to about 35%, about 10% to about 30%, about 10% to about 25%, about 10% to about 20%, about 10% to about 15%, about 10% to about 10%, about 10% to about 5% more antisense oligonucleotides to one or more tissues or regions of the body of the subject than administration of a linear antisense oligonucleotide that is not in an SNA.

In an embodiment, a second dose of SNA is administered one week to three weeks after the first dose of SNA, a third dose of SNA is administered one week to three weeks after the second dose of SNA, a fourth dose is administered two weeks to six weeks after the third dose of SNA, a fifth and subsequent doses of SNA are administered between two and six months after the preceding dose. In some embodiments, all the SNA doses are administered at the same or substantially the same time intervals. As disclosed herein, substantially the same time intervals refers to administration within three days of each other. In some embodiments, at least two of the SNA doses are administered at the same time interval and any remaining SNA doses at different time intervals, such as at any combination of the time intervals disclosed herein.

In some embodiments, any of the SNAs described herein are administered in an effective amount to deliver a stable level of the antisense oligonucleotides to the CNS of the subject. In some embodiments, the stable level of the antisense oligonucleotides is achieved when at least 50% of the antisense oligonucleotides are present in one or more tissues or one or more regions of the CNS of the subject within seven days of administration of the SNA to the subject, relative to the amount of antisense oligonucleotides present in the tissue of the CNS within one hour of administration of the SNA to the subject. In some embodiments, the stable level of the antisense oligonucleotides is achieved when at least 10%, 15%, 20%, 25%, 30%, 35%, 40%, 45%, 50%, 55%, 60%, 65%, 70%, 75%, 80%, 85%, 90%, 95%, or 100% of the antisense oligonucleotides are present in one or more tissues or one or more regions of the CNS of the subject within 6 hours, 18 hours, 24 hours, 48 hours, 72 hours, 4 days, 5 days, 6 days, 7 days, 8 days, 9 days, 10 days, 12 days, 14 days, 60 days, 18 days, 20 days, 22 days, 24 days, 26 days, 28 days, 30 days, 1.5 months, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 1 year, 1.5 years, 2 years, 2.5 years, 3 years, 3.5 years, 4 years, 4.5 years, 5 years, 6 years, 7 years, 8 years, 9 years, or 10 years of administration of the SNA to the subject, relative to the amount of antisense oligonucleotides present in the tissue of the CNS within 1 hour, 3 hours, 6 hours, 12 hours, or 24 hours of administration of the SNA to the subject.

In some embodiments less than 50% of the oligonucleotides or antisense oligonucleotides in any of the SNA disclosed herein are detectable within six hours of administration to the subject in one or both kidneys of the subject. In some embodiments, less than 100%, 95%, 90%, 85%, 80%, 75%, 70%, 65%, 60%, 55%, 50%, 45%, 40%, 35%, 30%, 25%, 20%, 15%, 10%, 5%, or 1% of the oligonucleotides or antisense oligonucleotides in any of the SNA described herein are detectable within 30 min., 1 hour, 2 hours, 3 hours, 4 hours, 5 hours, 6 hours, 12 hours, 18 hours, 24 hours, 48 hours, 72 hours, 4 days, 5 days, 6 days, 7 days, 8 days, 9 days, 10 days, 12 days, 14 days, 16 days, 18 days, 20 days, 22 days, 24 days, 26 days, 28 days, 30 days, 1.5 months, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 1 year, 1.5 years, 2 years, 2.5 years, 3 years, 3.5 years, 4 years, 4.5 years, 5 years, 6 years, 7 years, 8 years, 9 years, or 10 years of administration to the subject in one or both kidneys of the subject.

In some embodiments, the duration of the method for treating a disease or disorder disclosed herein with a SNA disclosed herein is for three months, for six months, for nine months, for one year, for 1.5 years, for two years, for 2.5 years, for 3 years, for 3.5 years, for 4 years, for 4.5 years, for 5 years, for 5.5 years, for 6 years, for 6.5 years, for 7 years, for 7.5 years, for 8 years, for 8.5 years, for 9 years, for 9.5 years, for 10 years, for 15 years, for 20 years or more than 20 years.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. The phrases “systemic administration,” “administered systemically”, “peripheral administration” and “administered peripherally” as used herein mean the administration of a pharmaceutical composition comprising at least an SNA as disclosed herein such that it enters the animal's system and, thus, is subject to metabolism and other like processes, for example, subcutaneous administration.

In some embodiments, a SNA described herein is administered to a cell in vitro or is administered to a subject in order for the SNA to come into contact with a cell of the subject in vivo. Non-limiting examples of a cell contemplated herein include a fibroblast, epithelial, endothelial, neuronal, adipose, cardiac, skeletal muscle, immune cell, hepatic, splenic, lung, circulating blood, gastrointestinal, renal, bone marrow, or pancreatic cell. The cell is from a somatic tissue including, but not limited to brain, liver, lung, gut, stomach, intestine, fat, muscle, uterus, skin, spleen, endocrine organ, bone, etc. In some embodiments, the cell is a mammalian cell. In some embodiments, the cell is a murine, bovine, simian, porcine, equine, ovine, or human cell.

In some embodiments, the cell is contacted with a SNA disclosed herein in vitro. In some embodiments, the cell is contacted with a SNA disclosed herein in vivo. In some embodiments, the cell is contacted with a SNA disclosed herein ex vivo.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with or related to an abnormality in splice modulation. The Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of a malignant disease, diminishment of extent of a malignant disease, stabilized (i.e., not worsening) state of a malignant disease, delay or slowing of progression of a malignant disease, amelioration or palliation of the malignant disease state, and remission (whether partial or total), whether detectable or undetectable. The term “treatment” of a disease also includes providing relief from the symptoms or side-effects of the disease (including palliative treatment).

The terms “significantly different than,” “statistically significant,” and similar phrases refer to comparisons between data or other measurements, wherein the differences between two compared individuals or groups are evidently or reasonably different to the trained observer, or statistically significant (if the phrase includes the term “statistically” or if there is some indication of statistical test, such as a p-value, or if the data, when analyzed, produce a statistical difference by standard statistical tests known in the art).

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage or effective amount can vary depending upon the dosage form employed, the route of administration utilized, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention without necessitating undue experimentation. The dose ratio between toxic and therapeutic effects is the therapeutic index and can be expressed as the ratio LD50/ED50. Compositions and methods that exhibit large therapeutic indices are preferred. A therapeutically effective dose or effective amount can be estimated initially from cell culture assays. Also, a dose can be formulated in animal models to achieve a circulating plasma concentration range that includes the 1C50 (i.e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, such as a bioassay known to one of ordinary skill in the art. The dosage can be determined by a physician and adjusted, as necessary, to suit observed effects of the treatment. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

As used herein, “ameliorates symptoms and/or defects” is improving any defect or symptom associated with a disease or disorder related to an abnormality in splice modulation, such as the diseases or disorders disclosed in Table 1. As compared with an equivalent untreated control, such reduction is by at least 5%, 10%, 20%, 40%, 50%, 60%, 80%, 90%, 95%, 99% or more as measured by any standard technique.

In another aspect, the present invention is directed to a kit including one or more of the components of a SNA previously discussed. A “kit,” as used herein, typically defines a package or an assembly including one or more of the compositions of the invention, and/or other compositions associated with the invention, for example, as previously described. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions that may be associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

In some embodiments, a kit associated with the invention includes one or more lipid cores. A kit can also include one or more oligonucleotides. A kit can also include one or more anchors or molecular species and/or linkers or linker moieties.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the use of the compositions, for example, for a particular use, e.g., to a sample. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein. As used herein, “promoting” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic. Internet, Web-based, etc.) that are clearly associated with the invention.

In one set of embodiments, the method of promotion may involve one or more instructions. As used herein. “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

All references, including patent documents, disclosed herein are incorporated by reference in their entirety.

According to some aspects, the SNA comprises a core and antisense oligonucleotides arranged in an oligonucleotide shell, wherein the oligonucleotides comprise a nucleotide backbone comprising a modification in one or more of the carbons in the five-carbon sugar, and wherein five nucleotides or fewer than five nucleotides do not comprise a modification in the five-carbon sugar. In some embodiments, four nucleotides or fewer than four nucleotides do not comprise a modification in the five-carbon sugar. In some embodiments, three nucleotides or fewer than three nucleotides do not comprise a modification in the five-carbon sugar. In some embodiments, two nucleotides or fewer than two nucleotides do not comprise a modification in the five-carbon sugar. In some embodiments, one nucleotide does not comprise a modification in the five-carbon sugar. As disclosed herein, a modification in the five-carbon sugar refers to the presence of at least one group, which is not a hydrogen (H) or a hydroxyl (OH) group at the 2′ position of the five-carbon sugar. In some embodiments, a modification in the five-carbon sugar refers to any of the modifications to the five-carbon sugar disclosed herein. In some embodiments, the five carbon sugar does not include a H and/or OH at the 2′-position of the five-carbon sugar.

In some embodiments, all of the nucleotides in the nucleotide backbone of the antisense oligonucleotides comprise a modification in one or more of the carbons in the five-carbon sugar.

In some embodiments, the modification is at the 2′-carbon of the five-carbon sugar. In some embodiments, the modification is a 2′-O-methylated nucleotide. In some embodiments, the antisense oligonucleotide comprises the nucleic acid sequence CCCACAGGGGCATGUAGU (SEQ ID NO: 58). In some embodiments, the antisense oligonucleotide comprises or consists of the nucleic acid sequence mCmCmCmAmCmAmGmG*mG*mG*mC*mA*mT*mGmUmAmGmU (SEQ ID NO: 59), wherein * is a phosphorothioate linkage and m is a 2′-O-methylated nucleotide. In some embodiments, the antisense oligonucleotide comprises the nucleic acid sequence mCmCmCmAmCmAmGmG*mG*mG*mC*mA*mT*mGmUmAmGmU/Spacer18/Spacer18/3 CholTEG (SEQ ID NO: 211), wherein * is a phosphorothioate linkage, m is a 2′-O-methylated nucleotide, Spacer18 is a hexa(ethylene glycol) spacer, and 3CholTEG is tri(ethylene glycol) bound to a cholesterol.

According to some aspects, the SNA comprises an antisense oligonucleotide comprised of 8 to 50 linked nucleosides in length targeted to a regulatory site of a pre-mRNA of interest and a linker comprising a molecular species at the 3′-end or the 5′-end of the antisense oligonucleotide, wherein the molecular species is a hydrophobic group comprising a stearyl.

In some embodiments, the stearyl is a distearyl.

The genomic nucleic acid sequence, pre-mRNA nucleic acid sequence, mRNA nucleic acid sequence and amino acid sequence of SMN2, for instance, which is a target related to a disease or disorder associated with an abnormality in spice modulation, are well known to one of ordinary skill in the art. Non-limiting examples include:

Homo sapiens genomic SMN2 nucleic acid sequence
NCBI Ref. Seq.: NG_008728.1
(SEQ ID NO: 12)
CCACAAATGT GGGAGGGCGA TAACCACTCG TAGAAAGCGT GAGAAGTTAC TACAAGCGGT
CCTCCCGGCC ACCGTACTGT TCCGCTCCCA GAAGCCCCGG GCGGCGGAAG TCGTCACTCT
TAAGAAGGGA CGGGGCCCCA CGCTGCGCAC CCGCGGGTTT GCTATGGCGA TGAGCAGCGG
CGGCAGTGGT GGCGGCGTCC CGGAGCAGGA GGATTCCGTG CTGTTCCGGC GCGGCACAGG
CCAGGTGAGG TCGCAGCCAG TGCAGTCTCC CTATTAGCGC TCTCAGCACC CTTCTTCCGG
CCCAACTCTC CTTCCGCAGC CTCGGGACAG CATCAAGTCG ATCCGCTCAC TGGAGTTGTG
GTCCGCGTTT TTCTACGTCT TTTCCCACTC CGTTCCCTGC GAACCACATC CGCAAGCTCC
TTCCTCGAGC AGTTTGGGCT CCTTGATAGC GTTGAGTGGA GGCCCTGCCG CGACTTGGCA
GTAGCTTATT TTGTTCACTC CTCTCTGGCT GGTGTGGGGG AGGTGGGGGC ATTAGGCCAG
GGTGAAGCAG GGGAACCACT TAGGAGTCTG TTAAGATGAT CTGAACTTCA GAACAAGATG
TTATTAACAG AGTGAAAGTA TTTGGATTCT GGGTATATTT TGAAATCGGA GGCAACAGGT
TTTTCAGATA GATTCGATAA CGGAGGTTAT CCTGAATAGT TGAAAAGATA AAGTTGCCTT
TTGCTGAGGT GGGAAAGAGA AGATTGCCAG TAGAGCAGGT TTCTCAGGAG TTCAGTCTTG
GGCATAGCAT GGTAGGGGTG AATTTGGCTG GAGTGAGTTG GAGAGTAGGA GAAGAGAAAT
CCAAGGCAAC ATTTGACCAG CCTGGGCAAC ATAGTGTGAC TCCGAGTCTG CAAAAATTAG
ACGGGTGTTG TGGTGCGCGT CTGTGGTCTC AGCTACCTGG AAGGTTCAGG CCTTGGAAGG
CTCAGGGAGG TGGAGGCTGC AGTGATCTGT GATTGCGCCT CTGCACTCCA GCCTGGGCGA
CAGAGCCAGA CCCTGTCTTA AAACAAAATA AACGGCCGGG CGCGGTGGCT CAAGCCTGTA
ATCCCAGCAC TTTGGGAGGC CGAGGCGGCC GGATGACAAG GTCAGGAGAT CGAGACCATC
CTGGCTAACA CGGTGAAACC CCGTCTCTAC TACAAATACA AAAAATTAGC CGGGCGTGGT
GACGGGCGCC TGTAGTCCCA GCTACTCGGG AGGCTGAGGC AGGAGAATGT CATGAAGCCG
GGAGGCGGAG CTTGCAGTGA GCCGAGATCG CGCCACTGCA CTCCAGCCTG GGCGATAGAG
CAAGACTCCG TCTCAAATAA ATAAATAAAT AAATAAATAA ATAATAAAAA CATCGGTAGG
CATATTTCAA GGAATTCTAT TTAAAAAAAA TTTTTTTAGA GACAAGTTCG CTCTCTGTGG
CCCAGGCTGG AGTACAGTGG CATGATCCTA GCCCATGGCA GCGTTGATCT CTTGGCCTCA
AGCGACCCTC CTTTGGAGTC GCTGGGCCTA AAGGAGTGAG CCACCACGAA ATTTTATTAT
AAATGGAGGG TAGAGAAATT GGGCAATAAA TGGAGGGGGA AGTGAGTTAA GAGGAATTTT
AATTATGTGT GTGTGGTTTT AAAAGAGGGG GGTCTTGCTC TGTTGCCCAG GCTGCTGGGG
TGCCAGTGGC GCAATCATGA ATCACTACAG CCTTGGACTC CTGGCCTCAA GCTATCCTCC
CACCTCTGCC TCCCAAAGTA CTGGGATTAC TAGTGTGAGC CACTGCACTA AGATAGGAGC
AACATGTTTC AGCATGTTTG TGGGTTGATA GGAAAGATGA GAATGGGAAA GTTGATGTCG
GAAAGAAGAC AATGGCTAGA GCAATGTCCT AGAGTAGGTA AGAAGGGATG GATTTGGCCT
TTGTTGGAAA CATTAGCGGT TCTTTTGGTG ACAGCTATAT AGTTAACACA TCTATGATAC
GTGAATGGGC AGATAGGATG GCAGGAGATT TTGAAAGTTC TCTTGATTCT TACTGTTCTC
TTAGTGAAAG AAGCAAGGTT ATCAGCTAGA AGCTGGGATG GGAGAGGAAA GAGAAGATGG
GAAGTAGATA GTTCTTTAGA AGAGTGGGCA AGGGTlGGAC TAGGGAAGTT TAGTGGAAAT
ATTGCTAGGC AACATAAAGA GCCTACTTGA GATTCGTGGT CATGAGTTGA AGGAGACCAG
ACAGCAAGAT TGTGTATGAG GGCACCCACA GAGTAAATGG AGAGTTGAAA TTAATGCAGT
TGTGATTTTA CCACGTGGAT ATGAAGAAGT GAGGGGGAGA AGTACAAAGG AGTTCTCTTA
ATGATTGACC ATGGAATTTA AGCTGGCTAA GAAAGGAAGT GAGAGGCCGG GCGCGGTGGC
TCACGCCTGT AATCCCAGCA CTTTGGGAGA CTGAGGTGGG TGGATTACCT GAGGTCAGGA
GTTTGAGACC AACCTGGCCG ATATGGCGAA ACCCCATCTC TAATAAAAAT ACAGAAAAAT
TAGCCGGGAA TGGTGGCAGG TGCCTGTAAT CCCAGCTACT CAAGAGGCTG TGGCAGGAGT
ATCCCTTGGA CCCAGGAGGT GGAGGTTGCA GTGAGCCGAG ATCACGCCAC TGTACTCCAG
CCTGGACGAT ATAGTGAGAC TTCACCTCAA AAAAAAAAAA AAAGAAAGGA AGTGAGGATT
TTAAGACCCT GAGAGACAGT TTAAAAAGTG GGAGGATCGG CCGGGCGCTG TGGCTGACAC
CTGTAATCCC AGCACTTTGG GAGGCCGAGT TGGGCAGATC ACAAGGTCAG GAGTTCGAGA
CCAGCCTGGC CAATATGGTG AAACCTTGTC TCTACTAAAA ATACAAAAAT TAGCCGGGCA
TGGTGTCACG TGTCTATAAT CCCAGCTACT CGGGAGGCTG AGGCAGAAAA ATTGCTTGAA
CCTGGGAGGC AGAGGTTGCA GACAGCTGAG ATCACTCCAT TGCACTCCAG CCTGGGCAAC
AAGAGCAAAA CTTTGTCTTT AAAAAAAAAA AAAAAAAAAG AATACAAAAA TTAGCCGGGC
GTGGTGGCGC GTGCCTATAA TCCCAGCTAC TTGGGAGGCT GAGGCAGGAG AATCAGTTGA
ACACGGGAGG CGAGGTTTGC AGTGAGCCGA GATTGCGCCA CTGCACTCCA GCCTGGGCGA
CAGAGCAGGA CTCCTCTTGG AAAAAAAAAA TTAGCTGGGC ATGGTGGCAG GTGCCTGTAG
TCTCAGCTAC TAGGGAGGCT GAGGCAGGAA AATCACTTGA ACCCGGGATG TGGAGTTTGC
AGTGACCCGA GATCGTGCCA CTGTACTCCA TCCTGGGCGA CAAAATGAGA CTCTGCCTCA
AAAAAAAAAA AAAAAAAAAG TGGGAGGATC AATGTACTGC CAGTCCTAAT GAAGTGGAAT
GATTGTCCCC ATCAAATCAC TAGTAGGAGT AAGTTGCAGA GCCTAGAAGG TGATGGTTAA
GAGAGTGGGA TTCTTGAAAC TGCATTTATG GAGAGGTTGT GGTTATTGGT TATAATAAAT
AAATACAGTT GAAGTGAGTG AGTAGCTGAG ATTTGGGGAT GTATCAGTTC ATTCTTACAC
TGCTACAAAG ACATACCTGA GACCAGGTAT TTATAAAGAT AAGAGGTTTA ATCAGCTCAC
AGTTCTGCTG CCTGTACAGG CTTCTCTTGT GGAGGCCTAA GGAAACTTAC AGTCATGGTG
GAAGGTGAAG GGGAAACAAG CACAGTCTTC ACATGGCCAG CAGGAGAGAG AGAGAAGGGG
GAAGTGCTAC ATACTTTAAA ACAACCAGAT CTTGTGAGAA CGCTTATCAG GAAACAGCAC
TTGGGGATGG TGCTAAATCA TTAGAAATCA CCCCCATGAT CCAGTCGCCT CCTACCATGC
CCACCTCCAA CACTGGGGAT CACAATTCAG CATGAGATTT GGGTAGGAAC ACAGAGCTGC
ACCACATCAG AGGATGTACA AGATTGTGGT GGAGAGGAGT TTAGAGACCT GCAAATATAG
GGTAATTGAA GGGATCATCT ACATGGATAT TTAAATCACC AAAAATTATG ACAGGAGTAG
TGTTGGAGAG AGAACTGCGA TGTAAACATT AAGGAATGAG GAAGAGTGAC TCGGTAGGCT
GTAGGTGACT GCAATAGGAA ACGATAATAG ACTGTGAGTC TGGTGACAAG ATTTTCCTTC
TTTCTTTTTT TCCCCCCCCC CGAGACAGGG CCTCTTTTTG TTGCCCAGGT GGGAGTGCAG
TGGCGCGATC ACGGCTCACT ACAACCTCCT CCCAAGCTCA AGGGATTCTC CCACTTCAGC
CTCTCAAGTA GCTGGAACTA CAGGTGCTGA CCACCATGCC TGGCTACTTT TTGTCAGGAT
TTTCAAGGCT GGGAATTTTG AGAGGGGAAT GGAGGAGAAT AATCTGAAAG TGCAAGTAAG
GAGCAGGGAA GATTTCTTTT TTCTTTTTTT TTTTTTTTTT TGAGTCGGAG TCTGGCTCAG
TCGCCCAGGC TGGAGTGCAG TGGCGAGATC TCCGCTCACT GCAAGCTCCG CCTCCCGTGT
TCACGCCATT CTCCTCCTTC AGCCTCCCGA GTAGCTGGGA CTACAGGCGC CCGCCACCAC
GCCCAGCTAA TTGTTTTTTT GTATTTTTAG TAGAGACGGG GTTTCACCGT GTTAGCCAGG
ATGGTCTCAA TCTCCTGACT TTGTGATCCG CCCACCCCGG CCTCCCAAAG CGCTTGGGAT
TACAGGCGTG AGCCACCGCG CCAGCCAGAG CAGGGAAGAT TTCTTCCCCA CATCTCCAGT
AGGTACAGTG ATATGAAGTG TGTGGAGGAG AAAAGAGGAA ACATCTATCA TTTGAGATGG
CTGCGAAAGG AAAAGGCATC CTCAGGGAGC TAGATTTTAC TTAGAGCAAG AAATGAAGGG
ATGATTCAGA GGTTAAAAGA GTGGATTTTA TGAATTACTC AAGGGAGCAC AGTGGAAGTT
TCAGGAAGTG GTAGGAGAAG GTAGAAGATG GCAGGGTGTT GGGAATAATT TGAGAAATCT
GAGCTACTGG AAATGACTGA GAATCAGATA TAAAGGCAGT CCTGGTGGTC CGTTCTGGCT
GCCGTTGCTG TGTAACGAAT CTGCCAAAAC TTAGTGGCTT GAAACAACAA AGAACATTTT
ATTATCTCTC ATTGTTTCTG TGGGTTAGGA ATTTGTGAGA GCCGTGCTGG GCAGTTTTCG
TGCGGCTGTC TCGTGGTTGC ACCTACATAG TTGCTAGAGC TACAGTAGCT GGGGACTGAG
CAGCTAGGGA TTGGCAGGCT ATCTCTTTTT TTCATGTAGT CTCATGAAGA TTTCTTTATG
TGGTTTCAAT GTGTGGGCTG GTTTGGATTT CCTTATAGCA TGGTGGCCTC AGTTGGATTG
CTGTTTTGTG ATCCTTTTCA TCCCTCCTTG TCCTGTCCCC AGACAACCAC TGATCTACTT
TCTGTCACCA TAGATTAGCC TGCATTTTTA AGAATTTTTA TAAACGTGGA ATGATAGAGT
ACCTTTTTTG TCACGTTTCT TTTATTTATC ATAGCTATTT TGATTTTCAT CCATTTTATT
GCTGAGTAGT ATCCCATTGC ATGTATATAC TATACTGTAT TCATTCGCTT GCTTGTGAAC
ATTTGGGCTT TTTCCAGTTT GGGACTGTTA ACAAGTAGAG CCACTATGAA TATTAGTGTA
TAAGACTTCA TATAGCCAAG GCTGGCAGAT CGCTTGAGCC CAGGAGTTTG AGACCAGCCT
GGGAAACATG GTGAAACCTC TATTTTTATT TTAAAATCAA AAATTAAAAA TTTTCTATAA
AAAATTTTAA AGAAGACTTT GTATAGACAT ACGCTTTCAT TTTTCTTGAG TGAATACTTA
GGTCTCAGGG TAGATGTATT TTAAGTCTTT AAGGAGCTGT CAAACTCTTC CTCAAAGTGG
TGGTTGTACC ATGTTACTTT TTAATATAAC AGAGATTAAT TGAGCAAAGA AAAATTCAAA
AGTTGGACAG CCCCCACAAC TAAATAGGTT CAGAACAGCT CCCCCATTTT GCATTTTGAC
CAGCAATGTA TGAAAGTTCC ATTTGCTCAG TGTCCCTGCA AACACCTGGT ATGGTCAGTC
TTTTTAATTT TAGGCATTAT AATAGATATA GTGGCTTCTT GTGATTTTAA TTAGCATTTC
CTAATGACCA GTGCTGCTGT TGATCATTTC ATGAGTGTAT TTGCCATCCG TATATCTTTT
TTGGTGAAGT GTCTATTCAA ATCATTTGGG TTTTTTTTTT TTTTGTTTTT TTTTTTTGGA
GACAGTGTCT CACTCTGTCA CCCAGGCTGT TGTGCAGTGG TGCAATCACA CAGCCTACTG
CAGCCTCCAC CTCCTGCGCT CAGTCTTCTT GTCTCAGCCT TCTGAGTAGC TGAAATTACG
AGCACACGCC ACAATGCCTG GCTAATTTTT TAAAATTTTG TAGAAACAAG GTCTCATTAT
GTTGCCTGGG CTTGTCGTGA ACTCCTGGGC TCAAGCAATC TTCCTGCCTC AGCCTCCCAA
AGATTGGGAT TGCAAGTATG AGCCACTGCA CCCGGCCAAC TTACCCATCT TTTAATTGAA
TTTTTTTGTT GTTGAGGTTT GAGAGTTCTT CATGTTTGCT GGGTACAATA TCTTTATCAG
ATAGGTAACT TGCATGTATT TTCTCCCGGT TTACACTTTG GTTTTTCATT TTGTTAACAA
CGTCTTTTTA AGAACAGAAA ATCTTAATTT TGCTGAAATC TAATTTTTCA GTTTTTTCTT
TGATGGTTTT GAGAGAGGAG GTAAAAAAAG ACTAGGTAAG CCGATAGTTA GACAGAGTCC
TCGGTAGAAC TTCCCTTCTA ACAAAAAGCA GCCCAAGAAA TCACTTCTCT TCTAACAAGG
AGCAGCCTGG AAGATCGGGC TGTAAACATG TATAAGGAAG CAGCTCTGGC ACAGAGuGGG
AGCTTCCTGG GTAATCAGCA AGCTTCACAT ACGTAAGGTG GGTATGTGAA GTAAACACAG
TATGTGAAGT AAACACAGTG GACCTTAGTA CATACTCAGA TAAGGAAGCT GGAAGCTTGC
ATGTTGTGAG TTGTTGGGGT TGCCTGCAGC TGCACGGAGA GAAAGGGGTA CCTGGGGCCA
GGCATGTCCA CCATGGTGGC TCCACCTCCC CTTATTTAGC ACATGCACAA TAGGAAAGAG
ATAAGCAATG TGGAGTAGCT CAGGCCAAGG ACCTGCCTGC ATAATAAAAG GTTGGGGTGG
GGGATGCCAG AGATTCACGC TCTGTGCAGA TGGCAACACC TGGTCCTAAC TGGTTTTTTG
CTCCCTATGT GTAGATAAGC TACCCCCTTC CCATTAGCTC ATTTATAAAA ATGCTTGCAT
TTCACTGTGG AATGGGAACT CTTTTCAGGA CCTCTCTCTG CAGGAGAGAG CTAGTCTCTT
TCTTTTGCCT ATTAAACTTC TGCTCTAGCC TCACACCCTT GGTGTGTCAG CGTCCTTGAT
TTCCTCAGCG TGAGACCAAG AACCTCGGGT GCCACCCCAG GCAACAAGGC CATTTCAGTT
TGTTCTTTTG TTATAGGCAA TCCATGATCA CAGATTTTTC TCTCTTTTTT TTTTTTACAC
AGTTTAGAGT TTTAGTTTTA CACTTAGGTC TGTAATCCAT TTTGTATTAA TTCTTATATG
TGGCTCAGTG TAGGTGGAAA TTTGGTTTGT TTTTGCATAA GGATTTCCAA TAGTTTTACC
ACCATTTCTT GAAACTACTA TGCTTTCTCT ATTAAACCAC ATTTGTAACT TTAGTTAAAA
TCAGTCACAT ATATCACAGG GCTATTTCTG ACTCTCAATT CTGTTACATT GTCTATTAGT
GTATATTGAT GTCAGTACTA CACTTTTAAT TACTATTGCT TCAGGGTATG TCTTGTAAAC
CAAAAATAAA ATTATAGGCC CCCCCCGCCC CTGCACAACC AACTGAATGG ACCCATCCTC
TCAGCCAAGG GCATTCCAAA ATTAACCTGA AAAACTAGTT CAAGCCATGA TGGGAAGGGG
GAGTTGGACA TGTCTCATCA CACCCTACTA CCTTTTGGAA TTACTGATAG AACAGACTCT
TAAAGTCTGA AAAGAAACAT TTACAACCTA CCCTCTCTGA AGCCTGCTAC CTGGGAGCTT
CATCTGCATG ATAAAACCTT GGTCTCCACA ACCCCTTATG GTAACCCAAA CATTCCTTTC
TGTTGATAAT AACTCTTTCA ACTAGTTGCC AATTAGAAAA TCTTTAAATC TTCCTATGAC
CTAGAAACCT CCCTACCCCC ACTTTGAGTT GTCCTGCCTT TCCTGACAGA ACTCATGTAC
ATCTTACATA TATTGATTGA TGCCTCATGT CTCCCTAAAA TGTATAAAAC AAAGCTGTAC
CCCACCACCT TGGGGACATG TCATCAGGAC CTCCTGTGGC TGTGTCATAG GAGCGTCTTT
AACTTTGGCA AAATAAACTT TCTAAATTGA TTGAAACCTG TCTTAGCTAC TTCTGGTTTA
CAGTCTTAAA GTTAGATAAT GTAAATTGTC CAGCTTTGGT TTATTTTTGT CCTTAGTAGT
TCCATATAAA TTTTAGAATC AGCTTTTCAA TTTAATACAC TACTTTCCTC TTAGATCCAC
AATTAAATAT ATTTGATGCT AACAATTCTG TTTTATGTTT TTCGTTTTTT TTTTTTGAGA
CAAGAGTTTC GCTCTTGTTG CCCAGGCTGG AGTGCAGTGG CGCGATCTTG GCTCACCACA
ACCTCCACCT CCCAGGTTCA AGCAATTCTT CTGCCTCAGC CTCCCGAGIA GCTGGGATTA
CAGGCATGCG CCACCACGCC CGGCTAATTT TGTATTTTTA GTAGAGACGG GGTTTCACCA
TGTTGATCAG GCTGGTCTTG AACTCCTGAC CTCAGGTGAT CCACCCACCT CGGCCTCCCA
AAGTGTTGGG ATTACAGGCG TGAACCACCA TGCCTGGCCA GTTCTGTTAT TTTTAAAACC
CAAGTTTCCC TGGTCATATC TTGGTTGGAT GAAGCGTATT TTCAATAGAT TACCCTGGAA
AGGCTAGTGA GTACGGTATT CTTCTACATT TTAGACTTTT CTTAGTCTTG CTACTTCAAG
GACAGCTAGG CTGCATATAA AATTCTTGGC TCATACTTTT TCCCCATAAA TTTCTATGAG
AAAGTCTAAT GATAACTGAT TTTCTTTATT TTGTAACTTA GTCTTTTTGC TTAGAGGCTC
TCTGAGGATG GGAGGGGGTT CTTCCTCCCA TCCCTAGGAA TTTTTCTTTT TTTTAAATTC
CTAATCACTA GACCACCAGG AAGATTGTTT GTTTTGTTTT GTTTTTATTC TTCAGGGACC
CCATTTATAC ATACGTTAAA TAAATACTGT TTGCCAATGT ATCAACCATT TTGCTTCTTA
TTTATTTTTG TTCCTTTGGT TCTTTTTCAT GGCTTTGCTT TGGTGCTCCT TAGATTTTCA
GTCAGATGTA TTTGTCCTTG GGTACCTTGT AATCAGTATT ACCTTTTCTT CTGTCGCTTT
GTTTTCTGTT CGTTTTGAAA TTACTTGTTT CCTGGTCTGG CAATAACAGT TGAGATATGA
GGAGTTTGAG CTGCCATCTG TCTATGTATC TTGCTTTAAG ACTGCACTCT TCTATTGATA
TCACTGGCCT TGATTTTGTG ATTTCTTTAT TTCTTCAGGA CCACCCTTCA TTTTCTACTG
TTTGCTTCCT TTTTTTTTGA GATGGAGTCT CACTCTGTCA CTCAGGCTGG AGTGCAGTGA
TCTTGGCTCA TTGCAACCTC TGCCTCCCGG GTTCCAGCAA TTCTCCTGCC TCAGCCTCCC
AAGTATCTGG GACTACAGGT GTGCACCACC ATGCCCGGCT AAGTTTTGTA TTITIAATAG
AGACGGGGTT TTGCCACATT GGCAGGCTGG TCTCAAACTC CTGATGTCAA GTGATCCACC
CACCCCACCC ACCTCTGCAT CCCAAAGTGC TGGGATTACA GGAATGAGCT GCCGTGCCCA
GCCTCCCCCC TACCCCCCTT TTTTTCTTTC GAGACAGAGA TTATAGGTGT GAGCCACTGG
ACCCAGCCTG TTTTTATTCC TTTTACCAAA TCTCCAAGGA ATATCTTCCC TTCCAAGTGC
GAATGTAACC TTAAGTCAGI TAACCTCTTT GTGATTACTT TTCTTATCTG CAAAGTGACT
TAATGATCTT AAGTACTTTT TTTTTTTGAG ACAGGGTCTC ACTGTCACCC TGGCTGGAGT
GCAGTGGCAC GATCTCTGAT CTCCACTCAC TGCAATCTCC TCTTCCCTGG TTCAAGCGGC
CCTCCCACCT TAGCCTTCTG GGTAGCTGGG ACTACAGATG TGAACCACCA CGCCGAGCTA
ATTTTTGTAC TTTTTGTAGA GATGGGGTTT TGCCATGTTG CCCAGGCTGG GATTATTAAG
TACTTTTTAT CATACAGCAA GATTGACATT TTATATTGGA ATACATTTGT CTCTATATAA
CGGAGATTAA CAGGAAAATG ACAAGCCTGG GTGCGGTGGA TCATGCCTGT AATCCCAGCA
CTTTGGGAGG CTGAGGTGGG AGGATCACTT GAGGTCAGGA GTTCGAGACC AGTTTTGCCA
AGATGATGAA AGCCCATGTC TACTAAAAAT ACAAAAATTA GCCCAGCTTG ATGGTGGGCG
CCTATAATCC CAGCTATTTG AGAGACTGAG GCAGGAGAAT CACTTGAACC TGGGCAGCAG
AGGTTGCAGT GAGCCGAGAT CATGCCACTG CACTCCAGCC TGGGTGGCAT AGCGAGACTC
TTGTCTCAAG AGAAAACAAA AGAAAACAAA AAAAAAACAG GAAAATGACA AAAAGTAATA
TTACAACTCA GTGAATTTTA TAACAAACTT TTTTGGAATT CATTGACTAA TACTATACCA
AATCCAAAAT ACTCTCTAGT ATACCAAATC CAACTCTACC CTATAGTATA AATTGGATTC
TATTTGGACT TGTCTCACTA ATCCCTCATA CAGTGTGTTT TATTTTTTAT TGAAGTAAAA
AAATTTGTCA TTTTAACCAT TTTTAAGTAT ATAGTTCAGT AATATTAAGT ATGTTCATGT
TGTTGCGCAA TAGATCTTCG GAAGTTTTTC GTCTTGCAAC CTGAAACTCT ACCCATTAGC
AAATTCCCAT TTCTCCTTAC ACTTAGCCCT TGGTAATCAT CATTCTTTTT TTTTTTTTTT
TGAGATGGAG TTTTACTCTT GTTGCCCAGG CTGGAGTGCA ATGGTGCAAT CTCGACTCAC
CACAACCTCC GCCTCCCAGG TTCAAGCAAT TCTACCTCAG CCTCCCGAGT AGCTGGGATT
ACAGTCATGC ACCACCACGC CCGGCTAATT TTGTATTTTT AGTAGAGAAG GGGTTTCTCC
ATGTTGAGGC TGGTCTCGAA CTCCTGACCT CAGGTGATCT GCCCACCTCG GCCTCCCAAA
GTGCTGGGAT TACAGGCGTG AGCCACTGCG CCTGGCCCAT TCTTTCTAAT TCTATAAATT
TGACTACTTA GTTACCTTAC ATAAATAAAT TCTTATAGTT AGTGTTATTT TTGCTTCCAT
GCCTTTTTTG TTGTTGTTCA TGCTCTTACT TGGAATGCGT TCTATTTTGT CTACCTATGC
ACATCCTGTT GGGTTTTTTT TTTTTTTGGG GGTTTTTTTT GTTTTTTTTT GTTTTTTTTT
CCCAGACAAG GTCTCAATTT GTTACCCAGG CTGGAGTGCA GCGGCGCCAT CTCCACTCAC
TGCATCCTCA ACTTCCTGGG CCCAGGTGAT CCTCTCGCCT CAGCCCCTGC AGGTAGCTGG
GACTATAGGC ATGTGCCACC ATGCCCAGCT AAATTTGGTT TTTTTGTTTG TTTGTTTTTG
AGACAGAGTC TCACTCTGTC ACCCAGGCTG GAGTGCAGTG GCACAATCTC AGCTCACTGC
AATCTCTGCC GCCCGGGTTC AAGTGATTCT CCTGCCTCAG CCTCCCAAGC AGCTGGGATT
ACAGGTGACT GCCACCACGC CAGCTAAGTT TTGTAGTTTT AGTAGAGATG GGGTTTCACC
TTGTTGGCCA TGCTGGTCTC GAACTCCTGA CCTCGTGATC TGCCTGCTTC TGCCTCCCAA
AGTGCTGGAA TTACAGGCAT GAGCCACCAC GCCCGGCCAG AATTTTTGTA TTTTTAGTAG
ACACAAGGTT CTTACCCTGT TGCCTAGGCT GGTCTAGGAG TCCTGGACTC AAGCAATTCA
CCTGCCTTGG CCTCCCAAAA TGCTGGGATT ACAAGCCACC ATGCCCGGCC TAAATCCTGT
TGTTTTGTTT TGTTTTATTT TGTTTTGTTT TGTTTTGTTT GTTTTTTGAG ACAGAGTCTC
GCTATGTCTC TCAGGCTGTA GTGCAGTGGC GCGATCTTGG CTCACTGCCA CCTCTGCCTC
CCAGGTTCAA GTGATTCTCC TGCCTCAGCC TCCCAAGTAG CTGGGATTAC AGGCATGTGC
TACTATGTCC GGCTAATTTT TGTATTTTTA GTAGAGACAG GGTTTCACCA TGTTGGCCAG
GCTGGTCTCG AACTCCTGAC CTCGTGATCC ACCCACCTCG GCCACCCAAA GTGCTGGGAT
TACAGGCGTG AGTGGTTTTT ATTTCTTAGG CCGGTTTCCT CCATATGATC TTGCAGTAGA
CATTAATTTC TTTCCTTTTT AATTAAAATA CTGTTTGTAT TTCACATTTT GATGTTTGTT
AAGATTTGTT TTATATTGTT TTTTGTTTTG TCTTGTGTGA TAGTCTTAAA TCCCTAGTTA
GATAATAACT GGAGAGTACC ATGTTTCTAT ATATCTCTCA GTGACTTGCA CAGTGCTAGC
AGATAGTGCT AAAAAATTAT TTATTATTAT TATTATTTTG TTATTGTTGT TGTTGTTGTT
AGACAGGGTC TTCCTCTGTC ACCCAGGCTA GAGGGCAATG GGATGATCAT AGCTTACTGC
AGCCTCCAAC AACTGGGCTC ATGTAATTCT CCTGCCTCAG CTTCCCAAGT AGCTGGGATT
ACAGGCATGA GCCACCATGT CTGGACAAAA ATATTTCCAG GTGCAGTGGC TCATGCCTGT
AATTCCCACA CTTGGGAGGC CGAGCGAGGC TGGAGGATCA CTTGAGCCTA GGAGTTCAAG
ACCAGCTTGG CTAAGATGGC GAGACCCCGT CCCTACAAAA AATTTTAAAA ACTAGCCAGG
CATGGTGGCA TGCACCTATA TTCCCAACTA CTCAGTGGGC TGAGGTGGGA GGGTCATTTG
AACACAGGAA TTTGAGGGGA GAAAAAAAGA AGAAAGAAAG AGAAGTGAAG GAAGGAAGAA
AGGAAGGAGG GAGGGAGAGA AGAAAGAAAC GAAAGAAAGG AAAAGAAAAG GAAGGAAAGA
AAATTGGTAC CAGGAAAGCA GGAAAGGGAA ATGGAAGTAA AAAAATAATA ATAATAATAA
AATGAAAATT GGTTAGTCAC TATTAACAAT TTGTATCCTT ATAATCTGGA AACATTATAA
TTTCAAAAGA AAAAATATTC TTTGGATCAT AGGTTCTGAG GTCAGAACAG CATTCCCGTA
GTCTAGATGA AGTCAAGTTT TATCTGATCT TAATTGAAAT AAATATAGCT GGCCTTGAAC
AAATCTACTC ATGGTATGTG GATAGGAATT AAATTGTAGG GGCATTCACT TGATGGCATT
CATTCTTAGA ACATTTACCT ATGTCTAGCT TTTGGAGTAA AGTCACATAA CCTCTAACCA
GGTAAGTTTC CTGTGGCTTT ATTTAGGATT TTAAATACTC ATTTTCAGTG TAATTTTGTT
ATGTGTGGAT TAAGATGACT CTTGGTACTA ACATACATTT TCTGATTAAA CCTATCTGAA
CATGAGTTGT TTTTATTTCT TACCCTTTCC AGAGCGATGA TTCTGACATT TGGGATGATA
CAGCACTGAT AAAAGCATAT GATAAAGCTG TGGCTTCATT TAAGGTATGA AATGCTTGCT
TAGTCGTTTT CTTATTTTCT CGTTATTCAT TTGGAAAGGA ATTGATAACA TACGATAAAG
TGTTAAAGTA CATGTTATTC AGTTTTCATT TTGAAGATTA GATGGTAGTA TGAGTTAGTT
AAATCAGGTG ATATCCTCCT TTAGAAGTTG ATAGCCTATA TATGTCATCC TTTGTGGAGG
CAATTTAAAT AAAATTTAAA ACATTTATTC CTGGCTGGGT ATGGTGGCTC ACTCCTGTAA
TCCCAGCACT TTGAGAGGCT GAGGCGGGTG GATCACCTGA GGTCAGGAGT TTGAGACCAG
CCTGGCCAAC ATGGTGAAAC CCCGTCTTTA CTAAAAATAC AAAAATTAGO CAAGCATGGT
GGCACGTGCC TGTAATCCCA GCTGCTTGGG ACACTGAGGC AGGAGAATTG CTTGAACCTG
GGGGGCAGAG GTTGCAATGA TTGCACCACT GCACTCCAGC CTGGGCGATA GAGTGAGACT
CCATCTCAGA AAACGAACAA ACAATGTATT CCTTTTAGTA TTTTTACATT GTATCAAACT
ATGGAAGTCC TCTAATTGAG ATTAATAAGA AAAAGACAAT CTGAATTATA ATTTTAAACA
TTTAACAAGC ATGTAGTAAA ATAATGATGA AGATAAATAG CATTAGTACA GCAATTAATA
TTTGTAGCAT GCTGACAGTG CTCTGTGTGC GTTTCATATA TTAAATTACT CTAATCATCC
CAAATCCTGT AAGTTGGGTA TCAATTCAAG TGTTCCTATT GGGTAGGAAT ATACAGTTCT
TTTAGGAAAT GTAGTATGGT TCTGTGTCTC AAACAGGACA CTTACACAGT TGGCCAACAT
CATCACCTTC TCCATTCTCT GAGATGTTTA GTCTTACTGA GCACTAAATA TGGGTCATCA
ATAGTCCAGA CTACCTTGAG CAAACAATAG TCCAGACTAC CTTGAGCAAA CAGAGCATAT
ACTCATACAG TGTATAAAGA GCACCAAGCA TACAGATTTC ATGTCTTTCT CATAGTTACT
CTTGTAACAT GAGCTAAAGA TCAGACCTCT ATGTCACCTT TGTAACTGAT TTCTAGATTT
TTTTTTTTTT TTGAGATGGG GTCTTGCCCT GTCACCCAGG CTGGAGTGTA GTGGCGTGAT
CATGCCTCAT TGGAGCCTTC AACTCATGAG CTCAAACAAT CCTCCTACCT CAGCTTCCTG
AGTAGTTGGG ACCACAGGTG TGTGCCACCA CACCCAGCTC ATTTTTGTAT TCTTTGTAGA
GATGCAGTCT CACCCTGTTG CCCAGGCTGG CCTGGAACTC CTGAGCTCAA AAGATCCCTC
CGCCTTGACC TTCCAAAGTG CTGGGATTAC AAGCATGAAC CACTGCACCC GGCCTAGATT
TTTAAATGTG CTTTCCAGTA TACACTGAAA CTAGAAGTCG ACTAAAGAAT TACCAAGAGA
ATTCTATAAA ATAGAGATTG AAATGGGGCT CGATGTGGGA TGGGTTGGTG ATATTGCAGG
GAGAAGTAAT CTGAGTAAAG GAGGAAAAGA ACTGATTTGG GAAAACGATA GTTTTAGTAG
TGAGTTTGAG TATGAATTAA GTTGAGATTG AATTTGAATT AAGTTGAGGT TGAATATGAA
TTAAGTTGAG GTTGAGTTTG AGGTATGAAT TAAGATGTGA AATTGATCAT TGGAAATGTT
AGATTGAGAA AAGTCACAGC TGGATTAATA GCTTCAGAAG TGTGTTTGCA GACAGTTGCA
ACTAAAGTAA TAAGAATAGA TGGCCTTGGC CGGGCGCGGT GGCTCACGCC TGTAATCCCA
GTACTTTGGG AGGCTGAGGC GAGCAAATCA CGAGGTCAGG AGTTCAAGAC CAGCCTGGCC
CACATGGTGA AACCCCGTCT TTATTAAAAA TACAAAAATT AGCTGTGCAC AGTGGTGCAC
GCCTGTAATC CCAGCTACTC GGGAGGCTGA GACAGGAGAA TCGCTTGAAC CTGGGAGGTG
GAGGTTGCAG TGAGCTGAGA TCAGTGTGAC TGCACTCCAG CCCGGTGACA GAGTGAGACT
CTGTGTAAAA AAATAAAATA AATAAAATAA TGGCCGTAAG CAAGTAAAGA AGGATGGCCA
GCTCTTATTG GGAATGCCTA AATCTAAGGC TTGATCAGAA GTAATGAAAC CGTTGGGGCC
CTACATTGCT ATGACATCCA AAGGGCCATG AATATCAGGA AGAAAGATAA TTAACAGGGT
CTAATGTTAC AGAGAGGTTG AGAGCAAGGA GATTTGATTA AAAGGGTCTT TAGAGCTGAT
GTCAGGTGTA TGATGCCTTT AAGAGCAGTT TTTATAGTGC AGGGGGTGGT CAAAAGAGAA
AATAGGTGCT TTCTGAGGTG ACGGAGCCTT GAGACTAGCT TATAGTAGTA ACTGGGTTAT
GTCGTGACTT TTATTCTGTG CACCACCCTG TAACATGTAC ATTTTTATTC CTATTTTCGT
AGCATGCTCT AAAGAATGGT GACATTTGTG AAACTTCGGG TAAACCAAAA ACCACACCTA
AAAGAAAACC TGCTAAGAAG AATAAAAGCC AAAAGAAGAA TACTGCAGCT TCCTTACAAC
AGGTTATTTT AAAATGTTGA GATTTAACTT CAAAGGATGT CTCATTAGTC CTTATTTAAT
AGTGTAAAAT GTCTTTAACT TAAGTGATTA GTACAGTGTT TCTATTGACA TATACTTATA
CAACTTCAAA AACAACTATT AAATTTTCTG TTATTTAGGA ACATGCATAT TAGTCATGAA
AGTATAAAGA ATTAGATGGG AATGATAAAT GCTAAAATCA GGACATGTGT TCCATTTGTG
AATGGAAGGC AGGGAGAAGG TGCCGTTTGG AAGGAGTACC CAAGAGCCGT AAGCTGAATT
GGCAGTGTTT TACATCTTAA GCTGAGAGAT AGATTTTTTT TTCCCCTTTT TCTTTAAAAA
CTCTAAAACT GTTAATTCCA AGGAACCCAG AAGTCTAGGT AGATTATTTC TGCTAGTTAA
AAGCAGTAGT CCTGAAAGCT GAATATTTTG GTGTCTTTTG AGCCAACTTT AGTTTCATCA
TTACCAAGGG GGAAGAGAGC TAACAGTTGA TGAGCACTTG CTCTAGGCCA GTCCAGAGTG
CTGGGCACCA TACGCATTTT ATCTCCCTCC CGCTATTCAC AACAAATATG GGAGGTAGTT
TATATTATAG CCATCTAATA AGATGGGGAA ACTAAGACTC AAAGAGATTC AGAAACTTGT
CCATGATTAT AAATGTAAGA GAGTTGGAAT TCAGATTTAT GTATTTAGAC CCCAAGCCTT
TCTCATTACA TCATTTTGCC TTCCAAATCT CTACCCTCTA TCCTTCACCT CCCCACTGAT
CAAAACGAGA TGATAGTTTG CCCTCTTCAA AAGAAATGTG TGCATGTATA TATCTTTGAT
TTCTTTTGTA GTGGAAAGTT GGGGACAAAT GTTCTGCCAT TTGGTCAGAA GACGGTTGCA
TTTACCCAGC TACCATTGCT TCAATTGATT TTAAGAGAGA AACCTGTGTT GTGGTTTACA
CTGGATATGG AAATAGAGAG GAGCAAAATC TGTCCGATCT ACTTTCCCCA ATCTGTGAAG
TAGCTAATAA TATAGAACAA AATGCTCAAG AGGTAAGGAT ACAAAAAAAA AAAAATTCAA
TTTCTGGAAG CAGAGACTAG ATGAGAAACT GTTAAACAGT ATACACAGTT GTCAGTTTGA
TCCACCGAGG CATTAATTTT TTCTTAATCA CACCCTTATA ACAAAAACCT GCATATTTTT
TCTTTTTAAA GAATGAAAAT GAAAGCCAAG TTTCTACAGA TGAAAGTGAG AACTCCAGGT
CTCCTGGAAA TAAATCAGAT AACATCAAGC CCAAATCTGC TCCATGGAAC TCTTTTCTCC
CTCCACCACC CCCCATGCCA GGGCCAAGAC TGGGACCAGG AAAGGTAAAC CTTCTATGAA
AGTTTTCCAG AAAATAGTTA ATGTCGGGAC ATTTAACCTC TCTGTTAACT AATTTGTAGC
TCTCCCATGA AACTTTTGTA GCTTAAATAC ACAAGAATTT TTTGAAAAGG AAATAAGATA
ATGATGCAAA ATAGTTAATT TTTTAAAAAA ATGTTAGACA CTGCAGTGGA TGCAACAAAA
TACTTTATAT GAAAGATTTA TCCAGTTAAC TTTTGTGGAG TATTAGGTAT TAGACTAATA
ATTTGCACAC TTACTTAAGT TAGAAAGTAT AATAATGCGC CGGACGCGGT AGCTCACGCC
TGTAATCCCA GCACTTTGGG AGGCCAAGGT GGGCGGATCA CAAGGTCAGG AGATCGAGAC
CATCCTGGCT AACACGGTGA AACCCCATCT CTACTGAAAA TAGAAAAAAA TTTGCCGGGC
GTGATGGCGG GCACCTGTAG TCCCAGCTAC TCGGGAGGCT GAGGCAGGAG GATGGTGTGA
ACCCCGGAGG CAGAGCTTGC AGTGAGTCAA GATCGTGCCA CTGCACTCCA ACCTGGGCGA
CAGAATGAGA CTCCATCTCA AACAAAAAAA CAAAACAAAA CAAAAAAAAG TGTAATAATA
ATTTATCATT AGCTGGATGA TATGCTGTTG TTTCCCATGT CACCTGTATA AGATATGTAA
AATAAGAACA CATTATTTAC ATCTAATATA GATAAAATCC TGAGGCGCTC TCAGATTGTT
TTGTAGAGTT CAAATGTAAA TATTGTTTTC ATTTATGGTC CTTTTGGTTA TAAGTAACAG
AAATCAACTC TAAAAAGATT TTTATTATAG GTTAGATTAT GTCATGGAAC CTTAAGGCTT
GTCCCTTTCT AGTTCTTTTG TGTAAAGCGG TGATTTCTTC CATGGAGGGA ATGGTATTTA
GGCAATTTTT TTTTTTTTTT CGAGATGGAG TCTTGCTCTG TCGCTCAGGC TGGAGTGCAG
TGGCACCATT TCAGCTCACT GCAACTTCCA CCTCCTGGGT TCAAGTGATT CTCCTGCTTC
AGCCTCCCAA GTAGCTGAGA TTAGAGGCAC CCGCCACCAC ACCCGGCTTA TTTTGTATTT
TTAGTAGAGA TGGGGTTTCA CCATGTTGGC CAGGCTGGTC TTGAACTCCT GACCTCAAGT
GATCTCCCCA CCTTGGCCTT CCAAAGTGCT AGGATTACAG GCGCCTAGCC TAGGCAGTCA
TTTTCAAAAA ACAAGCATGA CTCACCAAAA GTTTTAAGAT TTTCTGTGAT AATGTTCTTA
TTGAGGCTTA CATTATATTA CAGTTTCTTG AATCTAAAAT GATGTACCCT CTTAGAATAT
ATACATCATG CTTCATTGGT CTCAGGGGGC TGATTTTTAT AAGGAGAGAT TTGCTAGTTT
TCACAATATG TCCTCTAAGT TGGCATGTAT AGCTAAACAG GCTTTCATAA AAATATACAA
TTTAGTTAAT GAAATTTGGG ATATAGTCTT TTATGATTGA AATAATTTTG CTAAATAGAC
TGTCTCTGAT TTATTAGGTA ATCACCACTC TTATTTTGTT TTACTTCCTT AATGTCTACA
TAGAAAGGAA ATGAGAAAAA TCCAGAGGTT GTCATTTGAC TTATGAGTCT GTTTGACTTC
AGGATTTGGT ACATGAAATT TCACTTAATC TTTTTGATAT GTATAAAACA AATATTCTGG
GTAATTATTT TTATCCTTTT GGTTTTGAGT CCTTTTTATT CCTATCATAT TGAAATTGGT
AAGTTAATTT TCCTTTGAAA TATTCCTTAT AGCCAGGTCT AAAATTCAAT GGCCCAC+AC
CGCCACCGCC ACCACCACCA CCCCACTTAC TATCATGCTG GCTGCCTCCA TTTCCTTCTG
GACCACCAGT AAGTAAAAAA GAGTATAGGT TAGATTTTGC TTTCACATAC AATTTGATAA
TTAGCAGAAT AGAGGATTGT AAAATGTCAT TGTAGAACAT CCCTTGGGCC AGATTCTAAT
GGGTAGAAAT TTGAACTAAA CCTCTGGGTT TTGTTTGTTT TTAATGCCTT TCTGTTACCC
AGATGCAGTG CTCTTGTAGT CCCAAGTCTA AGCTCTAGGT TGCCTTCTTT CCTGGCAGAA
GTTGGTGTCT ATGCCATAAG GAGGTAGTTC CTGTTAGAAG GGATTTAATT ATACCTTATA
TAAGGAATTA GTGTTTGCCC TTCTAGGTAT AGTTGGATGT TAGCTTCTGA TGTAAACTGG
ATTTCTTTTT CTTTCTCTCT GTTTTTTTTT TTTTGTTTTG GAGGCAGAGT TTTGCCCTTG
TAGCCCAGGC TGGAGTGCAG TGGTGTGATC TCAGCTCACA GCAACCTCCG CCTCCTGGGT
TCAAGCAATT CTGCCTCGGC CTCCCAAGTA GCTGGGATTA CAGGCGACTG CCACCACACC
CGGCTAATTT TTGTTTTATT AGTAGAGATG GGGTTTCACC ATGTTGGCCA GACTGATCTT
GAACTCCTGA CCTCAGGTGA TCCACCCGCC TTGGCCTCCC AAAGCGCTGG GATTACAGGC
GTGAGCTGCC GCACCCAGCT GTAAACTGGA TTTCTAATGG TAGATTTTTA GGTATTAACA
ATAGATAAAA AGATACTTTT TGGCATACTG TGTATTGGGA TGGGGTTAGA ACAGGTGTTC
TACCCAAGAC ATTTACTTAA AATCGCCCTC GAAATGCTAT GTGAGCTGTG TGTGTGTGTG
TGTGTGTGTG TGTATTAAGG AAAAGCATGA AAGTATTTAT GCTTGATTTT TTTTTTTTAC
TCATAGCTTC ATAGTGGAAC AGATACATAG TCTAAATCAA AATGTTTAAA CTTTTTATGT
CACTTGCTGT CTTTTCGTCC TCGTTAAATT TAATTTTGTT GGTCTTTTGT TGTTATTGGT
TGGTTTTCTC CAAATGCTAG CTATGTTAAG AAATTTAAGG CCAGGTACAG TGGCTCATGC
CTGTAATCCC GGCATTTTAG AAGGCTGAGG CAGGAGGATC ACTTGAGCTC AGGAGTTTGA
GACCAGTCTG GGCAACATAG CAAGACCTCG TCTTTGTTTA GGGGAAAAAA AAGAAATTTA
AGTAGGAGAT TATATAAGCA AAAATACAAT TAATTTCCAG CATTCACTAT ATAATATAAA
TCTCCAGACT TTACTTTTTT GTTTACTGGA TATAAACAAT ATCTTTTTCT GTCTCCAGAT
AATTCCCCCA CCACCTCCCA TATGTCCAGA TTCTCTTGAT GATGCTGATG CTTTGGGAAG
TATGTTAATT TCATGGTACA TGAGTGGCTA TCATACTGGC TATTATATGG TAAGTAATCA
CTCAGCATCT TTTCCTGACA ATTTTTTTGT AGTTATGTGA CTTTGTTTTG TAAATTTATA
AAATACTACT TGCTTCTCTC TTTATATTAC TAAAAAATAA AAATAAAAAA ATACAACTGT
CTGAGGCTTA AATTACTCTT GCATTGTCCC TAAGTATAAT TTTAGTTAAT TTTAAAAAGC
TTTCATGCTA TTGTTAGATT ATTTTGATTA TACACTTTTG AATTGAAATT ATACTTTTTC
TAAATAATGT TTTAATCTCT GATTTGAAAT TGATTGTAGG GAATGGAAAA GATGGGATAA
TTTTTCATAA ATGAAAAATG AAATTCTTTT TTTTTTTTTT TTTTTTTTGA GACGGAGTCT
TGCTCTGTTG CCCAGGCTGG AGTGCAATGG CGTGATCTTG GCTCACAGCA AGCTCTGCCT
CCTGGATTCA CGCCATTCTC CTGCCTCAGC CTCAGAGGTA GCTGGGACTA CAGGTGCCTG
CCACCACGCC TGTCTAATTT TTTGTATTTT TTTGTAAAGA CAGGGTTTCA CTGTGTTAGC
CAGGATGGTC TCAATCTCCT GACCCCGTGA TCCACCCGCC TCGGCCTTCC AAGAGAAATG
AAATTTTTTT AATGCACAAA GATCTGGGGT AATGTGTACC ACATTGAACC TTGGGGAGTA
TGGCTTCAAA CTTGTCACTT TATACGTTAG TCTCCTACGG ACATGTTCTA TTGTATTTTA
GTCAGAACAT TTAAAATTAT TTTATTTTAT TTTATTTTTT TTTTTTTTTT GAGACGGAGT
CTCGCTCTGT CACCCAGGCT GGAGTACAGT GGCGCAGTCT CGGCTCACTG CAAGCTCCGC
CTCCCGGGTT CACGCCATTC TCCTGCCTCA GCCTCTCCGA GTAGCTGGGA CTAGAGGCGC
CCGCCACCAC GCCCGGCTAA TTTTTTTTTA TTTTTAGTAG AGACGGGGTT TCACCGTGGT
CTCGATCTCC TGACCTCGTG ATCCACCCGC CTCGGCCTCC CAAAGTGCTG GGATTACAAG
CGTGAGCCAC CGCGCCCGGC CTAAAATTAT TTTTAAAAGT AAGCTCTTGT GCCCTGCTAA
AATTATGATG TGATATTGTA GGCACTTGTA TTTTTAGTAA ATTAATATAG AAGAAACAAC
TGACTTAAAG GTGTATGTTT TTAAATGTAT CATCTGTGTG TGCCCCCATT AATATTCTTA
TTTAAAAGTT AAGGCCAGAC ATGGTGGCTT ACAACTGTAA TCCCAACAGT TTGTGAGGCC
GAGGCAGGCA GATCACTTGA GGTCAGGAGT TTGAGACCAG CCTGGCCAAC ATGATGAAAC
CTTGTCTCTA CTAAAAATAC CAAAAAAAAT TTAGCCAGGC ATGGTGGCAC ATGCCTGTAA
TCCGAGCTAC TTGGGAGGCT GTGGCAGGAA AATTGCTTTA ATCTGGGAGG CAGAGGTTGC
AGTGAGTTGA GATTGTGCCA CTGCACTCCA CCCTTGGTGA CAGAGTGAGA TTCCATCTCA
AAAAAAGAAA AAGGCCTGGC ACGGTGGCTC ACACCTATAA TCCCAGTACT TTGGGAGGTA
GAGGCATGTG GATCACTTGA GGTTAGGAGT TCAGGACCAG CCTGGCCAAC ATGGTGACTA
CTCCATTTCT ACTAAATACA CAAAACTTAG CCCAGTGGCG GGCAGTTGTA ATCCCAGCTA
CTTGAGAGGT TGAGGCAGGA GAATCACTTG AACCTGGGAG GCAGAGGTTG CAGTGAGCCG
AGATCACACC GCTGCACTCT AGCCTGGGCA ACAGAGTGAG AATTTGCGGA GGGAAAAAAA
AGTCACGCTT CAGTTGTTGT AGTATAACCT TGGTATATTG TATGTATCAT GAATTCCTCA
TTTTAATGAC CAAAAAGTAA TAAATCAACA GCTTGTAATT TGTTTTGAGA TCAGTTATCT
GACTGTAACA CTGTAGGCTT TTGTGTTTTT TAAATTATGA AATATTTGAA AAAAATACAT
AATGTATATA TAAAGTATTG GTATAATTTA TGTTCTAAAT AACTTTCTTG AGAAATAATT
CACATGGTGT GCAGTTTACC TTTGAAAGTA TACAAGTTGG CTGGGCACAA TGGCTCACGC
CTGTAATCCC AGCACTTTGG GAGGCCAGGG CAGGTGGATC ACGAGGTCAG GAGATCGAGA
CCATCCTGGC TAACATGGTG AAACCCCGTC TCTACTAAAA GTACAAAAAC AAATTAGCCG
GGCATGTTGG CGGGCACCTT TTGTCCCAGC TGCTCGGGAG GCTGAGGCAG GAGAGTGGCG
TGAACCCAGG AGGTGGAGCT TGCAGTGAGC CGAGATTGTG CCAGTGCACT CCAGCCTGGG
CGACAGAGCG AGACTCTGTC TCAAAAAATA AAATAAAAAA GAAAGTATAC AAGTCAGTGG
TTTTGGTTTT CAGTTATGCA ACCATCACTA CAATTTAAGA ACATTTTCAT CACCCCAAAA
AGAAACCCTG TTACCTTCAT TTTCCCCAGC CCTAGGCAGT CAGTACACTT TCTGTCTCTA
TGAATTTGTC TATTTTAGAT ATTATATATA AACGGAATTA TACGATATGT GGTCTTTTGT
GTCTGGCTTC TTTCACTTAG CATGCTATTT TCAAGATTCA TCCATGCTGT AGAATGCACC
AGTACTGCAT TCCTTCTTAT TGCTGAATAT TCTGTTGTTT GGTTATATCA CATTTTATCC
ATTCATCAGT TCATGGACAT TTAGGTTGTT TTTATTTTTG GGCTATAATG AATAATGTTG
CTATGAACAT TCGTTTGTGT TCTTTTTGTT TTTTTGGTTT TTTGGGTTTT TTTTGTTTTG
TTTTTGTTTT TGAGACAGTC TTGCTCTGTC TCCTAAGCTG GAGTGCAGTG GCATGATCTT
GGCTTACTGC AAGCTCTGCC TCCCGGGTTC ACACCATTCT CCTGCCTCAG CCCGACAAGT
AGCTGGGACT ACAGGCGTGT GCCACCATGC ACGGCTAATT TTTTGTATTT TTAGTAGAGA
TGGGGTTTCA CCGTGTTAGC CAGGATGGTC TCGATCTCCT GACCTCGTGA TCTGCCTGCC
TAGGCCTCCC AAAGTGCTGG GATTACAGGC GTGAGCCACT GCACCTGGCC TTAAGTGTTT
TTAATACGTC ATTGCCTTAA GCTAACAATT CTTAACCTTT GTTCTACTGA AGCCACGTGG
TTGAGATAGG CTCTGAGTCT AGCTTTTAAC CTCTATCTTT TTGTCTTAGA AATCTAAGCA
GAATGCAAAT GACTAAGAAT AATGTTGTTG AAATAACATA AAATAGGTTA TAACTTTGAT
ACTCATTAGT AACAAATCTT TCAATACATC TTACGGTCTG TTAGGTGTAG ATTAGTAATG
AAGTGGGAAG CCACTGCAAG CTAGTATACA TGTAGGGAAA GATAGAAAGC ATTGAAGCCA
GAAGAGAGAC AGAGGACATT TGGGCTAGAT CTGACAAGAA AAACAAATGT TTTAGTATTA
ATTTTTGACT TTAAATTTTT TTTTTATTTA GTGAATACTG GTGTTTAATG GTCTCATTTT
AATAAGTATG ACACAGGTAG TTTAAGGTCA TATATTTTAT TTGATGAAAA TAAGGTATAG
GCCGGGCACG GTGGCTCACA CCTGTAATCC CAGCACTTTG GGAGGCCGAG GCAGGCGGAT
CACCTGAGGT CGGGAGTTAG AGACTAGCCT CAACATGGAG AAACCCCGTC TCTACTAAAA
AAAATACAAA ATTAGGCGGG CGTGGTGGTG CATGCCTGTA ATCCCAGCTA CTCAGGAGGC
TGAGGCAGGA GAATTGCTTG AACCTGGGAG GTGGAGGTTG CGGTGAGCCG AGATCACCTC
ATTGCACTCC AGCCTGGGCA ACAAGAGCAA AACTCCATCT CAAAAAAAAA AAAATAAGGT
ATAAGCGGGC TCAGGAACAT CATTGGACAT ACTGAAAGAA GAAAAATCAG CTGGGCGCAG
TGGCTCACGC CGGTAATCCC AACACTTTGG GAGGCCAAGG CAGGCGAATC ACCTGAAGTC
GGGTGTTCCA GATCAGCCTG ACCAACATGG AGAAACCCTG TCTCTACTAA AAATACAAAA
CTAGCCGGGC ATGGTGGCGC ATGCCTGTAA TCCGAGCTAC TTGGGAGGCT GAGGCAGGAG
AATTGCTTGA ACCGAGAAGG CGGAGGTTGC GGTGAGCCAA GATTGCACCA TTGCACTCCA
GCCTGGGCAA CAAGAGCGAA ACTCCGTCTC AAAAAAAAAA GGAAGAAAAA TATTTTTTTA
AATTAATTAG TTTATTTATT TTTTAAGATG GAGTTTTGCC CTGTCACCCA GGCTGGGGTG
CAATGGTGCA ATCTCGGCTC ACTGCAACCT CCGCCTCCTG GGTTCAAGTG ATTCTCCTGC
CTCAGCTTCC CGAGTAGCTG TGATTACAGC wATATGCCAC CACGCCCAGC CAGTTTTGTG
TTTTGTTTTG TTTTTTGTTT TTTTTTTTTG AGAGGGTGTC TTGCTCTGTC CCCCAAGCTG
GAGTGCAGCG GCGCGATCTT GGCTCACTGC AAGCTCTGCC TCCCAGGTTC ACACCATTCT
CTTGCCTCAG CCTCCCGAGT AGCTGGGACT ACAGGTGCCC GCCACCACAC CCGGCTAATT
TTTTTGTGTT TTTAGTAGAG ATGGGGTTTC ACTGTGTTAG CCAGGATGGT CTCGATCTCC
TGACCTTTTG ATCCACCCGC CTCAGCCTCC CCAAGTGCTG GGATTATAGG CGTGAGCCAC
TGTGCCCGGC CTAGTCTTGT ATTTTTAGTA GAGTCGGGAT TTCTCCATGT TGGTCAGGCT
GTTCTCCAAA TCCGACCTCA GGTGATCCGC CCGCCTTGGC CTCCAAAAGT GCAAGGCAAG
GCATTACAGG CATGAGCCAC TGTGACCGGC AATGTTTTTA AATTTTTTAC ATTTAAATTT
TATTTTTTAG AGACCAGGTC TCACTCTATT GCTCAGGCTG GAGTGCAAGG GCACATTCAC
AGCTCACTGC AGCCTTGACC TCCAGGGCTC AAGCAGTCCT CTCACCTCAG TTTCCCGAGT
AGCTGGGACT ACAGTGATAA TGCCACTGCA CCTGGCTAAT TTTTATTTTT ATTTATTTAT
TTTTTTTTGA GACAGAGTCT TGCTCTGTCA CCCAGGCTGG AGTGCAGTGG TGTAAATCTC
AGCTCACTGC AGCCTCCGCC TCCTGGGTTC AAGTGATTCT CCTGCCTCAA CCTCCCAAGT
AGCTGGGATT AGAGGTCCCC ACCACCATGC CTGGCTAATT TTTTGTACTT TCAGTAGAAA
CGGGGTTTTG CCATGTTGGC CAGGCTGTTC TCGAACTCCT GAGCTCAGGT GATCCAACTG
TCTCGGCCTC CCAAAGTGCT GGGATTACAG GCGTGAGCCA CTGTGCCTAG CCTGAGCCAC
CACGCCGGCC TAATTTTTAA ATTTTTTGTA GAGACAGGGT CTCATTATGT TGCCCAGGGT
GGTGTCAAGC TCCAGGTCTC AAGTGATCCC CCTACCTCCG CCTCCCAAAG TTGTGGGATT
GTAGGCATGA GCCACTGCAA GAAAACCTTA ACTGCAGCCT AATAATTGTT TTCTTTGGGA
TAACTTTTAA AGTACATTAA AAGACTATCA ACTTAATTTC TGATCATATT TTGTTGAATA
AAATAAGTAA AATGTCTTGT GAAACAAAAT GCTTTTTAAC ATCCATATAA AGCTATCTAT
ATATAGCTAT CTATATCTAT ATAGCTATTT TTTTTAACTT CCTTTATTTT CCTTACAGGG
TTTTAGACAA AATCAAAAAG AAGGAAGGTG CTCACATTCC TTAAATTAAG GAGTAAGTCT
GCCAGCATTA TGAAAGTGAA TCTTACTTTT GTAAAACTTT ATGGTTTGTG GAAAACAAAT
GTTTTTGAAC ATTTAAAAAG TTCAGATGTT AGAAAGTTGA AAGGTTAATG TAAAACAATC
AATATTAAAG AATTTTGATG CCAAAACTAT TAGATAAAAG GTTAATCTAC ATCCCTACTA
GAATTCTCAT ACTTAACTGG TTGGTTGTGT GGAAGAAACA TACTTTCACA ATAAAGAGCT
TTAGGATATG ATGCCATTTT ATATCACTAG TAGGCAGACC AGCAGACTTT TTTTTATTGT
GATATGGGAT AACCTAGGCA TACTGCACTG TACACTCTGA CATATGAAGT GCTCTAGTCA
AGTTTAACTG GTGTCCACAG AGGACATGGT TTAACTGGAA TTCGTCAAGC CTCTGGTTCT
AATTTCTCAT TTGCAGGAAA TGCTGGCATA GAGCAGCACT AAATGACACC ACTAAAGAAA
CGATCAGACA GATCTGGAAT GTGAAGCGTT ATAGAAGATA ACTGGCCTCA TTTCTTCAAA
ATATCAAGTG TTGGGAAAGA AAAAAGGAAG TGGAATGGGT AACTCTTCTT GATTAAAAGT
TATGTAATAA CCAAATGCAA TGTGAAATAT TTTACTGGAC TCTATTTTGA AAAACCATCT
GTAAAAGACT GAGGTGGGGG TGGGAGGCCA GCACGGTGGT GAGGCAGTTG AGAAAATTTG
AATGTGGATT AGATTTTGAA TGATATTGGA TAATTATTGG TAATTTTATG AGCTGTGAGA
AGGGTGTTGT AGTTTATAAA AGACTGTCTT AATTTGCATA CTTAAGCATT TAGGAATGAA
GTGTTAGAGT GTCTTAAAAT GTTTCAAATG GTTTAACAAA ATGTATGTGA GGCGTATGTG
GCAAAATGTT ACAGAATCTA ACTGGTGGAC ATGGCTGTTC ATTGTACTGT TTTTTTCTAT
CTTCTATATG TTTAAAAGTA TATAATAAAA ATATTTAATT TTTTTTTAAA TTA
Homo sapiens SMN2 pre-mRNA nucleic acid sequence
(SEQ ID NO: 64)
CCACAAAUGU GGGAGGGCGA UAACCACUCG UAGAAAGCGU GAGAAGUUAC UACAAGCGGU
CCUCCCGGCC ACCGUACUGU UCCGCUCCCA GAAGCCCCGG GCGGCGGAAG UCGUCACUCU
UAAGAAGGGA CGGGGCCCCA CGCUGCGCAC CCGCGGGUUU GCUAUGGCGA UGAGCAGCGG
CGGCAGUGGU GGCGGCGUCC CGGAGCAGGA GGAUUCCGUG CUGUUCCGGC GCGGCACAGG
CCAGGUGAGG UCGCAGCCAG UGCAGUCUCC CUAUUAGCGC UCUCAGCACC CUUCUUCCGG
CCCAACUCUC CUUCCGCAGC CUCGGGACAG CAUCAAGUCG AUCCGCUCAC UGGAGUUGUG
GUCCGCGUUU UUCUACGUCU UUUCCCACUC CGUUCCCUGC GAACCACAUC CGCAAGCUCC
UUCCUCGAGC AGUUUGGGCU CCUUGAUAGC GUUGAGUGGA GGCCCUGCCG CGACUUGGCA
GUAGCUUAUU UUGUUCACUC CUCUCUGGCU GGUGUGGGGG AGGUGGGGGC AUUAGGCCAG
GGUGAAGCAG GGGAACCACU UAGGAGUCUG UUAAGAUGAU CUGAACUUCA GAACAAGAUG
UUAUUAACAG AGUGAAAGUA UUUGGAUUCU GGGUAUAUUU UGAAAUCGGA GGCAACAGGU
UUUUCAGAUA GAUUCGAUAA CGGAGGUUAU CCUGAAUAGU UGAAAAGAUA AAGUUGCCUU
UUGCUGAGGU GGGAAAGAGA AGAUUGCCAG UAGAGCAGGU UUCUCAGGAG UUCAGUCUUG
GGCAUAGCAU GGUAGGGGUG AAUUUGGCUG GAGUGAGUUG GAGAGUAGGA GAAGAGAAAU
CCAAGGCAAC AUUUGACCAG CCUGGGCAAC AUAGUGUGAC UCCGAGUCUG CAAAAAUUAG
ACGGGUGUUG UGGUGCGCGU CUGUGGUCUC AGCUACCUGG AAGGUUCAGG CCUUGGAAGG
CUCAGGGAGG UGGAGGCUGC AGUGAUCUGU GAUUGCGCCU CUGCACUCCA GCCUGGGCGA
CAGAGCCAGA CCCUGUCUUA AAACAAAAUA AACGGCCGGG CGCGGUGGCU CAAGCCUGUA
AUCCCAGCAC UUUGGGAGGC CGAGGCGGCC GGAUCACAAG GUCAGGAGAU CGAGACCAUC
CUGGCUAACA CGGUGAAACC CCGUCUCUAC UACAAAUACA AAAAAUUAGC CGGGCGUGGU
GACGGGCGCC UGUAGUCCCA GCUACUCGGG AGGCUGAGGC AGGAGAAUGU CAUGAAGCCG
GGAGGCGGAG CUUGCAGUGA GCCGAGAUCG CGCCACUGCA CUCCAGCCUG GGCGAUAGAG
CAAGACUCCG UCUCAAAUAA AUAAAUAAAU AAAUAAAUAA AUAAUAAAAA CAUCGGUAGG
CAUAUUUCAA GGAAUUCUAU UUAAAAAAAA UUUUUUUAGA GACAAGUUCG CUCUCUGUGG
CCCAGGCUGG AGUACAGUGG CAUGAUCCUA GCCCAUGGCA GCGUUGAUCU CUUGGCCUCA
AGOGACCCUC CUUUGGAGUC GCUGGGCCUA AAGGAGUGAG CCACCACGAA AUUUUAUUAU
AAAUGGAGGG UAGAGAAAUU GGGCAAUAAA UGGAGGGGGA AGUGAGUUAA GAGGAAUUUU
AAUUAUGUGU GUGUGGUUUU AAAAGAGGGG GGUCUUGCUC UGUUGCCCAG GCUGCUGGGG
UGCCAGUGGC GCAAUCAUGA AUCACUACAG CCUUGGACUC CUGGCCUCAA GCUAUCCUCC
CACCUCUGCC UCCCAAAGUA CUGGGAUUAC UAGUGUGAGC CACUGCACUA AGAUAGGAGC
AACAUGUUUC AGCAUGUUUG UGGGUUGAUA GGAAAGAUGA GAAUGGGAAA GUUGAUGUCG
GAAAGAAGAC AAUGGCUAGA GCAAUGUCCU AGAGUAGGUA AGAAGGGAUG GAUUUGGCCU
UUGUUGGAAA CAUUAGCGGU UCUUUUGGUG ACAGCUAUAU AGUUAACACA UCUAUGAUAC
GUGAAUGGGC AGAUAGGAUG GCAGGAGAUU UUGAAAGUUC UCUUGAUUCU UACUGUUCUC
UUAGUGAAAG AAGCAAGGUU AUCAGCUAGA AGCUGGGAUG GGAGAGGAAA GAGAAGAUGG
GAAGUAGAUA GUUCUUUAGA AGAGUGGGCA AGGGUUGGAC UAGGGAAGUU UAGUGGAAAU
AUUGCUAGGC AACAUAAAGA GCCUACUUGA GAUUCGUGGU CAUGAGUUGA AGGAGACCAG
ACAGCAAGAU UGUGUAUGAG GGCACCCACA GAGUAAAUGG AGAGUUGAAA UUAAUGCAGU
UGUGAUUUUA CCACGUGGAU AUGAAGAAGU GAGGGGGAGA AGUACAAAGG AGUUCUCUUA
AUGAUUGACC AUGGAAUUUA AGCUGGCUAA GAAAGGAAGU GAGAGGCCGG GCGCGGUGGC
UCACGCCUGU AAUCCCAGCA CUUUGGGAGA CUGAGGUGGG UGGAUUACCU GAGGUCAGGA
GUUUGAGACC AACCUGGCCG AUAUGGCGAA ACCCCAUCUC UAAUAAAAAU ACAGAAAAAU
UAGCCGGGAA UGGUGGCAGG UGCCUGUAAU CCCAGCUACU CAAGAGGCUG UGGCAGGAGU
AUCCCUUGGA CCCAGGAGGU GGAGGUUGCA GUGAGCCGAG AUCACGCCAC UGUACUCCAG
CCUGGACGAU AUAGUGAGAC UUCACCUCAA AAAAAAAAAA AAAGAAAGGA AGUGAGGAUU
UUAAGACCCU GAGAGACAGU UUAAAAAGUG GGAGGAUCGG CCGGGCGCUG UGGCUGACAC
CUGUAAUCCC AGCACUUUGG GAGGCCGAGU UGGGCAGAUC ACAAGGUCAG GAGUUCGAGA
CCAGCCUGGC CAAUAUGGUG AAACCUUGUC UCUACUAAAA AUACAAAAAU UAGCCGGGCA
UGGUGUCACG UGUCUAUAAU CCCAGCUACU CGGGAGGCUG AGGCAGAAAA AUUGCUUGAA
CCUGGGAGGC AGAGGUUGCA GACAGCUGAG AUCACUCCAU UGCACUCCAG CCUbGGCAAC
AAGAGCAAAA CUUUGUCUUU AAAAAAAAAA AAAAAAAAAG AAUACAAAAA UUAGCCGGGC
GUGGUGGCGC GUGCCUAUAA UCCCAGCUAC UUGGGAGGCU GAGGCAGGAG AAUCAGUUGA
ACACGGGAGG CGAGGUUUGC AGUGAGCCGA GAUUGCGCCA CUGCACUCCA GCCUGGGCGA
CAGAGCAGGA CUCCUCUUGG AAAAAAAAAA UUAGCUGGGC AUGGUGGCAG GUGCCUGUAG
UCUCAGCUAC UAGGGAGGCU GAGGCAGGAA AAUCACUUGA ACCCGGGAUG UGGAGUUUGC
AGUGACCCGA GAUCGUGCCA CUGUACUCCA UCCUGGGCGA CAAAAUGAGA CUCUGCCUCA
AAAAAAAAAA AAAAAAAAAG UGGGAGGAUC AAUGUACUGC CAGUCCUAAU GAAGUGGAAU
GAUUGUCCCC AUCAAAUCAC UAGUAGGAGU AAGUUGCAGA GCCUAGAAGG UGAUGGUUAA
GAGAGUGGGA UUCUUGAAAC UGCAUUUAUG GAGAGGUUGU GGUUAUUGGU UAUAAUAAAU
AAAUACAGUU GAAGUGAGUG AGUAGCUGAG AUUUGGGGAU GUAUCAGUUC AUUCUUACAC
UGCUACAAAG ACAUACCUGA GACCAGGUAU UUAUAAAGAU AAGAGGUUUA AUCAGCUCAC
AGUUCUGCUG CCUGUACAGG CUUCUCUUGU GGAGGCCUAA GGAAACUUAC AGUCAUGGUG
GAAGGUGAAG GGGAAACAAG CACAGUCUUC ACAUGGCCAG CAGGAGAGAG AGAGAAGGGG
GAAGUGCUAC AUACUUUAAA ACAACCAGAU CUUGUGAGAA CGCUUAUCAG GAAACAGCAC
UUGGGGAUGG UGCUAAAUCA UUAGAAAUCA CCCCCAUGAU CCAGUCGCCU CCUACCAUGC
CCACCUCCAA CACUGGGGAU CACAAUUCAG CAUGAGAUUU GGGUAGGAAC ACAGAGCUGC
ACCACAUCAG AGGAUGUACA AGAUUGUGGU GGAGAGGAGU UUAGAGACCU GCAAAUAUAG
GGUAAUUGAA GGGAUCAUCU ACAUGGAUAU UUAAAUCACC AAAAAUUAUG ACAGGAGUAG
UGUUGGAGAG AGAACUGCGA UGUAAACAUU AAGGAAUGAG GAAGAGUGAC UCGGUAGGCU
GUAGGUGACU GCAAUAGGAA ACGAUAAUAG ACUGUGAGUC UGGUGACAAG AUUUUCCUUC
UUUCUUUUUU UCCCCCCCCC CGAGACAGGG CCUCUUUUUG UUGCCCAGGU GGGAGUGCAG
UGGCGCGAUC ACGGCUCACU ACAACCUCCU CCCAAGCUCA AGGGAUUCUC CCACUUCAGC
CUCUCAAGUA GCUGGAACUA CAGGUGCUGA CCACCAUGCC UGGCUACUUU UUGUCAGGAU
UUUCAAGGCU GGGAAUUUUG AGAGGGGAAU GGAGGAGAAU AAUCUGAAAG UGCAAGUAAG
GAGCAGGGAA GAUUUCUUUU UUCUUUUUUU UUUUUUUUUU UGAGUCGGAG UCUGGCUCAG
UCGCCCAGGC UGGAGUGCAG UGGCGAGAUC UCCGCUCACU GCAAGCUCCG CCUCCCGUGU
UCACGCCAUU CUCCUCCUUC AGCCUCCCGA GUAGCUGGGA CUACAGGCGC CCGCCACCAC
GCCCAGCUAA UUGUUUUUUU GUAUUUUUAG UAGAGACGGG GUUUCACCGU GUUAGCCAGG
AUGGUCUCAA UCUCCUGACU UUGUGAUCCG CCCACCCCGG CCUCCCAAAG CGCUUGGGAU
UACAGGCGUG AGCCACCGCG CCAGCCAGAG CAGGGAAGAU UUCUUCCCCA CAUCUCCAGU
AGGUACAGUG AUAUGAAGUG UGUGGAGGAG AAAAGAGGAA ACAUCUAUCA UUUGAGAUGG
CUGCGAAAGG AAAAGGCAUC CUCAGGGAGC UAGAUUUUAC UUAGAGCAAG AAAUGAAGGG
AUGAUUCAGA GGUUAAAAGA GUGGAUUUUA UGAAUUACUC AAGGGAGCAC AGUGGAAGUU
UCAGGAAGUG GUAGGAGAAG GUAGAAGAUG GCAGGGUGUU GGGAAUAAUU UGAGAAAUCU
GAGCUACUGG AAAUGACUGA GAAUCAGAUA UAAAGGCAGU CCUGGUGGUC CGUUCUGGCU
GCCGUUGCUG UGUAACGAAU CUGCCAAAAC UUAGUGGCUU GAAACAACAA AGAACAUUUU
AUUAUCUCUC AUUGUUUCUG UGGGUUAGGA AUUUGUGAGA GCCGUGCUGG GCAGUUUUCG
UGCGGCUGUC UCGUGGUUGC ACCUACAUAG UUGCUAGAGC UACAGUAGCU GGGGACUGAG
CAGCUAGGGA UUGGCAGGCU AUCUCUUUUU UUCAUGUAGU CUCAUGAAGA UUUCUUUAUG
UGGUUUCAAU GUGUGGGCUG GUUUGGAUUU CCUUAUAGCA UGGUGGCCUC AGUUGGAUUG
CUGUUUUGUG AUCCUUUUCA UCCCUCCUUG UCCUGUCCCC AGACAACCAC UGAUCUACUU
UCUGUCACCA UAGAUUAGCC UGCAUUUUUA AGAAUUUUUA UAAACGUGGA AUGAUAGAGU
ACCUUUUUUG UCACGUUUCU UUUAUUUAUC AUAGCUAUUU UGAUUUUCAU CCAUUUUAUU
GCUGAGUAGU AUCCCAUUGC AUGUAUAUAC UAUACUGUAU UCAUUCGCUU GCUUGUGAAC
AUUUGGGCUU UUUCCAGUUU GGGACUGUUA ACAAGUAGAG CCACUAUGAA UAUUAGUGUA
UAAGACUUCA UAUAGCCAAG GCUGGCAGAU CGCUUGAGCC CAGGAGUUUG AGACCAGCCU
GGGAAACAUG GUGAAACCUC UAUUUUUAUU UUAAAAUCAA AAAUUAAAAA UUUUCUAUAA
AAAAUUUUAA AGAAGACUUU GUAUAGACAU ACGCUUUCAU UUUUCUUGAG UGAAUACUUA
GGUCUCAGGG UAGAUGUAUU UUAAGUCUUU AAGGAGCUGU CAAACUCUUC CUCAAAGUGG
UGGUUGUACC AUGUUACUUU UUAAUAUAAC AGAGAUUAAU UGAGCAAAGA AAAAUUCAAA
AGUUGGACAG CCCCCACAAC UAAAUAGGUU CAGAACAGCU CCCCCAUUUU GCAUUUUGAC
CAGCAAUGUA UGAAAGUUCC AUUUGCUCAG UGUCCCUGCA AACACCUGGU AUGGUCAGUC
UUUUUAAUUU UAGGCAUUAU AAUAGAUAUA GUGGCUUCUU GUGAUUUUAA UUAGCAUUUC
CUAAUGACCA GUGCUGCUGU UGAUCAUUUC AUGAGUGUAU UUGCCAUCCG UAUAUCUUUU
UUGGUGAAGU GUCUAUUCAA AUCAUUUGGG UUUUUUUUUU UUUUGUUUUU UUUUUUUGGA
GACAGUGUCU CACUCUGUCA CCCAGGCUGU UGUGCAGUGG UGCAAUCACA CAGCCUACUG
CAGCCUCCAC CUCCUGCGCU CAGUCUUCUU GUCUCAGCCU UCUGAGUAGC UGAAAUUACG
AGCACACGCC ACAAUGCCUG GCUAAUUUUU UAAAAUUUUG UAGAAACAAG GUCUCAUUAU
GUUGCCUGGG CUUGUCGUGA ACUCCUGGGC UCAAGCAAUC UUCCUGCCUC AGCCUCCCAA
AGAUUGGGAU UGCAAGUAUG AGCCACUGCA CCCGGCCAAC UUACCCAUCU UUUAAUUGAA
UUUUUUUGUU GUUGAGGUUU GAGAGUUCUU CAUGUUUGCU GGGUACAAUA UCUUUAUCAG
AUAGGUAACU UGCAUGUAUU UUCUCCCGGU UUACACUUUG GUUUUUCAUU UUGUUAACAA
CGUCUUUUUA AGAACAGAAA AUCUUAAUUU UGCUGAAAUC UAAUUUUUCA GUUUUUUCUU
UGAUGGUUUU GAGAGAGGAG GUAAAAAAAG ACUAGGUAAG CCGAUAGUUA GACAGAGUCC
UCGGUAGAAC UUCCCUUCUA ACAAAAAGCA GCCCAAGAAA UCACUUCUCU UCUAACAAGG
AGCAGCCUGG AAGAUCGGGC UGUAAACAUG UAUAAGGAAG CAGCUCUGGC ACAGAGGGGG
AGCUUCCUGG GUAAUCAGCA AGCUUCACAU ACGUAAGGUG GGUAUGUGAA GUAAACACAG
UAUGUGAAGU AAACACAGUG GACCUUAGUA CAUACUCAGA UAAGGAAGCQ GGAAGCUUGC
AUGUUGUGAG UUGUUGGGGU UGCCUGCAGC UGCACGGAGA GAAAGGGGUA CCUGGGGCCA
GGCAUGUCCA CCAUGGUGGC UCCACCUCCC CUUAUUUAGC ACAUGCACAA UAGGAAAGAG
AUAAGCAAUG UGGAGUAGCU CAGGCCAAGG ACCUGCCUGC AUAAUAAAAG GUUGGGGUGG
GGGAUGCCAG AGAUUCACGC UCUGUGCAGA UGGCAACACC UGGUCCUAAC UGGUUUUUUG
CUCCCUAUGU GUAGAUAAGC UACCCCCUUC CCAUUAGCUC AUUUAUAAAA AUGCUUGCAU
UUCACUGUGG AAUGGGAACU CUUUUCAGGA CCUCUCUCUG CAGGAGAGAG CUAGUCUCUU
UCUUUUGCCU AUUAAACUUC UGCUCUAGCC UCACACCCUU GGUGUGUCAG CGUCCUUGAU
UUCCUCAGCG UGAGACCAAG AACCUCGGGU GCCACCCCAG GCAACAAGGC CAUUUCAGUU
UGUUCUUUUG UUAUAGGCAA UCCAUGAUCA CAGAUUUUUC UCUCUUUUUU UUUUUUACAC
AGUUUAGAGU UUUAGUUUUA CACUUAGGUC UGUAAUCCAU UUUGUAUUAA UUCUUAUAUG
UGGCUCAGUG UAGGUGGAAA UUUGGUUUGU UUUUGCAUAA GGAUUUCCAA UAGUUUUACC
ACCAUUUCUU GAAACUACUA UGCUUUCUCU AUUAAACCAC AUUUGUAACU UUAGUUAAAA
UCAGUCACAU AUAUCACAGG GCUAUUUCUG ACUCUCAAUU CUGUUACAUU GUCUAUUAGU
GUAUAUUGAU GUCAGUACUA CACUUUUAAU UACUAUUGCU UCAGGGUAUG UCUUGUAAAC
CAAAAAUAAA AUUAUAGGCC CCCCCCGCCC CUGCACAACC AACUGAAUGG ACCCAUCCUC
UCAGCCAAGG GCAUUCCAAA AUUAACCUGA AAAACUAGUU CAAGCCAUGA UGGGAAGGGG
GAGUUGGACA UGUCUCAUCA CACCCUACUA CCUUUUGGAA UUACUGAUAG AACAGACUCU
UAAAGUCUGA AAAGAAACAU UUACAACCUA CCCUCUCUGA AGCCUGCUAC CUGGGAGCUU
CAUCUGCAUG AUAAAACCUU GGUCUCCACA ACCCCUUAUG GUAACCCAAA CAUUCCUUUC
UGUUGAUAAU AACUCUUUCA ACUAGUUGCC AAUUAGAAAA UCUUUAAAUC UUCCUAUGAC
CUAGAAACCU CCCUACCCCC ACUUUGAGUU GUCCUGCCUU UCCUGACAGA ACUCAUGUAC
AUCUUACAUA UAUUGAUUGA UGCCUCAUGU CUCCCUAAAA UGUAUAAAAC AAAGCUGUAC
CCCACCACCU UGGGGACAUG UCAUCAGGAC CUCCUGUGGC UGUGUCAUAG GAGCGUCUUU
AACUUUGGCA AAAUAAACUU UCUAAAUUGA UUGAAACCUG UCUUAGCUAC UUCUGGUUUA
CAGUCUUAAA GUUAGAUAAU GUAAAUUGUC CAGCUUUGGU UUAUUUUUGU CCUUAGUAGU
UCCAUAUAAA UUUUAGAAUC AGCUUUUCAA UUUAAUACAC UACUUUCCUC UUAGAUCCAC
AAUUAAAUAU AUUUGAUGCU AACAAUUCUG UUUUAUGUUU UUCGUUUUUU UUUUUUGAGA
CAAGAGUUUC GCUCUUGUUG CCCAGGCUGG AGUGCAGUGG CGCGAUCUUG GCUCACCACA
ACCUCCACCU CCCAGGUUCA AGCAAUUCUU CUGCCUCAGC CUCCCGAGUA GCUGGGAUUA
CAGGCAUGCG CCACCACGCC CGGCUAAUUU UGUAUUUUUA GUAGAGACGG GGUUUCACCA
UGUUGAUCAG GCUGGUCUUG AACUCCUGAC CUCAGGUGAU CCACCCACCU CGGCCUCCCA
AAGUGUUGGG AUUACAGGCG UGAACCACCA UGCCUGGCCA GUUCUGUUAU UUUUAAAACC
CAAGUUUCCC UGGUCAUAUC UUGGUUGGAU GAAGCGUAUU UUCAAUAGAU UACCCUGGAA
AGGCUAGUGA GUACGGUAUU CUUCUACAUU UUAGACUUUU CUUAGUCUUG CUACUUCAAG
GACAGCUAGG CUGCAUAUAA AAUUCUUGGC UCAUACUUUU UCCCCAUAAA UUUCUAUGAG
AAAGUCUAAU GAUAACUGAU UUUCUUUAUU UUGUAACUUA GUCUUUUUGC UUAGAGGCUC
UCUGAGGAUG GGAGGGGGUU CUUCCUCCCA UCCCUAGGAA UUUUUCUUUU UUUUAAAUUC
CUAAUCACUA GACCACCAGG AAGAUUGUUU GUUUUGUUUU GUUUUUAUUC UUCAGGGACC
CCAUUUAUAC AUACGUUAAA UAAAUACUGU UUGCCAAUGU AUCAACCAUU UUGCUUCUUA
UUUAUUUUUG UUCCUUUGGU UCUUUUUCAU GGCUUUGCUU UGGUGCUCCU UAGAUUUUCA
GUCAGAUGUA UUUGUCCUUG GGUACCUUGU AAUCAGUAUU ACCUUUUCUU CUGUCGCUUU
GUUUUCUGUU CGUUUUGAAA UUACUUGUUU CCUGGUCUGG CAAUAACAGU UGAGAUAUGA
GGAGUUUGAG CUGCCAUCUG UCUAUGUAUC UUGCUUUAAG ACUGCACUCU UCUAUUGAUA
UCACUGGCCU UGAUUUUGUG AUUUCUUUAU UUCUUCAGGA CCACCCUUCA UUUUCUACUG
UUUGCUUCCU UUUUUUUUGA GAUGGAGUCU CACUCUGUCA CUCAGGCUGG AGUGCAGUGA
UCUUGGCUCA UUGCAACCUC UGCCUCCCGG GUUCCAGCAA UUCUCCUGCC UCAGCCUCCC
AAGUAUCUGG GACUACAGGU GUGCACCACC AUGCCCGGCU AAGUUUUGUA UUUUUAAUAG
AGACGGGGUU UUGCCACAUU GGCAGGCUGG UCUCAAACUC CUGAUGUCAA GUGAUCCACC
CACCCCACCC ACCUCUGCAU CCCAAAGUGC UGGGAUUACA GGAAUGAGCU GCCGUGCCCA
GCCUCCCCCC UACCCCCCUU UUUUUCUUUC GAGACAGAGA UUAUAGGUGU GAGCCACUGG
ACCCAGCCUG UUUUUAUUCC UUUUACCAAA UCUCCAAGGA AUAUCUUCCC UUCCAAGUGC
GAAUGUAACC UUAAGUCAGU UAACCUCUUU GUGAUUACUU UUCUUAUCUG CAAAGUGACU
UAAUGAUCUU AAGUACUUUU UUUUUUUGAG ACAGGGUCUC ACUGUCACCC UGGCUGGAGU
GCAGUGGCAC GAUCUCUGAU CUCCACUCAC UGCAAUCUCC UCUUCCCUGG UUCAAGCGGC
CCUCCCACCU UAGCCUUCUG GGUAGCUGGG ACUACAGAUG UGAACCACCA CGCCCAGCUA
AUUUUUGUAC UUUUUGUAGA GAUGGGGUUU UGCCAUGUUG CCCAGGCUGG GAUUAUUAAG
UACUUUUUAU CAUACAGCAA GAUUGACAUU UUAUAUUGGA AUACAUUUGU CUCUAUAUAA
CGGAGAUUAA CAGGAAAAUG ACAAGCCUGG GUGCGGUGGC UCAUGCCUGU AAUCCCAGCA
CUUUGGGAGG CUGAGGUGGG AGGAUCACUU GAGGUCAGGA GUUCGAGACC AGUUUUGCCA
AGAUGAUGAA AGCCCAUGUC UACUAAAAAU ACAAAAAUUA GCCCAGCUUG AUGGUGGGCG
CCUAUAAUCC CAGCUAUUUG AGAGACUGAG GCAGGAGAAU CACUUGAACC UGGGCAGCAG
AGGUUGCAGU GAGCCGAGAU CAUGCCACUG CACUCCAGCC UGGGUGGCAU AGCGAGACUC
UUGUCUCAAG AGAAAACAAA AGAAAACAAA AAAAAAACAG GAAAAUGACA AAAAGUAAUA
UUACAACUCA GUGAAUUUUA UAACAAACUU UUUUGGAAUU CAUUGACUAA UACUAUACCA
AAUCCAAAAU ACUCUCUAGU AUACCAAAUC CAACUCUACC CUAUAGUAUA AAUUGGAUUC
UAUUUGGACU UGUCUCACUA AUCCCUCAUA CAGUGUGUUU UAUUUUUUAU UGAAGUAAAA
AAAUUUGUCA UUUUAACCAU UUUUAAGUAU AUAGUUCAGU AAUAUUAAGU AUGUUCAUGU
UGUUGCGCAA UAGAUCUUCG GAAGUUUUUC GUCUUGCAAC CUGAAACUCU ACCCAUUAGC
AAAUUCCCAU UUCUCCUUAC ACUUAGCCCU UGGUAAUCAU CAUUCUUUUU UUUUUUUUUU
UGAGAUGGAG UUUUACUCUU GUUGCCCAGG CUGGAGUGCA AUGGUGCAAU CUCGACUCAC
CACAACCUCC GCCUCCCAGG UUCAAGCAAU UCUACCUCAG CCUCCCGAGU AGCUGGGAUU
ACAGUCAUGC ACCACCACGC CCGGCUAAUU UUGUAUUUUU AGUAGAGAAG GGGUUUCUCC
AUGUUGAGGC UGGUCUCGAA CUCCUGACCU CAGGUGAUCU GCCCACCUCG GCCUCCCAAA
GUGCUGGGAU UACAGGCGUG AGCCACUGCG CCUGGCCCAU UCUUUCUAAU UCUAUAAAUU
UGACUACUUA GUUACCUUAC AUAAAUAAAU UCUUAUAGUU AGUGUUAUUU UUGCUUCCAU
GCCUUUUUUG UUGUUGUUCA UGCUCUUACU UGGAAUGCGU UCUAUUUUGU CUACCUAUGC
ACAUCCUGUU GGGUUUUUUU UUUUUUUGGG GGUUUUUUUU GUUUUUUUUU GUUUUUUUUU
CCCAGACAAG GUCUCAAUUU GUUACCCAGG CUGGAGUGCA GCGGCGCCAU CUCCACUCAC
UGCAUCCUCA ACUUCCUGGG CCCAGGUGAU CCUCUCGCCU CAGCCCCUGC AGGUAGCUGG
GACUAUAGGC AUGUGCCACC AUGCCCAGCU AAAUUUGGUU UUUUUGUUUG UUUGUUUUUG
AGACAGAGUC UCACUCUGUC ACCCAGGCUG GAGUGCAGUG GCACAAUCUC AGCUCACUGC
AAUCUCUGCC GCCCGGGUUC AAGUGAUUCU CCUGCCUCAG CCUCCCAAGC AGCUGGGAUU
ACAGGUGACU GCCACCACGC CAGCUAAGUU UUGUAGUUUU AGUAGAGAUG GGGUUUCACC
UUGUUGGCCA UGCUGGUCUC GAACUCCUGA CCUCGUGAUC UGCCUGCUUC UGCCUCCCAA
AGUGCUGGAA UUACAGGCAU GAGCCACCAC GCCCGGCCAG AAUUUUUGUA UUUUUAGUAG
ACACAAGGUU CUUACCCUGU UGCCUAGGCU GGUCUGGAAG UCCUGGACUC AAGCAAUUCA
CCUGCCUUGG CCUCCCAAAA UGCUGGGAUU ACAAGCCACC AUGCCCGGCC UAAAUCCUGU
UGUUUUGUUU UGUUUUAUUU UGUUUUGUUU UGUUUUGUUU GUUUUUUGAG ACAGAGUCUC
GCUAUGUCUC UCAGGCUGUA GUGCAGUGGC GCGAUCUUGG CUCACUGCCA CCUCUGCCUC
CCAGGUUCAA GUGAUUCUCC UGCCUCAGCC UCCCAAGUAG CUGGGAUUAC AGGCAUGUGC
UACUAUGUCC GGCUAAUUUU UGUAUUUUUA GUAGAGACAG GGUUUCACCA UGUUGGCCAG
GCUGGUCUCG AACUCCUGAC CUCGUGAUCC ACCCACCUCG GCCACCCAAA GUGCUGGGAU
UACAGGCGUG AGUGGUUUUU AUUUCUUAGG CCGGUUUCCU CCAUAUGAUC UUGCAGUAGA
CAUUAAUUUC UUUCCUUUUU AAUUAAAAUA CUGUUUGUAU UUCACAUUUU GAUGUUUGUU
AAGAUUUGUU UUAUAUUGUU UUUUGUUUUG UCUUGUGUGA UAGUCUUAAA UCCCUAGUUA
GAUAAUAACU GGAGAGUACC AUGUUUCUAU AUAUCUCUCA GUGACUUGCA CAGUGCUAGC
AGAUAGUGCU AAAAAAUUAU UUAUUAUUAU UAUUAUUUUG UUAUUGUUGU UGUUGUUGUU
AGACAGGGUC UUCCUCUGUC ACCCAGGCUA GAGGGCAAUG GGAUGAUCAU AGCUUACUGC
AGOCUCCAAC AACUGGGCUC AUGUAAUUCU CCUGCCUCAG CUUCCCAAGU AGCUGGGAUU
ACAGGCAUGA GCCACCAUGU CUGGACAAAA AUAUUUCCAG GUGCAGUGGC UCAUGCCUGU
AAUUCCCACA CUUGGGAGGC CGAGCGAGGC UGGAGGAUCA CUUGAGCCUA GGAGUUCAAG
ACCAGCUUGG CUAAGAUGGC GAGACCCCGU CCCUACAAAA AAUUUUAAAA ACUAGCCAGG
CAUGGUGGCA UGCACCUAUA UUCCCAACUA CUCAGUGGGC UGAGGUGGGA GGGUCAUUUG
AACACAGGAA UUUGAGGGGA GAAAAAAAGA AGAGAGAAAG AGAAGUGAAG GAAGGAAGAA
AGGAAGGAGG GAGGGAGAGA AGAAAGAAAC GAAAGAAAGG AAAAGAAAAG GAAGGAAAGA
AAAUUGGUAC CAGGAAAGCA GGAAAGGGAA AUGGAAGUAA AAAAAUAAUA AUAAUAAUAA
AAUGAAAAUU GGUUAGUCAC UAUUAACAAU UUGUAUCCUU AUAAUCUGGA AACAUUAUAA
UUUCAAAAGA AAAAAUAUUC UUUGGAUCAU AGGUUCUGAG GUCAGAACAG CAUUCCCGUA
GUCUAGAUGA AGUCAAGUUU UAUCUGAUCU UAAUUGAAAU AAAUAUAGCU GGCCUUGAAC
AAAUCUACUC AUGGUAUGUG GAUAGGAAUU AAAUUGUAGG GGCAUUCACU UGAUGGCAUU
CAUUCUUAGA ACAUUUACCU AUGUCUAGCU UUUGGAGUAA AGUCACAUAA CCUCUAACCA
GGUAAGUUUC CUGUGGCUUU AUUUAGGAUU UUAAAUACUC AUUUUCAGUG UAAUUUUGUU
AUGUGUGGAU UAAGAUGACU CUUGGUACUA ACAUACAUUU UCUGAUUAAA CCUAUCUGAA
CAUGAGUUGU UUUUAUUUCU UACCCUUUCC AGAGCGAUGA UUCUGACAUU UGGGAUGAUA
CAGCACUGAU AAAAGCAUAU GAUAAAGCUG UGGCUUCAUU UAAGGUAUGA AAUGCUUGCU
UAGUCGUUUU CUUAUUUUCU CGUUAUUCAU UUGGAAAGGA AUUGAUAACA UACGAUAAAG
UGUUAAAGUA CAUGUUAUUC AGUUUUCAUU UUGAAGAUUA GAUGGUAGUA UGAGUUAGUU
AAAUCAGGUG AUAUCCUCCU UUAGAAGUUG AUAGCCUAUA UAUGUCAUCC UUUGUGGAGG
CAAUUUAAAU AAAAUUUAAA ACAUUUAUUC CUGGCUGGGU AUGGUGGCUC ACUCCUGUAA
UCCCAGCACU UUGAGAGGCU GAGGCGGGUG GAUCACCUGA GGUCAGGAGU UUGAGACCAG
CCUGGCCAAC AUGGUGAAAC CCCGUCUUUA CUAAAAAUAC AAAAAUUAGC CAAGCAUGGU
GGCACGUGCC UGUAAUCCCA GCUGCUUGGG ACACUGAGGC AGGAGAAUUG CUUGAACCUG
GGGGGCAGAG GUUGCAAUGA UUGCACCACU GCACUCCAGC CUGGGCGAUA GAGUGAGACU
CCAUCUCAGA AAACGAACAA ACAAUGUAUU CCUUUUAGUA UUUUUACAUU GUAUCAAACU
AUGGAAGUCC UCUAAUUGAG AUUAAUAAGA AAAAGACAAU CUGAAUUAUA AUUUUAAACA
UUUAACAAGC AUGUAGUAAA AUAAUGAUGA AGAUAAAUAG CAUUAGUACA GCAAUUAAUA
UUUGUAGCAU GCUGACAGUG CUCUGUGUGC GUUUCAUAUA UUAAAUUACU CUAAUCAUCC
CAAAUCCUGU AAGUUGGGUA UCAAUUCAAG UGUUCCUAUU GGGUAGGAAU AUACAGUUCU
UUUAGGAAAU GUAGUAUGGU UCUGUGUCUC AAACAGGACA CUUACACAGU UGGCCAACAU
CAUCACCUUC UCCAUUCUCU GAGAUGUUUA GUCUUACUGA GCACUAAAUA UGGGUCAUCA
AUAGUCCAGA CUACCUUGAG CAAACAAUAG UCCAGACUAC CUUGAGCAAA CAGAGCAUAU
ACUCAUACAG UGUAUAAAGA GCACCAAGCA UACAGAUUUC AUGUCUUUCU CAUAGUUACU
CUUGUAACAU GAGCUAAAGA UCAGACCUCU AUGUCACCUU UGUAACUGAU UUCUAGAUUU
UUUUUUUUUU UUGAGAUGGG GUCUUGCCCU GUCACCCAGG CUGGAGUGUA GUGGCGUGAU
CAUGCCUCAU UGGAGCCUUC AACUCAUGAG CUCAAACAAU CCUCCUACCU CAGCUUCCUG
AGUAGUUGGG ACCACAGGUG UGUGCCACCA CACCCAGCUC AUUUUUGUAU UCUUUGUAGA
GAUGCAGUCU CACCCUGUUG CCCAGGCUGG CCUGGAACUC CUGAGCUCAA AAGAUCCCUC
CGCCUUGACC UUCCAAAGUG CUGGGAUUAC AAGCAUGAAC CACUGCACCC GGCCUAGAUU
UUUAAAUGUG CUUUCCAGUA UACACUGAAA CUAGAAGUCG ACUAAAGAAU UACCAAGAGA
AUUCUAUAAA AUAGAGAUUG AAAUGGGGCU CGAUGUGGGA UGGGUUGGUG AUAUUGCAGG
GAGAAGUAAU CUGAGUAAAG GAGGAAAAGA ACUGAUUUGG GAAAACGAUA GUUUUAGUAG
UGAGUUUGAG UAUGAAUUAA GUUGAGAUUG AAUUUGAAUU AAGUUGAGGU UGAAUAUGAA
UUAAGUUGAG GUUGAGUUUG AGGUAUGAAU UAAGAUGUGA AAUUGAUCAU UGGAAAUGUU
AGAUUGAGAA AAGUCACAGC UGGAUUAAUA GCUUCAGAAG UGUGUUUGCA GACAGUUGCA
ACUAAAGUAA UAAGAAUAGA UGGCCUUGGC CGGGCGUGGU GGCUCACGCC UGUAAUCCCA
GUACUUUGGG AGGCUGAGGC GAGCAAAUCA CGAGGUCAGG AGUUCAAGAC CAGCCUGGCC
CACAUGGUGA AACCCCGUCU UUAUUAAAAA UACAAAAAUU AGCUGUGCAC AGUGGUGCAC
GCCUGUAAUC CCAGCUACUC GGGAGGCUGA GACAGGAGAA UCGCUU3AAC CUGGGAGGUG
GAGGUUGCAG UGAGCUGAGA UCAGUGUGAC UGCACUCCAG CCCGGUGACA GAGUGAGACU
CUGUGUAAAA AAAUAAAAUA AAUAAAAUAA UGGCCGUAAG CAAGUAAAGA AGGAUGGCCA
GCUCUUAUUG GGAAUGCCUA AAUCUAAGGC UUGAUCAGAA GUAAUGAAAC CGUUGGGGCC
CUACAUUGCU AUGACAUCCA AAGGGCCAUG AAUAUCAGGA AGAAAGAUAA UUAACAGGGU
CUAAUGUUAC AGAGAGGUUG AGAGCAAGGA GAUUUGAUUA AAAGGGUCUU UAGAGCUGAU
GUCAGGUGUA UGAUGCCUUU AAGAGCAGUU UUUAUAGUGC AGGGGGUGGU CAAAAGAGAA
AAUAGGUGCU UUCUGAGGUG ACGGAGCCUU GAGACUAGCU UAUAGUAGUA ACUGGGUUAU
GUCGUGACUU UUAUUCUGUG CACCACCCUG UAACAUGUAC AUUUUUAUUC CUAUUUUCGU
AGCAUGCUCU AAAGAAUGGU GACAUUUGUG AAACUUCGGG UAAACCAAAA ACCACACCUA
AAAGAAAACC UGCUAAGAAG AAUAAAAGCC AAAAGAAGAA UACUGCAGCU UCCUUACAAC
AGGUUAUUUU AAAAUGUUGA GAUUUAACUU CAAAGGAUGU CUCAUUAGUC CUUAUUUAAU
AGUGUAAAAU GUCUUUAACU UAAGUGAUUA GUACAGUGUU UCUAUUGACA UAUACUUAUA
CAACUUCAAA AACAACUAUU AAAUUUUCUG UUAUUUAGGA ACAUGCAUAU UAGUCAUGAA
AGUAUAAAGA AUUAGAUGGG AAUGAUAAAU GCUAAAAUCA GGACAUGUGU UCCAUUUGUG
AAUGGAAGGC AGGGAGAAGG UGCCGUUUGG AAGGAGUACC CAAGAGCCGU AAGCUGAAUU
GGCAGUGUUU UACAUCUUAA GCUGAGAGAU AGAUUUUUUU UUCCCCUUUU UCUUUAAAAA
CUCUAAAACU GUUAAUUCCA AGGAACCCAG AAGUCUAGGU AGAUUAUUUC UGCUAGUUAA
AAGCAGUAGU CCUGAAAGCU GAAUAUUUUG GUGUCUUUUG AGCCAACUUU AGUUUCAUCA
UUACCAAGGG GGAAGAGAGC UAACAGUUGA UGAGCACUUG CUCUAGGCCA GUCCAGAGUG
CUGGGCACCA UACGCAUUUU AUCUCCCUCC CGCUAUUCAC AACAAAUAUG GGAGGUAGUU
UAUAUUAUAG CCAUCUAAUA AGAUGGGGAA ACUAAGACUC AAAGAGAUUC AGAAACUUGU
CCAUGAUUAU AAAUGUAAGA GAGUUGGAAU UCAGAUUUAU GUAUUUAGAC CCCAAGCCUU
UCUCAUUACA UCAUUUUGCC UUCCAAAUCU CUACCCUCUA UCCUUCACCU CCCCACUGAU
CAAAACGAGA UGAUAGUUUG CCCUCUUCAA AAGAAAUGUG UGCAUGUAUA UAUCUUUGAU
UUCUUUUGUA GUGGAAAGUU GGGGACAAAU GUUCUGCCAU UUGGUCAGAA GACGGUUGCA
UUUACCCAGC UACCAUUGCU UCAAUUGAUU UUAAGAGAGA AACCUGUGUU GUGGUUUACA
CUGGAUAUGG AAAUAGAGAG GAGCAAAAUC UGUCCGAUCU ACUUUCCCCA AUCUGUGAAG
UAGCUAAUAA UAUAGAACAA AAUGCUCAAG AGGUAAGGAU ACAAAAAAAA AAAAAUUCAA
UUUCUGGAAG CAGAGACUAG AUGAGAAACU GUUAAACAGU AUACACAGUU GUCAGUUUGA
UCCACCGAGG CAUUAAUUUU UUCUUAAUCA CACCCUUAUA ACAAAAACCU GCAUAUUUUU
UCUUUUUAAA GAAUGAAAAU GAAAGCCAAG UUUCAACAGA UGAAAGUGAG AACUCCAGGU
CUCCUGGAAA UAAAUCAGAU AACAUCAAGC CCAAAUCUGC UCCAUGGAAC UCUUUUCUCC
CUCCACCACC CCCCAUGCCA GGGCCAAGAC UGGGACCAGG AAAGGUAAAC CUUCUAUGAA
AGUUUUCCAG AAAAUAGUUA AUGUCGGGAC AUUUAACCUC UCUGUUAACU AAUUUGUAGC
UCUCCCAUGA AACUUUUGUA GCUUAAAUAC ACAAGAAUUU UUUGAAAAGG AAAUAAGAUA
AUGAUGCAAA AUAbUUAAUU UUUUAAAAAA AUGUUAGACA CUGCAGUGGA UGCAACAAAA
UACUUUAUAU GAAAGAUUUA UCCAGUUAAC UUUUGUGGAG UAUUAGGUAU UAGACUAAUA
AUUAGCACAC UUACUUAAGU UAGAAAGUAU AAUAAUGCGC CGGACGCGGU AGCUCACGCC
UGUAAUCCCA GCACUUUGGG AGGCCAAGGU GGGCGGAUCA CAAGGUCAGG AGAUCGAGAC
CAUCCUGGCU AACACUGUGA AACCCCAUCU CUACUGAAAA UACAAAAAAA UUUGCCGGGC
GUGAUGGCGG GCACCUGUAG UCCCAGCUAC UCGGGAGGCU GAGGCAGGAG GAUGGUGUGA
ACCCCGGAGG CAGAGCUUGC AGUGAGUCAA GAUCGUGCCA CUGCACUCCA ACCUGGGCGA
CAGAAUGAGA CUCCAUCUCA AACAAAAAAA CAAAACAAAA CAAAAAAAAG UGUAAUAAUA
AUUUAUCAUU AGOUGGAUGA UAUGCUGUUG UUUCCCAUGU CACCUGUAUA AGAUAUGUAA
AAUAAGAACA CAUUAUUUAC AUCUAAUAUA GAUAAAAUCC UGAGGCGCUC UCAGAUUGUU
UUGUAGAGUU CAAAUGUAAA UAUUGUUUUC AUUUAUGGUC CUUUUGGUUA UAAGUAACAG
AAAUCAACUC UAAAAAGAUU UUUAUUAUAG GUUAGAUUAU GUCAUGGAAC CUUAAGGCUU
GUCCCUUUCU AGUUCUUUUG UGUAAAGCGG UGAUUUCUUC CAUGGAGGGA AUGGUAUUUA
GGCAAUUUUU UUUUUUUUUU CGAGAUGGAG UCUUGCUCUG UCGCUCAGGC UGGAGUGCAG
UGGCACCAUU UCAGCUCACU GCAACUUCCA CCUCCUGGGU UCAAGUGAUU CUCCUGCUUC
AGCCUCCCAA GUAGCUGAGA UUACAGGCAC CCGCCACCAC ACCCGGCUUA UUUUGUAUUU
UUAGUAGAGA UGGGGUUUCA CCAUGUUGGC CAGGCUGGUC UUGAACUCCU GACCUCAAGU
GAUCUCCCCA CCUUGGCCUU CCAAAGUGCU AGGAUUACAG GCGCCUAGCC UAGGCAGUCA
UUUUCAAAAA ACAAGCAUGA CUCACCAAAA GUUUUAAGAU UUUCUGUGAU AAUGUUCUUA
UUGAGGCUUA CAUUAUAUUA CAGUUUCUUG AAUCUAAAAU GAUGUACCCU CUUAGAAUAU
AUACAUCAUG CUUCAUUGGU CUCAGGGGGC UGAUUUUUAU AAGGAGAGAU UUGCUAGUUU
UCACAAUAUG UCCUCUAAGU UGGCAUGUAU AGCUAAACAG GCUUUCAUAA AAAUAUACAA
UUUAGUUAAU GAAAUUUGGG AUAUAGUCUU UUAUGAUUGA AAUAAUUUUG CUAAAUAGAC
UGUCUCUGAU UOAUUAGGUA AUCACCACUC UUAUUUUGUU UUACUUCCUU AAUGUCUACA
UAGAAAGGAA AUGAGAAAAA UCCAGAGGUU GUCAUUUGAC UUAUGAGUCU GUUUGACUUC
AGGAUUUGGU ACAUGAAAUU UCACUUAAUC UUUUUGAUAU GUAUAAAACA AAUAUUCUGG
GUAAUUAUUU UUAUCCUUUU GGUUUUGAGU CCUUUUUAUU CCUAUCAUAU UGAAAUUGGU
AAGUUAAUUU UCCUUUGAAA UAUUCCUUAU AGCCAGGUCU AAAAUUCAAU GGCCCACCAC
CGCCACCGCC ACCACCACCA CCCCACUUAC UAUCAUGCUG GCUGCCUCCA UUUCCUUCUG
GACCACCAGU AAGUAAAAAA GAGUAUAGGU UAGAUUUUGC UUUCACAUAC AAUUUGAUAA
UUAGCAGAAU AGAGGAUUGU AAAAUGUCAU UGUAGAACAU CCCUUGGGCC AGAUUCUAAU
GGGUAGAAAU UUGAACUAAA CCUCUGGGUU UUGUUUGUUU UUAAUGCCUU UCUGUUACCC
AGAUGCAGUG CUCUUGUAGU CCCAAGUCUA AGCUCUAGGU UGCCUUCUUU CCUGGCAGAA
GUUGGUGUCU AUGCCAUAAG GAGGUAGUUC CUGUUAGAAG GGAUUUAAUU AUACCUUAUA
UAAGGAAUUA GUGUUUGCCC UUCUAGGUAU AGUUGGAUGU UAGCUUCUGA UGUAAACUGG
AUUUCUUUUU CUUUCUCUCU CUUUUUUUUU UUUUGUUUUG GAGGCAGAGU UUUGCCCUUG
UACCCCAGGC UGGAGUGCAG UGGUGUGAUC UCAGCUCACA GCAACCUCCG CCUCCUGGGU
UCAAGCAAUU CUGCCUCGGC CUCCCAAGUA GCUGGGAUUA CAGGCGACUG CCACCACACC
CGGCUAAUUU UUGUUUUAUU AGUAGAGAUG GGGUUUCACC AUGUUGGCCA GACUGAUCUU
GAACUCCUGA CCUCAGGUGA UCCACCCGCC UUGGCCUCCC AAAGCGCUGG GAUUACAGGC
GUGAGCUGCC GCACCCAGCU GUAAACUGGA UUUCUAAUGG UAGAUUUUUA GGUAUUAACA
AUAGAUAAAA AGAUACUUUU UGGCAUACUG UGUAUUGGGA UGGGGUUAGA ACAGGUGUUC
UACCCAAGAC AUUUACUUAA AAUCGCCCUC GAAAUGCUAU GUGAGCUGUG UGUGUGUGUG
UGUGUGUGUG UGUAUUAAGG AAAAGCAUGA AAGUAUUUAU GCUUGAUUUU UUUUUUUUAC
UCAUAGCUUC AUAGUGGAAC AGAUACAUAG UCUAAAUCAA AAUGUUUAAA CUUUUUAUGU
CACUUGCUGU CUUUUCGUCC UCGUUAAAUU UAAUUUUGUU GGUCUUUUGU UGUUAUUGGU
UGGUUUUCUC CAAAUGCUAG CUAUGUUAAG AAAUUUAAGG CCAGGUACAG UGGCUCAUGC
CUGUAAUCCC GGCAUUUUAG AAGGCUGAGG CAGGAGGAUC ACUUGAGCUC AGGAGUUUGA
GACCAGUCUG GGCAACAUAG CAAGACCUCG UCUUUGUUUA GGGGAAAAAA AAGAAAUUUA
AGUAGGAGAU UAUAUAAGCA AAAAUACAAU UAAUUUCCAG CAUUCACUAU AUAAUAUAAA
UCUCCAGACU UUACUUUUUU GUUUACUGGA UAUAAACAAU AUCUUUUUCU GUCUCCAGAU
AAUUCCCCCA CCACCUCCCA UAUGUCCAGA UUCUCUUGAU GAUGCUGAUG CUUUGGGAAG
UAUGUUAAUU UCAUGGUACA UGAGUGGCUA UCAUACUGGC UAUUAUAUGG UAAGUAAUCA
CUCAGCAUCU UUUCCUGACA AUUUUUUUGU AGUUAUGUGA CUUUGUUUUG UAAAUUUAUA
AAAUACUACU UGCUUCUCUC UUUAUAUUAC UAAAAAAUAA AAAUAAAAAA AUACAACUGU
CUGAGGCUUA AAUUACUCUU GCAUUGUCCC UAAGUAUAAU UUUAGUUAAU UUUAAAAAGC
UUUCAUGCUA UUGUUAGAUU AUUUUGAUUA UACACUUUUG AAUUGAAAUU AUACUUUUUC
UAAAUAAUGU UUUAAUCUCU GAUUUGAAAU UGAUUGUAGG GAAUGGAAAA GAUGGGAUAA
UUUUUCAUAA AUGAAAAAUG AAAUUCUUUU UUUUUUUUUU UUUUUUUUGA GACGGAGUCU
UGCUCUGUUG CCCAGGCUGG AGUGCAAUGG CGUGAUCUUG GCUCACAGCA AGCUCUGCCU
CCUGGAUUCA CGCCAUUCUC CUGCCUCAGC CUCAGAGGUA GCUGGGACUA CAGGUGCCUG
CCACCACGCC UGUCUAAUUU UUUGUAUUUU UUUGUAAAGA CAGGGUUUCA CUGUGUUAGC
CAGGAUGGUC UCAAUCUCGU GACCCCGUGA UCCACCCGCC UCGGCCUUCC AAGAGAAAUG
AAAUUUUUUU AAUGCACAAA GAUCUGGGGU AAUGUGUACC ACAUUGAACC UUGGGGAGUA
UGGCUUCAAA CUUGUCACUU UAUACGUUAG UCUCCUACGG ACAUGUUCUA UUGUAUUUUA
GUCAGAACAU UUAAAAUUAU UUUAUUUUAU UUUAUUUUUU UUUUUUUUUU GAGACGGAGU
CUCGCUCUGU CACCCAGGCU GGAGUACAGU GGCGCAGUCU CGGCUCACUG CAAGCUCCGC
CUCCCGGGUU CACGCCAUUC UCCUGCCUCA GCCUCUCCGA GUAGCUGGGA CUACAGGCGC
CCGCCACCAC GCCCGGCUAA UUUUUUUUUA UUUUUAGUAG AGACGGGGUU UCACCGUGGU
CUCGAUCUCC UGACCUCGUG AUCCACCCGC CUCGGCCUCC CAAAGUGCUG GGAUUACAAG
CGUGAGCCAC CGCGCCCGGC CUAAAAUUAU UUUUAAAAGU AAGCUCUUGU GCCCUGCUAA
AAUUAUGAUG UGAUAUUGUA GGCACUUGUA UUUUUAGUAA AUUAAUAUAG AAGAAACAAC
UGACUUAAAG GUGUAUGUUU UUAAAUGUAU CAUCUGUGUG UGCCCCCAUU AAUAUUCUUA
UUUAAAAGUU AAGGCCAGAC AUGGUGGCUU ACAACUGUAA UCCCAACAGU UUGUGAGGCC
GAGGCAGGCA GAUCACUUGA GGUCAGGAGU UUGAGACCAG CCUGGCCAAC AUGAUGAAAC
CUUGUCUCUA CUAAAAAUAC CAAAAAAAAU UUAGCCAGGC AUGGUGGCAC AUGCCUGUAA
UCCGAGCUAC UUGGGAGGCU GUGGCAGGAA AAUUGCUUUA AUCUGGGAGG CAGAGGUUGC
AGUGAGUUGA GAUUGUGCCA CUGCACUCCA CCCUUGGUGA CAGAGUGAGA UUCCAUCUCA
AAAAAAGAAA AAGGCCUGGC ACGGUGGCUC ACACCUAUAA UCCCAGUACU UUGGGAGGUA
GAGGCAGGUG GAUCACUUGA GGUUAGGAGU UCAGGACCAG CCUGGCCAAC AUGGUGACUA
CUCCAUUUCU ACUAAAUACA CAAAACUUAG CCCAGUGGCG GGCAGUUGUA AUCCCAGCUA
CUUGAGAGGU UGAGGCAGGA GAAUCACUUG AACCUGGGAG GCAGAGGUUG CAGUGAGCCG
AGAUCACACC GCUGCACUCU AGCCUGGCCA ACAGAGUGAG AAUUUGCGGA GGGAAAAAAA
AGUCACGCUU CAGUUGUUGU AGUAUAACCU UGGUAUAUUG UAUGUAUCAU GAAUUCCUCA
UUUUAAUGAC CAAAAAGUAA UAAAUCAACA GCUUGUAAUU UGUUUUGAGA UCAGUUAUCU
GACUGUAACA CUGUAGGCUU UUGUGUUUUU UAAAUUAUGA AAUAUUUGAA AAAAAUACAU
AAUGUAUAUA UAAAGUAUUG GUAUAAUUUA UGUUCUAAAU AACUUUCUUG AGAAAUAAUU
CACAUGGUGU GCAGUUUACC UUUGAAAGUA UACAAGUUGG CUGGGCACAA UGGCUCACGC
CUGUAAUCCC AGCACUUUGG GAGGCCAGGG CAGGUGGAUC ACGAGGUCAG GAGAUCGAGA
CCAUCCUGGC UAACAUGGUG AAACCCCGUC UCUACUAAAA GUACAAAAAC AAAUUAGCCG
GGCAUGUUGG CGGGCACCUU UUGUCCCAGC UGCUCGGGAG GCUGAGGCAG GAGAGUGGCG
UGAACCCAGG AGGUGGAGCU UGCAGUGAGC CGAGAUUGUG CCAGUGCACU CCAGCCUGGG
CGACAGAGCG AGACUCUGUC UCAAAAAAUA AAAUAAAAAA GAAAGUAUAC AAGUCAGUGG
UUUUGGUUUU CAGUUAUGCA ACCAUCACUA CAAUUUAAGA ACAUUUUCAU CACCCCAAAA
AGAAACCCUG UUACCUUCAU UUUCCCCAGC CCUAGGCAGU CAGUACACUU UCUGUCUCUA
UGAAUUUGUC UAUUUUAGAU AUUAUAUAUA AACGGAAUUA UACGAUAUGU GGUCUUUUGU
GUCUGGCUUC UUUCACUUAG CAUGCUAUUU UCAAGAUUCA UCCAUGCUGU AGAAUGCACC
AGUACUGCAU UCCUUCUUAU UGCUGAAUAU UCUGUUGUUU GGUUAUAUCA CAUUUUAUCC
AUUCAUCAGU UCAUGGACAU UUAGGUUGUU UUUAUUUUUG GGCUAUAAUG AAUAAUGUUG
CUAUGAACAU UCGUUUGUGU UCUUUUUGUU UUUUUGGUUU UUUGGGUUUU UUUUGUUUUG
UUUUUGUUUU UGAGACAGUC UUGCUCUGUC UCCUAAGCUG GAGUGCAGUG GCAUGAUCUU
GGCUUACUGC AAGCUCUGCC UCCCGGGUUC ACACCAUUCU CCUGCCUCAG CCCGACAAGU
AGCUGGGACU ACAGGCGUGU GCCACCAUGC ACGGCUAAUU UUUUGUAUUU UUAGUAGAGA
UGGGGUUUCA CCGUGUUAGC CAGGAUGGUC UCGAUCUCCU GACCUCGUGA UCUGCCUGCC
UAGGCCUCCC AAAGUGCUGG GAUUACAGGC GUGAGCCACU GCACCUGGCC UUAAGUGUUU
UUAAUACGUC AUUGCCUUAA GCUAACAAUU CUUAACCUUU GUUCUACUGA AGCCACGUGG
UUGAGAUAGG CUCUGAGUCU AGCUUUUAAC CUCUAUCUUU UUGUCUUAGA AAUCUAAGCA
GAAUGCAAAU GACUAAGAAU AAUGUUGUUG AAAUAACAUA AAAUAGGUUA UAACUUUGAU
ACUCAUUAGU AACAAAUCUU UCAAUACAUC UUACGGUCUG UUAGGUGUAG AUUAGUAAUG
AAGUGGGAAG CCACUGCAAG CUAGUAUACA UGUAGGGAAA GAUAGAAAGC AUUGAAGCCA
GAAGAGAGAC AGAGGACAUU UGGGCUAGAU CUGACAAGAA AAACAAAUGU UUUAGUAUUA
AUUUUUGACU UUAAAUUUUU UUUUUAUUUA GUGAAUACUG GUGUUUAAUG GUCUCAUUUU
AAUAAGUAUG ACACAGGUAG UUUAAGGUCA UAUAUUUUAU UUGAUGAAAA UAAGGUAUAG
GCCGGGCACG GUGGCUCACA CCUGUAAUCC CAGCACUUUG GGAGGCCGAG GCAGGCGGAU
CACCUGAGGU CGGGAGUUAG AGACUAGCCU CAACAUGGAG AAACCCCGUC UCUACUAAAA
AAAAUACAAA AUUAGGCGGG CGUGGUGGUG CAUGCCUGUA AUCCCAGCUA CUCAGGAGGC
UGAGGCAGGA GAAUUGCUUG AACCUGGGAG GUGGAGGUUG CGGUGAGCCG AGAUCACCUC
AUUGCACUCC AGCCUGGGCA ACAAGAGCAA AACUCCAUCU CAAAAAAAAA AAAAUAAGGU
AUAAGCGGGC UCAGGAACAU CAUUGGACAU ACUGAAAGAA GAAAAAUCAG CUGGGCGCAG
UGGCUCACGC CGGUAAUCCC AACACUUUGG GAGGCCAAGG CAGGCGAAUC ACCUGAAGUC
GGGAGUUCCA GAUCAGCCUG ACCAACAUGG AGAAACCCUG UCUCUACUAA AAAUACAAAA
CUAGCCGGGC AUGGUGGCGC AUGCCUGUAA UCCCAGCUAC UUGGGAGGCU GAGGCAGGAG
AAUUGCUUGA ACCGAGAAGG CGGAGGUUGC GGUGAGCCAA GAUUGCACCA UUGCACUCCA
GCCUGGGCAA CAAGAGCGAA ACUCCGUCUC AAAAAAAAAA GGAAGAAAAA UAUUUUUUUA
AAUUAAUUAG UUUAUUUAUU UUUOAAGAUG GAGUUUUGCC CUGUCACCCA GGCUGGGGUG
CAAUGGUGCA AUCUCGGCUC ACUGCAACCU CCGCCUCCUG GGUUCAAGUG AUUCUCCUGC
CUCAGCUUCC CGAGUAGCUG UGAUUACAGC CAUAUGCCAC CACGCCCAGC CAGUUUUGUG
UUUUGUUUUG UUUUUUGUUU UUUUUUUUUG AGAGGGUGUC UUGCUCUGUC CCCCAAGCUG
GAGUGCAGCG GCGCGAUCUU GGCUCACUGC AAGCUCUGCC UCCCAGGUUC ACACCAUUCU
CUUGCCUCAG CCUCCCGAGU AGCUGGGACU ACAGGUGCCC GCCACCACAC CCGGCUAAUU
UUUUUGUGUU UUUAGUAGAG AUGGGGUUUC ACUGUGUUAG CCAGGAUGGU CUCGAUCUCC
UGACCUUUUG AUCCACCCGC CUCAGCCUCC CCAAGUGCUG GGAUUAUAGG CGUGAGCCAC
UGUGCCCGGC CUAGUCUUGU AUUUUUAGUA GAGUCGGGAU UUCUCCAUGU UGGUCAGGCU
GUUCUCCAAA UCCGACCUCA GGUGAUCCGC CCGCCUUGGC CUCCAAAAGU GCAAGGCAAG
GCAUUACAGG CAUGAGCCAC UGUGACCGGC AAUGUUUUUA AAUUUUUUAC AUUUAAAUUU
UAUUUUUUAG AGACCAGGUC UCACUCUAUU GCUCAGGCUG GAGUGCAAGG GCACAUUCAC
AGCUCACUGC AGCCUUGACC UCCAGGGCUC AAGCAGUCCU CUCACCUCAG UUUCCCGAGU
AGCUGGGACU ACAGUGAUAA UGCCACUGCA CCUGGCUAAU UUUUAUUUUU AUUUAUUUAU
UUUUUUUUGA GACAGAGUCU UGCUCUGUCA CCCAGGCUGG AGUGCAGUGG UGUAAAUCUC
AGCUCACUGC AGCCUCCGCC UCCUGGGUUC AAGUGAUUCU CCUGCCUCAA CCUCCCAAGU
AGCUGGGAUU AGAGGUCCCC ACCACCAUGC CUGGCUAAUU UUUUGUACUU UCAGUAGAAA
CGGGGUUUUG CCAUGUUGGC CAGGCUGUUC UCGAACUCCU GAGCUCAGGU GAUCCAACU3
UCUCGGCCUC CCAAAGUGCU GGGAUUACAG GCGUGAGCCA CUGUGCCUAG CCUGAGCCAC
CACGCCGGCC UAAUUUUUAA AUUUUUUGUA GAGACAGGGU CUCAUUAUGU UGCCCAGGGU
GGUGUCAAGC UCCAGGUCUC AAGUGAUCCC CCUACCUCCG CCUCCCAAAG UUGUGGGAUU
GUAGGCAUGA GCCACUGCAA GAAAACCUUA ACUGCAGCCU AAUAAUUGUU UUCUUUGGGA
UAACUUUUAA AGUACAUUAA AAGACUAUCA ACUUAAUUUC UGAUCAUAUU UUGUUGAAUA
AAAUAAGUAA AAUGUCUUGU GAAACAAAAU GCUUUUUAAC AUCCAUAUAA AGCUAUCUAU
AUAUAGCUAU CUAUAUCUAU AUAGCUAUUU UUUUUAACUU CCUUUAUUUU CCUUACAGGG
UUUUAGACAA AAUCAAAAAG AAGGAAGGUG CUCACAUUCC UUAAAUUAAG GAGUAAGUCU
GCCAGCAUUA UGAAAGUGAA UCUUACUUUU GUAAAACUUU AUGGUUUGUG GAAAACAAAU
GUUUUUGAAC AUUUAAAAAG UUCAGAUGUU AGAAAGUUGA AAGGUUAAUG UAAAACAAUC
AAUAUUAAAG AAUUUUGAUG CCAAAACUAU UAGAUAAAAG GUUAAUCUAC AUCCCUACUA
GAAUUCUCAU ACUUAACUGG UUGGUUGUGU GGAAGAAACA UACUUUCACA AUAAAGAGCU
UUAGGAUAUG AUGCCAUUUU AUAUCACUAG UAGGCAGACC AGCAGACUUU UUUUUAUUGU
GAUAUGGGAU AACCUAGGCA UACUGCACUG UACACUCUGA CAUAUGAAGU GCUCUAGUCA
AGUUUAACUG GUGUCCACAG AGGACAUGGU UUAACUGGAA UUCGUCAAGC CUCUGGUUCU
AAUUUCUCAU UUGCAGGAAA UGCUGGCAUA GAGCAGCACU AAAUGACACC ACUAAAGAAA
CGAUCAGACA GAUCUGGAAU GUGAAGCGUU AUAGAAGAUA ACUGGCCUCA UUUCUUCAAA
AUAUCAAGUG UUGGGAAAGA AAAAAGGAAG UGGAAUGGGU AACUCUUCUU GAUUAAAAGU
UAUGUAAUAA CCAAAUGCAA UGUGAAAUAU UUUACUGGAC UCUAUUUUGA AAAACCAUCU
GUAAAAGACU GAGGUGGGGG UGGGAGGCCA GCACGGUGGU GAGGCAGUUG AGAAAAUUUG
AAUGUGGAUU AGAUUUUGAA UGAUAUUGGA UAAUUAUUGG UAAUUUUAUG AGCUGUGAGA
AGGGUGUUGU AGUUUAUAAA AGACUGUCUU AAUUUGCAUA CUUAAGCAUU UAGGAAUGAA
GUGUUAGAGU GUCUUAAAAU GUUUCAAAUG GUUUAACAAA AUGUAUGUGA GGCGUAUGUG
GCAAAAUGUU ACAGAAUCUA ACUGGUGGAC AUGGCUGUUC AUUGUACUGU UUUUUUCUAU
CUUCUAUAUG UUUAAAAGUA UAUAAUAAAA AUAUUUAAUU UUUUUUUAAA UUA
Homo sapiens SMN2 mRNA nucleic acid sequence
NCBI Ref. Seq.: NM_07411.3
(SEQ ID NO: 13)
CCACAAATGT GGGAGGGCGA TAACCACTCG TAGAAAGCGT GAGAAGTTAC TACAAGCGGT
CCTCCCGGCC ACCGTACTGT TCCGCTCCCA GAAGCCCCGG GCGGCGGAAG TCGTCACTCT
TAAGAAGGGA CGGGGCCCCA CGCTGCGCAC CCGCGGGTTT GCTATGGCGA TGAGCAGCGG
CGGCAGTGGT GGCGGCGTCC CGGAGCAGGA GGATTCCGTG CTGTTCCGGC GCGGCACAGG
CCAGAGCGAT GATTCTGACA TTTGGGATGA TACAGCACTG ATAAAAGCAT ATGATAAAGC
TGTGGCTTCA TTTAAGCATG CTCTAAAGAA TGGTGACATT TGTGAAACTT CGGGTAAACC
AAAAACCACA CCTAAAAGAA AACCTGCTAA GAAGAATAAA AGCCAAAAGA AGAATACTGC
AGCTTCCTTA CAACAGTGGA AAGTTGGGGA CAAATGTTCT GCCATTTGGT CAGAAGACGG
TTGCATTTAC CCAGCTACCA TTGCTTCAAT TGATTTTAAG AGAGAAACCT GTGTTGTGGT
TTACACTGGA TATGGAAATA GAGAGGAGCA AAATCTGTCC GATCTACTTT CCCCAATCTG
TGAAGTAGCT AATAATATAG AACAAAATGC TCAAGAGAAT GAAAATGAAA GCCAAGTTTC
AACAGATGAA AGTGAGAACT CCAGGTCTCC TGGAAATAAA TCAGATAACA TCAAGCCCAA
ATCTGCTCCA TGGAACTCTT TTCTCCCTCC ACCACCCCCC ATGCCAGGGC CAAGACTGGG
ACCAGGAAAG CCAGGTCTAA AATTCAATGG CCCACCACCG CCACCGCCAC CACCACCACC
CCACTTACTA TCATGCTGGC TGCCTCCATT TCCTTCTGGA CCACCAATAA TTCCCCCACC
ACCTCCCATA TGTCCAGATT CTCTTGATGA TGCTGATGCT TTGGGAAGTA TGTTAATTTC
ATGGTACATG AGTGGCTATC ATACTGGCTA TTATATGGGT TTTAGACAAA ATCAAAAAGA
AGGAAGGTGC TCACATTCCT TAAATTAAGG AGAAATGCTG GCATAGAGCA GCACTAAATG
ACACCACTAA AGAAACGATC AGACAGATCT GGAATGTGAA GCGTTATAGA AGATAACTGG
CCTCATTTCT TCAAAATATC AAGTGTTGGG AAAGAAAAAA GGAAGTGGAA TGGGTAACTC
TTCTTGATTA AAAGTTATGT AATAACCAAA TGCAATGTGA AATATTTTAC TbbACTCTAT
TTTGAAAAAC CATCTGTAAA AGACTGAGGT GGGGGTGGGA GGCCAGCACG GTGGTGAGGC
AGTTGAGAAA ATTTGAATGT GGATTAGATT TTGAATGATA TTGGATAATT ATTGGTAATT
TTATGAGCTG TGAGAAGGGT GTTGTAGTTT ATAAAAGACT GTCTTAATTT GCATACTTAA
GCATTTAGGA ATGAAGTGTT AGAGTGTCTT AAAATGTTTC AAATGGTTTA ACAAAATGTA
TGTGAGGCGT ATGTGGCAAA ATGTTACAGA ATCTAACTGG TGGACATGGC TGTTCATTGT
ACTGTTTTTT TCTATCTTCT ATATGTTTAA AAGTATATAA TAAAAATATT TAATTTTTTT
TTAAATTAAA AAAA
Homo sapiens SMN2 amino acid sequence
NCBI Ref. Seq.: NP_059107.1; UniProtKB: Q16637
(SEQ ID NO: 14)
MAMSSGGSGG GVPEQEDSVL FRRGTGQSDD SDIWDDTALI KAYDKAVASF KHALKNGDIC
ETSGKPKTTP KRKPAKKNKS QKKNTAASLQ QWKVGDKCSA IWSEDGCIYP ATIASIDFKR
ETCVVVYTGY GNREEQNLSD LLSPICEVAN NIEQNAQENE NESQVSTDES ENSRSPGNKS
DNIKPKSAPW NSFLPPPPPM PGPRLGPGKP GLKFNGPPPP PPPPPPHLLS CWLPPFPSGP
PIIPPPPPIC PDSLDDADAL GSMLISWYMS GYHTGYYMGF RQNQKEGRCS KSLN

As used herein, “SMN2 pre-mRNA” refers to an RNA sequence, including all exons, introns, and untranslated regions, transcribed from DNA encoding human SMN2.

As used herein, “intronic splicing silencer N1” or “ISS-N1” refers to an intronic splice silencing domain in intron 7 of the SMN2 gene or pre-mRNA (see e.g., Singh et al., Mol Cell Biol (2006) 26(4):1333-46). Splicing of acritical exon of human Survival Motor Neuron is regulated by a unique silencer element located in the last intron.

In some embodiments, ISS-N1 comprises the nucleic acid sequence:

CCAGCAUUAUGAAAG (SEQ ID NO: 15)

In some embodiments, the SMN2 pre-mRNA is targeted with one or more of the exemplary oligonucleotides disclosed in Tables 4-8 below in one or more SNAs. Unless indicated otherwise, the sequences contain phosphodiester internucleotide linkages.

TABLE 4

| | | |
|---|---|---|
| ASO 10-27- | 65 | moeT*/5- Me-moeC/moeA/5-Me-moeC/moeTmoeTmoeT/5-Me- |
| MOE-PS-Chol | | moeC/moeAmoeTmoeAmoeAmoeTmoeG/5-Me- |
| SMN-2PS | | moeC/moeTmoeG*moeG/iSp18//iSp18//3CholTEG/ |
| ASO 10-27- | 66 | moeT*/5-Me-moeC/moeA/5-Me-moeC/moeTmoeTmoeT/5-Me- |
| MOE-PS-Chol | | moeC/moeAmoeTmoeAmoeAmoeTmoeG*/5-Me- |
| SMN-3PS | | moeC/moeTmoeGmoeG/iSp18//iSp18//3CholTEG/ |
| ASO 10-27- | 67 | moeT*/5-Me-moeC/moeA/5 -Me-moeC/moeTmoeTmoeT/5 -Me- |
| MOE-PS-Chol | | moeC/*moeA*moeTmoeAmoeAmoeT*moeG*/5-Me- |
| SMN-5PS | | moeC/moeTmoeGmoeG/Sp18//iSp18//3CholTEG/ |
| ASO 10-27- | 68 | moeT*/5-Me-moeC/moeA/5-me-moeC/moeTmoeTmoeT/5- |
| MOE-PS-Chol | | MemoeC/*moeA*moeT*moeA*moeA*moeT*moeG*/5MemoeC/ |
| SMN-8PS | | moeTmoeGmoeG/iSp18//iSp18//3CholTEG/ |
| 5′-Chol ASO 10- | 69 | /5CholTEG//iSp18//iSp18/moeT*/5-Me-moeC/*nioeA*/5-Me- |
| 27 MOE PS | | moeC/*moeT*moeT*moeT*/5-Me- |
| SMN | | moeC/moeA*moeT*moeA*moeA*moeT*moeG*/5-Me- |
| | | moeC/*moeT*moeG*moeG |

*= phosphorothioate internucleotide linkage;
moe = 2′-O-(2-methoxyethy]);
iSp18 = internal hexaethylenegyleol spacer;
5-Me-moeC = 5′ methyl C with moe modification;
3CholTEG = 3′-Cholesteryl-TEG;
SCholTEG = 5′Cholesteryl-TEG;
TEG = tetraethylene glycol

TABLE 5

| | |
|---|---|
| ASO 10-27-Me- | mU*mC*mA*mC*mU*mU*mU*mC*mA* |
| PS-Chol | mU*mA*mA*mU*mG*mC*mU*mG* |
| SMN | mG/Sp18//Sp18//3CholTEG/ |
| | (SEQ ID NO: 70) |
| ASO 10-27- | mUmCmAmCmUmUmUmCmAmUmAmAm |
| Me-Chol | UmGmCmUmGmG/iSp18//iSp18/ |
| SMN all PO | /3CholTEG/ (SEQ ID NO: 71) |
| ASO 10-27- | mT*/5-Me-mC/*mA*/5-Me-mC/ |
| Me-PS-Chol | *mT*mT*mT*/5-Me-mC/*mA |
| SMN (5MeC and | *mT*mA*mA*mT*mG*/5-Me- |
| 5MeU) | mC/*mT*mG*mG/Sp18//Sp18 |
| | //3CholTEG/ |
| | (SEQ ID NO: 72) |
| Linear ASO | mT*/5-Me-mC/*mA*/5-Me- |
| 10-27-Me- | mC/*mT*mT*mT*/5-Me- |
| PS-SMN | mC/*mA*mT*mA*mA*mT*mG* |
| (5MeC and | /5-Me-mC/*mT*mG*mG |
| 5MeU) | (SEQ ID NO: 73) |
| Linear ASO | mUmCmAmCmUmUmUmCmAmUm |
| 10-27-Me- | AmAmUmGmCmUmGmG |
| SMN all PO | (SEQ ID NO: 74) |

*= phosphorothioate internucleotide linkage; m = 2′-O-methyl; iSp18 = internal hexaethylenegylcol spacer; 5-Me-moeC = 5′ methyl C with 2′-O-methyl modification; 3CholTEG = 3′-Cholesteryl-TEG; 3CholTEG = 3′-Cholesteryl-TEG; TEG = tetraethylene glycol

TABLE 6

| | |
|---|---|
| Morpholino | Chol5′-TCACTTTCATAATGCTGG- |
| SMN oligo | CholEG-3′ (SEQ ID NO: 75) |
| Linear | 5′-TCACTTTCATAATGCTGG-3′ |
| Morpholino | (SEQ ID NO: 76) |
| oligo | |

CholEG = Cholesterol with ethylene glycol spacer; all bases in the above two sequences contain morpholino sugar modification

TABLE 7

| | |
|---|---|
| ASO 10-27-ENA- | enT*/5-Me-enC/*enA*/5-Me-enC/*enT*enT*enT*/5-Me- |
| PS-Chol SMN | enC/*enA*enT*enA*enA*enT*enG*/5-Me- |
| | enC/*enT*enG*enG/isp18//isp18//3CholTEG/ (SEQ ID NO: 77) |
| Linear ASO 10- | enT*/5-Me-enC/*enA*/5-Me-enC/*enT*enT*enT*/5-Me- |
| 27-ENA-PS-SMN | enC/*enA*enT*enA*enA*enT*enG*/5-Me-enC/*enT*enG*enG (SEQ ID NO: 78) |

*= phosphorothioate internucleotide linkage;
en = 2′-O,4′-C-ethylene-bridged sugar modification;
iSp18 = internal hexaethylenegylcol spacer;
5-Me-enC = 5′ methyl C with en modification;
3CholTEG = 3′-Cholesteryl-TEG;
TEG = tetraethylene glycol

TABLE 8

| | |
|---|---|
| ASO 10-27-LNA- | lnT*/5-Me-lnC/*lnA*/5-Me-lnC/*lnT*lnT*lnT*/5-Me- |
| PS-Chol SMN | lnC/*lnA*lnT*lnA*lnA*lnT*lnG*/5-Me- |
| | lnC/*lnT*lnG*lnG/isp18//isp18//3CholTEG/(SEQ ID NO: 79) |
| Linear ASO 10-27- | lnT*/5-Me-lnC/*lnA*/5-Me-lnC/*lnT*lnT*lnT*/5-Me-lnC/*lnA* |
| LNA-PS-SMN | lnT*lnA*lnA*lnT*lnG*/5-Me-lnC/*lnT*lnG*lnG (SEQ ID NO: 80) |

*= phosphorothioate internucleotide linkage;
ln = LNA sugar modification;
iSp18 = internal hexaethylenegylcol spacer;
5-Me-lnC = 5′ methyl C with LNA sugar modification;
3CholTEG = 3′-Cholesteryl-TEG;
TEG = tetraethylene glycol

The term “percent identical” refers to sequence identity between two amino acid sequences or between two nucleotide sequences of oligonucleotides disclosed herein, such as antisense oligonucleotides. Identity can each be determined by comparing a position in each sequence which may be aligned for purposes of comparison. When an equivalent position in the compared sequences is occupied by the same base or amino acid, then the molecules are identical at that position, when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASP AR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

In order that the invention described herein may be more fully understood, the following examples are set forth. The examples described in this application are offered to illustrate the compounds, pharmaceutical compositions, and methods provided herein and are not to be construed in any way as limiting their scope.

### EXAMPLES

### Example 1. SMN2-Targeted SNA to Increase Expression of SMN2 mRNA and Protein for Treatment of Spinal Muscular Atrophy

Based on these unique properties of SNAs. SNAs have been developed targeting mRNA for down regulation of gene expression and TLR9 protein to activate the immune system. Antisense SNAs for dermal diseases and TLR9 agonist SNAs for immuno-oncology applications are in clinical development. A linear and a SNA version of Spinraza were compared for their effect on the inclusion of exon 7 in SMN2 mRNA in SMA patient-derived fibroblasts. The results show that in patient-derived fibroblasts, SNA version of Spinraza yields greater expression of exon 7 included SMN2 mRNA and protein compared with the linear version of Spinraza currently used to treat SMA patients.

### Methods

Linear oligonucleotides (linear ASO) and 3′-cholesterol attached linear oligonucleotides via two hexaethyleneglycol (spacer18) moieties for SNA were synthesized with 2′-methoxyethyl (2′-MOE) and phosphorothioate (PS) backbone modification. The oligonucleotide sequence is the same as that of Spinraza. SNAs (SNA-ASO) were prepared by loading 3′-cholesterol attached oligonucleotides onto DOPC liposomes at a ratio of 30 oligonucleotide molecules per 20 nm liposome.

Oligo sequence:
(SEQ ID NO: 1)
5′- TCA CTT TCA TAA TGC TGG - (Spacer 18)₂ -
3CholTEG

SMA patient fibroblast cells (GM03813C, GM09677C and GM00232D) were obtained from Coriell Institute for Medical Research. Cells were cultured in DMEM medium containing 10% FBS and 100 U/ml penicillin and 100 μg/ml streptomycin. Linear and SNA ASOs were added to the cell cultures without transfecting agents and incubated for 48 hours or 72 hours. Then the cells were collected at 48 hours for mRNA extraction and at 72 hours for protein isolation. The levels of SMN2 mRNA, SMNA7 mRNA, and total SMN mRNAs were measured by qPCR using the following set of probes and primers. SMN2 mRNA and SMNA7 mRNA primers were obtained from IDT and the probes were from Thermo Fisher Scientific, and the commercially available primers and probes for total SMN mRNA were purchased from Life Technologies (cat #Hs00165806_ml). SMN2 mRNA forward primer: 5′-GCTG ATGCTTTGGG AAGTATGTTA-3′ (SEQ ID NO: 2), SMN2 mRNA reverse primer: 5′-CACCTTCCTTCTTTTTGATTTTGTC-3′ (SEQ ID NO: 3), SMN2mRNA probe: 5′-6FAM-TACATGAGTGGCTATCATACTT-MGBNFQ-3′ (SEQ ID NO: 4), SMNA7 mRNA forward primer: 5′-TGGACCACCAATAATTCCCC-3′ (SEQ ID NO: 5), SMNA7 mRNA reverse primer: 5′-ATGCCAGCATTT CCATATAATAGCC-3′ (SEQ ID NO: 6) and SMNA7 mRNA probe: s′-6FAM-TACATGAGTGGCTATCATACT-MGBNFQ-3′ (SEQ ID NO: 7). The levels of SMN2 protein were measured by Western blotting using SMN antibody obtained from BD Biosciences (cat #610646) and the control GRP94 protein by the Grp94 (9G10) antibody obtained from Enzo Lifesciences (cat #ADI-SPA-850). The fold increase of SMN2 mRNA over SMNA7 mRNA was calculated by dividing the values of % SMN2 mRNA expression with % SMNA7 mRNA expression.

### Results

ASO-SNAs and Linear ASOs targeting ISS-N1 site of the SMN2 mRNA were tested at various concentrations in three different SMA patient-derived fibroblasts. In addition, phenylbutyrate (PBA, a known small molecule compound, positive control) and negative controls (control SNA and control linear) were included in the assays for comparison. The results showed that ASO-SNA treatment led to greater inclusion of exon 7 in SMN2 mRNA compared with linear ASO. ASO-SNA treatment resulted in up to 45-fold increase in the inclusion of exon 7 over SMNA7 mRNA depending on the source of fibroblasts. Whereas, linear ASO resulted in about 2.5-fold higher inclusion of exon 7 over SMNA7 mRNA (FIGS. 1A and 1B). The results shown in FIGS. 1A and 1B are representative of one experiment of the three independent experiments carried out.

Next, the upregulation of SMN2 protein was measured by ASO-SNA and linear ASO at 72 hours by Western blotting. GM09677C cells (SMA patient-derived fibroblasts) were treated with SNAs for 72 hours and, then assessed by western blot and qRT-PCR. ASO-SNA treatment resulted in greater expression of SMN2 protein compared with linear ASO in GM09677C (FIGS. 2A and 2B), which is consistent with the results from the mRNA levels from above. FIG. 2A shows a Western blot of total SMN protein and loading control GRP94. GRP94 protein loading control was detected with ADI-SPA-850-F (Enzo Life Sciences). SMN was detected with VMA00249 (Bio-Rad). FIG. 2B is a densitometric quantification of SMN western blot (solid bars) and qRT-PCR of full-length SMN mRNA (hashed bars) from identically treated wells. SMN qRT-PCR was performed on SMA patient fibroblasts (GM09677C) that were plated in 96-well plates and treated in triplicate with SNAs in complete media. After cell lysis, cDNA was derived from extracted RNA and assessed by qRT-PCR with technical duplicates for each sample. Full-length SMN2 was measured relative to GAPDH.

### Conclusion

ASO-SNA treatment of SMA patient-derived fibroblasts facilitates increased of exon 7 inclusion and SMN2 protein expression compared with the same sequence of linear ASO (Spinraza). Previous studies have shown that oligonucleotides in SNA are taken up by cells to greater extent than linear oligonucleotides and function as potent antisense agents at mRNA level in the cytoplasm to down regulate gene expression. The current results are the first demonstration of SNAs interacting with pre-mRNA in the nucleus facilitating exon 7 inclusion in SMN2 mRNA in SMA patient-derived fibroblasts.

Thus, these in vitro studies showed that SNAs are several-fold more potent in generating exon7 included SMN2 mRNA and full-length protein compared with linear oligo.

Based on the data from patient-derived fibroblasts, next step is to test in vivo in mouse model to evaluate the potency of SMN2-targeted SNA in comparison with a linear MOE-ASO. Tolerability of SNA compounds can be evaluated by intrathecal (IT) or intracerebroventricular injection (ICV). Spinraza is administered to patients using IT administration so ideal comparison will be to use IT administration in mouse models as well. It would be a great improvement to be able to deliver the therapeutic SNA into central nervous system using other administration modalities, such subcutaneous, intramuscular, intravenous, oral, ophthalmic, topical delivery in the ear as ear drops or similar forms, transtympanic administration, etc. These other administrations are less invasive compared to intrathecal administration and may improve patient comfort. Spinraza is administered in 5 mL volume (2.4 mg/mL); in mice this volume would be much smaller, on the order of a few microliters. It is aimed to test as high a dose of oligonucleotide as practical. In animal models, survival and other parameters such as SMN mRNA and protein levels, might be sufficient especially for modelling severe SMA. Electromyograms (EMG) can also be recorded for compound muscle action potential (CMAP) as well as motor unit number estimation. These parameters are reduced in SMA. If SMN levels are normalized by therapeutic interventions, these values have been observed to recover. In human clinical trials, CMAP is observed to correlate well with motor function and has the potential value as a relevant surrogate for disease status. This is one of the only measures that can be made in humans and mouse models.

### Example 2. Spherical Nucleic Acids (SNAs) are Versatile Facilitators of Oligonucleotide-Mediated Splice Modulation

Spherical nucleic acids (SNAs) are dense, radial arrangements of oligonucleotides around a nanoparticle core. Studies have shown the utility of SNAs for immune stimulation using CpG oligonucleotides and for gene knockdown mediated by antisense oligonucleotides or siRNA. Recently, SNAs have shown promise as effectors of splice modulation through delivery of splice-switching oligonucleotides (SSOs). This work investigates the suitability of SNAs broadly for splice modulation.

Alternative splicing of pre-mRNA leads to production of divergent mature mRNA products and is a major source of proteome diversity. Indeed, at least 70% of human genes are predicted to undergo alternatively splicing and it is estimated that 60% of human genetic diseases arise from mutations that effect splicing. Clearly, broadly applicable methods to influence splicing are of significant therapeutic value. Thus, when considering SNAs for splice modulation, it is critical to evaluate their efficacy across splicing contexts and targets.

SNAs were designed to facilitate exon skipping of signal transducer and activator of transcription 3 (STAT3), RE1 silencing transcription factor (REST), interleukin 17 receptor α (IL17RA), or IL1 receptor accessory protein (IL1RAP), and their efficacy was compared to that of linear SSOs. The results establish SNAs as comprehensively more effective (by 2- to 3-fold) at inducing preferential production of a natural transcript variant (IL17RA, REST), a novel, stable splice variant (IL1RAP), and a novel, frame-shifted variant that leads to nonsense-mediated decay (STAT3). SNAs exhibited this potency in several relevant cell lines and, in the case of IL17RA-targeted SNAs, in human skin biopsies. Additionally, survival of motor neuron 2 (SMN2)-targeted SNAs dramatically improved exon inclusion in human patient fibroblasts, exhibiting a more than 10-fold potency improvement over linear SSOs.

A unique feature of SNAs is that they can be designed to deliver multiple oligonucleotides to the same cell, enabling synergistic targeting of a single pre-mRNA with disparate SSOs or facilitating modulation of multiple therapeutic targets simultaneously by targeting different genes. Proof-of-concept work targeting two loci on the IL1RAP gene demonstrates the power of this approach. In this context, a bispecific SNA comprised of two SSOs targeting IL1RAP outperformed either SSO delivered on SNA individually.

In summary, the data reveal the potential of SNAs for mRNA reprogramming through splice modulation of pre-mRNA in a variety of modes, across therapeutic targets, and at multiple genetic loci. This establishes SNAs as potentially transformative therapies for diseases ranging from inflammatory skin conditions to cancer to rare neurological disorders.

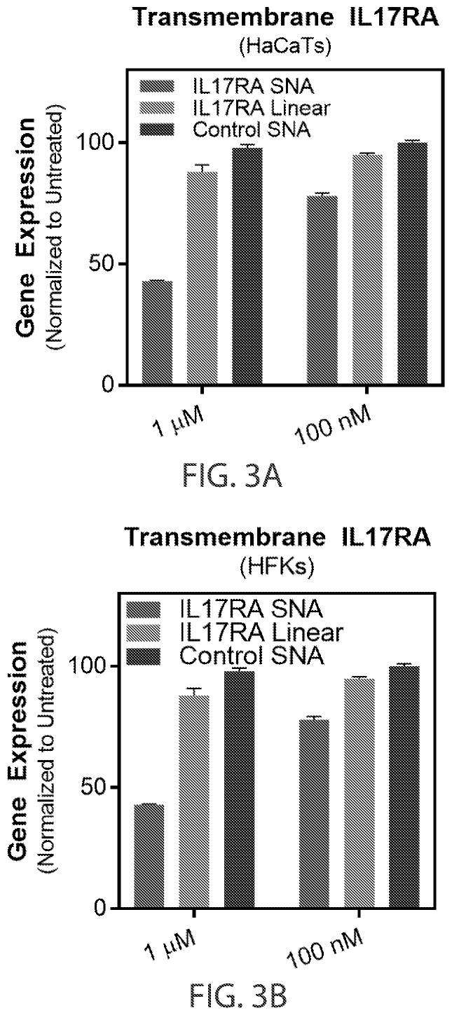

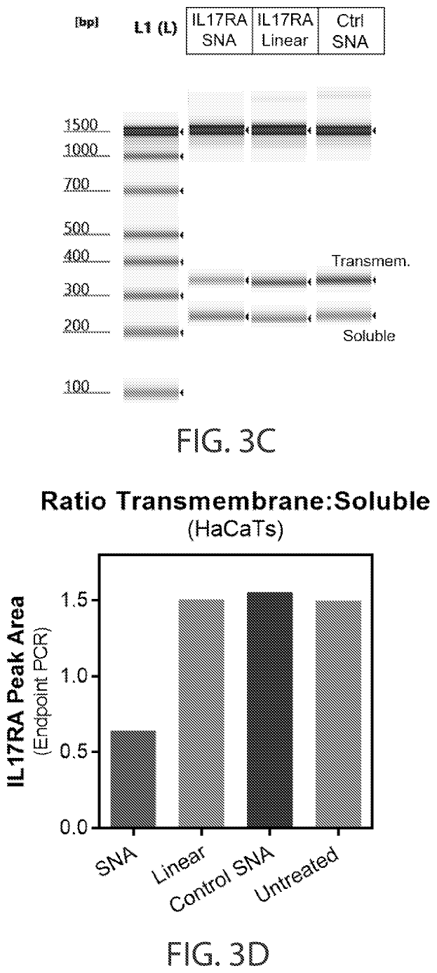

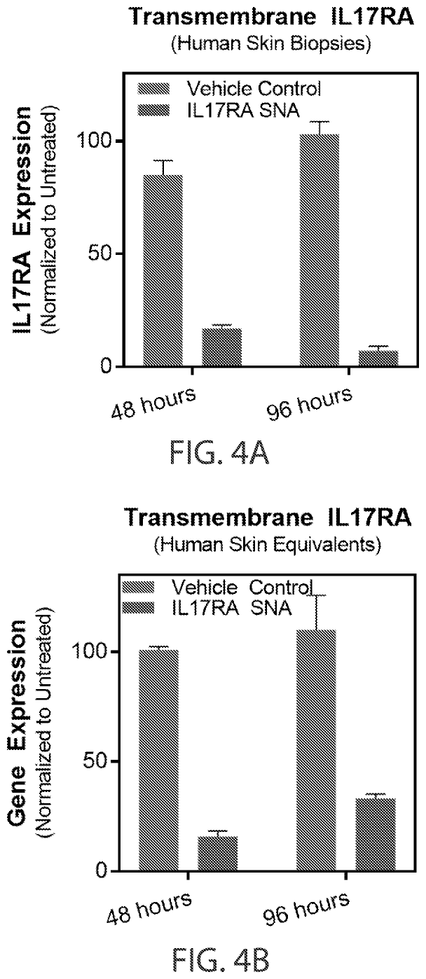

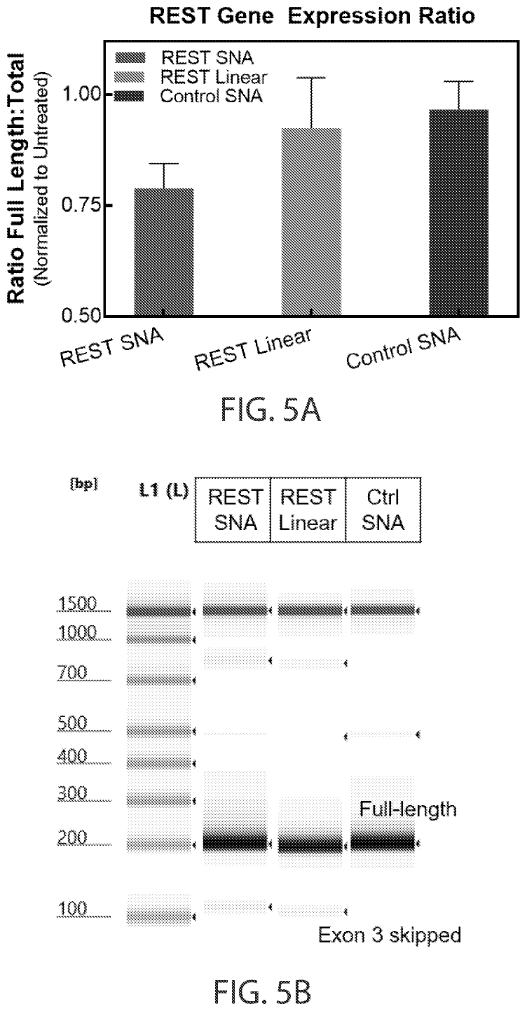

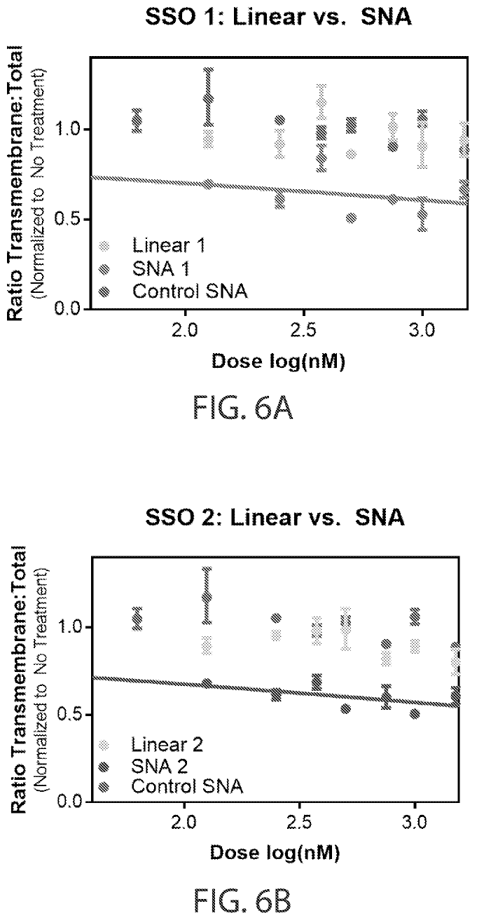

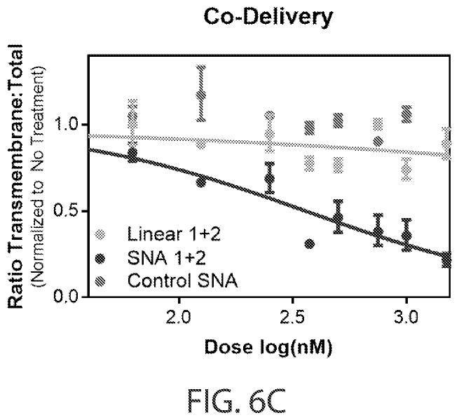

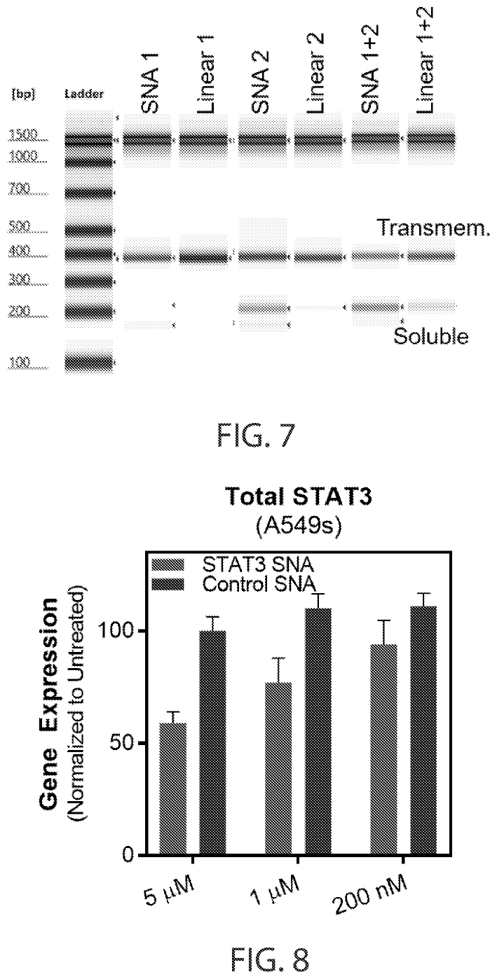

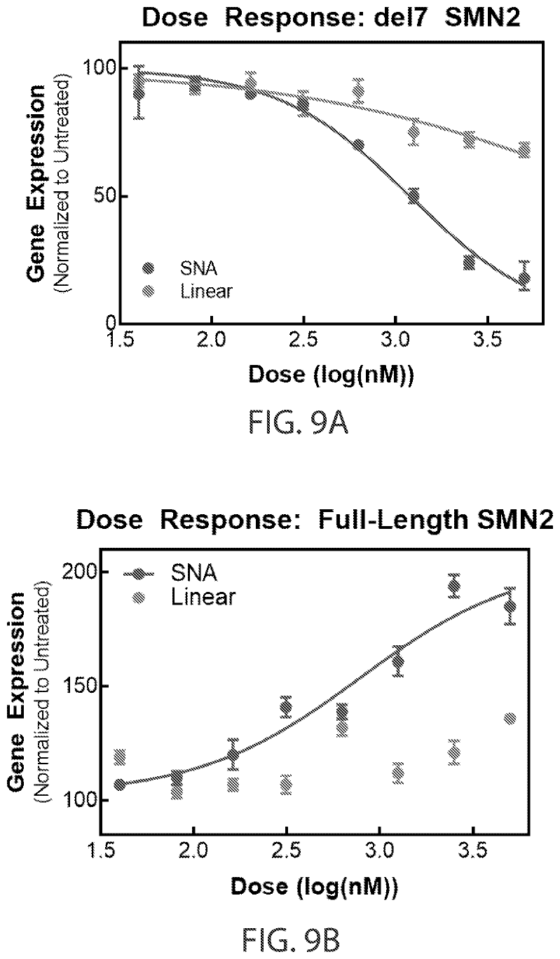

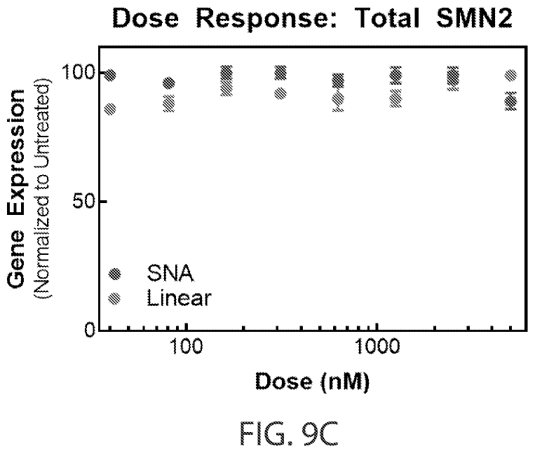

Endpoint PCR evaluation and real time PCR quantification of genetic variants were conducted in vitro in several distinct cell types. Disease modification in vivo in the SMNA7 mouse model was also conducted. Results show the utility of SNAs for this strategy using the examples of IL17RA, REST, IL1RAP, and STAT3. SNAs enable splice modulation via exon skipping to a natural variant in the case of IL17RA. It was found that SNAs reduce transmembrane IL17RA in two dermal cell lines, shown by qPCR quantification of transmembrane IL17RA (see FIGS. 3A-3D). It was elucidated that this reduction of IL17RA occurs via splice modulation to generate a soluble variant through endpoint PCR. Endpoint PCR shows a switch from transmembrane to soluble IL17RA (see FIGS. 3C-3D). SNAs also reduce transmembrane IL17RA in relevant ex vivo models. Human skin biopsies and human skin equivalents were evaluated by qPCR for transmembrane IL17RA (see FIGS. 4A-4B). Furthermore, SNAs improve exon 3 skipping of REST. SNAs enable exon skipping of REST in free uptake superior to that of the linear oligonucleotide. This is supported by qPCR and endpoint PCR data, with 5 μM dose in MEF cells (see FIGS. 5A-5B). It was also found that bispecific SNAs induce splice modulation of IL1RAP, qPCR data shows that two different oligonucleotides (ONs) work synergistically to induce splice modulation of IL1RAP (see FIGS. 7 and 6A-6C). Endpoint PCR data support this synergistic effect (see FIGS. 9A-9C). SNAs facilitate exon skipping of STAT3; SNA comprised of the fully 2′-MOE modified oligonucleotide ST7 reduced total STAT3 levels, indicating production of an out-of-frame transcript that undergoes nonsense-mediated decay (see FIG. 8). Additionally, SNA treatment causes superior exon 7 inclusion in SMN2. An example of qPCR in vitro data supporting exon 7 inclusion in SMN2 is shown in FIG. 9A-9C.

Cumulatively, the data reveals the potential of SNAs to powerfully influence pre-mRNA splicing in a variety of modes, across numerous genetic targets, and at multiple genetic targets or loci (see FIGS. 3A-9C). Thus, SNAs have the potential to be transformative therapies for diseases ranging from inflammatory skin conditions to cancer to rare neurological disorders.

### Example 3. SMN2-Targeted Antisense Spherical Nucleic Acid (SNA) Treatment of SMA in a Mouse Model

The constructs were tested in vivo in a mouse model to evaluate the potency of SMN2-targeted SNA in comparison with a linear MOE-ASO. Tolerability of SNA compounds can be evaluated by intrathecal (IT) or intracerebroventricular injection (ICV). Spinraza is administered to patients using IT administration so one ideal comparison will involve IT administration in mouse models. It would be a great improvement to be able to deliver the therapeutic SNA into central nervous system using other administration modalities, such subcutaneous, intramuscular, intravenous, oral, ophthalmic, topical delivery in the ear, such as ear drops or similar forms, transtympanic administration, etc. These other administration routes are less invasive compared to intrathecal administration and may improve patient comfort. Spinraza is administered in 5 mL volume (2.4 mg/mL); in mice this volume would be much smaller, on the order of a few microliters. In animal models, survival and other parameters such as SMN mRNA and protein levels, might be sufficient especially for modelling severe SMA. Electromyograms (EMG) can also be recorded for compound muscle action potential (CMAP) as well as motor unit number estimation. These parameters are reduced in SMA. If SMN levels are normalized by therapeutic interventions, these values have been observed to recover. In human clinical trials, CMAP is observed to correlate well with motor function and has the potential value as a relevant surrogate for disease status. This is one of the only measures that can be made in humans and mouse models.

It has previously been shown that morpholino antisense treatment directed at the negative regulatory ISS-N1 in SMN2 results in increased incorporation of SMN2 exon7 and increased levels of SMN protein. It has further been shown that the second hnRNP A1 site at −85-109 in intron7 can also be blocked to give an equivalent level of SMN to blocking ISS-N1. However due to delivery to critical cells the latter therapy was not as effective when used as a morpholino as ISS-N1. The blocking of ISS-N1 as well as −85-109 results in increased survival and function of SMA model mice. In addition, there is significant recovery of the electrophysiologic function. The latter is critical as regards SMA treatment in humans as in SMA there is clear decrement of motor neuron function in human and no critical evidence for a role of the periphery. Indeed, patients treated early using either antisense oligonucleotide or gene therapy show remarkable improvement in phenotype achieving milestones never observed in SMA patients.

An ASO targeting ISS-N1 site of SMN2 mRNA (−10-27) with MOE chemistry (Spinraza) has been recently approved by the FDA. When using an intrathecal delivery system in human SMA the antisense oligonucleotide is showing good effect when treatment is given pre-symptomatically. In preclinical work, the MOE-ASO did show toxicity in mice and the MOE-ASO could not be used at the same concentration as the morpholino. Passini et al used a single dose of MOE-ASO via intracerebral ventricular (ICV) injection up to 8 μg and obtained a survival improvement from 14 days to 23 days, Hua et al used a ICV dose of 20 μg with no adverse effect and in a different SMA animal model had an increased survival from 10 days to 16 days. Hua et al also obtained further improvement by giving the ASO into the periphery. This contrasts with the morpholino data that showed survival beyond 100 days in the delta7 SMA mice whereas mice without treatment lived for 13 days.

### Methods

Linear oligonucleotides (linear ASO) and 3′-cholesterol attached linear oligonucleotides via two hexaethyleneglycol (spacer18) moieties for SNA were synthesized with 2′-methoxyethyl (2′-MOE) and phosphorothioate (PS) backbone modification. The oligonucleotide sequence is the same as that of Spinraza. SNAs (SNA-ASO) were prepared by loading 3′-cholesterol attached oligonucleotides onto DOPC liposomes at a ratio of 30 oligonucleotide molecules per 20 nm liposome particle.

Compounds were administered to mice by intracerebro-ventricular injections as described previously (P. N. Porensky, et al, Hum. Mol. Genet. 21, 1625-1638, 2012). Briefly, P0 pup was cryo-anesthetized and hand-mounted over a back-light to visualize the intersection of the coronal and sagittal cranial sutures (bregma). A fine-drawn capillary needle with injection assembly was inserted 1 mm lateral and 1 mm posterior to bregma, and then tunneled 1 mm deep to the skin edge (approximating) ipsilateral lateral ventricle. An opaque tracer (Evans Bluet 0.04%) was added to the reagent to visualize the borders of the lateral ventricle after injection of 2 or 3 μl of SNA-ASO or linear ASO. A single dose of SNA-ASO or linear ASO at 10, 20 or 30 μg dose/mouse administered by ICV at age P0. Following administration of compounds, mouse survival and body weights were recorded.

Spinal cords of SMA mice treated with 30 μg dose of SNA-ASO on P0 and untreated control mice were collected on P0 and measured full-length SMN2 mRNA transcript by digital droplet PCR as described previously by P. N. Porensky, et al, Hum. Mol. Genet. 21, 1625-1638, 2012. Table 9 outlines the compounds used.

TABLE 9
Protocol for the study of SMN2 antisense SNA and MOE-ASO (linear oligo) in SMN^{-/-}
SMN2 D7 SMA mice

| | V olume/ | Dose/ | | | Animals/ | |
|---|---|---|---|---|---|---|
| Compound | dossing | animal | Phenotype | Genotype | group | Outcome |
| -10-27 linear | 2 μl once | 10 μg | SMA | Tox trial | 5 | Tox |
| oligo | | | | | | |
| -10-27 linear | 2 μl once | 20 μg | SMA | Tox trial | 5 | Tox |
| oligo | | | | | | |
| -10-27 SNA | 2 μl once | 10 μg | SMA | Tox trial | 5 | Tox |
| -10-27 SNA | 2 μl once | 20 μg | SMA | Tox trial | 5 | Tox |
| -10-27 linear | 2 μl once | 10 μg | SMA | Smn^{-/-} | 13 | Survival/EMG |
| oligo | | | | SMN2 D7 | | |
| -10-27 linear | 2 μl once | 20 μg | SMA | Smn^{-/-} | 13 | Survival/EMG |
| oligo | | | | SMN2 D7 | | |
| -10-27 linear | 3 μl once | 30 μg | SMA | Smn^{-/-} | 13 | Survival/EMG |
| oligo | | | | SMN2 D7 | | |
| -10-27 SNA | 2 μl once | 10 μg | SMA | Smn^{-/-} | 13 | Survival/EMG |
| | | | | SMN2 D7 | | |
| -10-27 SNA | 2 μl once | 20 μg | SMA | Smn^{-/-} | 13 | Survival/EMG |
| | | | | SMN2 D7 | | |
| -10-27 SNA | 3 μl once | 30 μg | SMA | Smn^{-/-} | 13 | Survival/EMG |
| | | | | SMN2 D7 | | |
| ISS-N1 PMO | 2 μl once | 40 μg | SMA | Smn^{-/-} | 13 | Survival/EMG |
| oligo | | | | SMN2 D7 | | |
| Control SNA | 3 μl once | 30 μg | SMA | Smn^{-/-} | 13 | Survival/EMG |
| | | | | SMN2 D7 | | |
| RNA testing | 2 μl | | SMA | Smn^{-/-} | 5 × 6 = 30 | ddPCR |
| | | | | SMN2 D7 | | |
| SMN protein | 2 μl | | SMA | Smn^{-/-} | 5 × 6 | Western |
| | | | | SMN2 D7 | | |
| RNA testing | 2 μl | | Carrier | Smn+/− | 5 × 6 = 30 | ddPCR |
| | | | | SMN2 D7 | | |
| SMN protein | 2 μ1 | | Carrier | Smn+/− | 5 × 6 | Western |
| | | | | SMN2 D7 | | |

The pharmacodynamic activity of the compounds is followed by survival of mice in each group compared with untreated mice. In a previous study, morpholino ASO prolonged the Smn^{-/-} SMN2 D7 mice survival over 100 days, which serves as a reference for the current study. Further, the EMGs will be recorded for muscle action potential (CMAP) as well as motor unit number estimation. Both these parameters are reduced in SMA at 6 days and beyond. When SMN levels are corrected due to the action of the test compounds, these values recover and when mice live out can reach normal levels. This is an important measure as it shows that the motor neuron has recovered and the muscle is innervated correctly. It is one of the only measures that can be made in man and mouse and is altered in human SMA.

The measures of SMN protein and RNA give a measure of the increased incorporation of SMN exon7 and the amount of SMN protein. In the cases of the carrier mice tested only the human SMN is detected thus the increase can be seen on a background where no cell loss is occurring.

### Results

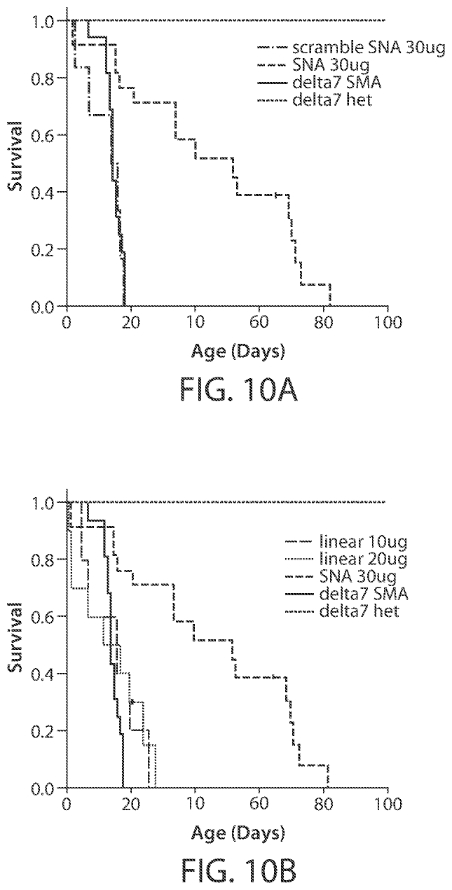

A single dose of SNA-ASO or linear ASO was injected to mice on P0 at 10, 20 or 30 μg. The Kaplan-Meier survival plots of SMA mice treated with SNA-ASO and linear ASO and untreated mice are shown in FIGS. 10A-10B. Mice were genotyped at P0 (day of birth) and injected via Intracerebroventricular injection (ICV) on P0. The recorder of events was blinded to genotype and treatment. Control untreated mice died within 18 days with a median survival of about 14 days. Mice treated with linear ASO showed a median survival of 16, 17 and 2 days at 10, 20 and 30 μg doses, respectively with a maximal survival prolongation of about 28 days. SNA-ASO treatment lead to increased survival of SMA mice at all dose levels compared with linear ASO. The median survival of SNA-ASO treated mice was 26, 69 and 70 days at 10, 20, and 30 μg doses, respectively. The survival of SMA mice was prolonged up to about 117 days in 20 μg SNA-ASO dose group and the mice in 30 μg dose group have not reached end point. These results clearly demonstrate that SNA-ASO prolongs survival of SMA mice to a greater extent than linear ASO. Additionally, early death of mice in 30 μg dose linear ASO group suggest possible toxicity. These results suggest that SNA-ASO treatment is safe and well tolerated up to 30 μg dose level in SMA mice.

FIG. 10A shows Δ7SMA mice treated with the 30 μg dose Nusinersen-SNA had increased survival to a maximum of 82 days while scramble SNA has no effect on survival. FIG. 10B shows that linear Nusinersen improved survival of Δ7 SMA mice to a maximum of 28 days. The data is also summarized in the table below.

| Treatment | | Mean | Maximum | |
|---|---|---|---|---|
| with | # of | survival | survival | Log rank |
| Nusinersen | mice | (days) | (days) | p value |
| Linear 10 μg | 5 | 14.8 ± 4.0 | 26 | NS |
| Linear 20 μg | 10 | 14.0 ± 3.0 | 28 | NS (censored) |
| Linear 30 μg | 5 | 2.2 ± 0.1 | 2 | Toxicity |
| SNA 10 μg | 8 | 25.7 ± 3.3 | 40 | 0.00064 |
| SNA 20 μg | 9 | 57.0 ± 14.0 | 115 | .002 |
| | | | | censored |
| SNA 30 μg | 23 | 45.6 ± 6.1 | 82 | 0.000017 |
| | | | | censored |
| Scrambled SNA 30 μg | 6 | 12.5 ± 2.5 | 18 | NS |
| untreated Δ7SMA | 16 | 14.3 ± 0.7 | 18 | Tested against |

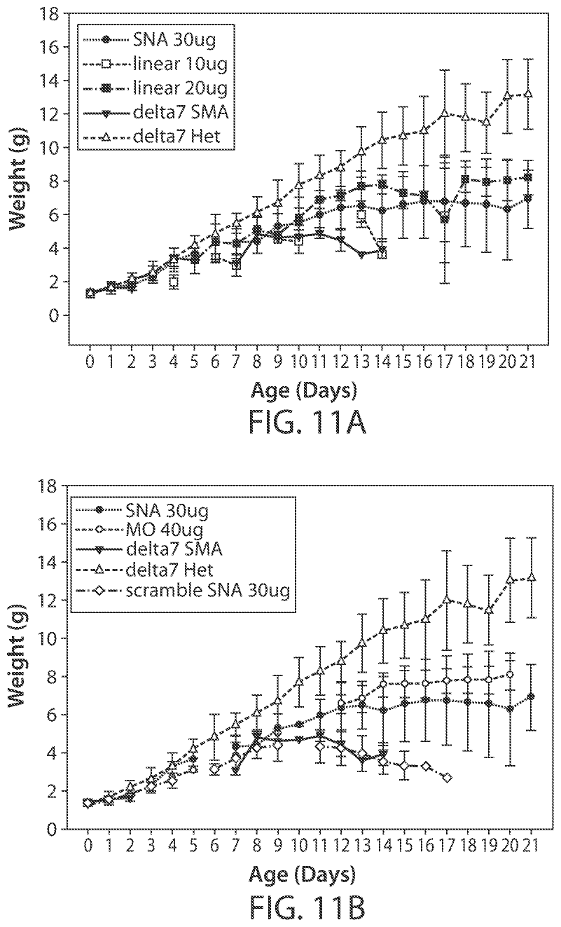

Phenotypic changes, including weight changes, on the treated mice were assessed. Weight curves to 21 days of age in treated and untreated control mice are shown in FIGS. 11A and 11B. Mice were weighed each day. FIG. 11A shows that weights are similar in Δ7SMA mice treated with linear or Nusinersen-SNA treated mice. FIG. 11B shows that weights are similar in Δ7SMA mice treated with morpholino to ISS-N1 or Nusinersen-SNA. The scramble-SNA did not alter the weight of the Δ7SMA mice.

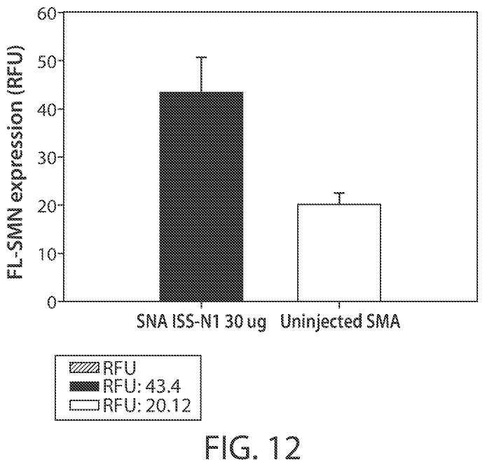

To examine if the treatment of SMA mice with SNA-ASO lead to increased levels of SMN2 full-length mRNA transcript, spinal cords were collected on P10 from mice treated with 30 μg SNA-ASO and untreated control mice, and measured SMN2 mRNA transcript levels by digital droplet PCR. The results shown in FIG. 12 demonstrate that SNA-ASO treatment increased the full-length SMN2 mRNA transcript in SMA mice compared with untreated mice on P10.

Thus, treatment of SMA mice with a single ICV dose of ASO-SNA increased exon 7 inclusion. Moreover, the treatment of SMA mice with ASO-SNA resulted in increased median survival of up to 69/70 days with a prolongation of survival beyond 100 days compared with linear ASO. Further the SNA-ASOs are safe and well tolerated in SMA mice compared with linear ASO. These animal model studies support delivery of SNA to CNS and for neuromuscular disease treatment.

The SNAs increased uptake of MOE Nusinersen in cell models lacking SMN1 but containing SMN2, resulting in increased amounts of full-length mRNA and SMN protein from SMN2. Additionally, SNAs when delivered to CSF in the Δ7SMA mouse model allow increased dosing of Nusinersen and increased efficacy with prolonged survival of SMA mice. SNAs when delivered to CSF in the Δ7SMA mouse model also have increased full-length SMN mRNA levels in spinal cord tissue. In view of these data demonstrating the enhanced use of SNA relative to Nusinersen, the therapeutic utility of the SNA is substantial.

Additional experiments for further analysis include: Performing EMG, compound muscle action potential (CMAP) and motor unit number estimation (MUNE) to assess the extent of motor neuron correction and determining Nusinersen-SNA bio-distribution and SMN levels in all treatment groups using ELISA and Western blot.

### Example 4. Comparative Analysis of ¹²⁵I-Oligonucleotides by SPECT/CT Imagine in Sprague Dawley Rats

Example 1 illustrates that, compared to linear nusinersen, the SNA version of nusinersen has superior splice modulating activity in cell culture in SMA-patient derived fibroblasts. The examples above also illustrate that, in mouse models of SMA, in comparison to linear nusinersen, the SNA version of nusinersen increases median survival and has lower toxicity at higher doses.

The central nervous system (CNS) distribution of intrathecally administrated oligonucleotides (linear ASO and SNA-ASO) was characterized using single-photon emission computed tomography combined with computed tomography (SPECT/CT) imaging in Sprague Dawley rats. Linear ASO and 3′-cholesterol attached linear oligonucleotides via two hexaethyleneglycol (spacer18) moieties for SNA were synthesized with 2′-methoxyethyl (2′-MOE) and phosphorothioate (PS) backbone modifications as described below. The oligonucleotide sequence is the same as that of Spinraza. The oligonucleotides were further modified on the 5′ terminus with amino modified to enable eventual attachment of iodine-125 radio-label element. SNAs (SNA-ASO) were prepared by loading 3′-cholesterol attached oligonucleotides onto DOPC liposomes at a ratio of 30 oligonucleotide molecules per 20 nm liposome particle. The oligonucleotides in both linear ASO and SNA-ASO groups were labeled with iodine-125. The radio-labeled compounds were injected into SD rats (up to 3 rats per group) and whole body SPECT/CT was performed at 0, 0.25, 0.5, 0.75, 6, 24, 72 and 168 hours after injection, 0 hours after injection is essentially immediately after injection of the radio-labeled compounds. Each rat received 180 μg of radio-labeled oligonucleotide in single bolus injection via intrathecal administration in the lower lumbar region, around the 6′ lumbar vertebra.

| Compound | SEQ ID NO | Sequence |
|---|---|---|
| Linear ASO | 81 | /5AmMC6/*moeT*/5-Me-moeC/*moeA*/5-Me- |
| | | moeC/*moeT*moeT*moeT*/5-Me- |
| | | moeC/*moeA*moeT*moeA*moeA*moeT*moeG*/5-Me- |
| | | moeC/*moeT*moeG*moeG |
| SNA-ASO | 82 | /5AmMC6/*moeT*/5-Me-moeC/*moeA*/5-Me- |
| | | moeC/*moeT*moeT*moeT*/5-Me- |
| | | moeC/*moeA*moeT*moeA*moeA*moeT*moeG*/5-Me- |
| | | moeC/*moeT*moeG*moeG/isp18//isp18//3CholTEG/ |

*= phosphorothioate internucleotide linkage;
moe = 2′-O-(2-methoxyethyl);
iSp18 = internal hexaethylenegylcol spacer;
5-Me-moeC = 5′ methyl C with moe modification;
3CholTEG = 3′-Cholesteryl-TEG;
5CholTEG = 5′Cholesteryl-TEG;
TEG = tetraethylene glycol;
5AmMC6 = 5′-Amino-Modifier C6

The SPECT/CT image analyses show that there is a profound difference between the distribution and persistence of linear ASO compared with SNA-ASO. The linear ASO rapidly distributes from the site of administration in lower lumbar region to the other areas of the spinal cord. Within 1-6 hours, noticeable amount of signal from the iodine label is present in the brain as well. Over the course of the 7-day monitoring period, starting as early as 6-hours post-administration, the oligonucleotide signal is decreasing in many regions of the CNS, and is being observed via the kidneys.

By contrast. SNA-ASO distributed away from the site of administration relatively slowly. During the first hour, oligonucleotide is detectable in the spinal cord but not in the brain. Starting at 6 hours post-administration, high amount of oligonucleotide is present in the brain along with the spinal cord. This strong signal remains present in the brain and parts of the spinal cord through the 7-day monitoring period. Less SNA-ASO is observed in the kidney, which likely indicates a relatively slow clearance rate of SNA-ASO from the CNS. Overall, SNA-ASO is persistent in the CNS longer and at higher levels compared to linear ASO.

The whole body images were further analyzed to determine percent of injected dose per gram of tissue present in various regions of interest. Regions of interest, including 13 regions of rat brain, were placed onto each image using automated software tools or approximate anotomical location. Fixed volume regions of interest were used for regions with limited signal. Iodine-125 levels were measured, converted to units of activity, decay corrected and corrected for background radiation. The values were converted to percent injected dose per gram of tissue. Comparison between linear ASO and SNA-ASO shows that following intrathecal injection, SNA delivers approximately 34-71% more oligonucleotide to the whole brain compared to linear ASO. For various regions of the brain, generally linear ASO shows higher oligonucleotide levels at early time points, usually at 0 hours. Unexpectedly, SNA generally shows higher oligonucleotide levels at later time points, typically starting at 6 hours but often earlier for many regions of the brain. Regions with relatively higher distribution for SNA ASO include amygdala (approximately 41-75% higher), basal ganglia (approximately 26-37% higher), cerebellum (approximately 25-78% higher), corpus callosum (approximately 6-149% higher), cortex (approximately 14-73% higher), hippocampus (approximately 6-102% higher), hypothalamus (approximately 41-72% higher), midbrain (approximately 32-73% higher), olfactory (approximately 61-102% higher), ventricles (approximately 8-79% higher), septal area (approximately 19% higher), thalamus (approximately 2-92% higher), and white matter (approximately 27-72% higher). Similar data are also observed in the cervical and thoracic CSF where SNA shows approximately 7-77% and 92-103% higher distribution respectively. In the lumbar CSF, where the compounds are administered, SNA ASO shows higher distribution at nearly all time points (approximately 27-59% higher). Surprisingly, the linear ASO appears at high levels in the kidneys whereas SNA ASO shows high levels in the liver, and superficial and deep cervical lymph nodes, which indicates distinctly different distribution and clearance profiles for SNA ASO compared to linear ASO.

The longer persistence of SNA-ASO suggests that SNA-based therapy could be administered less frequently compared to linear ASO. Since the examples also illustrate that SNA version of nusinersen is not toxic at high doses, in contrast to linear nusinersen which has high toxicity at 30 μg dose in SMA mouse model, higher absolute amount of therapy can also be administered. The combination of higher persistence and lower toxicity can potentially further reducing dosing frequency. The higher distribution in various regions of the brain could enable intrathecal administration of therapies that target diseases of regions of the brain that are quite distal from site of administration.

The subjects were male Sprague Dawley rats (n=9 injected; n=6^{b} on study). The modalities were whole body SPECT/CT. Image agents were formulated with artificial cerebrospinal fluid (aCSF) for intrathecal (IT) injection. The test article is ¹²⁵I-ASO 10-27-MOE-PS SNA, spherical nucleic acid (SNA) composed of an oligonucleotide labeled with iodine-125. The control article is ¹²⁵I-ASO 10-27-MOE-PS, linear ASO labeled with iodine-125. The study design is summarized in Table 10.

TABLE 10

| | | Radiolabeled Test Article | | | | | | |
|---|---|---|---|---|---|---|---|---|
| | | | Dose | Dose | Dose | | Gamma | |
| | Number of | Test | Level | Volume, | Radioactivity | SPECT/CT | Counting | Acquisition |
| Group | Animals/Sex | Article | (μg) | Route | (μCi) | Timepoints | Samples | Timeline |
| Group 1 | 3M | 125I- | ~180 | 30 μL + 40 μL | 263.5 ± 94.5 | 0-1, 6, 24, | Whole | Aug. 28, 2018- |
| | | ASO | | flush aCSF, | | 72, and | Blood | Sep. 4, 2018 |
| | | Linear | | IT | | 168 hours | Plasma | Sep. 4, 2018- |
| Group 2 | 3M | 125I- | ~180 | 30 μL + 40 μL | 241.1 ± 65.0 | | | Sep. 11, 2018^{a} |
| | | ASO | | flush aCSF, | | | | |
| | | SNA | | IT | | | | |

^{a}Imaging dates for animal A4007 (Linear ASO Group). Animal maintains same imaging timepoints as first cohort.
^{b}Only five animals will be used in quantitative analysis.

### Analysis Methods

### Image Analysis

SPECT images were co-registered to CT images and resampled to uniform voxel sizes (0.3 mm³). Regions of Interest (ROIs) were defined using various methods in VivoQuant software. Invicro's 13-region rat brain atlas was placed automatically onto each image using the 3D Brain Atlas Tool in VivoQuant. Fixed volume ellipsoidal ROIs were placed in the center of the liver, kidneys and spleen to encompass areas of representative concentration for each respective region. The superficial and deep cervical lymph nodes, and thyroid were identified using the SPECT. For subjects with limited SPECT activity in these regions, ROIs were placed in the approximate anatomical location using the CT for reference. Fixed volume spherical ROIs were placed in the left and right sides of each of these regions. The CSF was defined using connected thresholding and then split into three regions based on identification of vertebrae: lumbar, thoracic, cervical.

### Gamma Counting

1000 μL aliquots of blood and plasma were collected at 168 hours, placed in tubes and assayed for radioactivity in a gamma counter. The measured count rate, counts per minute (CPM), was converted to units of activity (μCi) using an efficiency value of 0.673 counts per decay for ¹²⁵I. Activities were decay corrected from the time of measurement to the time of injection and corrected for background radiation.

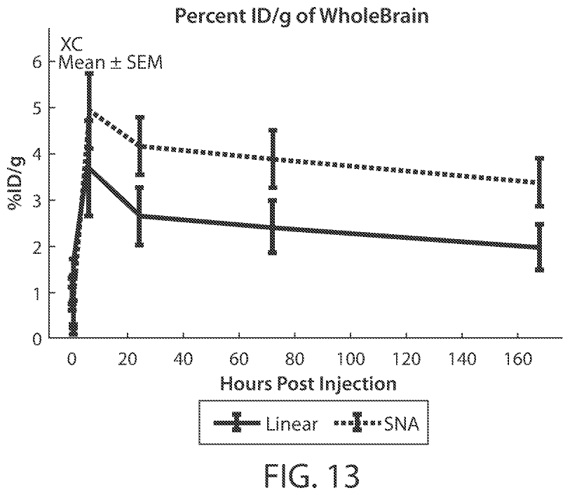

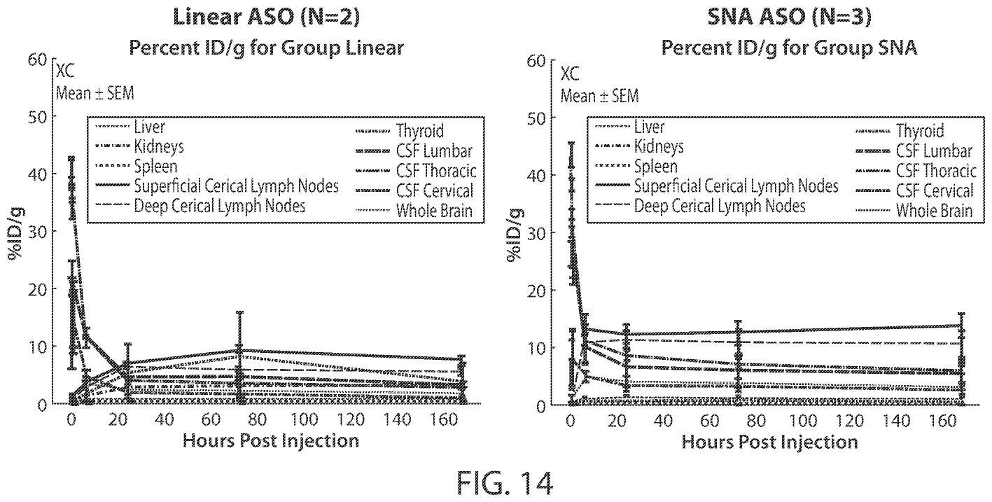

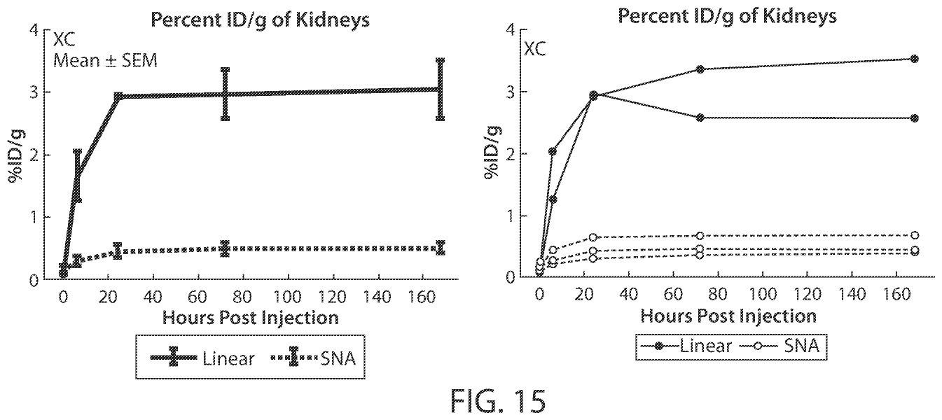

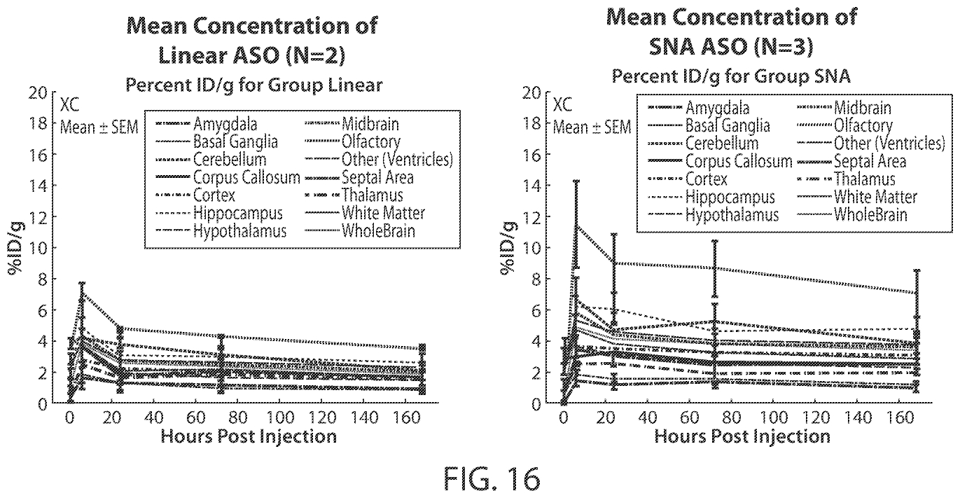

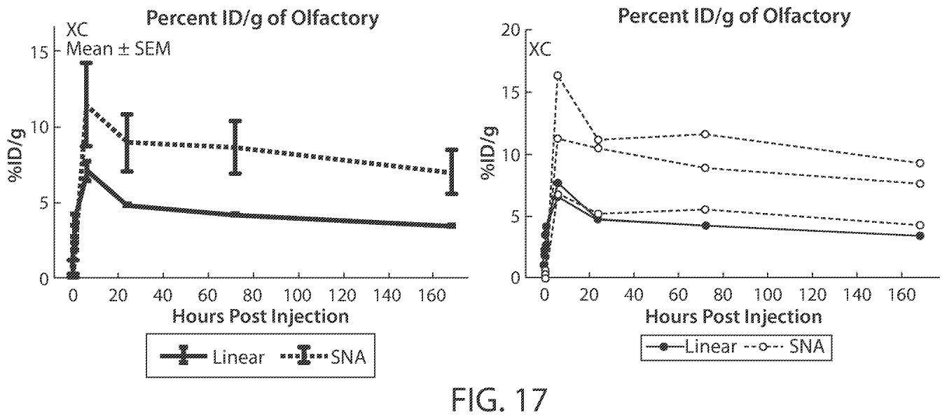

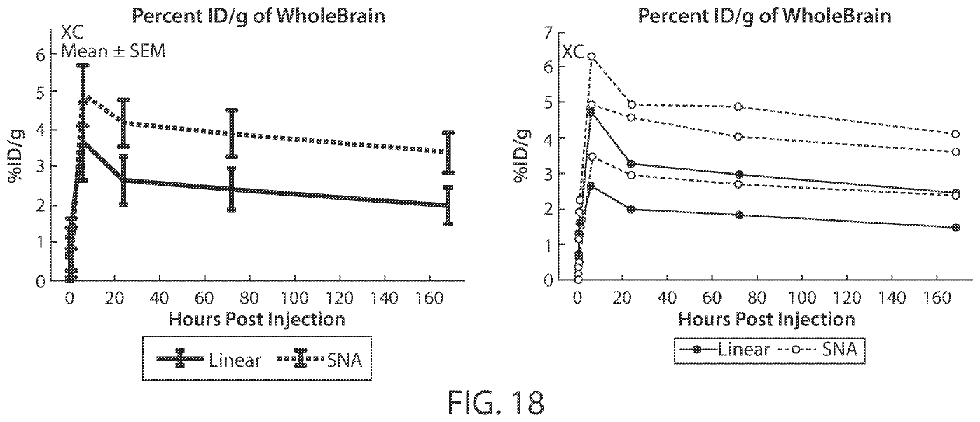

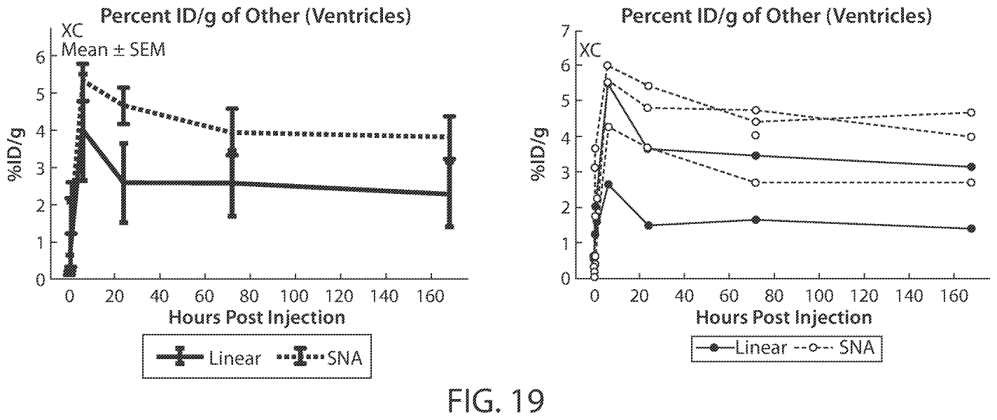

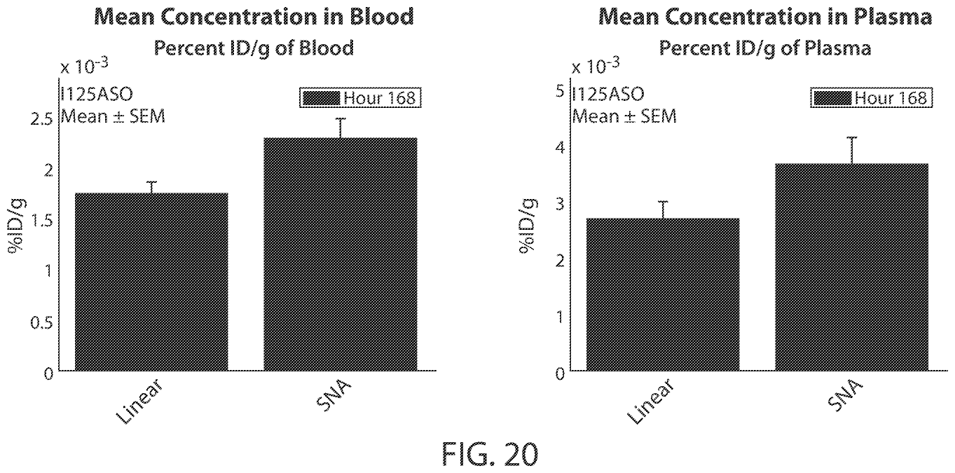

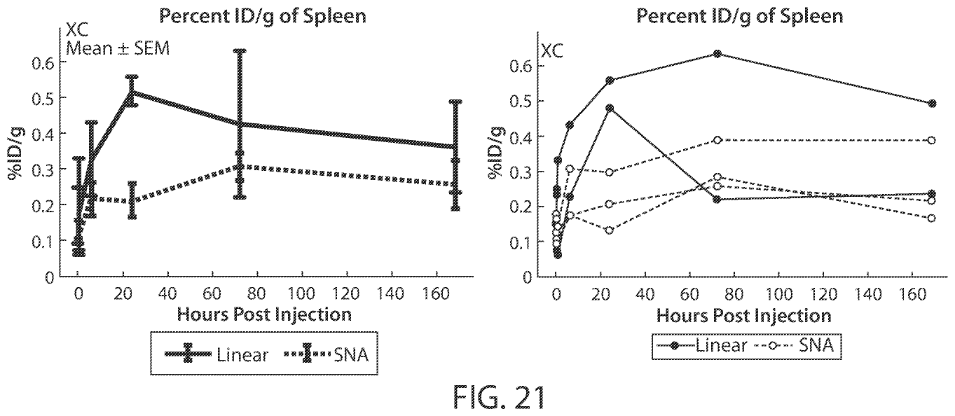

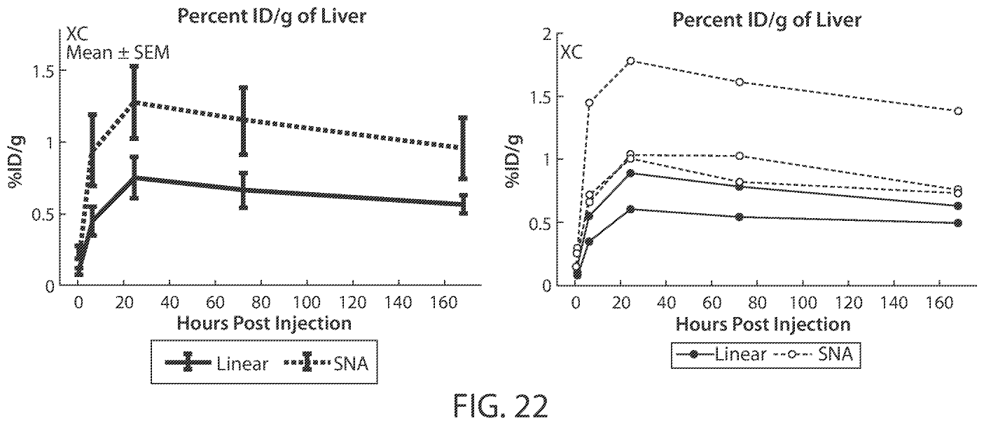

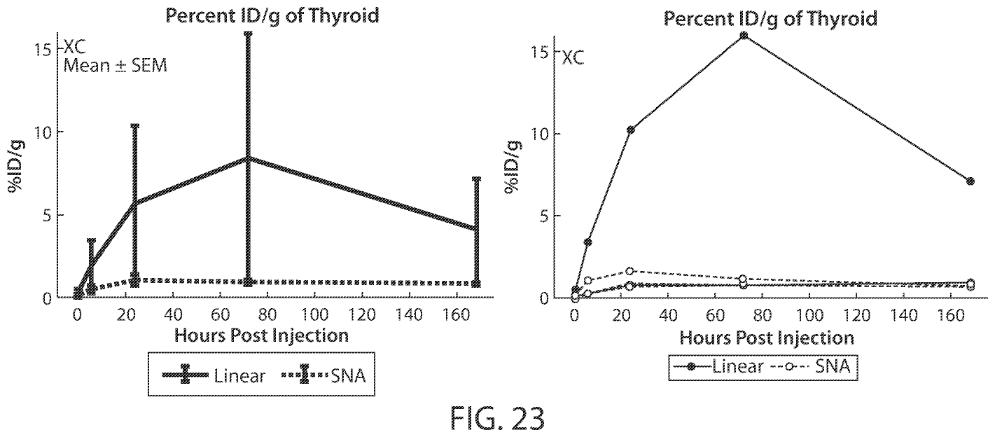

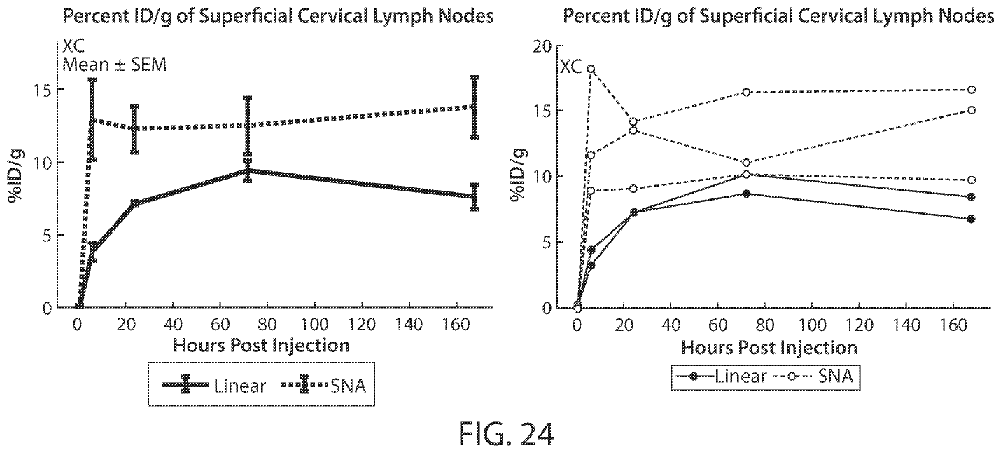

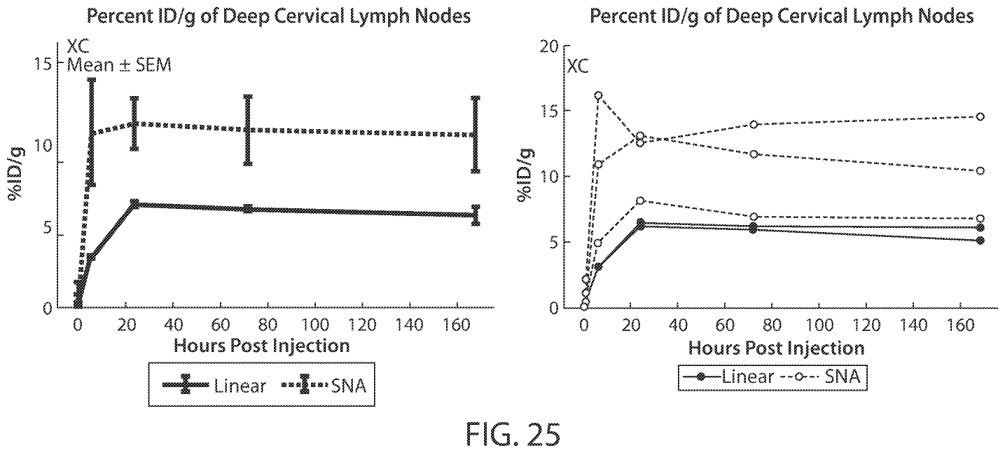

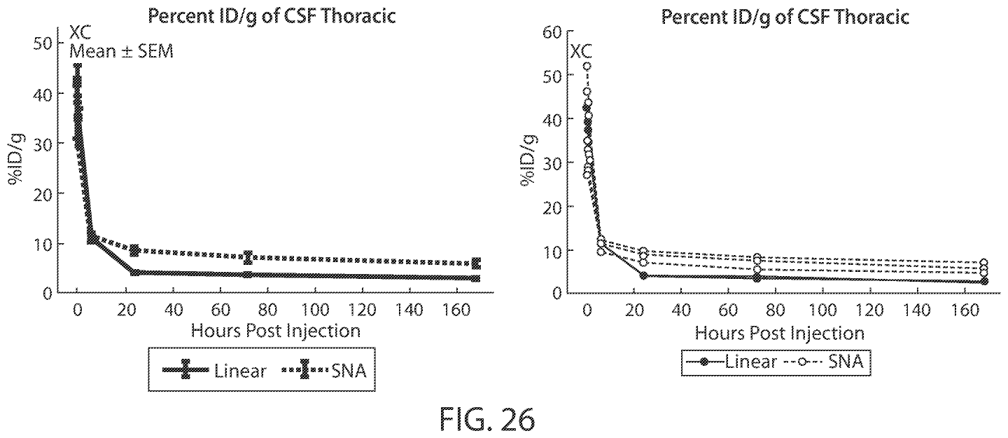

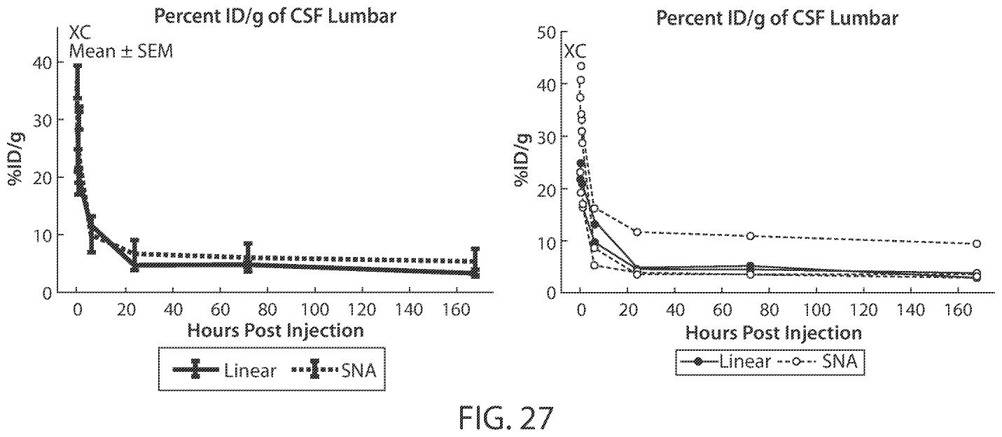

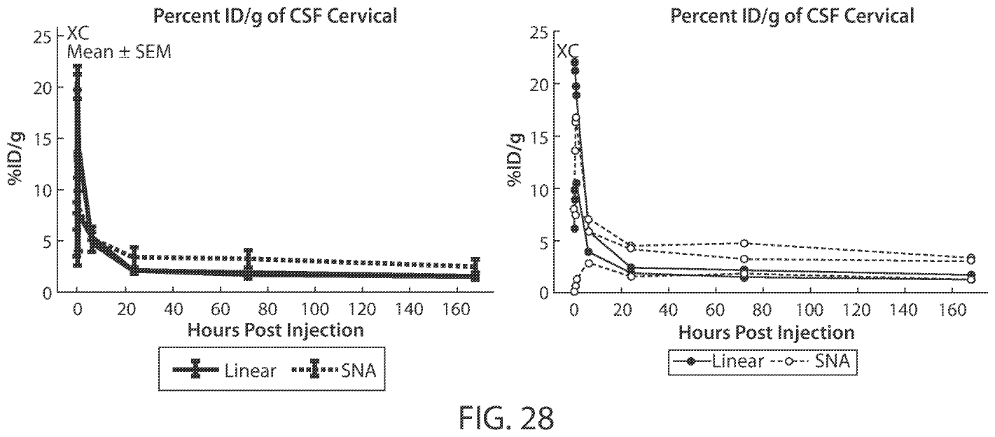

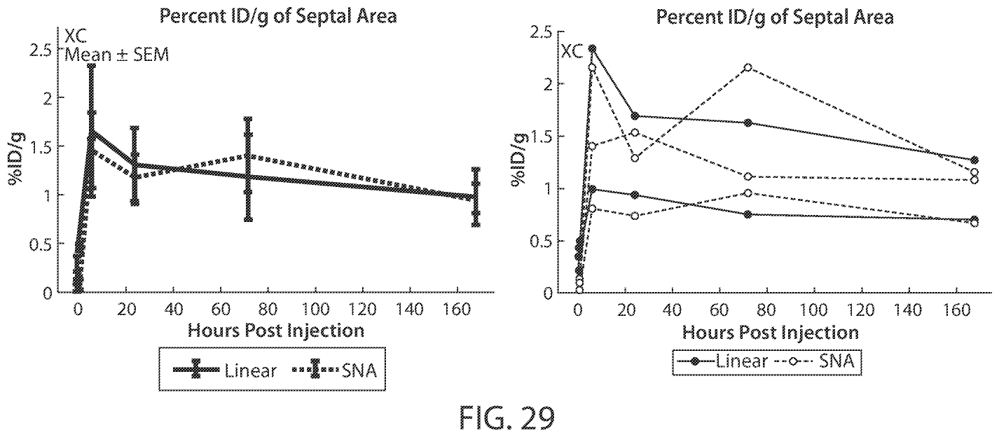

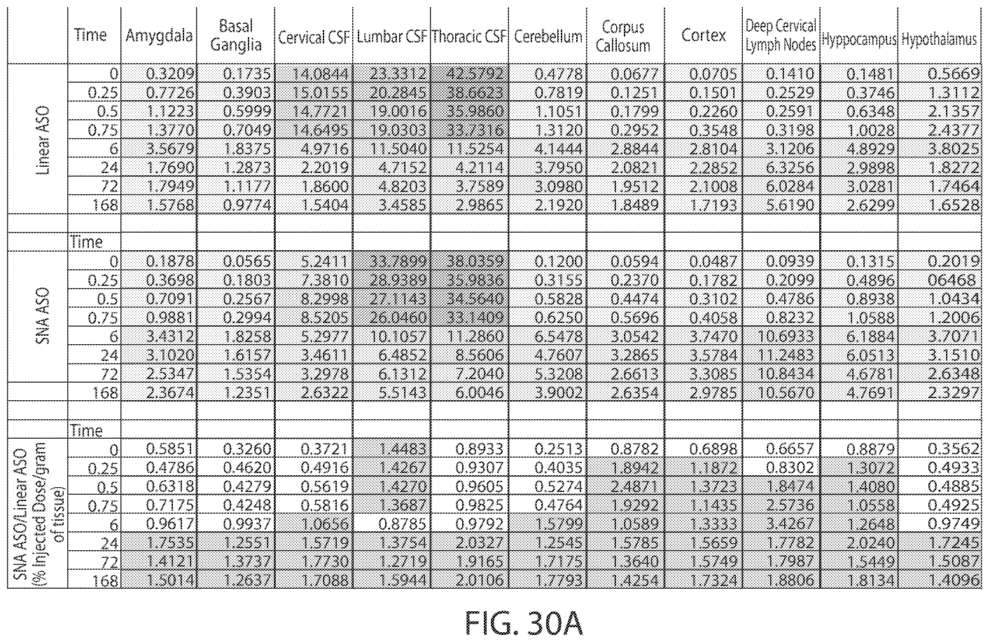

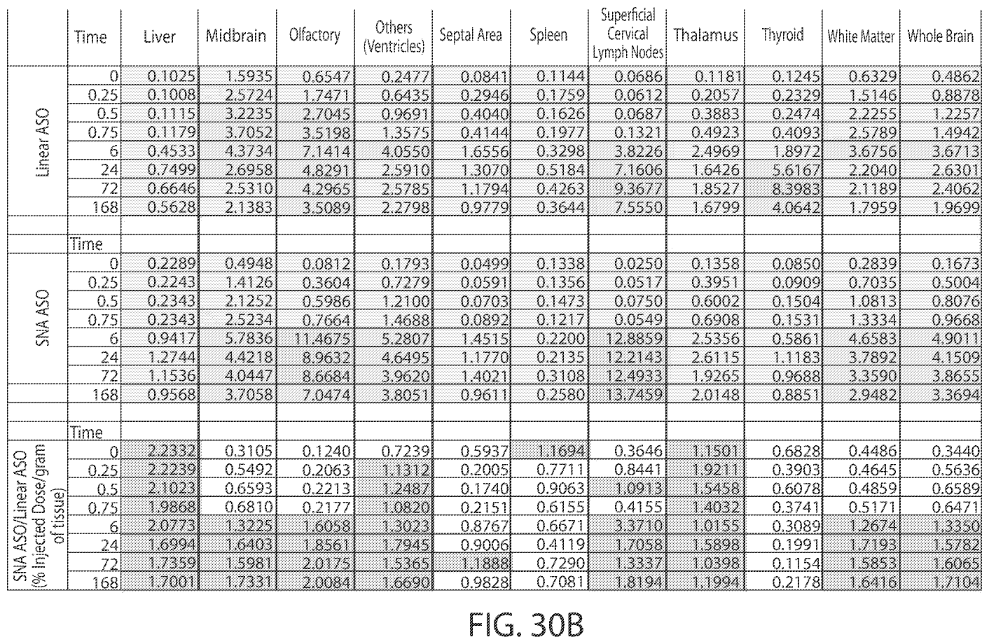

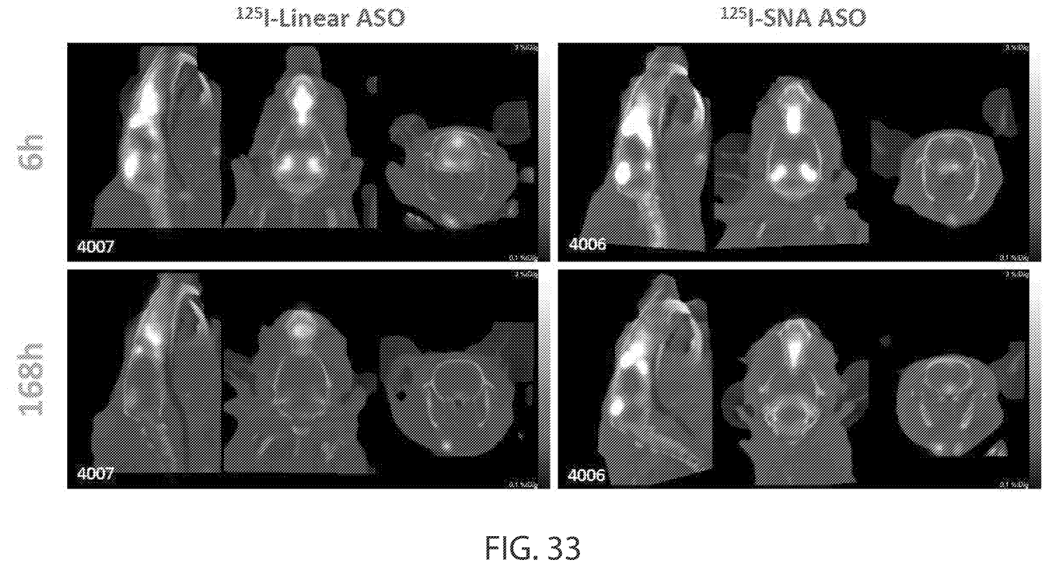

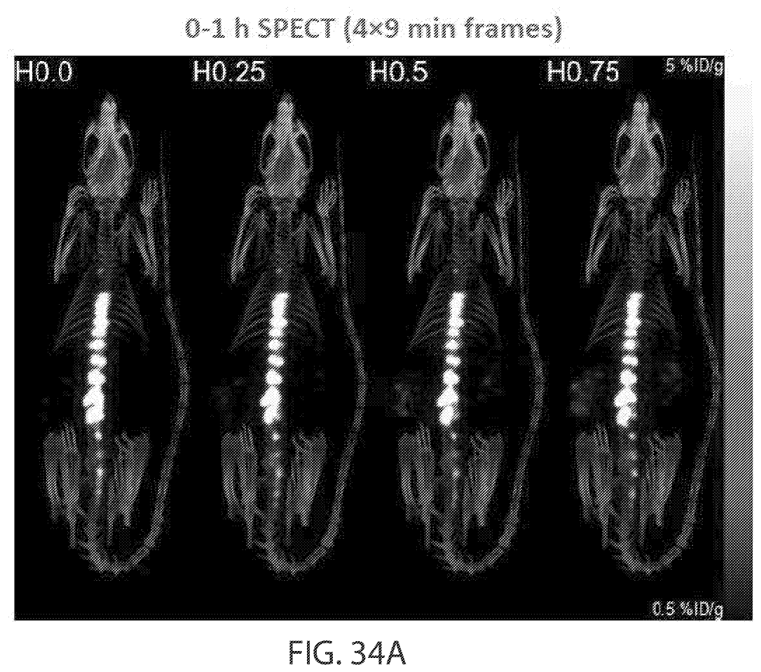

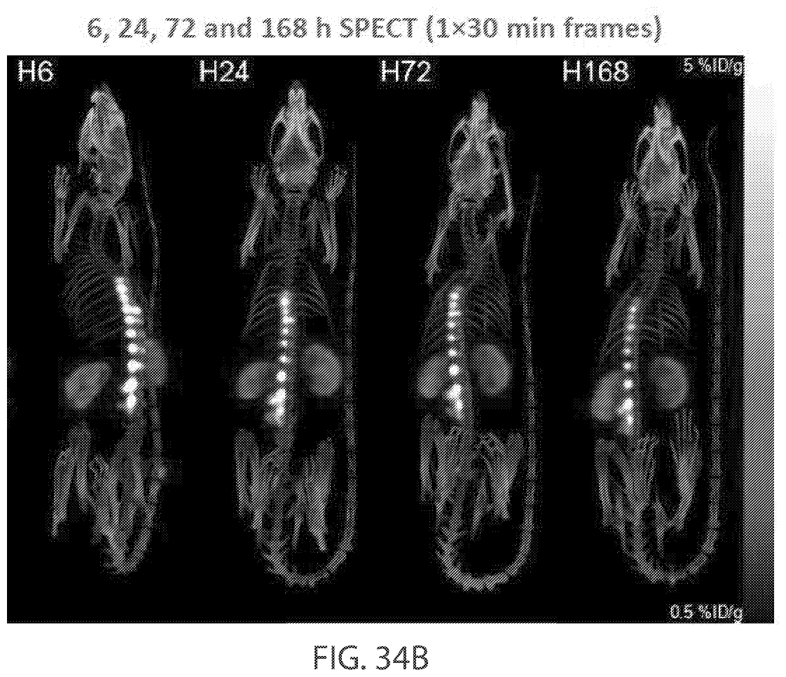

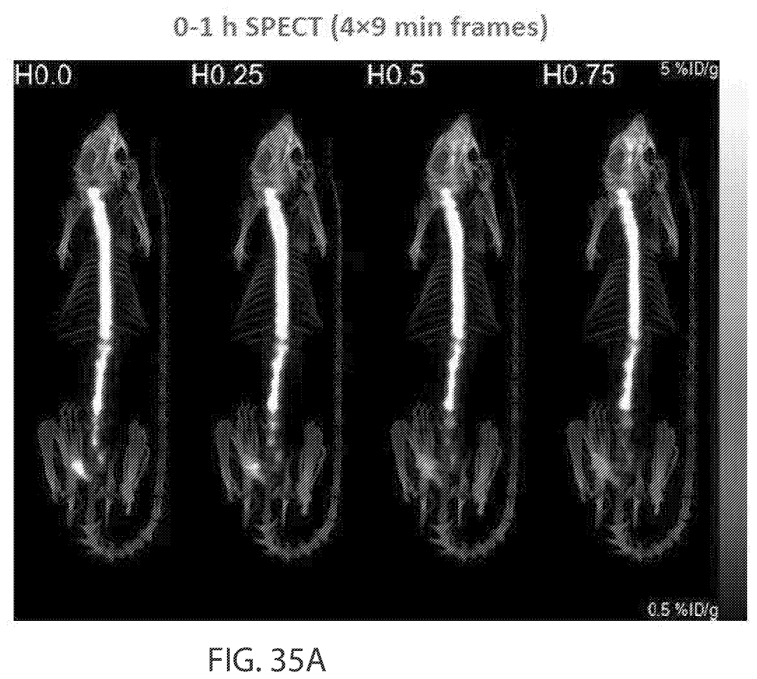

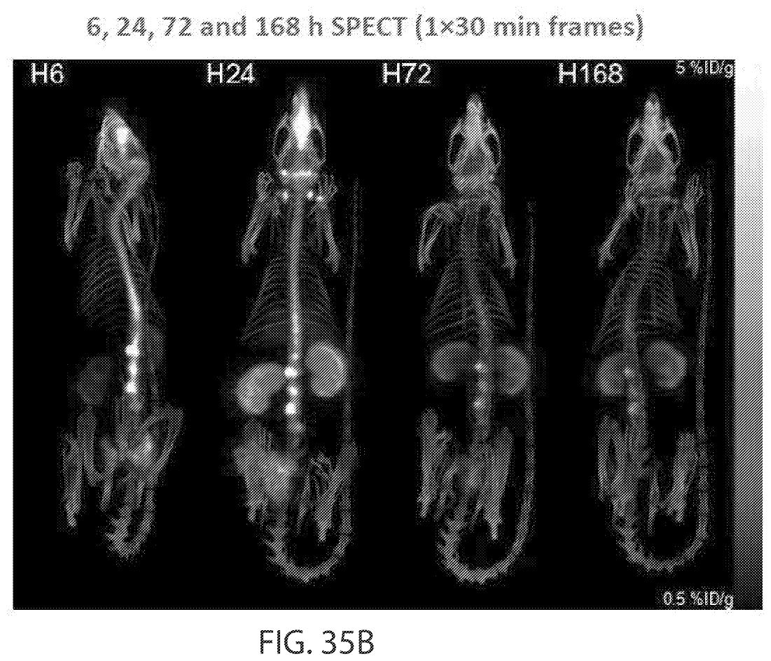

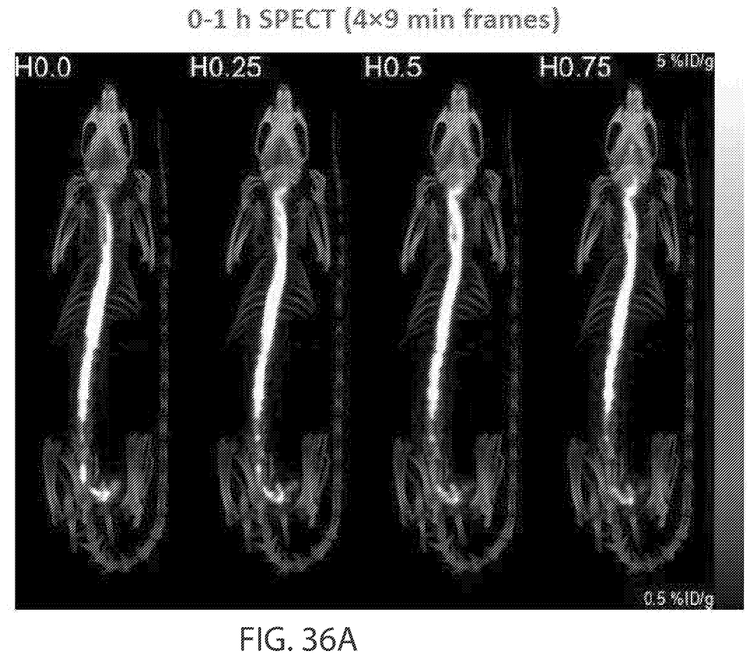

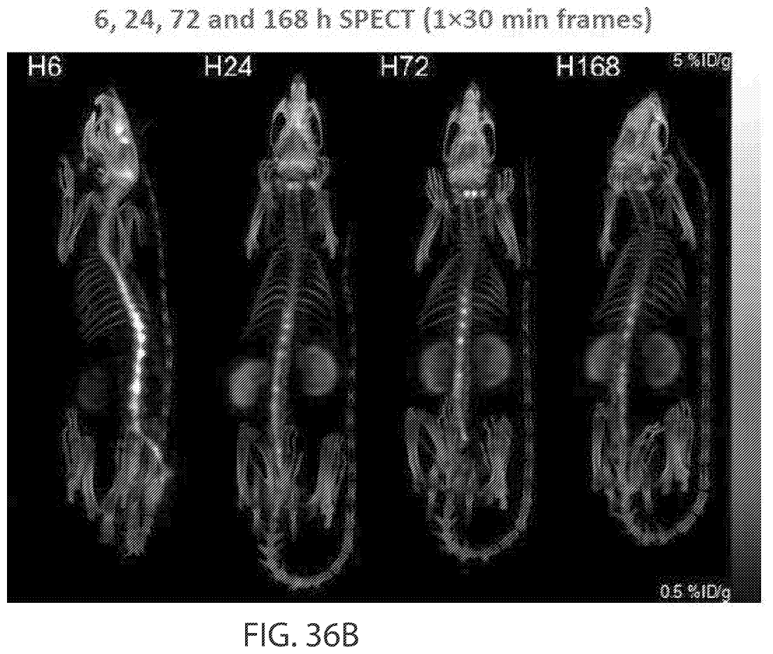

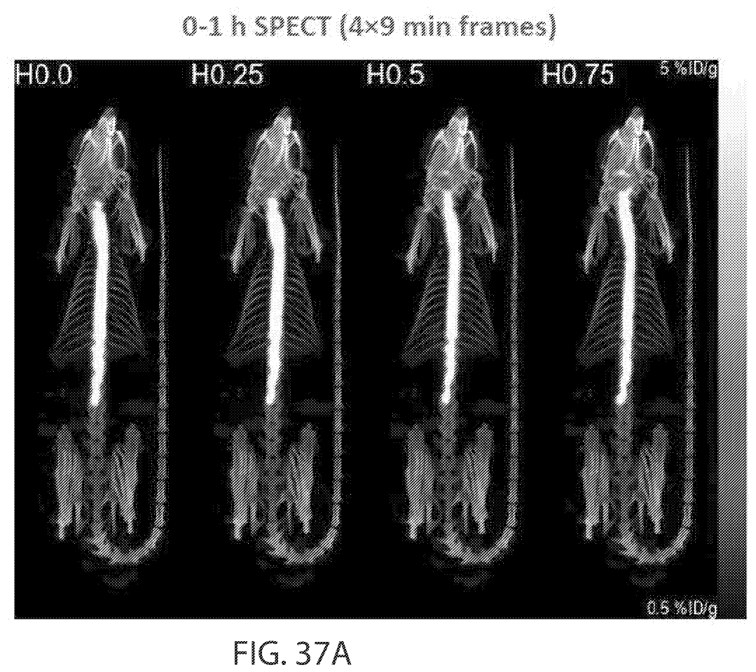

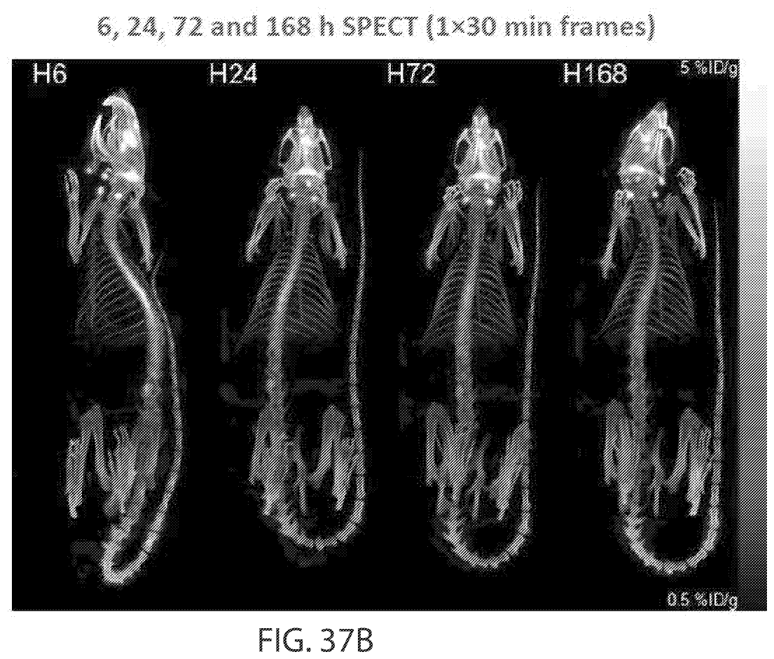

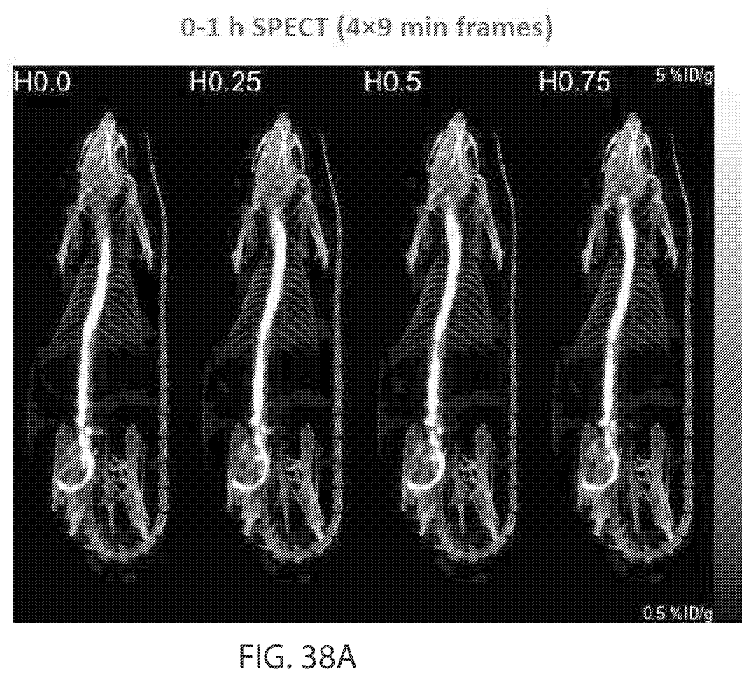

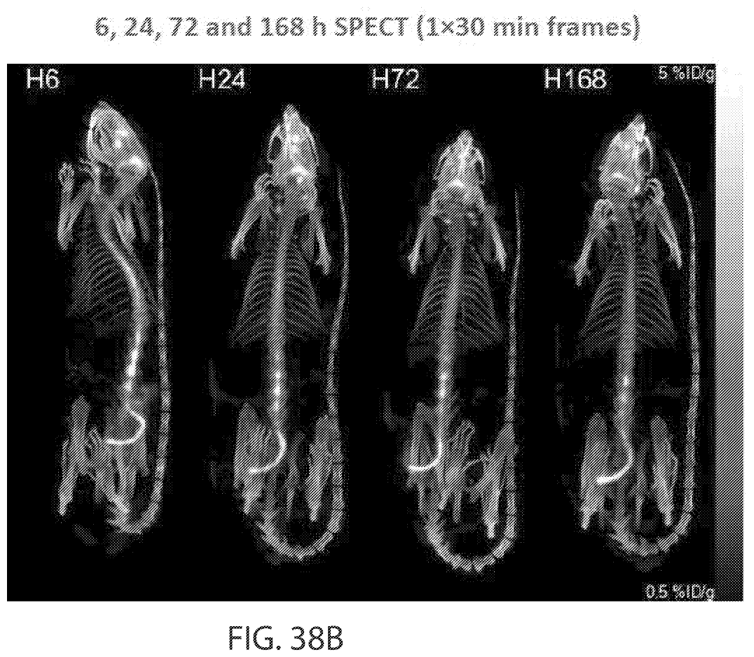

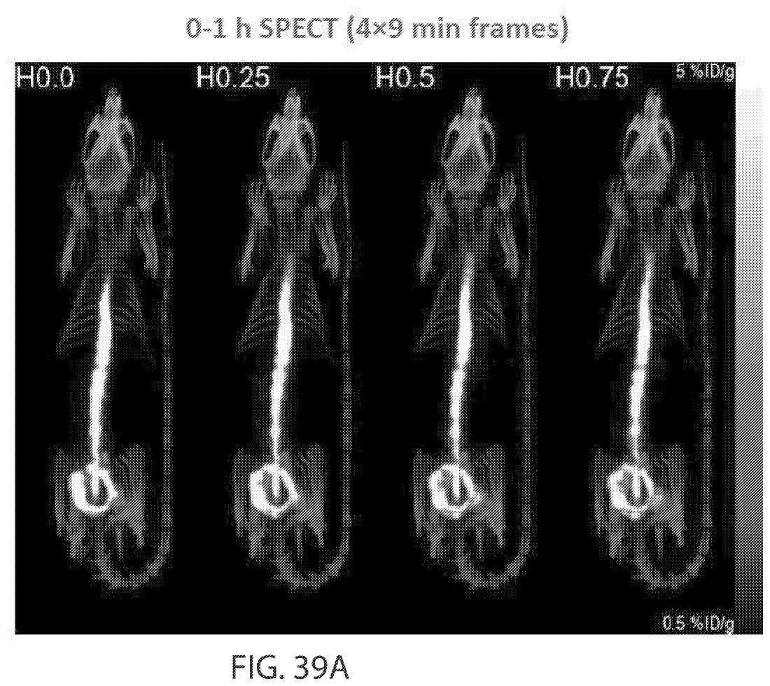

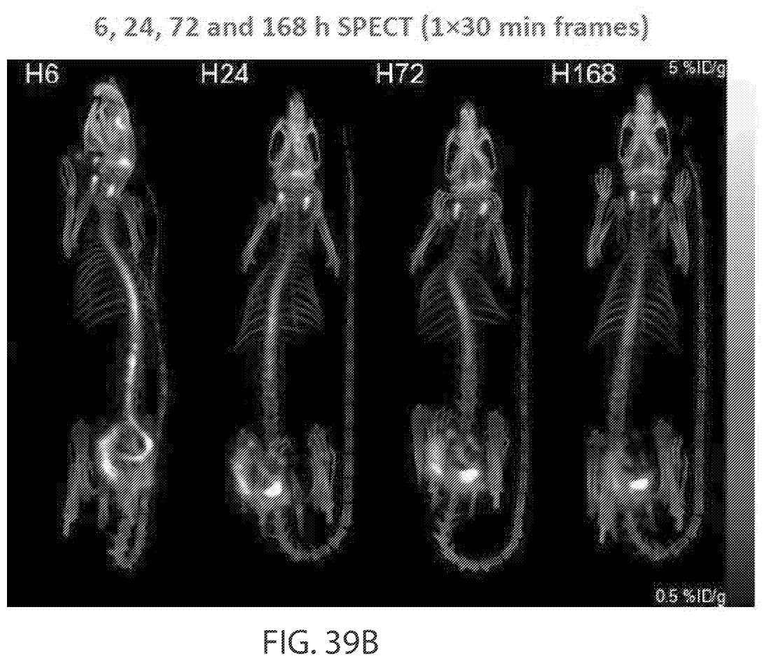

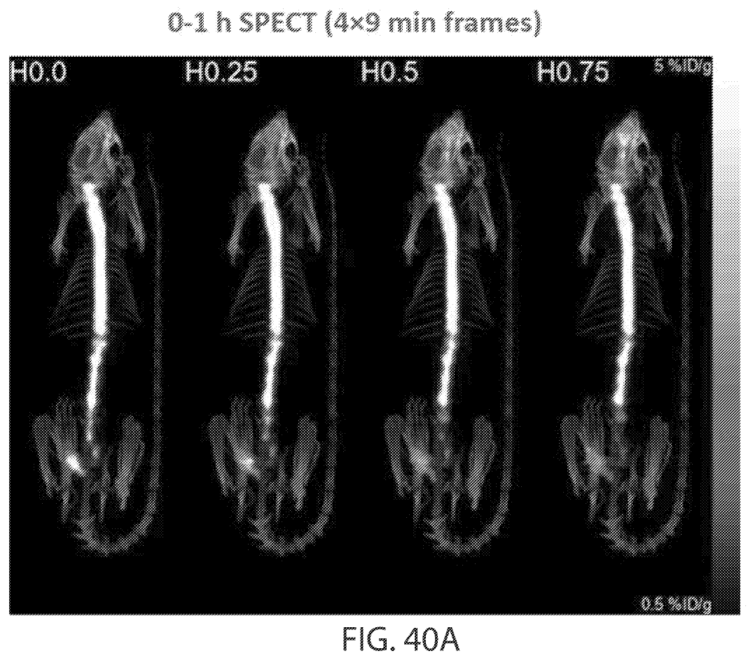

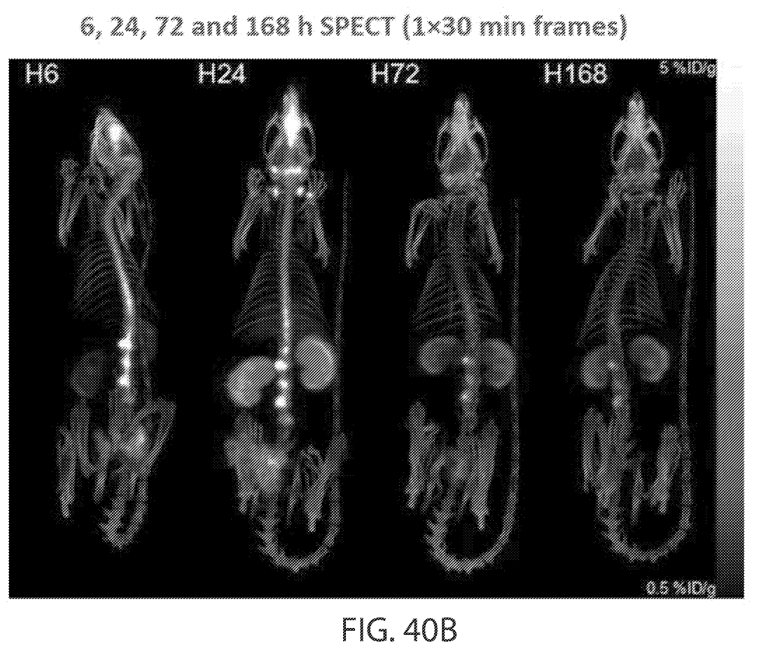

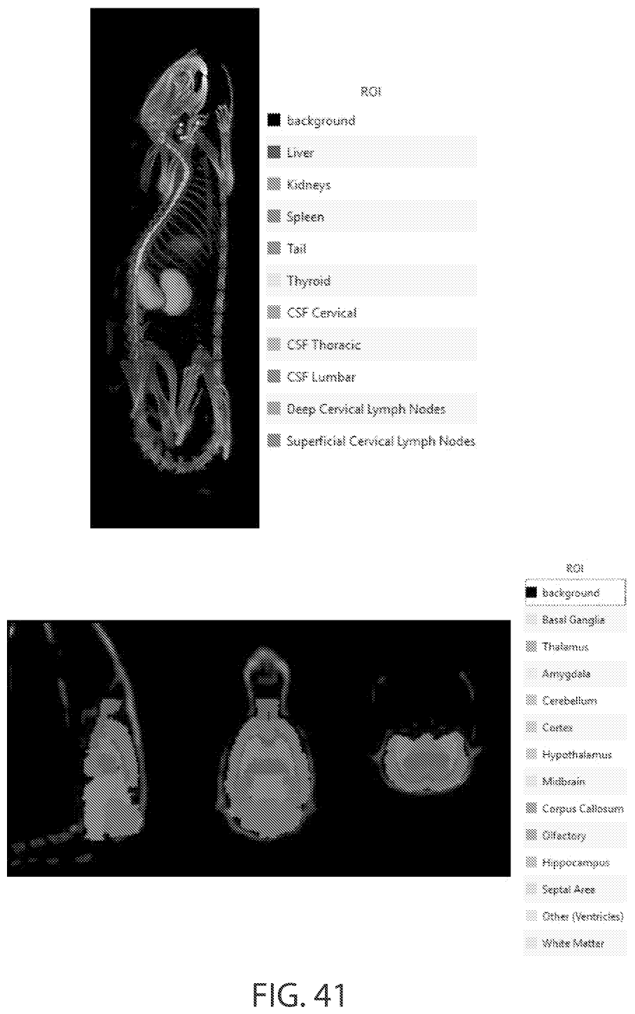

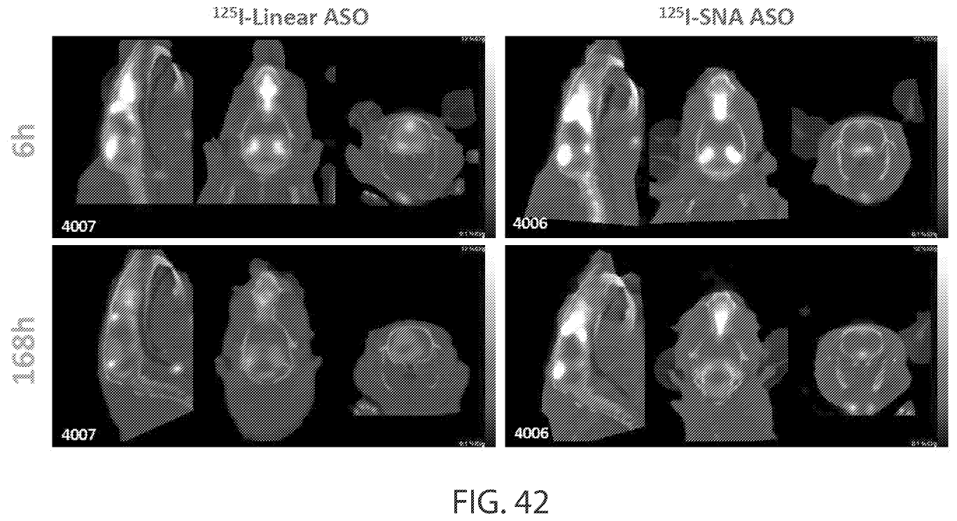

SPEC/CT images of rats 168 hours post-intrathecal administration of ¹²⁵I-ASOs are shown in FIG. 32. The concentration (% ID/g) of ¹²⁵I-Linear ASO was significantly greater than ¹²⁵I-SNA ASO in the kidneys at 6, 24, 72, 168h (FIG. 14). The injection of subject 4001 appeared to be in the epidural space and was thus excluded from quantitative analysis. SPECT/CT images of IT injection of ¹²⁵I-ASOs in rat is shown at 6 hours and 168 hours in FIG. 33. Preliminary images of ¹¹²I-ASO maximum intensity projections (MIPs) for all subjects are shown in FIGS. 34-40. The concentration (% ID/g) of ¹²⁵I-Linear ASO was significantly greater than ¹²⁵I-SNA ASO in the kidneys at 6, 24, 72, 168 h (p<0.05, IS t-test) (FIG. 15). SPECT/CT images at 6 and 168h are shown in rats after IT injection of ¹²⁵I-ASOs (FIG. 42). Graphs of further results are shown in FIGS. 13-30. ¹²⁵I measurements were quantified in organs and regions of the brain (i.e., to generate the data shown in FIGS. 13, 14, 15, 16, 17, 18, 19, 21, 22, 23, 24, 25, 26, 27, 28, 29 and 30) according to the region of interest (ROI) analysis key shown in FIG. 41.

### Example 5. Effects of SNA-ASO, Gold (Au)-SNAs and Linear Oligonucleotides Comprising the Sequence of Spinraza on SMN2 and SMN2Δ7 Levels in Fibroblasts

### Methods

Linear oligonucleotides with the same sequence as spinraza were synthesized with 2-methoxyethyl (2′-MOE) and phosphorothioate (PS) backbone modifications. These oligonucleotides contained 3′ cholesterol, distearyl, monothiol ordithiol modifications attached via hexaethyleneglycol (spacer18) moieties. Nonsense control sequences were also synthesized to compare efficacy. Table 11 contains information on oligonucleotide sequence and modifications. SNAs (SNA-ASO) were prepared by loading oligonucleotides containing 3′ cholesterol ordistearyl onto DOPC liposomes. Oligonucleotides containing monothiol and dithiol modifications were functionalized onto gold nanoparticles to produce gold SNAs (Au-SNA). SNA core size and oligonucleotide loading densities per particle are described in Table 12.

TABLE 11

| 2′-OMOE | SEQ | |
|---|---|---|
| Chemistry | ID NO | Sequence |
| ASO 10-27- | 83 | moeT*/5-Me-moeC/*moeA*/5-Me- |
| MOE-PS-Chol | | moeC/*moeT*moeT*moeT*/5-Me- |
| | | moeC/*moeA*moeT*moeA*moeA*moeT*moeG*/5-Me- |
| | | moeC/*moeT*moeG*moeG/isp18//isp18//3CholTEG/ |
| SMN Control- | 84 | moeT*moeG*moeT*moeA*moeT*/5-Me- |
| 1-MOE-PS- | | moeC/*moeT*/5-Me- |
| Chol | | moeC/*moeA*moeT*moeT*moeG*moeT*moeA*moeG/isp |
| | | 18//isp18//3CholTEG/ |
| ASO 10-27- | 85 | moeT*/5-Me-moeC/*moeA*/5-Me- |
| MOE-PS | | moeC/*moeT*moeT*moeT*/5-Me- |
| Distearyl | | moeC/*moeA*moeT*moeA*moeA*moeT*moeG*/5-Me- |
| | | moeC/*moeT*moeG*moeG/iSP18//iSP18//branch//STA/ |
| SMN Control- | 86 | moeT*moeG*moeT*moeA*moeT*/5-Me- |
| MOE-PS- | | moeC/*moeT*/5-Me- |
| Distearyl | | moeC/*moeA*moeT*moeT*moeG*moeT*moeA*moeG/iSp |
| | | 18//iSp18//branch//STA/ |
| ASO 10-27- | 87 | moeT*/5-Me-moeC/*moeA*/5-Me- |
| MOE-PS | | moeC/*moeT*moeT*moeT*/5-Me- |
| Monothiol | | moeC/*moeA*moeT*moeA*moeA*moeT*moeG*/5-Me- |
| | | moeC/*moeT*moeG*moeG/iSP18//iSP18//3ThioMC6-D/ |
| SMN Control- | 88 | moeT*moeG*moeT*moeA*moeT*/5-Me- |
| MOE-PS | | moeC/*moeT*/5-Me- |
| Monothiol | | moeC/*moeA*moeT*moeT*moeG*moeT*moeA*moeG/iSp |
| | | 18//iSp18//3ThioMC6-D/ |
| ASO 10-27- | 89 | moeT*/5-Me-moeC/*moeA*/5-Me- |
| MOE-PS | | moeC/*moeT*moeT*moeT*/5-Me- |
| Dithiol | | moeC/*moeA*moeT*moeA*moeA*moeT*moeG*/5-Me- |
| | | moeC/*moeT*moeG*moeG/iSP18//iSP18//3SerinolDTPA/ |
| SMN Control- | 90 | moeT*moeG*moeT*moeA*moeT*/5-Me- |
| MOE-PS | | moeC/*moeT*/5-Me- |
| Dithiol | | moeC/*moeA*moeT*moeT*moeG*moeT*moeA*moeG/iSp |
| | | 18//iSp18//3SerinolDTPA/ |

*= phosphorothioate internucleotide linkage;
moe = 2′-O-(2-methoxyethyl);
iSp18 = internal hexaethyleneglycol spacer;
5-Me-moeC = 5′ methyl C with moe modification;
3CholTEG = 3′-Cholesteryl-TEG;
5CholTEG = 5′Cholestery-TEG;
TEG = tetraethylene glycol;
branch = symmetrical branching;
STA = stearyl;
3ThioMC6-D = Monothiol;
3SerinolDTPA = Dithiol serinol

TABLE 12

| | SNA Core | Oligonucleotides |
|---|---|---|
| SNA Oligonucleotide | (core diameter in nm) | per Core |
| ASO 10-27-MOE-PS-Chol | DOPC (20) | 30 |
| SMN Control-MOE-PS-Chol | DOPC (20) | 30 |
| ASO 10-27-MOE-PS Distearyl | DOPC (20) | 30 |
| SMN Control-MOE-PS- | DOPC (20) | 30 |
| Distearyl | | |
| ASO 10-27-MOE-PS Monothiol | Gold (13) | 198 |
| SMN Control -MOE-PS | Gold (13) | 197 |
| Monothiol | | |
| ASO 10-27-MOE-PS Dithiol | Gold (13) | 147 |
| SMN Control -MOE-PS Dithiol | Gold (13) | 155 |

SMA patient fibroblast cells (GM09677C) were obtained from Coriell Institute for Medical Research and cultured in EMEM medium containing 15% FBS. Fibroblasts were plated in a 96-well plate at a density of 10,000 cells per well. SNA-ASOs, Au—SNAs or linear cholesterol/distearyl oligonucleotides were added to the culture media in triplicate. After 48 hours of treatment the cells were collected for mRNA extraction. The levels of SMN2, SMN2Δ7, and total SMN2 mRNAs were measured by RT-PCR using assays from ThermoFisher Scientific. SMN2 mRNA, SMNA7 mRNA primer and probe sequences were: SMN2 mRNA forward primer: 5′-GCTG ATGCTTIGGG AAGTATG1TA-3′ (SEQ ID NO: 2), SMN2 mRNA reverse primer: 5′-CACCT′CCTTCTITITGATTITGTC-3′ (SEQ ID NO: 3), SMN2 mRNA probe: 5-6FAM-TACATGAGTGGCTATCATACT′-MGBNFQ-3′ (SEQ ID NO: 4), SMN2Δ7 mRNA forward primer: 5′-TGGACCACCAATAATTCCCC-3′ (SEQ ID NO: 5), SMN2Δ7 mRNA reverse primer: 5′-ATGCCAGCATTT CCATATAATAGCC-3′ (SEQ ID NO: 6) and SMN2Δ7 mRNA probe: 5′-6FAM-TACATGAGTGGCTATCATACT-MGBNFQ-3′ (SEQ ID NO: 7).

Total SMN2 mRNAs were measured using a commercial gene expression assay (cat #Hs00165806_ml). Fold changes in SMN2 and SMN2Δ7 transcripts were calculated and normalized to untreated fibroblasts expression levels.

### Results

SNA-ASO, Au-SNAs and linear oligonucleotides consisting of the spinraza or control sequence were tested in SMA patient fibroblasts. SNAs were tested at 5, 1 and 0.2 μM, while linear cholesterol or distearyl oligonucleotides were tested at 1 μM. Fibroblasts were treated for 48 hours prior to processing.

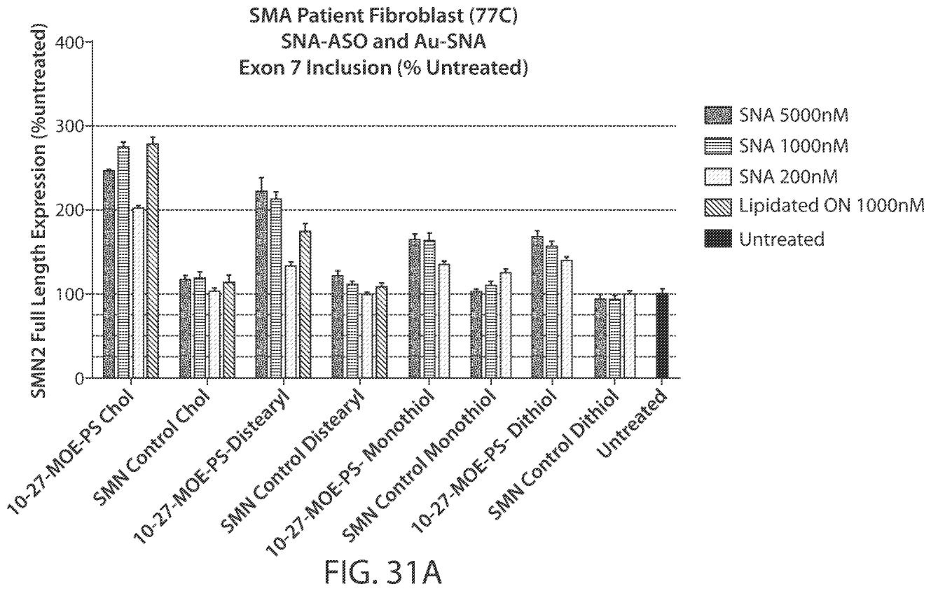

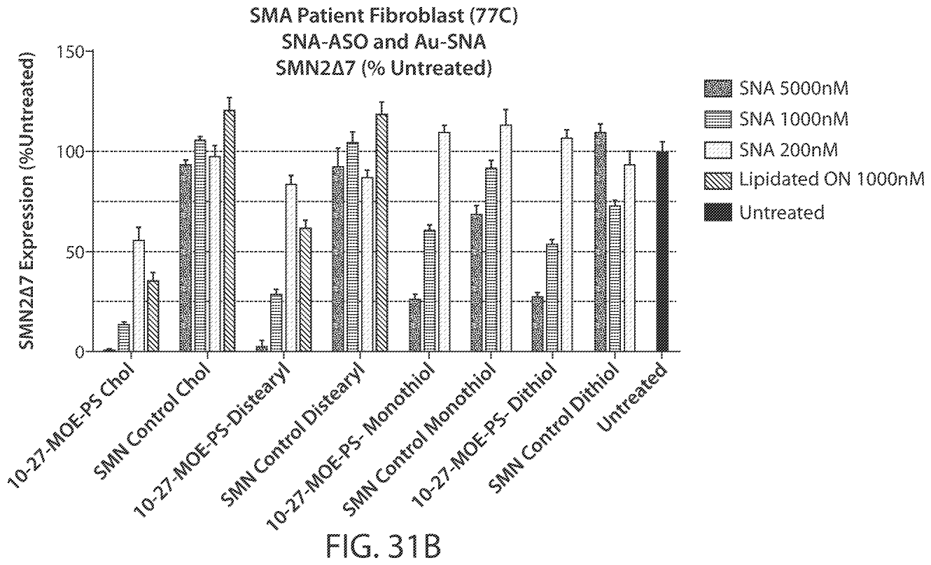

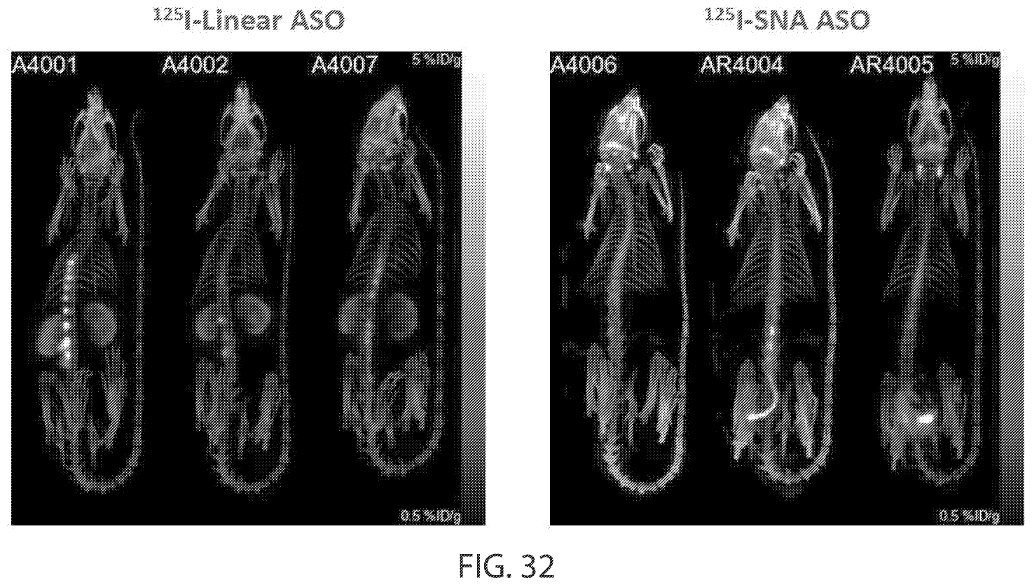

Data are included in FIG. 31A (SNA-ASO and Au-SNA: Full-length SMN2 mRNA) and FIG. 31B (SNA-ASO and Au-SNA: Δ7 SMN2 mRNA). All SNAs that contained the spinraza sequence showed SMN2 exon 7 inclusion and an associated SMN2Δ7 transcript reduction. In general, SNA-ASO outperformed Au-SNAs but compound efficacy varied. SNAs with 3′ distearyl or cholesterol showed approximately a 2-2.5-fold increase in full-length SMN2 mRNA relative to untreated at the highest concentration. In comparison, monothiol and dithiol Au-SNAs only produced a 1.5-fold increase. SNA-ASOs also showed greater reduction in SMN2Δ7 mRNA as expected. Linear versions of the cholesterol/distearyl oligonucleotides caused SMN2 exon 7 inclusion and Δ7 reduction, but showed reduced activity compared to SNAs. This is evident in FIG. 31B where greater SMN2Δ7 reduction was seen with the SNA compared to the linear oligonucleotides at 1 μM.

### Conclusion

It was previously shown that SNA-ASOs containing the spinraza sequence modified at the 3′ end with cholesterol were able to cause SMN2 exon 7 inclusion in patient fibroblasts. In the current study, a SNA-ASO containing the distearyl-modified oligonucleotide and two different Au-SNAs consisting of oligonucleotides covalently attached to gold nanoparticles also showed splice-switching activity. SNAs with distearyl or thiol modifications had different efficacies but the SNA with the cholesterol-modified oligonucleotide outperformed both. Differences in the bond strength between the oligonucleotide modification and SNA core may have played a role. This is the first indication that liposomal SNAs containing distearyl-modified oligonucleotides and gold SNAs are able to target the 1SS-N1 region of the SMN2 pre-mRNA in the nucleus. Cholesterol-modified oligonucleotide consisting spinraza sequence also showed similar level of full-length SMN2 expression as the same oligonucleotide in SNA. Surprisingly, the SNA version showed greater reduction in the Δ7 variant of SMN2 mRNA compared to cholesterol-modified oligonucleotide.

### SEQUENCES

TABLE 13
Nucleic acid and amino acid sequences of genes, mRNA and protein variants of
IL17RA, REST, IL1RAP, and STAT3

| | | NCBI Reference |
|---|---|---|
| SEQ ID NO: | Name | Sequence |
| 91 | Homo sapiens IL17RA, RefSeqGene | NG_028257.1 |
| | (LRG_355) on Chromosome 22 | |
| 92 | Homo sapiens IL17RA, transcript variant 1, mRNA | NM_014339.6 |
| 93 | Homo sapiens IL17RA, transcript variant 2, mRNA | NM_001289905.1 |
| 94 | Homo sapiens IL17RA isoform 1 precursor | NP_055154.3 |
| 95 | Homo sapiens IL17RA isoform 2 precursor | NP_001276834.1 |
| 96 | Homo sapiens REST, RefSeqGene on chromosome 4 | NG_029447.1 |
| 97 | Homo sapiens REST, transcript variant 1, mRNA | NM_005612.5 |
| 98 | Homo sapiens REST, transcript variant 2, mRNA | NM_001193508.1 |
| 99 | Homo sapiens REST, transcript variant 3, mRNA | NM_001363453.2 |
| 100 | PREDICTED: Homo sapiens REST, | XM_017008527.1 |
| | transcript variant X2, mRNA | |
| 101 | Homo sapiens REST | NP_005603.3 |
| 102 | Homo sapiens REST isoform X2 | XP_016864016.1 |
| 103 | Homo sapiens IL1RAP, RefSeqGene on chromosome 3 | NG_029105.2 |
| 104 | Homo sapiens IL1RAP, transcript variant 1, mRNA | NM_002182.4 |
| 105 | Homo sapiens IL1RAP, transcript variant 5, mRNA | NM_001167930.2 |
| 106 | Homo sapiens IL1RAP, transcript variant 8, mRNA | NM_001364880.2 |
| 107 | Homo sapiens IL1RAP isoform 1 precursor | NP_002173.1 |
| 108 | Homo sapiens IL1RAP isoform 2 precursor | NP_001161402.1 |
| 109 | Homo sapiens IL1RAP isoform 3 precursor | UniProtKB: |
| | | Q9NPH3-3 |
| 110 | Homo sapiens IL1RAP isoform 4 precursor | NP_001351809.1 |
| 111 | Homo sapiens IL1RAP isoform 4 precursor | UniProtKB: |
| | | Q9NPH3-5 |
| 112 | Homo sapiens STAT3, RefSeqGene | NG_007370.1 |
| | (LRG_112) on chromosome 17 | |
| 113 | Homo sapiens STAT3 isoform 1, | NM_001369512.1 |
| | transcript variant 4, mRNA | |
| 114 | Homo sapiens STAT3 isoform 2, | NM_001369514.1 |
| | transcript variant 6, mRNA | |
| 115 | Homo sapiens STAT3 isoform 3, | NM_001369517.1 |
| | transcript variant 8, mRNA | |
| 116 | Homo sapiens STAT3 isoform 1 | NP_001356441.1 |
| 117 | Homo sapiens STAT3 isoform 2 | NP_001356443.1 |
| 118 | Homo sapiens STAT3 isoform 3 | NP_001356446.1 |

Homo sapiens REST isoform X2
NCB1 Reference Sequence: XP_016864016.1
1069 aa
(SEQ ID NO: 102)

| | |
|---|---|
| 1 | MATQVMGQSS GGGGLFTSSG NIGMALPNDM YDLHDLSKAE |
| | LAAPQLIMLA NVALTGEVNG |
| 61 | SCCDYLVGEE RQMAELMPVG DNNFSDSEEG EGLEESADIK |
| | GEPHGLENME LRSLELSVVE |
| 121 | PQPVFEASGA PDIYSSNKDL PPETPGAEDK GKSSKTKPFR |
| | CKPCQYEAES EEQFVHHIRV |
| 181 | HSAKKFFVEE SAEKQAKARE SGSSTAEEGD FSKGPIRCDR |
| | CGYNTNRYDH YTAHLKHHTR |
| 241 | AGDNERVYKC IICTYTTVSE YHWRKHLRNH FPRKVYTCGK |
| | CNYFSDRKNN YVQHVRTHTG |
| 301 | EKPFKCDQCS YVASNQHEVT RHARQVHNGP KPLNCPHCDY |
| | KTADRSNFKK HVELHVNPRQ |
| 361 | FNCPVCDYAA SKKCNLQYHF KSKHPTCPNK TMDVSKVKLK |
| | KTKKREADLP DNITNEKTEI |
| 421 | EQTKIKGDVA GKKNEKSVKA EKRDVSKEKK PSNNVSVIQV |
| | TTRTRKSVTE VKEMDVHTG3 |
| 481 | NSEKFSKTKK SKRKLEVDSH SLHGPVNDEE SSTKKKKKVE |
| | SKSKNNSQEV PKGDSKVEEN |
| 541 | KKQNTCMKKS TKKKTLKNKS SKKSSKPPQK EPVEKGSAQM |
| | DPPQMGPAPT EAVQKGPVQV |
| 601 | EPPPPMEHAQ MEGAQIRPAP DEPVQMEVVQ EGPAQKELLP |
| | PVEPAQMVGA QIVLAHMELP |
| 661 | PPMETAQTEV AQMGPAPMEP AQMEVAQVES APMQVVQKEP |
| | VQMELSPPME VVQKEPVQIE |
| 721 | LSPPMEVVQK EPVKIELSPP LEVVQKEPVQ MELSPPMGVV |
| | QKEPAQREPP PPREPPLHME |
| 781 | PISKKPPLRK DKKEKSNMQS ERARKEQVLI EVGLVPVKDS |
| | WLLKESVSTE DLSPPSPPLP |
| 841 | KENLREEASG DQKLLNTGEG NKEAPLQKVG AEEADESLPG |
| | LAANINESTH ISSSGQNLNT |
| 901 | PEGETLNGKE QTDSIVCEMK MDTDQNTREN LTGINSTVEE |
| | PVSPMLPPSA VEEREAVSKT |
| 961 | ALASPPATMA ANESQEIDED EGIHSHEGSD LSDNMSEGSD |
| | DSGLHGARPV PQESSRKNAK |
| 1021 | EALAVKAAKG DFVCIFCDRS FRKGKDYSKH LNRHLVNVYY |
| | LEEAAQGQE |

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A spherical nucleic acid (SNA) for regulating pre-mRNA splicing, comprising
a core and an antisense oligonucleotide comprised of 8 to 50 linked nucleosides in length targeted to a region in a pre-mRNA of interest to regulate pre-mRNA splicing, and wherein the antisense oligonucleotide is attached to the core and forms an oligonucleotide shell.

2. The SNA of claim 1, wherein the pre-mRNA of interest is obtained from the genomic sequence of interleukin 17 receptor A (IL17RA), RE1 Silencing Transcription Factor (REST), IL1 receptor accessory protein (IL1RAP), or signal transducer and activator of transcription 3 (STAT3).

3. The SNA of any one of claims 1-2, wherein the core is a solid core or a hollow core.

4. The SNA of any one of claims 1-3, wherein the core is a liposomal core.

5. The SNA of any one of claims 1-4, wherein the core has a diameter of or about 5 nm to about 150 nm.

6. The SNA of any one of claims 1-5, wherein the core has a diameter of or about 5 nm, of or about 6 nm, of or about 7 nm, of or about 8 nm, of or about 9 nm, of or about 10 nm, of or about 11 nm, of or about 12 nm, of or about 13 nm, of or about 14 nm, of or about 15 nm, of or about 16 nm, of or about 17 nm, of or about 18 nm, of or about 19 nm, of or about 20 nm, of or about 21 nm, of or about 22 nm, of or about 23 nm, of or about 24 nm, of or about 25 nm, of or about 26 nm, of or about 27 nm, of or about 28 nm, of or about 29 nm, of or about 30 nm, of or about 31 nm, of or about 32 nm, of or about 33 nm, of or about 34 nm, of or about 35 nm, of or about 36 nm, of or about 37 nm, of or about 38 nm, of or about 39 nm, of or about 40 nm, of or about 41 nm, of or about 42 nm, of or about 43 nm, of or about 44 nm, of or about 45 nm, of or about 46 nm, of or about 47 nm, of or about 48 nm, of or about 49 nm, of or about 50 nm, of or about 55 nm, of or about 60 nm, of or about 65 nm, of or about 70 nm, of or about 75 nm, of or about 80 nm, of or about 85 nm, of or about 90 nm, of or about 95 nm, of or about 100 nm, of or about 110 nm, of or about 120 nm, of or about 130 nm, of or about 140 nm, of or about 150 nm, of or about 160 nm, of or about 170 nm, of or about 180 nm, of or about 190 nm, of or about 200 nm, of or about 210 nm, of or about 220 nm, of or about 230 nm, of or about 240 nm, of or about 250 nm, of or about 260 nm, of or about 270 nm, of or about 280 nm, of or about 290 nm, of or about 300 nm, of more than about 300 nm, of about 15 nm to about 100 nm, of about 20 nm to about 100 nm, of about 25 nm to about 100 nm, of about 15 nm to about 50 nm, of about 20 nm to about 50 nm, of about 10 nm to about 70 nm, of about 15 nm to about 70 nm, of about 20 nm to about 70 nm, of about 10 nm to about 30 nm, of about 15 nm to about 30 nm, of about 20 nm to about 30 nm, of about 10 nm to about 40 nm, of about 15 nm to about 40 nm, of about 20 nm to about 40 nm, of about 10 nm to about 80 nm, of about 15 nm to about 80 nm, or of about 20 nm to about 80 nm.

7. The SNA of any one of claims 1-5, wherein the SNA has a diameter of or about 5 nm, of or about 6 nm, of or about 7 nm, of or about 8 nm, of or about 9 nm, of or about 10 nm, of or about 11 nm, of or about 12 nm, of or about 13 nm, of or about 14 nm, of or about 15 nm, of or about 16 nm, of or about 17 nm, of or about 18 nm, of or about 19 nm, of or about 20 nm, of or about 21 nm, of or about 22 nm, of or about 23 nm, of or about 24 nm, of or about 25 nm, of or about 26 nm, of or about 27 nm, of or about 28 nm, of or about 29 nm, of or about 30 nm, of or about 31 nm, of or about 32 nm, of or about 33 nm, of or about 34 nm, of or about 35 nm, of or about 36 nm, of or about 37 nm, of or about 38 nm, of or about 39 nm, of or about 40 nm, of or about 41 nm, of or about 42 nm, of or about 43 nm, of or about 44 nm, of or about 45 nm, of or about 46 nm, of or about 47 nm, of or about 48 nm, of or about 49 nm, of or about 50 nm, of or about 55 nm, of or about 60 nm, of or about 65 nm, of or about 70 nm, of or about 75 nm, of or about 80 nm, of or about 85 nm, of or about 90 nm, of or about 95 nm, of or about 100 nm, of or about 110 nm, of or about 120 nm, of or about 130 nm, of or about 140 nm, of or about 150 nm, of or about 160 nm, of or about 170 nm, of or about 180 nm, of or about 190 nm, of or about 200 nm, of or about 210 nm, of or about 220 nm, of or about 230 nm, of or about 240 nm, of or about 250 nm, of or about 260 nm, of or about 270 nm, of or about 280 nm, of or about 290 nm, of or about 300 nm, of more than about 300 nm, of about 15 nm to about 100 nm, of about 20 nm to about 100 nm, of about 25 nm to about 100 nm, of about 15 nm to about 50 nm, of about 20 nm to about 50 nm, of about 10 nm to about 70 nm, of about 15 nm to about 70 nm, of about 20 nm to about 70 nm, of about 10 nm to about 30 nm, of about 15 nm to about 30 nm, of about 20 nm to about 30 nm, of about 10 nm to about 40 nm, of about 15 nm to about 40 nm, of about 20 nm to about 40 nm, of about 10 nm to about 80 nm, of about 15 nm to about 80 nm, or of about 20 nm to about 80 nm.

8. The SNA of any one of claims 1-7, wherein the region is a regulatory site or a site at which a splicing factor interacts.

9. The SNA of any one of claims 4-8, wherein the liposomal core comprises a lipid bilayer and the antisense oligonucleotide is attached to the lipid bilayer.

10. The SNA of any one of claims 1-9, wherein the antisense oligonucleotide is eight to 100 linked nucleosides in length, eight linked nucleosides in length, nine linked nucleosides in length, 10 linked nucleosides in length, 11 linked nucleosides in length, 12 linked nucleosides in length, 13 linked nucleosides in length, 14 linked nucleosides in length, 15 linked nucleosides in length, 16 linked nucleosides in length, 17 linked nucleosides in length, 18 linked nucleosides in length, 19 linked nucleosides in length, 20 linked nucleosides in length, 21 linked nucleosides in length, 22 linked nucleosides in length, 23 linked nucleosides in length, 24 linked nucleosides in length, 25 linked nucleosides in length, 26 linked nucleosides in length, 27 linked nucleosides in length, 28 linked nucleosides in length, 29 linked nucleosides in length, 30 linked nucleosides in length, 31 linked nucleosides in length, 32 linked nucleosides in length, 33 linked nucleosides in length, 34 linked nucleosides in length, 35 linked nucleosides in length, 36 linked nucleosides in length, 37 linked nucleosides in length, 38 linked nucleosides in length, 39 linked nucleosides in length, 40 linked nucleosides in length, 41 linked nucleosides in length, 42 linked nucleosides in length, 43 linked nucleosides in length, 44 linked nucleosides in length, 45 linked nucleosides in length, 46 linked nucleosides in length, 47 linked nucleosides in length, 49 linked nucleosides in length, 50 linked nucleosides in length, 52 linked nucleosides in length, 54 linked nucleosides in length, 56 linked nucleosides in length, 58 linked nucleosides in length, 60 linked nucleosides in length, 62 linked nucleosides in length, 64 linked nucleosides in length, 66 linked nucleosides in length, 68 linked nucleosides in length, 70 linked nucleosides in length, 72 linked nucleosides in length, 74 linked nucleosides in length, 76 linked nucleosides in length, 78 linked nucleosides in length, 80 linked nucleosides in length, 82 linked nucleosides in length, 84 linked nucleosides in length, 86 linked nucleosides in length, 88 linked nucleosides in length, 90 linked nucleosides in length, 92 linked nucleosides in length, 94 linked nucleosides in length, 96 linked nucleosides in length, 100 linked nucleosides in length, or any range or combination thereof.

11. The SNA of any one of claims 1-10, wherein less than all of the internucleoside linkages in the antisense oligonucleotide are phosphodiester linkages.

12. The SNA of any one of claims 1-10, wherein all of the internucleoside linkages in the antisense oligonucleotide are phosphodiester linkages.

13. The SNA of any one of claims 1-11, wherein 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 99% of the internucleoside linkages in the antisense oligonucleotide are phosphodiester linkages.

14. The SNA of any one of claims 1-11 or 13, wherein the antisense oligonucleotide has phosphorothioate internucleoside linkages.

15. The SNA of claim 14, wherein less than all of the internucleoside linkages in the antisense oligonucleotide are phosphorothioate linkages.

16. The SNA of claim 14, wherein all of the internucleoside linkages in the antisense oligonucleotide are phosphorothioate linkages.

17. The SNA of any one of claims 14-16, wherein 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 99% of the internucleoside linkages in the antisense oligonucleotide are phosphorothioate linkages.

18. The SNA of any one of claims 1-17, wherein the antisense oligonucleotide has 2′O methyl or 2′ O methoxyethyl modifications.

19. The SNA of claim 18, wherein less than all of the nucleotides in the antisense oligonucleotide include a 2′O methyl or 2′ O methoxyethyl modification.

20. The SNA of claim 18, wherein all of the nucleotides in the antisense oligonucleotide include a 2′O methyl or 2′ O methoxyethyl modification.

21. The SNA of claim 18 or 19, wherein 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 99% of the nucleotides in the antisense oligonucleotide include a 2′O methyl modification.

22. The SNA of any one of claims 1-21, wherein the antisense oligonucleotide is comprised of 18 to 21 linked nucleosides in length.

23. The SNA of any one of claims 4-22, wherein the antisense oligonucleotides of the oligonucleotide shell are directly attached to the lipid bilayer of the liposomal core.

24. The SNA of any one of claims 4-22, wherein the antisense oligonucleotides of the oligonucleotide shell are indirectly attached to the lipid bilayer of the liposomal core through a linker moiety.

25. The SNA of claim 24, wherein the linker moiety comprises a molecular species at the 3′ or 5′ terminus of the antisense oligonucleotide, wherein the molecular species is positioned in the liposomal core and the antisense oligonucleotide extends radially from the liposomal core.

26. The SNA of claim 25, wherein the molecular species is at the 5′ terminus of the antisense oligonucleotide.

27. The SNA of any one of claims 24-26, wherein the molecular species is attached to the linker moiety.

28. The SNA of any one of claims 13-27, wherein the molecular species is a hydrophobic group.

29. The SNA of claim 28, wherein the hydrophobic group is selected from the group consisting of cholesterol, a cholesteryl or modified cholesteryl residue, tocopherol, adamantine, dihydrotesterone, long chain alkyl, long chain alkenyl, long chain alkynyl, olely-lithocholic, cholenic, oleoyl-cholenic, decane, dodecane, docosahexaenoyl, palmityl, C6-palmityl, heptadecyl, myrisityl, arachidyl, stearyl, behenyl, linoleyl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen.

30. The SNA of claim 28, wherein the hydrophobic group is cholesterol.

31. The SNA of any one of claims 24-30, wherein the linker moiety comprises a non-nucleotidic linker moiety attached to the molecular species.

32. The SNA of claim 31, wherein the non-nucleotidic linker moiety is selected from the group consisting of an abasic residue (dSpacer), oligoethyleneglycol, triethyleneglycol, hexaethylenegylcol, alkane-diol, or butanediol.

33. The SNA of claim 31, wherein the non-nucleotidic linker moiety is a double linker.

34. The SNA of claim 33, wherein the double linker is two oligoethyleneglycols.

35. The SNA of claim 34, wherein the two oligoethyleneglycols are triethyleneglycol.

36. The SNA of claim 34, wherein the two oligoethyleneglycols are hexaethylenegylcol.

37. The SNA of claim 33, wherein the double linker is two alkane-diols.

38. The SNA of claim 37, wherein the two alkane-diols are butanediol.

39. The SNA of any one of claims 33-38, wherein the double linker is linked in the center by a phosphodiester, phosphorothioate, methylphosphonate, or amide linkage.

40. The SNA of claim 31, wherein the non-nucleotidic linker moiety is a triple linker.

41. The SNA of claim 40, wherein the triple linker is three oligoethyleneglycols.

42. The SNA of claim 41, wherein the three oligoethyleneglycols are triethyleneglycol.

43. The SNA of claim 41, wherein the three oligoethyleneglycols are hexaethylenegylcol.

44. The SNA of claim 40, wherein the triple linker is three alkane-diols.

45. The SNA of claim 44, wherein the three alkane-diols are butanediol.

46. The SNA of any one of claims 40-45, wherein the triple linker is linked in between each single linker by a phosphodiester, phosphorothioate, methylphosphonate, or amide linkage.

47. The SNA of any one of claims 1-46, wherein the antisense oligonucleotides comprise the entire SNA such that no other structural components are part of the SNA and wherein the antisense oligonucleotide includes a molecular species and non-nucleotidic linker moiety that form the core, with the oligonucleotides extending radially from the core.

48. The SNA of any one of claims 1-47, wherein the SNA is free of lipids, polymers or solid cores.

49. A spherical nucleic acid (SNA), comprising a core and a first antisense oligonucleotide comprised of 8 to 50 linked nucleosides in length targeted to a first region in a pre-mRNA of interest and a second antisense oligonucleotide comprised of 8 to 50 linked nucleosides in length targeted to second region in a pre-mRNA of interest to regulate pre-mRNA splicing, and wherein the antisense oligonucleotides are attached to the core and form an oligonucleotide shell.

50. The SNA of claim 49, wherein the first region in the pre-mRNA of interest is a regulatory site.

51. The SNA of claim 49 or 50, wherein the second region in the pre-mRNA of interest is a long non-coding RNA (lncRNA).

52. The SNA of any one of claims 1-51, wherein the oligonucleotide shell has a surface density of 5-1,000 oligonucleotides per SNA.

53. The SNA of any one of claims 1-51, wherein the oligonucleotide shell has a surface density of 100-1,000 oligonucleotides per SNA.

54. The SNA of any one of claims 1-51, wherein the oligonucleotide shell has a surface density of 500-1,000 oligonucleotides per SNA.

55. The SNA of any one of claims 1-51, wherein the oligonucleotide shell has a surface density of at least 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 oligonucleotides per SNA.

56. The SNA of any one of claims 1-51, wherein the oligonucleotide shell has a surface density of about 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 oligonucleotides per SNA.

57. The SNA of any one of claims 1-56, wherein the lipid bilayer comprises one or more lipids selected from the group consisting of: sphingolipids, ceramides, phospholipids, and sterols of different lengths, saturation states, and derivatives thereof.

58. The SNA of any one of claims 1-56, wherein the lipid bilayer comprises one or more lipids selected from the group consisting of: sphingosine, sphingosine phosphate, methylated sphingosine, methylated sphinganine, ceramide phosphate, 1-0 acyl ceramide, dihydroceramide, 2-hydroxy ceramide, sphingomyelin, glycosylated sphingolipid, sulfatide, ganglioside, phosphosphingolipid, phytosphingosine, phosphatidylcholine, lysophosphatidylcholine, phosphatidic acid, lysophosphatidic acid, cyclic LPA, phosphatidylethanolamine, lysophosphatidylethanolamine, phosphatidylglycerol, lysophosphatidylglycerol, phosphatidylserine, lysophosphatidylserine, phosphatidylinositol, inositol phosphate, LPI, cardiolipin, lysocardiolipin, bis(monoacylglycero) phosphate, (diacylglycero) phosphate, ether lipid, diphytanyl ether lipid, plasmalogen, cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipid, ether cationic lipid, lanthanide chelating lipid, A-ring substituted oxysterol, B-ring substituted oxysterol, D-ring substituted oxysterol, side-chain substituted oxysterol, double substituted oxysterol, cholestanoic acid derivative, fluorinated sterol, fluorescent sterol, sulfonated sterol, phosphorylated sterol, and polyunsaturated sterol of different lengths, saturation states, and derivatives thereof.

59. The SNA of any one of claims 1-56, wherein the lipid bilayer is comprised of 1,2-dioleoyl-sn-glycero-3-phosphocholine (DOPC).

60. A spherical nucleic acid (SNA), comprising a core and a first antisense oligonucleotide comprised of 8 to 50 linked nucleosides in length targeted to a regulatory site and a second antisense oligonucleotide comprised of 8 to 50 linked nucleosides in length targeted to a region of a lncRNA, and wherein the antisense oligonucleotides are attached to the core and form an oligonucleotide shell.

61. A spherical nucleic acid (SNA) comprising a core and antisense oligonucleotides arranged in an oligonucleotide shell, wherein the oligonucleotides comprise a nucleotide backbone comprising a modification in one or more of the carbons in the five-carbon sugar, and wherein five nucleotides or fewer than five nucleotides do not comprise a modification in the five-carbon sugar.

62. The SNA of claim 61, wherein four nucleotides or fewer than four nucleotides do not comprise a modification in the five-carbon sugar.

63. The SNA of claim 61, wherein three nucleotides or fewer than three nucleotides do not comprise a modification in the five-carbon sugar.

64. The SNA of claim 61, wherein two nucleotides or fewer than two nucleotides do not comprise a modification in the five-carbon sugar.

65. The SNA of claim 61, wherein one nucleotide does not comprise a modification in the five-carbon sugar.

66. The SNA of claim 61, wherein all of the nucleotides in the nucleotide backbone of the antisense oligonucleotides comprise a modification in one or more of the carbons in the five-carbon sugar.

67. The SNA of any one of claims 61-66, wherein the modification is at the 2′-carbon of the five-carbon sugar.

68. The SNA of any one of claims 61-67, wherein the modification is a 2′-O-methylated nucleotide.

69. The SNA of any one of claims 61-68, wherein the antisense oligonucleotide comprises the nucleic acid sequence CCCACAGGGGCATGUAGLU (SEQ ID NO: 58).

70. The SNA of any one of claims 61-69, wherein the antisense oligonucleotide comprises or consists of the nucleic acid sequence mCmCmCmAmCmAmGmG*mG*mG*mC*mA*mT*mGmUmAmGmU (SEQ ID NO: 59), wherein * is a phosphorothioate linkage and m is a 2′-O-methylated nucleotide.

71. The SNA of any one of claims 61-70, wherein the antisense oligonucleotide comprises the nucleic acid sequence mCmCmCmAmCmAmGmG*mG*mG*mC*mA*mT*mGmUmAmGmU/Spacer18/Spacer18/3 CholTEG (SEQ ID NO: 211), wherein * is a phosphorothioate linkage, m is a 2′-O-methylated nucleotide, Spacer18 is a hexa(ethylene glycol) spacer, and 3CholTEG is tri(ethylene glycol) bound to a cholesterol.

72. A spherical nucleic acid (SNA), comprising an antisense oligonucleotide comprised of 8 to 50 linked nucleosides in length targeted to a regulatory site of a pre-mRNA of interest and a linker moiety comprising a molecular species at the 3′-end or the 5′-end of the antisense oligonucleotide, wherein the molecular species is a hydrophobic group comprising a stearyl.

73. The SNA of claim 72, wherein the stearyl is a distearyl.

74. A spherical nucleic acid (SNA) for regulating pre-mRNA splicing, comprising
a core and an antisense oligonucleotide comprised of 8 to 50 linked nucleosides in length targeted to a regulator of splicing of a pre-mRNA of interest to regulate pre-mRNA splicing, and wherein the antisense oligonucleotide is attached to the core and forms an oligonucleotide shell.

75. The SNA of claim 74, wherein the regulator regulates the inclusion of exons and/or introns in a mRNA of interest.

76. The SNA of claim 74, wherein the regulator is an RNA binding protein, a splicing factor or a ribonucleoprotein.

77. A composition comprising:
a SNA in a pharmaceutically acceptable carrier, wherein the SNA is a SNA of any one of claims 1-76.

78. A composition comprising:
a first spherical nucleic acid (SNA) comprising a core and a first antisense oligonucleotide comprised of 10 to 40 linked nucleosides in length targeted to a first region in a pre-mRNA of interest, and a second SNA comprising a core and a second antisense oligonucleotide comprised of 10 to 40 linked nucleosides in length targeted to a second region in the pre-mRNA of interest.

79. A method for treating a subject having a disease or disorder related to an abnormality in splice modulation, comprising
administering to a subject having the disease or disorder related to an abnormality in splice modulation a spherical nucleic acid (SNA) in an effective amount to increase expression levels of a protein of interest over a baseline level in the subject in order to treat the disease or disorder related to an abnormality in splice modulation.

80. The method of claim 79, wherein the disease or disorder related to an abnormality in splice modulation is Stargardt Disease (Juvenile Macular Degeneration), Usher Syndrome, X-Linked Retinoschisis, macular corneal dystrophy, Congenital stromal corneal dystrophy, Congenital hereditary endothelial corneal dystrophy, Fleck corneal dystrophy, lattice corneal dystrophy type I, lattice corneal dystrophy type 11, granular corneal dystrophy type 1, granular corneal dystrophy type II (Avellino), Epithelial recurrent erosion dystrophy, Stocker-Holt corneal dystrophy, Duchennes Muscular Dystrophy, Leber Congenital Amaurosis, B-Thalassemia, Meesmann Endothelial Corneal Dystrophy, Menkes Disease, Nijmegen Breakage Syndrome, Hutchison-Gilford Progeria Syndrome, Gelatinous droplike corneal dystrophy, Reis-Buckler corneal dystrophy, Schnyder crystalline corneal dystrophy, Subepithelial mucinous corneal dystrophy, Lisch corneal dystrophy, Posterior amorphous corneal dystrophy, X-linked endothelial corneal dystrophy, Thiel-Behnke corneal dystrophy, Posterior polymorphous corneal dystrophy, Pompe, Familial Hypertrophic Cardiomyopathy, X-Linked Aggamaglobulinemia, Oculopharyngeal Muscular Dystrophy, Dilated Cardiomyopathy, Frontotemporal Dementia, epithelial basement membrane corneal dystrophy, Alzheimer's Disease, Familial Hypercholesterolemia, a pro-inflammatory disease, Huntington's disease, Fukuyama congenital muscular dystrophy, Myotonic Dystrophy Type I or II, Cancer, Neovascularization, Ataxia telangiectasia, Congenital disorder of glycosylation, FTD/Parkinsonism linked to chr17, Niemann Pick disease type C, Neurofibromatosis type 1, Neurofibromatosis type 2, Megalencephalic leukoencephalopathy with subcortical cysts type 1, Pelizaeus-Merzbacher disease, Spinocerebellar Ataxia Type 7, Spinocerebellar Ataxia Type 17, Huntington's disease, Spinocerebellar Ataxia Type 1, Spinocerebellar Ataxia Type 12, Spinal and bulbar muscular atrophy, Spinocerebellar Ataxia Type 2, Spinocerebellar Ataxia Type 6, Dentatorubral-pallidoluysian atrophy, Spinocerebellar Ataxia Type 3, Spinocerebellar Ataxia Type 8, Huntington disease-like-2, Myotonic Dystrophy Type I, Fuchs Endothelial Corneal Dystrophy, Fragile X syndrome, fragile X-associated tremor/ataxia syndrome, Fragile XE syndrome, Friedreich ataxia, Myotonic Dystrophy Type II, Spinocerebellar Ataxia Type 10, Spinocerebellar Ataxia Type 31, Spinocerebellar Ataxia Type 36, C9orf72-ALS/FTD, or Prader-Willi syndrome.

81. The method of claim 79, wherein the disease or disorder related to an abnormality in splice modulation is Duchennes Muscular Dystrophy, Leber Congenital Amaurosis, B-Thalassemia, a pro-inflammatory disease, Huntington's disease, Spinocerebellar Ataxia Type 7, Spinocerebellar Ataxia Type 17, Huntington's disease, Spinocerebellar Ataxia Type 1, Huntington disease-like-2, or Prader-Willi syndrome.

82. The method of any one of claims 79-81, wherein the baseline level is the level of the protein of interest in the subject prior to treatment with the SNA.

83. The method of claim 82, wherein the baseline level is the level of the protein of interest in a subject having the disease or disorder related to an abnormality in splice modulation and treated with a linear antisense oligonucleotide targeted to a region in a pre-mRNA of interest to regulate pre-mRNA splicing.

84. The method of any one of claims 79-83, wherein the SNA is delivered by a route of administration selected from the group consisting of intrathecal, oral, nasal, sublingual, intravenous, subcutaneous, mucosal, respiratory, direct injection, and dermal route of administration.

85. The method of any one of claims 79-84, wherein the SNA is a SNA of any one of claims 1-76.

86. A method for treating a subject having a disease or disorder related to an abnormality in splice modulation, comprising
administering to a subject having a disease or disorder related to an abnormality in splice modulation at least two doses of a spherical nucleic acid (SNA), in an effective amount to increase expression levels of a protein of interest or corrected mRNA over a baseline level in the subject in order to treat the disease or disorder related to an abnormality in splice modulation, wherein the second dose is administered about 3 months to 2 years after the first dose, and wherein the SNA comprises a core and an antisense oligonucleotide comprised of 8 to 50 linked nucleosides in length targeted to a region in a pre-mRNA of interest, such that a level of a protein of interest or a level of a corrected mRNA relative to a defective mRNA associated with the disease or disorder related to an abnormality in splice modulation in the subject is enhanced,
wherein the oligonucleotides are attached to the core and thus form an oligonucleotide shell, and
wherein the corrected mRNA produces a functional protein of interest to treat the subject having the disease or disorder related to an abnormality in splice modulation.

87. The SNA of claim 86, wherein the region is a regulatory region.

88. The method of claim 86, wherein the disease or disorder related to an abnormality in splice modulation is Stargardt Disease (Juvenile Macular Degeneration), Usher Syndrome, X-Linked Retinoschisis, macular corneal dystrophy, Congenital stromal corneal dystrophy, Congenital hereditary endothelial corneal dystrophy, Fleck corneal dystrophy, lattice corneal dystrophy type 1, lattice corneal dystrophy type II, granular corneal dystrophy type I, granular corneal dystrophy type II (Avellino), Epithelial recurrent erosion dystrophy, Stocker-Holt corneal dystrophy, Duchennes Muscular Dystrophy, Leber Congenital Amaurosis, B-Thalassemia, Meesmann Endothelial Corneal Dystrophy, Menkes Disease, Nijmegen Breakage Syndrome, Hutchison-Gilford Progeria Syndrome, Gelatinous droplike corneal dystrophy, Reis-Buckler corneal dystrophy, Schnyder crystalline corneal dystrophy, Subepithelial mucinous corneal dystrophy, Lisch corneal dystrophy, Posterior amorphous corneal dystrophy, X-linked endothelial corneal dystrophy, Thiel-Behnke corneal dystrophy, Posterior polymorphous corneal dystrophy, Pompe, Familial Hypertrophic Cardiomyopathy, X-Linked Aggamaglobulinemia, Oculopharyngeal Muscular Dystrophy, Dilated Cardiomyopathy, Frontotemporal Dementia, epithelial basement membrane corneal dystrophy, Alzheimer's Disease, Familial Hypercholesterolemia, a pro-inflammatory disease, Huntington's disease, Fukuyama congenital muscular dystrophy, Myotonic Dystrophy Type I or II, Cancer, Neovascularization, Ataxia telangiectasia, Congenital disorder of glycosylation, FTD/Parkinsonism linked to chr17, Niemann Pick disease type C, Neurofibromatosis type 1, Neurofibromatosis type 2, Megalencephalic leukoencephalopathy with subcortical cysts type 1, Pelizaeus-Merzbacher disease, Spinocerebellar Ataxia Type 7, Spinocerebellar Ataxia Type 17, Huntington's disease, Spinocerebellar Ataxia Type 1, Spinocerebellar Ataxia Type 12, Spinal and bulbar muscular atrophy, Spinocerebellar Ataxia Type 2, Spinocerebellar Ataxia Type 6, Dentatorubral-pallidoluysian atrophy, Spinocerebellar Ataxia Type 3, Spinocerebellar Ataxia Type 8, Huntington disease-like-2, Myotonic Dystrophy Type I, Fuchs Endothelial Corneal Dystrophy, Fragile X syndrome, fragile X-associated tremor/ataxia syndrome, Fragile XE syndrome, Friedreich ataxia, Myotonic Dystrophy Type II, Spinocerebellar Ataxia Type 10, Spinocerebellar Ataxia Type 31, Spinocerebellar Ataxia Type 36, C9orf72-ALS/FTD, or Prader-Willi syndrome.

89. The method of claim 86, wherein the disease or disorder related to an abnormality in splice modulation is Duchennes Muscular Dystrophy, Leber Congenital Amaurosis, B-Thalassemia, a pro-inflammatory disease, Huntington's disease, Spinocerebellar Ataxia Type 7, Spinocerebellar Ataxia Type 17, Huntington's disease, Spinocerebellar Ataxia Type 1, Huntington disease-like-2, or Prader-Willi syndrome.

90. A method of enhancing a level of a corrected mRNA relative to a defective mRNA associated with an abnormality in splice modulation in a cell, comprising contacting the cell with an spherical nucleic acid (SNA) comprising a core and an antisense oligonucleotide comprised of 10 to 40 linked nucleosides in length targeted to a region in a pre-mRNA of interest, such that the level of a corrected mRNA relative to a defective mRNA in the cell is enhanced.

91. The method of claim 90, wherein the SNA is a SNA of any one of claims 1-76.

92. A method for treating a subject having a disease or disorder related to an abnormality in splice modulation, comprising
administering to a subject having the disease or disorder related to an abnormality in splice modulation a spherical nucleic acid (SNA) in an effective amount to decrease expression levels of a protein of interest under a baseline level in the subject in order to treat the disease or disorder related to an abnormality in splice modulation.

93. The method of claim 92, wherein the SNA is a SNA of any one of claims 1-76.

94. The method of claim 92 or 93, wherein the antisense oligonucleotide has locked nucleic acid (LNA) modifications.

95. The method of claim 94, wherein less than all of the nucleotides in the antisense oligonucleotide include a LNA modification.

96. The method of claim 94, wherein all of the nucleotides in the antisense oligonucleotide include a LNA modification.

97. The method of claim 92 or 93, wherein the antisense oligonucleotide has morpholino modifications.

98. The method of claim 103, wherein less than all of the nucleotides in the antisense oligonucleotide include a morpholino modification.

99. The method of claim 103, wherein all of the nucleotides in the antisense oligonucleotide include a morpholino modification.

100. A method of producing a splice variant susceptible to nonsense-mediated decay, the method comprising:
contacting a cell with a spherical nucleic acid (SNA) comprising oligonucleotides arranged in an oligonucleotide shell and a core in an affective amount to produce a splice variant susceptible to nonsense-mediated decay.

101. A method of treating a disease or disorder in a subject, the method comprising:
administering to a subject an effective amount of a spherical nucleic acid (SNA) comprising oligonucleotides arranged in an oligonucleotide shell and a core to produce a splice variant susceptible to nonsense-mediated decay in order to treat the disease or disorder in the subject.

102. The method of claim 101, wherein the SNA is administered to the subject by an administration route selected from the group consisting of intrathecal, oral, nasal, sublingual, intravenous, subcutaneous, mucosal, respiratory, direct injection, and dermal route of administration.

103. The method of any one of claims 100-102, wherein the SNA is a SNA of any one of claims 1-76.

104. The method of any one of claims 101-103, wherein the disease or disorder is cancer.

105. The method of claim 104, wherein the cancer is selected from the group consisting of melanoma, renal cancer, clear cell carcinoma, prostate cancer, hormone refractory prostate adenocarcinoma, breast cancer, colon cancer, lung cancer, non-small cell lung cancer, small cell lung cancer, bone cancer, pancreatic cancer, pancreatic adenocarcinoma, skin cancer, cancer of the head or neck, cutaneous or intraocular malignant melanoma, uterine cancer, ovarian cancer, rectal cancer, stomach cancer, testicular cancer, thyroid cancer, anaplastic thyroid cancer, uterine cancer, carcinoma of the fallopian tubes, carcinoma of the endometrium, carcinoma of the cervix, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, biliary tract cancer, brain cancer, breast cancer, cervical cancer, choriocarcinoma, esophageal cancer, gastric cancer, an intraepithelial neoplasm, lymphoma, liver cancer, neuroblastoma, oral cancer, sarcoma, hairy cell leukemia, chronic myelogenous leukemia, cutaneous T-cell leukemia, multiple myeloma, renal cell carcinoma, lymphoma, bladder cancer, glioblastoma multiforme, Merkel cell carcinoma, cutaneous squamous cell carcinoma, melanoma or squamous cell carcinoma of the head and neck,

106. The method of claim 104, wherein the cancer is selected from the group consisting of pleomorphic sarcoma, gastrointestinal stromal tumor (GIST), liposarcoma, leiomyosarcoma, synovial sarcoma, malignant peripheral nerve sheath tumor, rhabdomyosarcoma, angiosarcoma, fibrosarcoma, dermatofibrosarcoma protuberans, epithelioid sarcoma, myxoma, mesenchymoma, vascular sarcoma, neurilemmoma, bone sarcoma, osteosarcoma, Ewing's sarcoma, chondrosarcoma, Kaposi sarcoma, solitary fibrous tumor, chordoma, desmoid-type fibromatosis, fibroblastic sarcoma, giant cell tumor of the bone, gynaecological sarcoma, soft tissue sarcoma, angioleiomyoma, leiomyoma, smooth muscle sarcoma, fibrohistiocytic sarcoma, sebaceous cell carcinoma and eccrine carcinoma.

107. The method of any one of claims 101-103, wherein the disease or disorder is an inflammatory disease or disorder.

108. The method of claim 107, wherein the inflammatory disease or disorder is selected from the group consisting of an autoimmune disease, an infectious disease, transplant rejection or graft-versus-host disease, a pulmonary disorder, an intestinal disorder, a cardiac disorder, sepsis, a spondyloarthropathy, a metabolic disorder, a hepatic disorder, a skin disorder and a nail disorder.

109. The method of claim 107, wherein the inflammatory disease or disorder is selected from the group consisting of atopic dermatitis, epidermolysis bullosa, uveitis, gout, polymyalgia rheumatica, osteoarthritis, systemic-onset juvenile idiopathic arthritis, schnitzler syndrome, familial mediterranean fever, cryopyrin-associated periodic syndrome (CAPS), hyper-igd syndrome (HIDS), TNF receptor-associated periodic syndrome (TRAPs), type 2 diabetes, proliferative diabetic retinopathy, wet age-related macular degeneration, chronic obstructive pulmonary disease, type I diabetes, pyoderma gangrenosum, dry eye syndrome, and acne vulgaris, rheumatoid arthritis, psoriasis, psoriatic arthritis, psoriasis in combination with psoriatic arthritis, ulcerative colitis, Crohn's disease, vasculitis, Behcet's disease, ankylosing spondylitis, asthma, chronic obstructive pulmonary disorder (COPD), idiopathic pulmonary fibrosis (IPF), restenosis, anemia, pain and hepatitis C virus infection.

110. The method of claim 108, wherein the autoimmune disease is selected from the group consisting of rheumatoid arthritis, rheumatoid spondylitis, osteoarthritis, gouty arthritis, allergy, multiple sclerosis, autoimmune uveitis, and nephritic syndrome.

111. A method of increasing the levels of a soluble variant of a transmembrane receptor in a cell, the method comprising:
contacting a cell with an effective amount of a spherical nucleic acid (SNA) that modulates splicing of the pre-mRNA of a transmembrane receptor to produce a soluble variant of the transmembrane receptor such that the level of soluble variant of the transmembrane receptor is increased relative to a cell that has not been contacted with the SNA or relative to a cell contacted with the corresponding linear oligonucleotide not in a SNA, wherein the levels of the mRNA encoding the transmembrane receptor are not decreased through RNAse-H mediated degradation.

112. The method of claim 111, wherein the transmembrane receptor is an ion channel linked receptor, and enzyme-linked receptor, or a G protein-coupled receptor.

113. The method of claim 111, wherein the transmembrane receptor is an adrenergic receptor, an olfactory receptor, a receptor tyrosine kinase, an epidermal growth factor receptor, an insulin receptor, a fibroblast growth factor receptor, a neurotrophin receptor, an ephrin receptor, an integrin, a low affinity nerve growth factor receptor, a N-methyl-D-aspartate (NMDA) receptor, or an immune receptor.

114. The method of claim 111, wherein the transmembrane receptor is a toll-like receptor, a T-cell receptor, a cluster of differentiation 28 (CD28), or a csk-interacting membrane (SCIMP) protein.

115. The method of claim 113, wherein the immune receptor is a pattern recognition receptor, a killer activated receptor, a killer inhibitor receptor, a complement receptor, an Fc receptor, a B cell receptor, a T cell receptor, or a cytokine receptor.

116. The method of any one of claims 111-115, wherein the SNA is a SNA of any one of claims 1-76.

117. The method of any one of claims 111-116, wherein the SNA is in a solution at a concentration of between about 100 nM to 1 μM.

118. The method of claim 111-116, wherein the SNA is in a solution at a concentration of or about 0.5 μM, of or about 1 μM, or of or about 5 μM.

119. The method of any one of claims 111-118, wherein the cell is brain cell, liver cell, lung cell, gut cell, stomach cell, intestine cell, fat cell, muscle cell, uterine cell, skin cell, spleen cell, endocrine organ cell, or bone cell.

120. The method of any one of claims 111-119, wherein the SNA is in a pharmaceutically acceptable carrier that is a gel formulation.

121. The method of any one of claims 111-120, wherein the cell is contacted with the SNA in vitro.

122. The method of any one of claims 111-120, wherein the cell is contacted with the SNA in vivo.

123. The method of any one of claims 111-120, wherein the cell is contacted with the SNA ex vivo.

124. A method of treating a disease or disorder in a subject, the method comprising administering to a subject with a disease or disorder associated with abnormal transmembrane receptor activity or abnormal transmembrane receptor expression in a cell of the subject an effective amount of a spherical nucleic acid (SNA) to produce or increase the levels of a soluble variant of the transmembrane receptor in the subject relative to a subject with a disease or disorder associated with abnormal transmembrane receptor activity or abnormal transmembrane receptor expression who has not been administered a SNA or relative to a subject with a disease or disorder associated with abnormal transmembrane receptor activity or abnormal transmembrane receptor expression who has been administered a corresponding linear oligonucleotide that is not in a SNA, in order to treat the disease or disorder in the subject.

125. The method of claim 124, wherein the total levels of the transmembrane receptor in the cell of the subject remain stable or wherein the total levels of the mRNA encoding the transmembrane receptor are not decreased through RNAse-H mediated degradation.

126. The method of claim 124 or 125, wherein the SNA is a SNA of any one of claims 1-76.

127. The method of any one of claims 124-126, wherein the disease or disorder is a topic dermatitis or psoriasis

128. The method of any one of claims 124-126, wherein the transmembrane receptor is interleukin 17 receptor α (IL17RA) or interleukin 1 receptor accessory protein (IL1RAP).

129. The method of any one of claims 124-128, wherein the SNA is administered to the subject by an administration route selected from the group consisting of intrathecal, oral, nasal, sublingual, intravenous, subcutaneous, mucosal, respiratory, direct injection, and dermal route of administration.

130. A method of increasing the levels of a mRNA of interest in a cell, the method comprising contacting the cell with a SNA of any one of claims 1-76, wherein the levels of the mRNA of interest in the cell is increased relative to a cell that has not been contacted with the SNA or relative to a cell contacted with the corresponding linear oligonucleotide not in a SNA.

131. A method of inducing exon skipping in a pre-mRNA of interest in a cell, the method comprising contacting a cell with a SNA of any one of claims 1-76 to induce exon skipping in a pre-mRNA of interest in the cell.

132. A method of inducing exon inclusion in a pre-mRNA of interest in a cell, the method comprising contacting a cell with a SNA of any one of claims 1-76 to induce exon inclusion in a pre-mRNA of interest in the cell.

133. A method for delivering a stable level of antisense oligonucleotides to a central nervous system (CNS) of a subject having a CNS disease or disorder, the method comprising:
administering to a subject having a neurodegenerative disease or disorder a spherical nucleic acid (SNA) in an effective amount to deliver antisense oligonucleotides to the CNS of the subject,
wherein the administration of SNA delivers about 2% to about 150% more antisense oligonucleotides to one or more tissues or regions of the CNS of the subject than administration of linear antisense oligonucleotides that are not in a SNA,
wherein the SNA comprises a core and antisense oligonucleotides comprised of 10 to 60 linked nucleosides in length, wherein the antisense oligonucleotides are attached to the core and thus form an oligonucleotide shell,
wherein the CNS disease or disorder is not autism, Alzheimer's disease, Parkinson's disease, spinal muscular atrophy, or characterized by muscle wasting and loss of muscle function.

134. The method of claim 133, wherein the CNS disease or disorder is encephalitis, poliomyelitis, essential tremor, multiple sclerosis, cancer of the nervous system, addiction, attention deficit/hyperactivity disorder (ADHD), bipolar disorder, catalepsy, depression, epilepsy/seizures, infection, locked-in syndrome, meningitis, migraine, myelopathy or Tourette's syndrome.

135. The method of claim 133 or 134, wherein the SNA is administered intrathecally (IT).

136. The method of claim 133 or 134, wherein the SNA is administered in the lower lumbar region.

137. The method of claim 133 or 134, wherein the SNA is IT-administered through a lumbar puncture.

138. The method of any one of claims 133-137, wherein the subject is a mammal.

139. The method of any one of claims 133-137, wherein the subject is a rat or mouse.

140. The method of any one of claims 133-137, wherein the subject is a human.

141. The method of any one of claims 133-140, wherein a stable level is achieved when at least 50% of the antisense oligonucleotides are present in a tissue of the CNS within three days of administration of the SNA to the subject, relative to the amount of antisense oligonucleotides present in the tissue of the CNS within one hour of administration of the SNA to the subject.

142. The method of any one of claims 133-140, wherein a stable level is achieved when at least 50% of the antisense oligonucleotides are present in a tissue of the CNS within 48 hours of administration of the SNA to the subject, relative to the amount of antisense oligonucleotides present in the tissue of the CNS within one hour of administration of the SNA to the subject.

143. The method of any one of claims 133-140, wherein a stable level is achieved when at least 50% of the antisense oligonucleotides are present in a tissue of the CNS within 24 hours of administration of the SNA to the subject, relative to the amount of antisense oligonucleotides present in the tissue of the CNS within one hour of administration of the SNA to the subject.

144. The method of any one of claims 133-143, wherein less than 50% of the antisense oligonucleotides are detectable within six hours of administration to the subject in one or both kidneys of the subject.

145. The method of any one of claims 133-143, wherein less than 40% of the antisense oligonucleotides are detectable within six hours of administration to the subject in one or both kidneys of the subject.

146. The method of any one of claims 133-143, wherein less than 30% of the antisense oligonucleotides are detectable within six hours of administration to the subject in one or both kidneys of the subject.

147. The method of any one of claims 133-143, wherein less than 20% of the antisense oligonucleotides are detectable within six hours of administration to the subject in one or both kidneys of the subject.

148. The method of any one of claims 133-143, wherein less than 10% of the antisense oligonucleotides are detectable within six hours of administration to the subject in one or both kidneys of the subject.

149. The method of any one of claims 133-143, wherein less than 5% of the antisense oligonucleotides are detectable within six hours of administration to the subject in one or both kidneys of the subject.

150. The method of any one of claims 133-143, using the SNA of any one of claims 1-76.

151. The method of any one of claims 133-143, wherein the SNA is in a formulation and wherein the formulation comprises artificial cerebral spinal fluid (aCSF).

152. The method of any one of claims 133-151, wherein the one or more tissues or regions of the CNS is one or more regions of the brain.

153. The method of claim 152, wherein the one or more regions of the brain is selected from the group consisting of the amygdala, basal ganglia, cerebellum, corpus callosum, cortex, hippocampus, hypothalamus, midbrain, olfactory region, one or more ventricles, septal area, white matter and thalamus.

154. The method of any one of claims 133-151, wherein the one or more tissues or regions of the CNS are the cervical cerebral spinal fluid (CSF) or thoracic CSF.

155. The method of any one of claims 133-154, wherein the antisense oligonucleotides in the SNA have different routes of distribution and clearance from the corresponding linear antisense oligonucleotides that are not in a SNA.

156. A method of increasing expression of a mRNA of interest in a cell, the method comprising contacting the cell with a first spherical nucleic acid (SNA) comprising a core and a first antisense oligonucleotide comprised of 10 to 40 linked nucleosides in length targeted to a first region in a pre-mRNA of interest, and contacting the cell with a second SNA comprising a core and a second antisense oligonucleotide comprised of 10 to 40 linked nucleosides in length targeted to a second region in the pre-mRNA of interest, wherein the first antisense oligonucleotide in the first SNA and the second antisense oligonucleotide in the second SNA modulate splicing of the pre-mRNA of interest to increase the levels of the mRNA of interest in the cell relative to a cell that has not been contacted with the SNA or relative to a cell contacted with the corresponding linear oligonucleotide not in a SNA.

157. The method of claim 156, wherein the first antisense oligonucleotide in the first SNA and the second antisense oligonucleotide in the second SNA work synergistically.

158. The method of claim 156 or 157, wherein the first SNA or the second SNA is a SNA of any one of claims 1-76.

159. The method of claim 156 or 157, wherein the first SNA and the second SNA is a SNA of any one of claims 1-76.

160. A method of increasing the levels of a mRNA of interest in a cell, the method comprising contacting the cell with a spherical nucleic acid (SNA) comprising a core and a first antisense oligonucleotide comprised of 10 to 40 linked nucleosides in length targeted to a first region in a pre-mRNA of interest and a second antisense oligonucleotide comprised of 10 to 40 linked nucleosides in length targeted to a second region in the pre-mRNA of interest, wherein the first antisense oligonucleotide and the second antisense oligonucleotide modulate splicing of the pre-mRNA of interest to increase the levels of the mRNA of interest in the cell, relative to a cell that has not been contacted with the SNA or relative to a cell contacted with the corresponding linear oligonucleotide not in a SNA.

161. The method of claim 160, wherein the first antisense oligonucleotide and the second antisense oligonucleotide work synergistically.

162. The method of claim 160 or 161, wherein the SNA is a SNA of any one of claims 1-76.

163. A method for delivering a stable level of antisense oligonucleotides to a central nervous system (CNS) of a subject having a CNS disease or disorder, the method comprising:
administering to a subject having a neurodegenerative disease or disorder a spherical nucleic acid (SNA) in an effective amount to deliver a first antisense oligonucleotide and a second antisense oligonucleotide to the CNS of the subject,
wherein the administration of SNA delivers about 2% to about 150% more antisense oligonucleotides to one or more tissues or regions of the CNS of the subject than administration of linear antisense oligonucleotides that are not in a SNA,
wherein the SNA comprises a core and antisense oligonucleotides comprised of 10 to 60 linked nucleosides in length, wherein the antisense oligonucleotides are attached to the core and thus form an oligonucleotide shell.

164. The method of claim 163, wherein the CNS disease or disorder is SMA.

165. A method for delivering a stable level of antisense oligonucleotides to a central nervous system (CNS) of a subject having a CNS disease or disorder, the method comprising:
administering to a subject having a neurodegenerative disease or disorder a first spherical nucleic acid (SNA) in an effective amount to deliver a first antisense oligonucleotide and a second SNA to deliver a second antisense oligonucleotide to the CNS of the subject,
wherein the administration of SNA delivers about 2% to about 150% more antisense oligonucleotides to one or more tissues or regions of the CNS of the subject than administration of linear antisense oligonucleotides that are not in a SNA,
wherein the SNA comprises a core and antisense oligonucleotides comprised of 10 to 60 linked nucleosides in length, wherein the antisense oligonucleotides are attached to the core and thus form an oligonucleotide shell.

166. The method of claim 163, wherein the CNS disease or disorder is SMA.
